(19) United States
(12) Patent Application Publication
(10) Pub. No.: US 2006/0235222 A1
(43) Pub. Date: Oct. 19, 2006

(54) Indole-derivative modulators of steroid hormone nuclear receptors

(75) Inventors: Michael Gregory Bell, Indianapolis, IN (US); Konstantinos Gavardinas, Monrovia, IN (US); Douglas Linn Gernert, Indianapolis, IN (US); Timothy Alan Grese, Indianapolis, IN (US); Prabhakar Kondaji Jadhav, Zionsville, IN (US); Peter Ambrose Lander, Indianapolis, IN (US); Mitchell Irvin Steinberg, South Bend, IN (US)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY, Indianapolis, IN (US)

(21) Appl. No.: 10/542,621

(22) PCT Filed: Jan. 20, 2004
(86) PCT No.: PCT/US04/00017

§ 371 Date: Jul. 15, 2005

Related U.S. Application Data
(60) Provisional application No. 60/441,947, filed on Jan. 22, 2003.

(51) Int. Cl. A61K 31/506; C07D 413/02; C07D 417/02; C07D 409/02; C07D 405/02; C07D 403/02

(52) U.S. Cl. 544/238; 544/333; 544/405; 546/114; 546/176; 548/305.4; 548/217; 548/152; 548/207; 548/257; 548/241; 548/360.1; 548/454; 548/455

(57) ABSTRACT

The present invention provides a compound of formula I or a pharmaceutically acceptable salt thereof, pharmaceutical compositions comprising an effective amount of a compound of Formula I in combination with a suitable carrier, diluent, or excipient, and methods for treating physiological disorders, particularly congestive heart disease, comprising administering to a patient in thereof an effective amount of a compound of Formula I.

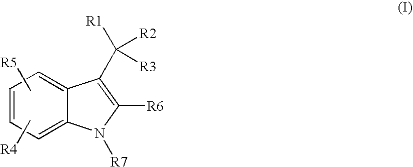

### BACKGROUND OF THE INVENTION

Nuclear hormone receptors are an evolutionarily conserved class of intracellular receptor proteins which have been termed “ligand dependent transcription factors”. Evans et al., SCIENCE, 240: 889 (1988). The nuclear hormone receptor gene superfamily encodes structurally-related receptor proteins for glucocorticoids (e.g. cortisol, corticosterone, cortisone), androgens, mineralocorticoids (e.g. aldosterone), progestins, estrogen, and thyroid hormone. Also included within this superfamily of nuclear receptors are receptor proteins for vitamin D, retinoic acid, 9-cis retinoic acid, as well as those receptors for which no cognate ligands have been identified (“orphan receptors”) Ribeiro et al., Annual Rev. Med., 46:443-453 (1995). Steroid hormone receptors represent a subset of the nuclear hormone receptor superfamily. So named according to the cognate ligand which complexes with the receptor in its native state, the steroid hormone nuclear receptors include the glucocorticoid receptor (GR), the androgen receptor (AR), the mineralocorticoid receptor (MR), the estrogen receptor (ER), and the progesterone receptor (PR). Tenbaum et al., Int. J. Biochem. Cell. Bio., 29(12):1325-1341(1997).

In contrast to membrane bound receptors, nuclear hormone receptors encounter their respective ligands following entry of the ligand into the cell. Once ligand binding occurs, the ligand-receptor complex modulates transcription of target genes within the cell nucleus. For example, most ligand-free nuclear receptors are bound in a complex with heat shock proteins (HSPs) in the cytoplasm. Following entry of circulating hormone into the cell, binding elicits a conformational change in the receptor, dissociating the receptor from the hsp. The ligand bound receptors translocate to the nucleus, where they as monomers as well as hetero- and homodimers in binding to particular hormone response elements (HREs) in the promoter regions of target genes. The HRE-receptor complex then, in turn, regulates transcription of proximally-located genes. (see Ribeiro et. al., supra.). On the other hand, thyroid hormone receptors (TRs) and other non-steroid receptors such as vitamin D receptor (VDR) and retinoic acid receptors (RAR) are bound to their respective HRE in the absence of HSPs and/or cognate ligand. Hormones released from the circulation enter the cell, binding in the nucleus to these receptors which, in turn, hetero-dimerize to other nuclear receptors such as 9-cis retinoic acid (RXR). As with the steroid hormone nuclear receptors, following ligand binding, the ligand-bound receptor complex again regulates transcription of neighboring genes.

Mineralocorticoids and glucocorticoids exert profound influences on a multitude of physiological functions by virtue of their diverse roles in growth, development, and maintenance of homeostasis. The actions are mediated by the MR and GR which share approximately 94% homology in their respective DNA binding regions, and approximately 57% homology in their respective ligand-binding domains. Kino et al., J. of Endocrinology, 169, 437-445 (2001). In visceral tissues, such as the kidney and the gut, MR regulates sodium retention, potassium excretion, and water balance in response to aldosterone. In addition, MR expression in the brain appears to play a role in the control of neuronal excitability, in the negative feedback regulation of the hypothalamic-pituitary-adrenal axis, and in the cognitive aspects of behavioral performance. Castren et al., J. of Neuroendocrinology, 3, 461-466 (1993). GR, which is ubiquitously expressed in almost all tissues and organ systems, is crucial for the integrity of central nervous system function and the maintenance of cardiovascular, metabolic, and immune homeostasis. Kino et al., J. of Endocrinology, 169, 437-445 (2001).

Elevations in aldosterone levels, or excess stimulation of mineralocorticoid receptors, are linked to several physiological disorders or pathologic disease states including, Conn's Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Bartter's Syndrome, and disorders associated with excess catecholamine levels. Hadley, M. E., ENDOCRINOLOGY, 2^{nd} Ed., pp. 366-381, (1988); and Brilla et al., Journal of Molecular and Cellular Cardiology, 25 (5), pp. 563-575 (1993). Additionally, elevated aldosterone levels have been increasingly implicated with congestive heart failure (CHF). In CHF, the failing heart triggers hormonal mechanisms in other organs in response to the attending reductions in blood flow and blood pressure seen with CHF. In particular, the kidney activates the renin-angiotensin-aldosterone system (RAAS) causing an increase in aldosterone production by the adrenals which, in turn, promotes water and sodium retention, potassium loss, and further edema. Although historically it was believed that aldosterone participated in the etiology of CHF only as a result of its salt retaining effects, several recent studies have implicated elevated aldosterone levels with events in extra-adrenal tissues and organs, such as myocardial and vascular fibrosis, direct vascular damage, and baroreceptor dysfunction. Pitt et al., New Eng. J. Med., 341:709-717 (1999). These findings are particularly significant since angiotensin converting enzyme (ACE) inhibitors, which were once thought to completely abolish aldosterone production, are now believed to only transiently suppress aldosterone production which has been shown to occur in extra-adrenal tissues including the heart and vasculature. Weber, New Eng. J. Med., 341:753-755 (1999); Fardella and Miller, Annu. Rev. Nutr., 16:443-470 (1996).

The involvement of aldosterone acting via MR in CHF was confirmed in the recently completed RALES (Randomized Aldactone Evaluation Study) study. Pitt et al., New Eng. J. Med., 341:709-717 (1999). The RALES study demonstrated that the use of Aldactone™ (spironolactone), a well-known competitive MR antagonist, in combination with standard CHF therapy, reduced cardiac related mortality by 30% and frequency of hospitalization by 35% in patients suffering from advanced CHF. However, spironolactone therapy has also been associated with attending side effects such as gastric bleeding, diarrhea, azotemia, hyperchloremic metabolic acidosis and type-4 renal tubule acidosis, nausea, gynecomastia, erectile dysfunction, hyperkalemia, and irregular menses. Thus, the mineralocorticoid receptor represents a viable target for CHF therapy either alone or in combination with conventional CHF therapies such as vasodilators (ACE inhibitors), inotropics (digoxin), diuretics, or beta blockers. Molecules, preferably non-steroids, which bind to the mineralocorticoid receptor and modulate receptor activity without the attending side effects of current therapies would be particularly desirable.

Finally, published international PCT application WO 02/17895 discloses that aldosterone antagonists are useful in the treatment of subjects suffereing from one or more cognitive dysfunctions including, but not limited to psychoses, cognitive disorders (such as memory disturbances), mood disorders (such as depression and bipolar disorder), anxiety disorders, and personality disorders.

Glucocorticoids (e.g. cortisol, corticosterone, and cortisone), and the glucocorticoid receptor, have also been implicated in the etiology of a variety of physiological disorders or pathologic disease states. For example, cortisol hyposecretion is implicated in the pathogenesis of Addison's Disease and may result in muscle weakness, increased melanin pigmentation of the skin, weight loss, hypotension, and hypoglycemia. On the other hand, excessive or prolonged secretion of glucocorticoids has been correlated to Cushing's Syndrome and may also result in obesity, hypertension, glucose intolerance, hyperglycemia, diabetes mellitus, osteoporosis, polyuria, and polydipsia. Hadley, M. E., ENDOCRINOLOGY, 2^{nd} Ed., pp. 366-381, (1988). Further, Coghlan et al., U.S. Pat. No. 6,166,013, issued Dec. 26, 2000, discloses that GR selective agents could modulate GR activity and, thus, be useful in the treatment of inflammation, tissue rejection, auto-immunity, malignancies such as leukemias and lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hyperglycemia, acute adrenal insufficiency, chronic primary adrenal insufficiency, secondary adrenal insufficiency, congenital adrenal hyperplasia, cerebral edema, thrombocytopenia, and Little's syndrome. Coghlan et al. also discloses that GR modulators are especially useful in disease states involving systemic inflammation such as inflammatory bowel disease, systemic lupus erythematosus, polyartitis nodosa, Wegener's granulomatosis, giant cell arthritis, rheumatoid arthritis, osteoarthritis, hay fever, allergic rhinitis, urticaria, angioneutotic edema, chronic obstructive pulmonary disease, asthma, tendonitis, bursitis, Crohn's disease, ulcerative colitis, autoimmune chronic active hepatitis, organ transplantation, hepatitis, and cirrhosis; and that GR modulating compounds have been used as immunostimulants, repressors, and as wound healing and tissue repair agents.

In addition, Coghlan et al. discloses that GR modulators have also found use in a variety of topical diseases such as inflammatory scalp alopecia, panniculitis, psoriasis, discoid lupus erythematosus, inflamed cysts, atopic dermatitis, pyoderma gangrenosum, pemphigus vulgaris, bullous pemphigoid, systemic lupus erythematosus, dermatomyositis, eosinophilic fasciitis, relapsing polychondritis, inflammatory vasculitis, sarcoidosis, Sweet's disease, type 1 reactive leprosy, capillary hemangiomas, contact dermatitis, atopic dermatitis, lichen planus, exfoliative dermatitis, erythema nodosum, acne, hirsutism, toxic epidermal necrolysis, erythema multiform, and cutaneous T-cell lymphoma.

Thus, it is clear that a ligand which has affinity for steroid hormone nuclear receptors, and particularly for MR and/or GR, could be used to modulate (i.e. repress, antagonize, agonize, partially antagonize, partially agonize) receptor activity and target gene expression, thereby influencing a multitude of physiological functions related to alterations in steroid hormone levels and/or steroid hormone receptor activity. In this regard, such ligands could be useful to treat a wide range of physiological disorders susceptible to steroid hormone nuclear receptor modulation.

Published literature references disclose indole derivative molecules useful in a broad range of indications from electroluminescent agents to marine anti-fouling agents. Further, indole-derivative compounds have also been disclosed as having pharmacological utility as, inter alia, serotonin 5HT-6 receptor modulators, anticoagulant agents, antiangiogenics, antiparasitics, integrin inhibitors, phospholipase inhibitors, endothelian receptor antagonists, antiarrhythmics, and dopamine antagonists. Surprisingly, however, and in accordance with the present invention, applicants have discovered a series of non-steroidal indole derivative compounds, particularly 3-substituted indole derivatives, with affinity for steroid hormone nuclear receptors, and particularly for MR and GR. Such compounds could modulate nuclear receptor activity and, therefore, have utility in treating physiological disorders related to alterations in steroid hormone level and/or to alterations in steroid hormone nuclear receptor activity. Furthermore, such compounds could address a long felt and continuing need for safe and effective pharmaceutical interventions without the attending side effects of steroidal-type agents. The treatment of steroid hormone related disorders is hereby furthered.

The following references describe examples of the state of the art as it relates to the present invention.

Published International PCT Application WO 96/19458 and U.S. Pat. Nos. 5,696,130; 5,994,544; 6,017,924, and 6,121,450 disclose quinoline derivative analogs as steroid hormone receptor modulators.

Published International PCT Application WO 00/06137 and U.S. Pat. No. 6,166,013 disclose triphenylmethane compounds as glucocorticoid receptor modulators.

U.S. Pat. No. 6,147,066 discloses anti-mineralocorticoid receptor compounds for use in treating drug withdrawal syndrome.

U.S. Pat. Nos. 6,008,210 and 6,093,708 disclose spirolactone compounds, such as spironolactone and epoxymexrenone, with affinity for the mineralocorticoid receptor for use in the treatment of myocardial fibrosis.

Published International PCT Application WO 02/17895 discloses that aldosterone antagonists are useful in the treatment of subjects suffereing from one or more cognitive dysfunctions.

Published International PCT Application WO 02/09683 discloses aldosterone blockers useful to treat inflammation disoders.

Published International PCT Application WO 02/051832 discloses heterocyclalkylindoles as 5HT-6 ligands.

Published International PCT Application WO 02/016348 discloses indole derivatives molecules as antiangiogenic agents.

Published International PCT Application WO 02/012227 discloses nine- and ten-membered bicyclic heteroaryl molecules as angiogenesis inhibitors.

Published International PCT Application WO 01/058893 discloses indol-3-yl propionates as integrin inhibitors.

Published International PCT Application WO 99/43672 discloses indole derivatives as phospholipase enzyme inhibitors.

Published International PCT Application WO 98/42696 and related family members disclose inhibitors of nitric oxide synthase.

Published International PCT Application WO 97/43260 and related family members disclose indole derivatives useful as endothelin receptor antagonists.

Published International PCT Application WO 96/03377 and related family members disclose heterocyclic compounds useful as allosteric effectors of muscarinic receptors.

European Patent EP683166 dislcoses 1-(3-indolylalkyl)-4-(3-indolyl)piperidines as dopamine agonists or antagonists.

Japanese Patents JP 05339565 and JP 3229654 disclose indole derivatives for electroluminescent devices.

U.S. Pat. No. 5,342,547 dislcoses indole derivatives for controlling underwater fouling.

Whitehead and Whitesitt, Journal of Medicinal Chemistry (1974), 17(12), 1298-304 discloses the effects of lipohilic substituents on biological properties of indoles.

### SUMMARY OF THE INVENTION

The present invention is directed to the discovery that certain indole-derivative compounds, as defined below, are modulators of steroid hormone nuclear receptors and, therefore, may have utility as pharmaceutical agents. Accordingly, the present invention provides a compound of the formula: 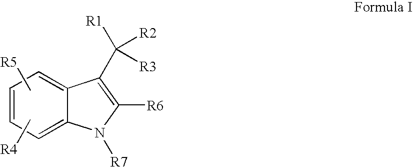
wherein,

R¹ represents (C₃-C₇)cycloalkyl, (C₂-C₆)alkynyl, aryl, heterocycle, fused heterocycle, or a substituted aryl, heterocycle, or fused heterocycle;

R² represents (C₁-C₆)alkyl, (C₃-C₇)cycloalkyl, aryl, substituted aryl, heterocycle, substituted heterocycle, (C₁-C₄)alkyl-(C₃-C₇)cycloalkyl, (C₁-C₄)alkyl-heterocycle, (C₁-C₄)alkyl-substituted heterocycle, (C₁-C₄)alkyl-aryl, (C₁-C₄)alkyl-substituted aryl, halo(C₁-C₆)alkyl, (C₁-C₄)alkyl-(C₁-C₆)alkoxy, (C₂-C₆)alkenyl, (C₂-C₆)alkynyl, cyano(C₁-C₆)alkyl, nitro(C₁-C₆)alkyl, amino(C₁-C₆)alkyl, NH(C₁-C₄)alkylamine, N,N—(C₁-C₄)dialkylamine(C₁-C₄)alkyl-NH(C₁-C₄)alkylamine, or (C₁-C₄)alkyl-N,N—(C₁-C₄)dialkylamine;

R³ represents (C₁-C₆)alkyl, halo(C₁-C₆)alkyl, (C₃-C₇)cycloalkyl, (C₁-C₄)alkyl-(C₃-C₇)cycloalkyl, (C₁-C₆)alkoxy, (C₁-C₄)alkyl-(C₁-C₆)alkoxy, aryl or R² and R³ together with the carbon atom to which they are attached form a (C₃-C₇)cycloalkyl or heterocycle group;

R⁴ represents hydrogen, halo, hydroxyl, amino, nitro, cyano, difluoromethyl, triflouromethyl, difluoromethoxy, triflouromethoxy, (C₁-C₆)alkyl, hydroxy(C₁-C₆)alkyl, (C₁-C₆)alkoxy, (C₃-C₇)cycloalkyl, (C₁-C₄)alkyl-(C₃-C₇)cycloalkyl, aryl, haloaryl, heterocycle, NH(C₁-C₄)alkylamine, N,N—(C₁-C₄)dialkylamine, NHSO₂R⁸, N(CH₃)SO₂R⁸, NHCOR¹², SO₂R⁹, CHO, or OR¹⁰;

R⁵ represents hydrogen, halo, hydroxyl, amino, nitro, cyano, difluoromethyl, triflouromethyl, difluoromethoxy, triflouromethoxy, (C₁-C₆)alkyl, or OR¹¹;

R⁶ represents hydrogen, halo, (C₁-C₆)alkyl, or (C₃-C₇)cycloalkyl;

R⁷ represents hydrogen, (C₁-C₆)alkyl, (C₃-C₇)cycloalkyl, (C₁-C₄)alkyl-CONH₂, COOH, (C₁-C₄)alkyl-COOH, COOCH₃, (C₁-C₄)alkyl-COOCH₃, or SO₂-phenyl;

R⁸ and R⁹ each independently represent at each occurrence amino, (C₁-C₆)alkyl, (C₃-C₇)cycloalkyl, aryl, substituted aryl, (C₁-C₄)alkyl-aryl, (C₁-C₄)alkyl-substituted aryl, heterocycle, substituted heterocycle, (C₁-C₄)alkyl-heterocycle, (C₁-C₄)alkyl-substituted heterocycle, NH(C₁-C₄)alkylamine, or N,N—(C₁-C₄)dialkylamine;

R¹⁰ and R¹¹ each independently represent (C₃-C₇)cycloalkyl, aryl, substituted aryl, (C₁-C₄)alkyl-aryl, (C₁-C₄)alkyl-substituted aryl, heterocycle, substituted heterocycle, (C₁-C₄)alkyl-heterocycle, or (C₁-C₄)alkyl-substituted heterocycle; and

R¹² represents (C₁-C₆)alkyl,

provided that where R¹ through R³ all represent aryl, then at least one of R⁴, R⁵ or R⁷ is other than hydrogen;

or a pharmaceutically acceptable salt thereof.

As another aspect, the present invention provides a method of treating a physiological disorder susceptible to steroid hormone nuclear receptor modulation comprising administering to a patient in need thereof an effective amount of a compound of Formula I as described herein and above. Examples of such disorders include Conn's Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Bartter's Syndrome, disorders associated with excess catecholamine levels, diastolic and systolic congestive heart failure (CHF), peripheral vascular disease, diabetic nephropathy, cirrhosis with edema and ascites, esophageal varicies, Addison's Disease, muscle weakness, increased melanin pigmentation of the skin, weight loss, hypotension, hypoglycemia, Cushing's Syndrome, obesity, hypertension, glucose intolerance, hyperglycemia, diabetes mellitus, osteoporosis, polyuria, polydipsia, inflammation, autoimmune disorders, tissue rejection associated with organ transplant, malignancies such as leukemias and lymphomas, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hyperglycemia, acute adrenal insufficiency, chronic primary adrenal insufficiency, secondary adrenal insufficiency, congenital adrenal hyperplasia, cerebral edema, thrombocytopenia, and Little's syndrome, systemic inflammation, inflammatory bowel disease, systemic lupus erythematosus, discoid lupus erythematosus, polyartitis nodosa, Wegener's granulomatosis, giant cell arthritis, rheumatoid arthritis, osteoarthritis, hay fever, allergic rhinitis, contact dermatitis, atopic dermatitis, exfoliative dermatitis, urticaria, angioneurotic edema, chronic obstructive pulmonary disease, asthma, tendonitis, bursitis, Crohn's disease, ulcerative colitis, autoimmune chronic active hepatitis, hepatitis, cirrhosis, inflammatory scalp alopecia, panniculitis, psoriasis, inflamed cysts, pyoderma gangrenosum, pemphigus vulgaris, bullous pemphigoid, dermatomyositis, eosinophilic fasciitis, relapsing polychondritis, inflammatory vasculitis, sarcoidosis, Sweet's disease, type 1 reactive leprosy, capillary hemangiomas, lichen planus, erythema nodosum, acne, hirsutism, toxic epidermal necrolysis, erythema multiform, cutaneous T-cell lymphoma, psychoses, cognitive disorders (such as memory disturbances), mood disorders (such as depression and bipolar disorder), anxiety disorders, and personality disorders.

As a further aspect, the present invention provides a method of treating a physiological disorder susceptible to mineralocorticoid or glucocorticoid receptor modulation comprising administering to a patient in need thereof an effective amount of a compound of Formula I as described herein and above. As a more particular aspect, the present invention provides a method of treating a physiological disorder susceptible to mineralocorticoid or glucocorticoid receptor antagonism comprising administering to a patient in need thereof an effective amount of a compound of Formula I. As an even more particular aspect the present invention provides a method of treating hypertension (isolated systolic and combined systolic/diastolic), systolic and/or diastolic congestive heart failure, or inflammation comprising administering to a patient in need thereof an effective amount of a compound of Formula I as described herein and above.

As a separate aspect, the present invention also provides a method of modulating a steroid hormone nuclear recpetor comprising contacting said receptor with an effective amount of a compound of Formula I. More particularly, the present invention provides a method of modulating the mineralocorticoid or glucocorticoid receptor comprising contacting said receptor with an effective amount of a compound of Formula I. More particularly still, the present invention provides a method of antagonizing the mineralocorticoid or glucocorticoid receptor comprising contacting said receptor with an effective amount of a compound of Formula I, as described herein and above.

In addition, the present invention provides pharmaceutical compositions of compounds of Formula I, including any pharmaceutically acceptable salts and hydrates thereof, comprising a compound of Formula I in combination with a pharmaceutically acceptable carrier, diluent or excipient. This invention also encompasses novel intermediates, and processes for the synthesis of the compounds of Formula I.

The present invention also provides the use of a compound of Formula I, or a pharmaceutically acceptable salt thereof, for the manufacture of a medicament for treating a physiological disorder susceptible to steroid hormone nuclear receptor modulation. More particularly, the present invention provides the use of a compound of Formula I, or a pharmaceutically acceptable salt thereof, for the manufacture of a medicament for treating hypertension, congestive heart failure, or inflammation.

### DETAILED DESCRIPTION OF THE INVENTION

The present invention provides compounds of Formula I with affinity for steroid hormone nuclear receptors, particularly MR and/or GR, which could be used to modulate (i.e. repress, antagonize, agonize, partially antagonize, partially agonize) nuclear receptor activity and target gene expression, thereby influencing physiological functions related to steroid hormone levels and/or steroid hormone receptor activity. In this regard, compounds of Formula I are believed to be useful in treating or preventing a multitude of physiological disorders susceptible to steroid hormone nuclear receptor modulation. Thus, methods for the treatment or prevention of physiological disorders susceptible to steroid hormone nuclear receptor modulation constitute another important embodiment of the present invention. As a particular aspect, the present invention provides compounds useful as mineralocorticoid or glucocorticoid receptor modulators. As a more particular aspect, the present invention provides compounds useful as mineralocorticoid or glucocorticoid receptor antagonists.

As will be understood by the skilled artisan, some of the compounds useful for the methods of the present invention may be available for prodrug formulation. As used herein, the term “prodrug” refers to a compound of Formula I which has been structurally modified such that in vivo the prodrug is converted, for example, by hydrolytic, oxidative, reductive, or enzymatic cleavage, into the parent molecule (“drug”) as given by Formula I. Such prodrugs may be, for example, metabolically labile ester derivatives of the parent compound where said parent molecule bears a carboxylic acid group. Conventional procedures for the selection and preparation of suitable prodrugs are well known to one of ordinary skill in the art.

It is also understood that many of the steroid hormone nuclear receptor modulators of the present invention may exist as pharmaceutically acceptable salts and, as such, pharmaceutically acceptable salts are therefore included within the scope of the present invention. The term “pharmaceutically acceptable salt” as used herein, refers to salts of the compounds of Formula I, which are substantially non-toxic to living organisms. Typical pharmaceutically acceptable salts include those salts prepared by reaction of the compounds of the present invention with a pharmaceutically acceptable mineral or organic acid or an organic or inorganic base. Such salts are known as acid addition and base addition salts. It is further understood by the skilled reader that salt forms of pharmaceutical compounds are commonly used because they are often more readily crystallized, or more readily purified, than are the free bases. In all cases, the use of the pharmaceutical compounds of the present invention as salts is contemplated in the description herein. Hence, it is understood that where compounds of Formula I are capable of forming salts, the pharmaceutically acceptable salts and isoforms thereof are encompassed in the names provided herein.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, hydroiodide, dihydroiodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulfonate, phenyl acetate, phenyl propionate, phenyl butyrate, citrate, lactate, α-hydroxybutyrate, glycolate, tartrate, methanesulfonate, propanesulfonate, naphthalene-1-sulfonate, napththalene-2-sulfonate, mandelate and the like. Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The compounds of the present invention may have one or more chiral centers and may, therefore, exist in a variety of stereoisomeric configurations. As a consequence of these chiral centers the compounds of the present invention may occur as racemates, mixtures of enantiomers, and as individual enantiomers as well as diastereomers and mixtures of diastereomers. All such racemates, enantiomers, and diastereomers are within the scope of the present invention. Enantiomers of the compounds provided by the present invention can be resolved, for example, by one of ordinary skill in the art using standard techniques such as those described by J. Jacques, et al., “Enantiomers, Racemates, and Resolutions”, John Wiley and Sons, Inc., 1981.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond from the chiral carbon toward the lowest priority group. The term “S” (sinister) refers to that configuration of a chiral center with a counterclockwise relationship of group priorities (highest to second lowest) when viewed along the bond from the chiral carbon toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

The specific stereoisomers and enantiomers of compounds of Formula I can be prepared by one of ordinary skill in the art utilizing well known techniques and processes, such as those disclosed by Eliel and Wilen, “Stereochemistry of Organic Compounds”, John Wiley & Sons, Inc., 1994, Chapter 7; Separation of Stereoisomers, Resolution, Racemization; and by Collet and Wilen, “Enantiomers, Racemates, and Resolutions”, John Wiley & Sons, Inc., 1981. For example, specific stereoisomers and enantiomers can be prepared by stereospecific syntheses using enantiomerically and geometrically pure, or enantiomerically or geometrically enriched starting materials. In addition, the specific stereoisomers and enantiomers can be resolved and recovered by techniques such as chromatography on chiral stationary phases, enzymatic resolution or fractional recrystallization of addition salts formed by reagents used for that purpose.

As appreciated by one of ordinary skill in the art, suitable oxygen or nitrogen protecting groups are used as needed. Suitable oxygen or nitrogen protecting groups, as used herein, refers to those groups intended to protect or block the oxygen or nitrogen group against undesirable reactions during synthetic procedures. The suitability of the oxygen or nitrogen protecting group used will depend upon the conditions that will be employed in subsequent reaction steps wherein protection is required, and is well within the knowledge of one of ordinary skill in the art. Commonly used protecting groups suitable for practicing the present invention are disclosed in “Protective Groups in Organic Synthesis, 3^{rd} Edition” by Theodara Greene, Peter G. M. Wuts, John Wiley & Sons, New York (1999).

As used herein the term “(C₁-C₄)alkyl” refers to a straight or branched, monovalent, saturated aliphatic chain of 1 to 4 carbon atoms and includes, but is not limited to methyl, ethyl, n-propyl, isopropyl, n-butyl, isobutyl and the like.

As used herein the term “(C₁-C₆)alkyl” refers to a straight or branched, monovalent, saturated aliphatic chain of 1 to 6 carbon atoms and includes, but is not limited to methyl, ethyl, n-propyl, isopropyl, n-butyl, isobutyl, t-butyl, n-pentyl, n-hexyl, and the like. It is understood that the term “(C₁-C₄)alkyl” is included within the definition of “(C₁-C₆)alkyl”.

As used herein the term “(C₁-C₁₀)alkyl” refers to a straight or branched, monovalent, saturated aliphatic chain of 1 to 10 carbon atoms and includes, but is not limited to methyl, ethyl, propyl, isopropyl, n-butyl, isobutyl, tertiary butyl, pentyl, isopentyl, hexyl, 2,3-dimethyl-2-butyl, heptyl, 2,2-dimethyl-3-pentyl, 2-methyl-2-hexyl, octyl, 4-methyl-3-heptyl and the like. It is understood that the terms “(C₁-C₄)alkyl” and “(C₁-C₆)alkyl” are included within the definition of “(C₁-C₁₀)alkyl”.

As used herein, the terms “Me”, “Et”, “Pr”, “l-Pr”, “Bu” and “t-Bu” refer to methyl, ethyl, propyl, isopropyl, butyl and tert-butyl respectively.

As used herein, the term “(C₁-C₄)alkoxy” refers to an oxygen atom bearing a straight or branched, monovalent, saturated aliphatic chain of 1 to 4 carbon atoms and includes, but is not limited to methoxy, ethoxy, n-propoxy, isopropoxy, n-butoxy, and the like. As used herein the term “(C₁-C₆)alkoxy” refers to an oxygen atom bearing a straight or branched, monovalent, saturated aliphatic chain of 1 to 6 carbon atoms and includes, but is not limited to methoxy, ethoxy, n-propoxy, isopropoxy, n-butoxy, n-pentoxy, n-hexoxy, and the like. It is understood that the term “(C₁-C₄)alkoxy” is included within the definition of “(C₁-C₆)alkoxy”.

As used herein, the term “hydroxy(C₁-C₄)alkyl” refers to a straight or branched, monovalent, saturated aliphatic chain of 1 to 4 carbon atoms bearing a hydroxyl group attached to one of the carbon atoms. As used herein, the term “hydroxy(C₁-C₆)alkyl” refers to a straight or branched, monovalent, saturated aliphatic chain of 1 to 6 carbon atoms bearing a hydroxyl group attached to one of the carbon atoms. It is understood that the term “hydroxy(C₁-C₄)alkyl” is included within the definition of “hydroxy(C₁-C₆)alkyl”. As used herein, the term “hydroxy(C₁-C₄)alkoxy” refers to an oxygen atom bearing a straight or branched, monovalent, saturated aliphatic chain of 1 to 4 carbon atoms, further bearing a hydroxyl group attached to one of the carbon atoms. As used herein, the term “hydroxy(C₁-C₆)alkoxy” refers to an oxygen atom bearing a straight or branched, monovalent, saturated aliphatic chain of 1 to 6 carbon atoms, further bearing a hydroxyl group attached to one of the carbon atoms. It is understood that the term “hydroxy(C₁-C₄)alkoxy” is included within the definition of “hydroxy(C₁-C₆)alkoxy”.

As used herein, the term “(C₁-C₆)alkyl-(C₁-C₆)alkoxy” (or “(C₁-C₆)alkoxy(C₁-C₆)alkyl”) refers to a straight or branched, monovalent, saturated aliphatic chain of 1 to 6 carbon atoms which has a (C₁-C₆)alkoxy group attached to the aliphatic chain. The term “(C₁-C₆)alkoxymethylene” refers to a methylene group bearing a (C₁-C₆)alkoxy group. “(C₁-C₆)alkoxy(C₁-C₆)alkoxy-methylene refers to a methylene group bearing a (C₁-C₆)alkoxy group which, in turn, bears an additional (C₁-C₆)alkoxy group attached to the aliphatic chain.

As used herein, the terms “halo”, “halide” or “hal” of “Hal” refer to a chlorine, bromine, iodine or fluorine atom, unless otherwise specified herein.

As used herein, the term “halo(C₁-C₄)alkyl” refers to a straight or branched, monovalent, saturated aliphatic chain of 1 to 4 carbon atoms bearing one or more halo groups attached to one or more of the carbon atoms. As used herein, the term “halo(C₁-C₆)alkyl” refers to a straight or branched, monovalent, saturated aliphatic chain of 1 to 6 carbon atoms bearing one or more halo groups attached to one or more of the carbon atoms. It is understood that the term “halo(C₁-C₄)alkyl” is included within the definition of “halo(C₁-C₆)alkyl”. As used herein, the term “halo(C₁-C₄)alkoxy” refers to an oxygen atom bearing a straight or branched, monovalent, saturated aliphatic chain of 1 to 4 carbon atoms, further bearing one or more halo groups attached to one or more of the carbon-atoms. As used herein, the term “halo(C₁-C₆)alkoxy” refers to an oxygen atom bearing a straight or branched, monovalent, saturated aliphatic chain of 1 to 6 carbon atoms, further bearing one or more halo groups attached to one or more of the carbon atoms. It is understood that the term “halo(C₁-C₄)alkoxy” is included within the definition of “halo(C₁-C₆)alkoxy”.

As used herein the term “(C₂-C₆)alkenyl” refers to a straight or branched, monovalent, unsaturated aliphatic chain having from two to six carbon atoms and having a double bond. Typical (C₂-C₆)alkenyl groups include ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, 2-pentenyl, and the like.

As used herein the term “(C₂-C₆)alkynyl” refers to a straight or branched, monovalent, unsaturated aliphatic chain having from two to six carbon atoms and having a triple bond. Typical (C₂-C₆)alkynyl groups include propynyl, ethynyl, and the like

As used herein, the term “acyl” refers to a hydrogen or a (C₁-C₆)alkyl group attached to a carbonyl group. Typical acyl groups include formyl, acetyl, propionyl, butyryl, valeryl, and caproyl.

As used herein, the term “aryl” refers to a monovalent carbocyclic group containing one or more fused or non-fused phenyl rings and includes, for example, phenyl, 1- or 2-naphthyl, 1,2-dihydronaphthyl, 1,2,3,4-tetrahydronaphthyl, and the like. The term “substituted aryl” refers to an aryl group optionally substituted with one to three moieties, preferably one or two, chosen from the group consisting of acyl, halogen, hydroxy, cyano, nitro, amino, (C₁-C₆)alkyl, (C₁-C₄)alkylsulfonyl, (C₁-C₄)alkylsulfinyl, (C₁-C₆)alkoxy, aryl(C₁-C₆)alkoxy, halo(C₁-C₆)alkoxy, (C₁-C₆)alkylthio, (C₃-C₇)cycloalkyl, (C₁-C₄)alkyl-(C₃-C₇)cycloalkyl, aryl, (C₁-C₄)alkyl-aryl, heterocycle, (C₁-C₄)alkyl-heterocycle, (C₁-C₄)alkoxy-heterocycle, (C₁-C₆)alkoxycarbonyl, N,N(C₁-C₆)dialkylamine, NH(C₁-C₆)alkylamine, NHSO₂(C₁-C₄)alkyl, (C₁-C₄)alkyl-N,N—(C₁-C₆)dialkylamine, (C₁-C₄)alkoxy-N,N—(C₁-C₆)dialkylamine difluoromethyl, difluoromethoxy, trifluoromethyl, trifluoromethoxy, CF₂CF₃, benzoyl, phenoxy, benzyloxy, or an aryl or heterocycle group further substituted with one to two moieties selected from the group consisting of:
(C₁-C₄)alkyl,
(C₃-C₇)cycloalkyl,
halo,
hydroxy,
(C₁-C₄)alkoxy,
CF₃,
OCF₃,
CHF₂,
OCHF₂,
CF₂CF₃,
cyano,
nitro,
amino,
NH(C₁-C₄)alkylamine, and
N,N—(C₁-C₄)dialkylamine;

As used herein, the term “(C₁-C₆)alkyl-aryl” (or “aryl(C₁-C₆)alkyl) refers to a straight or branched, monovalent, saturated aliphatic chain of 1 to 6 carbon atoms which has an aryl group attached to the aliphatic chain. “(C₁-C₄)alkyl-aryl” (or “aryl(C₁-C₄)alkyl) refers to a straight or branched, monovalent, saturated aliphatic chain of 1 to 4 carbon atoms which has an aryl group attached to the aliphatic chain. It is understood that the term “(C₁-C₄)alkyl-aryl” is included within the definition of “(C₁-C₆)alkyl-aryl. Examples of “(C₁-C₆)alkyl-aryl” include the following: 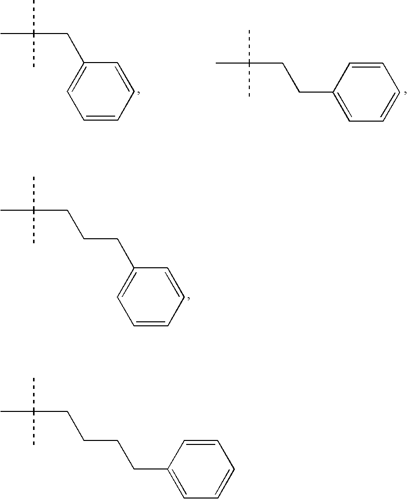
and the like.

As used herein, the term “(C₁-C₄)alkyl-substituted aryl” refers to a straight or branched, monovalent, saturated aliphatic chain of 1 to 4 carbon atoms which has an optionally substituted aryl group, as described above; attached to the aliphatic chain. Examples of “(C₁-C₄)alkyl-substituted aryl” include methylbenzyl, phenylbenzyl, nitrobenzyl, methoxybenzyl, chlorobenzyl, bromobenzyl, dimethlybenzyl, aminobenzyl, dichlorobenzyl, and the like.

As used herein, the term “aryl(C₁-C₆)alkoxy” (or “(C₁-C₆)alkoxy-aryl”) refers to an oxygen atom bearing a straight or branched, monovalent, saturated aliphatic chain of 1 to 6 carbon atoms wherein said aliphatic chain, in turn, bears an aryl group. Examples of “aryl(C₁-C₆)alkoxy” include benzyloxy, phenyl ethoxy, and the like.

As used herein the term “(C₃-C₁₀)cycloalkyl” refers to a saturated hydrocarbon ring structure composed of one or more fused or unfused rings containing from three to ten carbon atoms. Typical (C₃-C₁₀)cycloalkyl groups include cyclopropyl, cyclobutyl, cyclopentyl, cyclohexyl, cycloheptyl, cyclooctyl, adamantanyl, and the like. “(C₃-C₇)cycloalkyl” refers to a saturated hydrocarbon ring structure composed of one or more fused or unfused rings containing from three to seven carbon atoms. It is understood that the definition of “(C₃-C₇)cycloalkyl” is included within the definition of “(C₃-C₁₀)cycloalkyl”. The term “substituted (C₃-C₇)cycloalkyl” refers to a “(C₃-C₇)cycloalkyl group optionally substituted with one or two moieties chosen from the group consisting of halogen, hydroxy, cyano, nitro, amino, (C₁-C₆)alkyl, (C₁-C₆)alkoxy, (C₁-C₄)alkyl-(C₃-C₁₀)cycloalkyl, (C₁-C₄)alkyl-aryl, (C₁-C₆)alkoxycarbonyl, N,N(C₁-C₆)dialkylamine, NH(C₁-C₆)alkylamine, (C₁-C₄)alkyl-N,N—C₁-C₆dialkylamine, difluoromethyl, difluoromethoxy, trifluoromethyl, and trifluoromethoxy.

As used herein, the term “(C₁-C₄)alkyl-(C₃-C₇)cycloalkyl” refers to a straight or branched, monovalent, saturated aliphatic chain of 1 to 4 carbon atoms which has a (C₃-C₇)cycloalkyl attached to the aliphatic chain. Included within the term “(C₁-C₄)alkyl-(C₃-C₇)cycloalkyl” are the following: 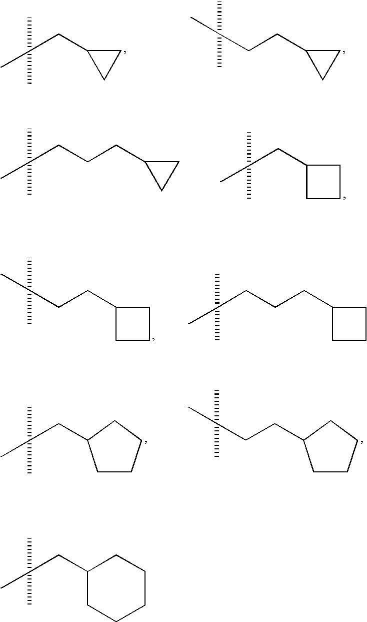
and the like. As used herein, the term “(C₁-C₄)alkyl-substituted (C₃-C₇)cycloalkyl” refers to a straight or branched, monovalent, saturated aliphatic chain of 1 to 4 carbon atoms bearing an optionally substituted (C₃-C₇)cycloalkyl group attached to the aliphatic chain.

As used herein the term “(C₃-C₇)cycloalkoxy” refers to an oxygen atom bearing a saturated hydrocarbon ring structure composed of one or more fused or unfused rings containing from three to seven carbon atoms.

As used herein, the term “(C₁-C₆)alkoxycarbonyl” refers to a carbonyl group having a (C₁-C₆)alkyl group attached to the carbonyl carbon through an oxygen atom. Examples of this group include t-butoxycarbonyl, methoxycarbonyl, ethoxycarbonyl and the like. It is understood that the term “(C₁-C₄)alkoxycarbonyl” is included within the definition of “(C₁-C₆)alkoxycarbonyl”.

As used herein the term “heterocycle” refers to a saturated or unsaturated, five- or six-membered ring, which contains one to four heteroatoms selected from the group consisting of oxygen, sulfur, and nitrogen. It is understood that the remaining atoms are carbon and that the heterocycle may be attached at any point which provides for a stable structure. Examples of heterocycle groups include thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, and the like.

As used herein, the term “fused-heterocyclic ring” or “fused heterocycle” refers to a bicyclic ring system consisting of a saturated, partially unsaturated, or unsaturated five- or six-membered ring fused to a six-membered aromatic ring wherein said bicyclic ring system contains one to four heteroatoms selected from the group consisting of oxygen, sulfur, and nitrogen. It is understood that the remaining atoms of the bicyclic ring system are carbon and that the fused heterocycle may be attached at any point on either of the fused rings which provides for a stable structure. Typical structures of “fused heterocycles”, as used herein, are given by the following: 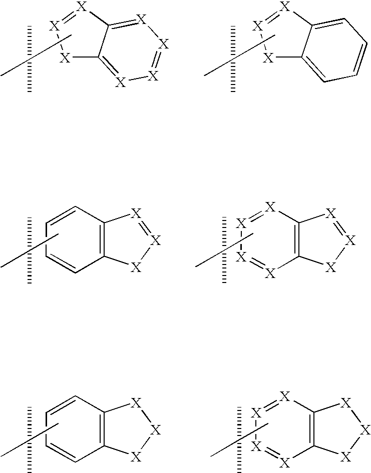
In the structures above, “X” represents independently at each occurrence a carbon atom or a heteroatom selected from nitrogen, oxygen and sulfur, provided however that no more than four heteroatoms may be present in any given bicyclic system at a given time. Representative “fused heterocyclic rings” include benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, quinoline, and the like.

The term “substituted heterocycle” represents a heterocycle group optionally substituted with one or two moieties chosen from the group consisting of acyl, halogen, hydroxy, cyano, nitro, amino, (C₁-C₆)alkyl, (C₁-C₄)alkylsulfonyl, (C₁-C₆)alkoxy, halo(C₁-C₆)alkoxy, aryl(C₁-C₆)alkoxy, (C₁-C₆)alkylthio, (C₃-C₇)cycloalkyl, (C₁-C₄)alkyl-(C₃-C₇)cycloalkyl, aryl, (C₁-C₄)alkyl-aryl, heterocycle, (C₁-C₄)alkyl-heterocycle, (C₁-C₄)alkoxy-heterocycle, (C₁-C₆)alkoxycarbonyl, N,N(C₁-C₆)dialkylamine, NH(C₁-C₆)alkylamine, NHSO₂(C₁-C₄)alkyl, (C₁-C₄)alkyl-N,N—C₁-C₆dialkylamine, (C₁-C₄)alkoxy-N,N—C₁-C₆dialkylamine, difluoromethyl, difluoromethoxy, trifluoromethyl, trifluoromethoxy, CF₂CF₃, or an aryl or heterocycle group further substituted with one to two moieties selected from the group consisting of:
(C₁-C₄)alkyl,
(C₃-C₇)cycloalkyl,
halo,
hydroxy,
(C₁-C₄)alkoxy,
CF₃,
OCF₃,
CHF₂,
OCHF₂,
CF₂CF₃,
cyano,
nitro,
amino,
NH(C₁-C₄)alkylamine, and
N,N—(C₁-C₄)dialkylamine;

Examples of substituted heterocycle include 2-chlorothiophene, 2-bromothiophene, 2-methylthiophene, 2-fluorothiophene, and the like.

The term “substituted fused heterocyclic ring” or “substituted fused heterocycle” represents a “fused heterocycle”, as defined herein, optionally substituted with one or two moieties chosen from the group consisting of hydroxy, cyano, nitro, amino, halo, (C₁-C₆)alkyl, (C₁-C₆)alkoxy, difluoromethyl, difluoromethoxy, trifluoromethyl, trifluoromethoxy, hydroxy(C₁-C₆)alkyl, (C₃-C₇)cycloalkyl, (C₁-C₄)alkyl-(C₃-C₇)cycloalkyl, aryl, haloaryl, heterocycle, N,N(C₁-C₆)dialkylamine, or NH(C₁-C₆)alkylamine. Examples of “substituted fused heterocycle” include 5-chloro-benzofuran-2-yl, 5-methoxy benzofuran-2-yl, 7-methoxy benzofuran-2-yl, 7-fluoro benzofuran-2-yl, 5-fluoro benzofuran-2-yl, 5-chloro-7-fluoro benzofuran-2-yl, 2,2-difluoro-benzo[1,3]dioxol-5-yl, 6-chloro benzo(b)thiophen-2-yl, 4-chloro benzo(b)thiophen-2-yl, 4-trifluoromethyl benzo(b)thiophen-2-yl, 5-trifluoromethyl benzo(b)thiophen-2-yl, 6-trifluoromethyl benzo(b)thiophen-2-yl, 7-trifluoromethyl benzo(b)thiophen-2-yl, 4-fluoro benzo(b)thiophen-2-yl, 5-fluoro benzo(b)thiophen-2-yl, 7-fluoro benzo(b)thiophen-2-yl, 3-methyl-4-fluoro benzo(b)thiophen-2-yl, 3-methyl-7-fluoro benzo(b)thiophen-2-yl, and the like.

As used herein, the term “(C₁-C₄)alkyl-heterocycle” refers to a straight or branched, monovalent, saturated aliphatic chain of 1 to 4 carbon atoms which has a heterocycle group attached to the aliphatic chain. Examples of “(C₁-C₄)alkyl-heterocycle” include:

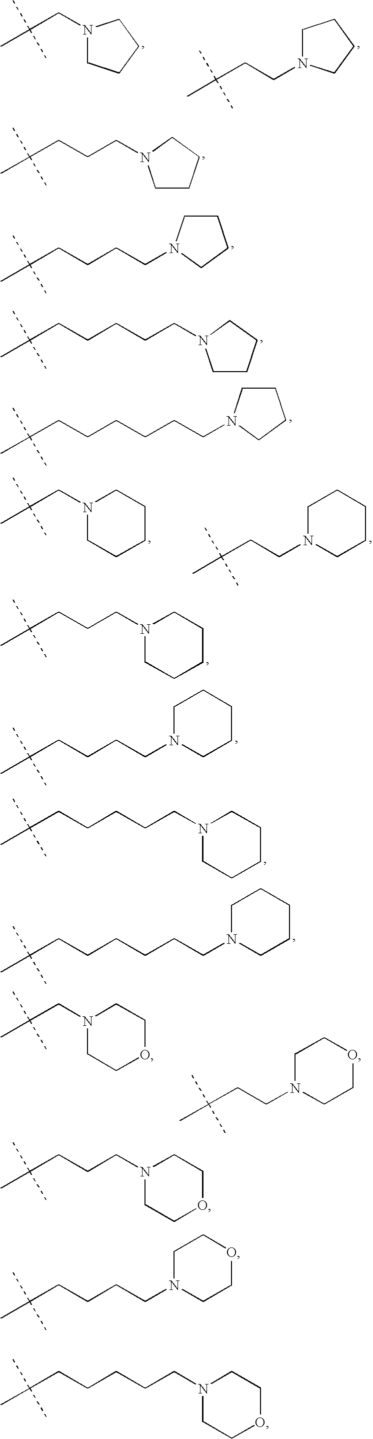

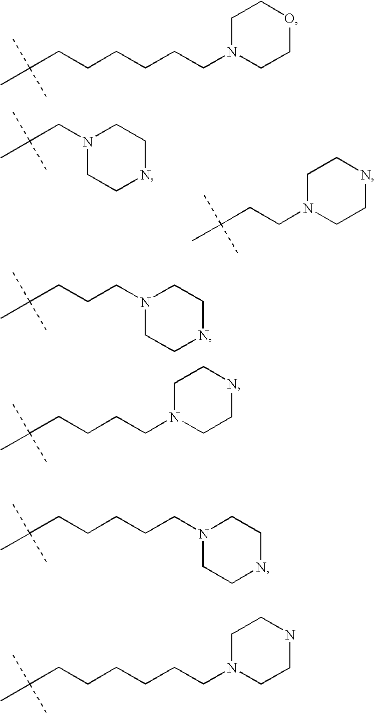

and the like.

The term “(C₁-C₄)alkyl-substituted heterocycle” refers to a straight or branched, monovalent, saturated aliphatic chain of 1 to 4 carbon atoms bearing an optionally substituted heterocycle group attached to the aliphatic chain.

As used herein, the term “(C₁-C₄)alkoxy-heterocycle” refers to an oxygen atom bearing a straight or branched, monovalent, saturated aliphatic chain of 1 to 4 carbon atoms which has a heterocycle group attached to the aliphatic chain. Examples of “(C₁-C₄)alkoxy-heterocycle” include: 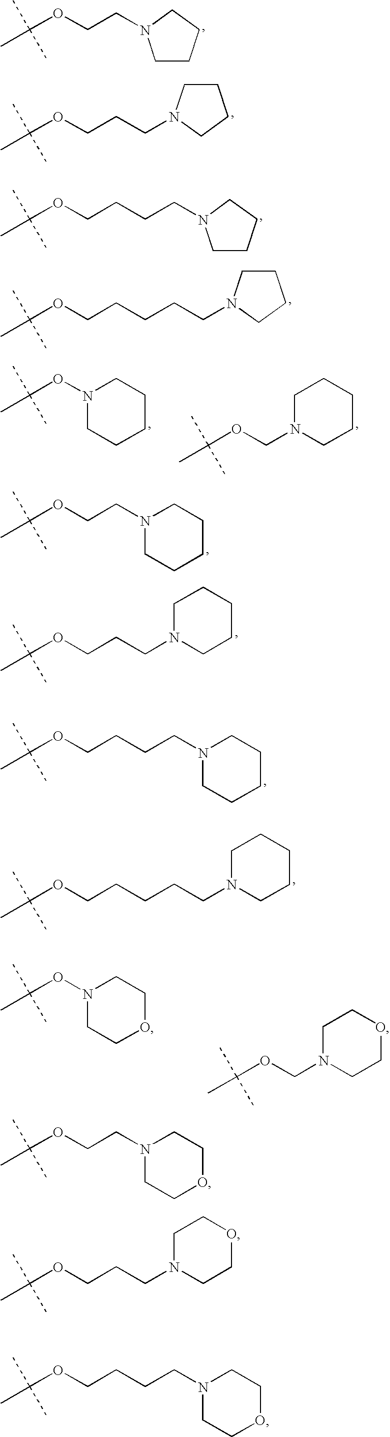 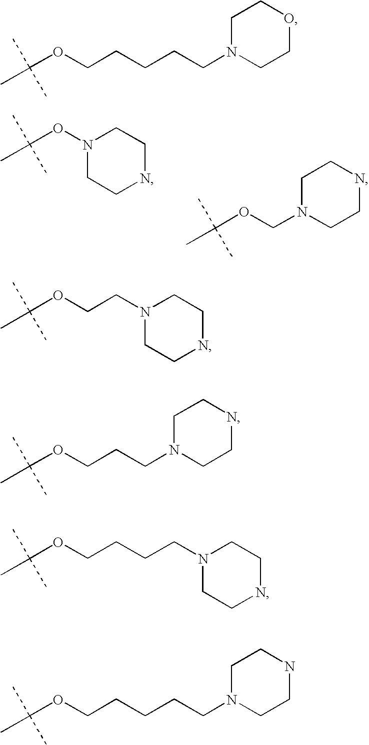
and the like.

As used herein the term “NH(C₃-C₇)cycloalkyl” refers to an amino group substituted with a saturated hydrocarbon ring structure composed of one or more fused or unfused rings containing from three to seven carbon atoms.

As used herein, the term “NH—(C₁-C₆)alkylamine” refers to a nitrogen atom substituted with a straight or branched, monovalent, saturated aliphatic chains of 1 to 6 carbon atoms. Included within the term “NH—(C₁-C₆)alkylamine” are —NH(CH₃), —NH(CH₂CH₃), —NH(CH₂CH₂CH₃), —NH(CH₂CH₂CH₂CH₃), and the like.

As used herein the term “N,N—(C₁-C₆)dialkylamine” refers to a nitrogen atom substituted with two straight or branched, monovalent, saturated aliphatic chains of 1 to 6 carbon atoms. Included within the term “N,N—(C₁-C₆)dialkylamine” are —N(CH₃)₂, —N(CH₂CH₃)₂, —N(CH₂CH₂CH₃)₂, —N(CH₂CH₂CH₂CH₃)₂, and the like.

As used herein the term “(C₁-C₆)alkyl-N,N—C₁-C₆dialkylamine” refers to straight or branched, monovalent, saturated aliphatic chain of 1 to 6 carbon atoms which has an N,N—(C₁-C₆)dialkylamine attached to the aliphatic chain. Included within the term “(C₁-C₆)alkyl-N,N—(C₁-C₆)dialkylamine” are the following: 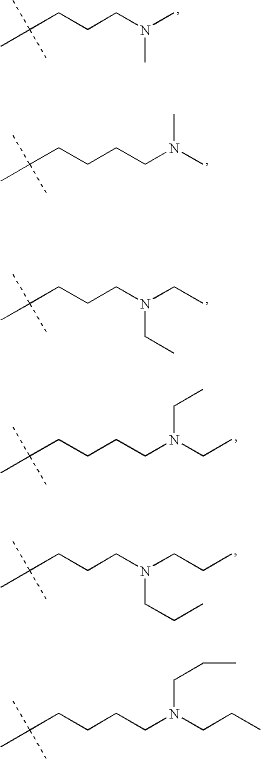
and the like.

As used herein the term “(C₁-C₆)alkoxy-N,N—(C₁-C₆)dialkylamine” refers to an oxygen atom bearing a straight or branched, monovalent, saturated aliphatic chain of 1 to 6 carbon atoms which has an N,N—C₁-C₆ dialkylamine attached to the aliphatic chain. Included within the term “C₁-C₆ alkoxy-N,N—(C₁-C₆)dialkylamine” are the following: 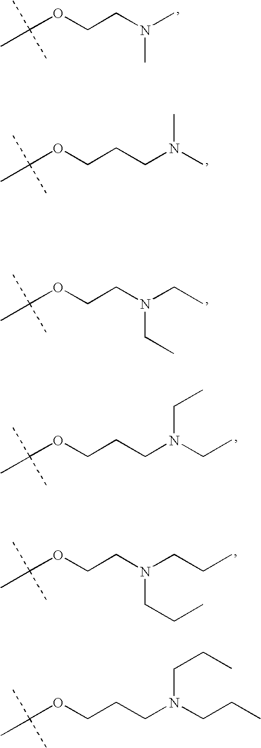
and the like.

The designation [symbol] refers to a bond that protrudes forward out of the plane of the page.

The designation [symbol] refers to a bond that protrudes backward out of the plane of the page.

As used herein, the term “steroid hormone nuclear receptor modulator” refers to those nuclear hormone receptor ligands which bind to any one of GR, MR, AR, ER, or PR, of the larger class of nuclear hormone receptors, and either agonize, antagonize, partially agonize, or partially antagonize the receptor's activity.

As used herein the term “mineralocorticoid receptor” or “MR” refers to the mineralocorticoid receptor subtype, of the larger class of nuclear hormone receptors, which binds the mineralocorticoid hormone aldosterone, as its cognate ligand. The term “mineralocorticoid receptor modulator” or “mineralocorticoid modulator” or “MR modulator” as used herein, refers to those nuclear hormone receptor ligands which bind to the mineralocorticoid receptor subtype and modulate (i.e. agonize, antagonize, partially agonize, or partially antagonize) the receptor activity. As a particular embodiment, the present invention provides antagonists of MR activity

As used herein the term “glucocorticoid receptor” or “GR” refers to the glucocorticoid receptor subtype, of the larger class of nuclear hormone receptors, which binds the glucocorticoid hormones cortisol, corticosterone, or cortisone as its cognate ligand. The term “glucocorticoid receptor modulator” or “glucocorticoid modulator” or “GR modulator”, as used herein, refers to those nuclear hormone receptor ligands which bind to the glucocorticoid receptor subtype and modulate (i.e. agonize, antagonize, partially agonize, or partially antagonize) the receptor activity.

As used herein, the term “disorder susceptible to steroid hormone nuclear receptor modulation” refers to any physiological disorder, of any origin, known or believed to be responsive to administration of a modulator (i.e. agonist, antagonist, partial agonist, or partial antagonist) of a steroid hormone nuclear receptor. Such disorders include Conn's Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Bartter's Syndrome, disorders associated with excess catecholamine levels, diastolic and systolic congestive heart failure (CHF), peripheral vascular disease, diabetic nephropathy, cirrhosis with edema and ascites, esophageal varicies, Addison's Disease, muscle weakness, increased melanin pigmentation of the skin, weight loss, hypotension, hypoglycemia, Cushing's Syndrome, obesity, hypertension, glucose intolerance, hyperglycemia, diabetes mellitus, osteoporosis, polyuria, polydipsia, inflammation, autoimmune disorders, tissue rejection associated with organ transplant, malignancies such as leukemias and lymphomas, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hyperglycemia, acute adrenal insufficiency, chronic primary adrenal insufficiency, secondary adrenal insufficiency, congenital adrenal hyperplasia, cerebral edema, thrombocytopenia, and Little's syndrome, systemic inflammation, inflammatory bowel disease, systemic lupus erythematosus, discoid lupus erythematosus, polyartitis nodosa, Wegener's granulomatosis, giant cell arthritis, rheumatoid arthritis, osteoarthritis, hay fever, allergic rhinitis, contact dermatitis, atopic dermatitis, exfoliative dermatitis, urticaria, angioneurotic edema, chronic obstructive pulmonary disease, asthma, tendonitis, bursitis, Crohn's disease, ulcerative colitis, autoimmune chronic active hepatitis, hepatitis, cirrhosis, inflammatory scalp alopecia, panniculitis, psoriasis, inflamed cysts, pyoderma gangrenosum, pemphigus vulgaris, bullous pemphigoid, dermatomyositis, eosinophilic fasciitis, relapsing polychondritis, inflammatory vasculitis, sarcoidosis, Sweet's disease, type 1 reactive leprosy, capillary hemangiomas, lichen planus, erythema nodosum, acne, hirsutism, toxic epidermal necrolysis, erythema multiform, cutaneous T-cell lymphoma, psychoses, cognitive disorders (such as memory disturbances), mood disorders (such as depression and bipolar disorder), anxiety disorders, and personality disorders.

As used herein the term “congestive heart failure” (CHF) or “congestive heart disease” refers to a disease state of the cardiovascular system whereby the heart is unable to efficiently pump an adequate volume of blood to meet the requirements of the body's tissues and organ systems. Typically, CHF is characterized by left ventricular failure (systolic dysfunction) and fluid accumulation in the lungs, with the underlying cause being attributed to one or more heart or cardiovascular disease states including coronary artery disease, myocardial infarction, hypertension, diabetes, valvular heart disease, and cardiomyopathy. The term “diastolic congestive heart failure” refers to a state of CHF characterized by impairment in the ability of the heart to properly relax and fill with blood. Conversely, the term “systolic congestive heart failure” refers to a state of CHF characterized by impairment in the ability of the heart to properly contract and eject blood.

As appreciated by one of skill in the art, physiological disorders may present as a “chronic” condition, or an “acute” episode. The term “chronic”, as used herein, means a condition of slow progress and long continuance. As such, a chronic condition is treated when it is diagnosed and treatment continued throughout the course of the disease. Conversely, the term “acute” means an exacerbated event or attack, of short course, followed by a period of remission. Thus, the treatment of physiological disorders contemplates both acute events and chronic conditions. In an acute event, compound is administered at the onset of symptoms and discontinued when the symptoms disappear. As described above, a chronic condition is treated throughout the course of the disease.

As used herein the term “patient” refers to a mammal, such a mouse, gerbil, guinea pig, rat, dog or human. It is understood, however, that the preferred patient is a human. As used herein, the terms “treating”, “treatment”, or “to treat” each mean to alleviate symptoms, eliminate the causation of resultant symptoms either on a temporary or permanent basis, and to prevent, slow the appearance, or reverse the progression or severity of resultant symptoms of the named disorder. As such, the methods of this invention encompass both therapeutic and prophylactic administration.

As used herein the term “effective amount” refers to the amount or dose of the compound, upon single or multiple dose administration to the patient, which provides the desired effect in the patient under diagnosis or treatment. An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the degree of involvement or the severity of the disease involved; the response of the individual patient; the particular compound administered; the mode of administration; the bioavailability characteristics of the preparation administered; the dose regimen selected; the use of concomitant medication; and other relevant circumstances.

A typical daily dose will contain from about 0.01 mg/kg to about 100 mg/kg of each compound used in the present method of treatment. Preferably, daily doses will be about 0.05 mg/kg to about 50 mg/kg, more preferably from about 0.1 mg/kg to about 25 mg/kg.

Oral administration is a preferred route of administering the compounds employed in the present invention whether administered alone, or as a combination of compounds capable of acting as a mineralocorticoid receptor modulator. Oral administration, however, is not the only route, nor even the only preferred route. Other preferred routes of administration include transdermal, percutaneous, pulmonary, intravenous, intramuscular, intranasal, buccal, sublingual, or intrarectal routes. Where the steroid hormone nuclear receptor modulator is administered as a combination of compounds, one of the compounds may be administered by one route, such as oral, and the other may be administered by the transdermal, percutaneous, pulmonary, intravenous, intramuscular, intranasal, buccal, sublingual, or intrarectal route, as particular circumstances require. The route of administration may be varied in any way, limited by the physical properties of the compounds and the convenience of the patient and the caregiver.

The compounds employed in the present invention may be administered as pharmaceutical compositions and, therefore, pharmaceutical compositions incorporating compounds of Formula I are important embodiments of the present invention. Such compositions may take any physical form that is pharmaceutically acceptable, but orally administered pharmaceutical compositions are particularly preferred. Such pharmaceutical compositions contain, as an active ingredient, an effective amount of a compound of Formula I, as described herein and above, including the pharmaceutically acceptable salts and hydrates thereof, which effective amount is related to the daily dose of the compound to be administered. Each dosage unit may contain the daily dose of a given compound, or may contain a fraction of the daily dose, such as one-half or one-third of the dose. The amount of each compound to be contained in each dosage unit depends on the identity of the particular compound chosen for the therapy, and other factors such as the indication for which it is given. The pharmaceutical compositions of the present invention may be formulated so as to provide quick, sustained, or delayed release of the active ingredient after administration to the patient by employing well known procedures. The following discussion provides typical procedures for preparing pharmaceutical compositions incorporating the compounds of the present invention. However, the following is in no way intended to limit the scope of the pharmaceutical compositions provided by the present invention.

Compositions are preferably formulated in a unit dosage form, each dosage containing from about 1 to about 500 mg of each compound individually or in a single unit dosage form, more preferably about 5 to about 300 mg (for example 25 mg). The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the type of composition to be used. The amount of the compound, however, is best defined as the “effective amount”, that is, the amount of each compound which provides the desired dose to the patient in need of such treatment. The activity of the compounds employed in the present invention do not depend on the nature of the composition, hence, the compositions are chosen and formulated solely for convenience and economy.

Capsules are prepared by mixing the compound with a suitable diluent and filling the proper amount of the mixture in capsules. The usual diluents include inert powdered substances such as starches, powdered cellulose especially crystalline and microcrystalline cellulose, sugars such as fructose, mannitol and sucrose, grain flours, and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

Tablets are often coated with sugar as a flavor and sealant. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

A lubricant is often necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acid contents of the stomach. Such formulations are created by coating a solid dosage form with a film of a polymer which is insoluble in acid environments, and soluble in basic environments. Exemplary films are cellulose acetate phthalate, polyvinyl acetate phthalate, hydroxypropyl methyl cellulose phthalate and hydroxypropyl methylcellulose acetate succinate.

When it is desired to administer the compound as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches have become popular recently. Typically they comprise a resinous composition in which the drugs will dissolve, or partially dissolve, which is held in contact with the skin by a film which protects the composition. Many patents have appeared in the field recently. Other, more complicated patch compositions are also in use, particularly those having a membrane pierced with innumerable pores through which the drugs are pumped by osmotic action.

It is understood by one of ordinary skill in the art that the procedures as described above can also be readily applied to a method of treating physiological disorders susceptible to steroid hormone nuclear receptor modulation, and particularly congestive heart failure.

Particular Aspects of the Compounds and Methods of the Invention

The following list sets out several groupings of particular substituents for compounds of Formula I. It will be understood that compounds of Formula I having such particular substituents, and the methods employing such compounds, represent particular aspects of the present invention. It will be further understood that each of these groupings of particular substituents may be combined with other provided groupings, to create still additional particular aspects of the compounds of the present invention

Therefore, a particular aspect of the present invention is one wherein the compound of Formula I, is one wherein:
(a) R¹ represents phenyl, (C₂-C₆)alkynyl, heterocycle, fused heterocycle, or a substituted phenyl, heterocycle, or fused heterocycle;
(b) R¹ represents phenyl, ethynyl, propynyl, thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzodioxine, benzodioxepine, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline; or a substituted phenyl, thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzodioxine, benzodioxepine, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline.
(c) R¹ represents phenyl, ethynyl, propynyl, thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline; or a substituted phenyl, thiophenyl, furanyl, tetrahydrofuryl, pyrrolyl, imidazolyl, pyrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline.
(d) R¹ represents phenyl, ethynyl, propynyl, thiophenyl, furanyl, pyridinyl, benzofuranyl, 2,3 dihydro-benzofuranyl, furopyridinyl, benzothiophenyl, indolyl, benzodioxole, quinolinyl, benzoxazole, benzimidazole, benzothiophene, benzothiazole, indazole, benzoisoxazole, benzotriazole, benzodioxine, or benzodioxepine or a substituted phenyl, thiophenyl, furanyl, pyridinyl, benzofuranyl, 2,3 dihydro-benzofuranyl, furopyridinyl, benzothiophenyl, indolyl, benzodioxole, quinolinyl, benzoxazole, benzimidazole, benzothiophene, benzothiazole, indazole, benzoisoxazole, benzotriazole, benzodioxine, or benzodioxepine;
(e) R¹ represents phenyl, ethynyl, propynyl, thiophenyl, furanyl, pyridinyl, benzofuranyl, 2,3 dihydro-benzofuranyl, furopyridinyl, benzothiophenyl, indolyl, benzodioxole, or quinolinyl, or a substituted phenyl, thiophenyl, furanyl, pyridinyl, benzofuranyl, 2,3 dihydro-benzofuranyl, furopyridinyl, benzothiophenyl, indolyl, benzodioxole, or quinolinyl;
(f) R¹ represents phenyl;
(g) R¹ represents ethynyl or propynyl;
(h) R¹ represents thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzodioxine, benzodioxepine, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline;
(i) R¹ represents thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole; benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline;
(j) R¹ represents thiophenyl, furanyl, pyridinyl, benzofuranyl, 2,3 dihydro-benzofuranyl, furopyridinyl, benzothiophenyl, indolyl, benzodioxole, quinolinyl, benzoxazole, benzimidazole, benzothiophene, benzothiazole, indazole, benzoisoxazole, benzotriazole, benzodioxine, or benzodioxepine;
(k) R¹ represents thiophenyl, furanyl, pyridinyl, benzofuranyl, 2,3 dihydro-benzofuranyl, furopyridinyl, benzothiophenyl, indolyl, benzodioxole, or quinolinyl,
(l) R¹ represents thiophen-3-yl, thiophen-2-yl, furan-2-yl, furan-3-yl, pyridin-3-yl, pyridin-2-yl, benzofuran-2-yl, 2,3-dihydro-benzofuran-5-yl, benzo[b]thiophen-2-yl, benzo[b]thiophen-3-yl, quinolin-6-yl, furo[3,2-b]pyridin-2-yl, benzo[1,3]dioxol-5-yl, 1H-indol-3-yl, 1H-Benzoimidazol-5-yl, 1-Benzo[b]thiophen-5-yl, 1-Benzooxazol-6-yl, 1H-indazol-5-yl, 1-Benzo[b]thiophen-6-yl, 1-Benzothiazol-5-yl, 1-Benzooxazol-5-yl, 1-Benzothiazol-6-yl, 3H-Benzotriazol-5-yl, 1H-indol-5-yl, 1H-indol-6-yl, 2,3-Dihydro-benzo[1,4]dioxin-6-yl, or 3,4-dihydro-2H-benzo[b][1,4]dioxepin-7-yl;
(m) R¹ represents thiophen-3-yl, thiophen-2-yl, furan-2-yl, furan-3-yl, pyridin-3-yl, pyridin-2-yl, benzofuran-2-yl, 2,3-dihydro-benzofuran-5-yl, benzo[b]thiophen-2-yl, benzo[b]thiophen-3-yl, quinolin-6-yl, furo[3,2-b]pyridin-2-yl, benzo[1,3]dioxol-5-yl, or 1H-indol-3-yl,
(n) R¹ represents phenyl substituted one or two times with a moiety selected from the group consisting of (C₁-C₆)alkyl, hydroxy, halo, (C₁-C₆)alkoxy, (C₁-C₄)alkylsulfonyl, (C₁-C₄)alkylsulfinyl, (C₁-C₄)alkylthio, aryl(C₁-C₆)alkoxy, trifluoromethyl, difluoromethyl, trifluoromethoxy, difluoromethoxy, phenyl, and halophenyl;
(o) R¹ represents 2-methyl phenyl, 3-methyl-phenyl, 4-methyl phenyl, 4-ethyl phenyl, 2,4-dimethyl phenyl, 3,4-dimethyl phenyl, 3-hydroxy phenyl, 4-hydroxy phenyl, 3,5-dimethyl-4-hydroxy phenyl, 2-fluoro phenyl, 3-fluoro phenyl. 4-fluoro phenyl, 2,4-difluoro phenyl, 3,4-difluorophenyl, 4-methyl 2-fluoro phenyl, 4-chloro phenyl, 2-methoxy phenyl, 3-methoxy phenyl, 4-methoxy phenyl, 4-methanesulfonyl phenyl, 4-methanesulfinyl phenyl, 4-methanesulfanyl phenyl, 4-trifluoromethyl phenyl, 4-trifluoromethoxy phenyl, 2-biphenyl, 4-biphenyl, 3-(4-fluorophenyl)phenyl, 4-benzyloxy phenyl; 3-Chloro-4-methoxy-phenyl, 3-fluoro-4-methoxy-phenyl, 4-fluoro-3-methoxy-phenyl, 4-Chloro-3-methoxy-phenyl;
(p) R¹ represents 2-methyl phenyl, 3-methyl-phenyl, 4-methyl phenyl, 4-ethyl phenyl, 2,4-dimethyl phenyl, 3,4-dimethyl phenyl, 3-hydroxy phenyl, 4-hydroxy phenyl, 3,5-dimethyl-4-hydroxy phenyl, 2-fluoro phenyl, 3-fluoro phenyl. 4-fluoro phenyl, 2,4-difluoro phenyl, 3,4-difluorophenyl, 4-methyl 2-fluoro phenyl, 4-chloro phenyl, 2-methoxy phenyl, 3-methoxy phenyl, 4-methoxy phenyl, 4-methanesulfonyl phenyl, 4-methanesulfinyl phenyl, 4-methanesulfanyl phenyl, 4-trifluoromethyl phenyl, 4-trifluoromethoxy phenyl, 2-biphenyl, 4-biphenyl, 3-(4-fluorophenyl)phenyl, or 4-benzyloxy phenyl;
(q) R¹ represents substituted thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, or thiomorpholinyl;
(r) R¹ represents thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, or thiomorpholinyl substituted one or two times with a moiety selected from the group consisting of halo, (C₁-C₆)alkyl, (C₁-C₆)alkoxy, and trifluoromethyl.
(s) R¹ represents thiophenyl, furanyl, pyridinyl substituted one or two times with a moiety selected from the group consisting of halo, (C₁-C₆)alkyl, (C₁-C₆)alkoxy, and trifluoromethyl.
(t) R¹ represents substituted benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzodioxine, benzodioxepine, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline;
(u) R¹ represents substituted benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline;
(v) R¹ represents benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzodioxine, benzodioxepine, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline substituted one or two times with a moiety selected from the group consisting of halo, (C₁-C₆)alkyl, (C₁-C₆)alkoxy, trifluoromethyl, acyl, and amino;
(w) R¹ represents benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline substituted one or two times with a moiety selected from the group consisting of halo, (C₁-C₆)alkyl, (C₁-C₆)alkoxy, and trifluoromethyl;
(x) R¹ represents benzofuranyl, 2,3 dihydro-benzofuranyl, furopyridinyl, benzothiophenyl, indolyl, benzodioxole, quinolinyl, benzoxazole, benzimidazole, benzothiophene, benzothiazole, indazole, benzoisoxazole, benzotriazole, benzodioxine, or benzodioxepine substituted one or two times with a moiety selected from the group consisting of halo, (C₁-C₆)alkyl, (C₁-C₆)alkoxy, trifluoromethyl, acyl, and amino;
(y) R¹ represents benzofuranyl, 2,3 dihydro-benzofuranyl, furopyridinyl, benzothiophenyl, indolyl, benzodioxole, quinolinyl, substituted one or two times with a moiety selected from the group consisting of halo, (C₁-C₆)alkyl, (C₁-C₆)alkoxy, and trifluoromethyl; or
(z) R¹ represents 5-chloro-benzofuran-2-yl, 5-methoxy benzofuran-2-yl, 7-methoxy benzofuran-2-yl, 7-fluoro benzofuran-2-yl, 5-fluoro benzofuran-2-yl, 5-chloro-7-fluoro benzofuran-2-yl, 2,2-difluoro-benzo[1,3]dioxol-5-yl, 6-chloro benzo(b)thiophen-2-yl, 4-chloro benzo(b)thiophen-2-yl, 4-trifluoromethyl benzo(b)thiophen-2-yl, 5-trifluoromethyl benzo(b)thiophen-2-yl, 6-trifluoromethyl benzo(b)thiophen-2-yl, 7-trifluoromethyl benzo(b)thiophen-2-yl, 4-fluoro benzo(b)thiophen-2-yl, 5-fluoro benzo(b)thiophen-2-yl, 7-fluoro benzo(b)thiophen-2-yl, 3-methyl-4-fluoro benzo(b)thiophen-2-yl, 3-methyl-7-fluoro benzo(b)thiophen-2-yl, 2-methyl-benzooxazol-6-yl, 2-methyl-benzothiazol-5-yl, 2-Amino-benzothiazol-5-yl, 3-Amino-benzo[d]isoxazol-6-yl, 2-Amino-benzothiazol-6-yl, 2-methyl-benzooxazol-5-yl, 2-Chloro-benzothiazol-6-yl, 2-trifluoromethyl-3H-benzoimidazol-5-yl, 3-Amino-benzo[d]isoxazol-5-yl, 2-methyl-3H-benzoimidazol-5-yl, 2-methyl-benzofuran-5-yl, 1-Acetyl-1H-indol-5-yl, 1-Acetyl-1H-indol-6-yl, 2-methyl-benzofuran-4-yl, 2-Chloro-benzothiazol-5-yl, 1,2-Dimethyl-1H-benzoimidazol-5-yl, or 2-methyl-benzofuran-6-yl;
(aa) R¹ represents 5-chloro-benzofuran-2-yl, 5-methoxy benzofuran-2-yl, 7-methoxy benzofuran-2-yl, 7-fluoro benzofuran-2-yl, 5-fluoro benzofuran-2-yl, 5-chloro-7-fluoro benzofuran-2-yl, 2,2-difluoro-benzo[1,3]dioxol-5-yl, 6-chloro benzo(b)thiophen-2-yl, 4-chloro benzo(b)thiophen-2-yl, 4-trifluoromethyl benzo(b)thiophen-2-yl, 5-trifluoromethyl benzo(b)thiophen-2-yl, 6-trifluoromethyl benzo(b)thiophen-2-yl, 7-trifluoromethyl benzo(b)thiophen-2-yl, 4-fluoro benzo(b)thiophen-2-yl, 5-fluoro benzo(b)thiophen-2-yl, 7-fluoro benzo(b)thiophen-2-yl, 3-methyl-4-fluoro benzo(b)thiophen-2-yl, or 3-methyl-7-fluoro benzo(b)thiophen-2-yl.
(bb) R² represents (C₁-C₆)alkyl, (C₃-C₇)cycloalkyl, aryl, substituted aryl, heterocycle, substituted heterocycle, (C₁-C₄)alkyl-(C₃-C₇)cycloalkyl, (C₁-C₄)alkyl-heterocycle, (C₁-C₄)alkyl-substituted heterocycle, (C₁-C₄)alkyl-aryl, (C₁-C₄)alkyl-substituted aryl, halo(C₁-C₆)alkyl, (C₁-C₄)alkyl-(C₁-C₆)alkoxy, nitro(C₁-C₆)alkyl, amino(C₁-C₆)alkyl, NH(C₁-C₄)alkylamine, N,N—(C₁-C₄)dialkylamine(C₁-C₄)alkyl-NH(C₁-C₄)alkylamine, or (C₁-C₄)alkyl-N,N—(C₁-C₄)dialkylamine;
(cc) R² represents (C₁-C₆)alkyl, (C₃-C₇)cycloalkyl, aryl, substituted aryl, heterocycle, substituted heterocycle, halo(C₁-C₆)alkyl, (C₁-C₄)alkyl-(C₁-C₆)alkoxy, nitro(C₁-C₆)alkyl, amino(C₁-C₆)alkyl, NH(C₁-C₄)alkylamine, or N,N—(C₁-C₄)dialkylamine;
(dd) R² represents (C₁-C₆)alkyl, (C₃-C₇)cycloalkyl, aryl, substituted aryl, heterocycle, substituted heterocycle, halo(C₁-C₆)alkyl, or (C₁-C₄)alkyl-(C₁-C₆)alkoxy;
(ee) R² represents (C₁-C₆)alkyl, (C₃-C₇)cycloalkyl, aryl, substituted aryl, halo(C₁-C₆)alkyl, or (C₁-C₄)alkyl-(C₁-C₆)alkoxy;
(ff) R² represents (C₁-C₆)alkyl;
(gg) R² represents methyl, ethyl, propyl, isopropyl, or butyl;
(hh) R² represents (C₃-C₇)cycloalkyl;
(ii) R² represents cyclopropyl;
(jj) R² represents aryl;
(kk) R² represents phenyl;
(ll) R² represents phenyl substituted one or two times with a moiety selected from the group consisting of (C₁-C₆)alkyl, (C₁-C₆)alkoxy, and halo;
(mm) R² represents 4-methyl phenyl, 4-methoxy phenyl, 3-methoxy phenyl, 4-fluoro phenyl, 3-fluoro phenyl, 2-fluoro phenyl, or 3,5-dimethyl phenyl;
(nn) R² represents 4-fluoro phenyl;
(oo) R² represents halo(C₁-C₆)alkyl;
(pp) R² represents (C₁-C₄)alkyl-(C₁-C₆)alkoxy;
(qq) R² represents methoxy methyl;
(rr) R³ represents represents (C₁-C₆)alkyl, halo(C₁-C₆)alkyl, (C₃-C₇)cycloalkyl, or aryl;
(ss) R³ represents represents (C₁-C₆)alkyl, halo(C₁-C₆)alkyl, or aryl;
(tt) R³ represents represents (C₁-C₆)alkyl;
(uu) R³ represents represents methyl, ethyl, propyl, isopropyl, or butyl;
(vv) R³ represents represents halo(C₁-C₆)alkyl; or
(ww) R³ represents represents phenyl.
(xx) R² and R³, together with the carbon atom to which they are attached, form a cyclohexyl, cyclopentyl, or pyranyl group; or
(yy) R² and R³, together with the carbon atom to which they are attached, form cyclohexyl, cyclopentyl, or pyran-4-yl.
(zz) R⁴ represents hydrogen, halo, amino, nitro, difluoromethyl, triflouromethyl, difluoromethoxy, triflouromethoxy, (C₁-C₆)alkyl, hydroxy(C₁-C₆)alkyl, (C₁-C₆)alkoxy, NH(C₁-C₄)alkylamine, N,N—(C₁-C₄)dialkylamine, NHCOR¹², NHSO₂R⁸, N(CH₃)SO₂R⁸, SO₂R⁹, or CHO;
(aaa) R⁴ represents hydrogen, halo, amino, nitro, (C₁-C₆)alkyl, hydroxy(C₁-C₆)alkyl, (C₁-C₆)alkoxy, NHCOR¹², NHSO₂R⁸, N(CH₃)SO₂R⁸, SO₂R⁹, or CHO;
(bbb) R⁴ represents halo, amino, nitro, (C₁-C₆)alkyl, hydroxy(C₁-C₆)alkyl, (C₁-C₆)alkoxy, NHCOR¹², NHSO₂R⁸, N(CH₃)SO₂R⁸, SO₂R⁹, or CHO;
(ccc) R⁴ represents hydrogen;
(ddd) R⁴ represents halo, amino, or nitro;
(eee) R⁴ represents fluoro, amino, or nitro;
(fff) R⁴ represents (C₁-C₆)alkyl, hydroxy(C₁-C₆)alkyl, or (C₁-C₆)alkoxy;
(ggg) R⁴ represents methyl, ethyl, hydroxymethyl, or methoxy;
(hhh) R⁴ represents NHCOR¹²;
(iii) R⁴ represents NHCOR¹², wherein R¹² represents methyl;
(jjj) R⁴ represents NHSO₂R⁸;
(kkk) R⁴ represents NHSO₂R⁸ wherein R⁸ represents (C₁-C₆)alkyl or aryl;
(lll) R⁴ represents NHSO₂R⁸ wherein R⁸ represents methyl, ethyl, propyl, isopropyl, or phenyl;
(mmm) R⁴ represents NHSO₂R⁸ wherein R8 represents methyl;
(nnn) R⁴ represents N(CH₃)SO₂R⁸;
(ooo) R⁴ represents. N(CH₃)SO₂R⁸ wherein R⁸ represents methyl;
(ppp) R⁴ represents SO₂R⁹;
(qqq) R⁴ represents SO₂R⁹ wherein R⁹ represents methyl; or
(rrr) R⁴ represents CHO.
(sss) R⁵ represents hydrogen, halo, hydroxyl, amino, difluoromethyl, triflouromethyl, difluoromethoxy, triflouromethoxy, or (C₁-C₆)alkyl;
(ttt) R⁵ represents hydrogen, halo, or hydroxyl;
(uuu) R⁵ represents hydrogen or fluoro;
(vvv) R⁵ represents hydrogen; or
(www) R⁵ represents fluoro.
(xxx) R⁶ represents hydrogen, halo, or (C₁-C₆)alkyl;
(yyy) R⁶ represents hydrogen, fluoro, or methyl;
(zzz) R⁶ represents hydrogen or fluoro;
(aaaa) R⁶ represents hydrogen or methyl; or
(bbbb) R⁶ represents hydrogen.
(cccc) R⁷ represents hydrogen, (C₁-C₆)alkyl, (C₃-C₇)cycloalkyl, (C₁-C₄)alkyl-CONH₂, COOH, (C₁-C₄)alkyl-COOH, or (C₁-C₄)alkyl-COOCH₃;
(dddd) R⁷ represents hydrogen, (C₁-C₆)alkyl, (C₁-C₄)alkyl-COOH;
(eeee) R⁷ represents hydrogen, (C₁-C₆)alkyl, CH₂—COOH or CH₂CH₂—COOH;
(ffff) R⁷ represents hydrogen, methyl, CH₂—COOH or CH₂CH₂—COOH;
(gggg) R⁷ represents hydrogen;
(hhhh) R⁷ represents methyl; or
(iiii) R⁷ represents CH₂—COOH or CH₂CH₂—COOH.

In addition, as yet another particular embodiment of the present invention, the compounds of Formula I have the following configuration

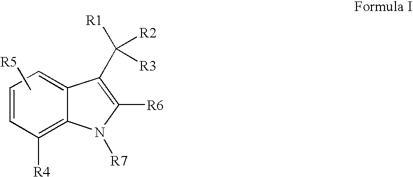

Compounds of Formula I can be chemically prepared, for example, by following the synthetic routes set forth in the Schemes below. However, the following discussion is not intended to be limiting to the scope of the present invention in any way. For example, the specific synthetic steps for the routes described herein may be combined in different ways, or with steps from different schemes, to prepare additional compounds of Formula I. Further, it should be recognized that the sequence in which the synthetic reactions take place is not implied and can be done in any fashion to achieve the desired final product. All substituents, unless otherwise indicated, are as previously defined. The reagents and starting materials are readily available to one of ordinary skill in the art. For example, certain reagents or starting materials can be prepared by one of ordinary skill in the art following procedures disclosed in Nordvall et al., J. Med. Chem. (1996), 39, 3269-3277; Chem. Rev. 1995, 95, 2457-2483; and J. Am. Chem. Soc., 122, 4280-4285 (2000). Other necessary reagents and starting materials may be made by procedures which are selected from standard techniques of organic and heterocyclic chemistry, techniques which are analogous to the syntheses of known structurally similar compounds, and the procedures described in the Preparations and Examples below, including any novel procedures. In addition, one of ordinary skill will appreciate that many of the necessary reagents or starting materials can be readily obtained from commercial suppliers.

Compounds of Formula I can be synthesized by coupling the appropriately substituted or unsubstituted indole with the appropriately substituted or unsubstituted carbinol according to procedures as generally described in Scheme I, below. Any subsequent modifications deemed necessary to produce the final product of Formula I, including but not limited to deprotection reactions, can be readily performed by one of ordinary skill in the art. The carbinols for use in the following procedures are either purchased from commercial suppliers, or synthesized as described in Schemes II-VI, below. The indoles for use in the following procedures are also either purchased from commercial suppliers, or synthesized in the manner as described in Schemes VII-IX.

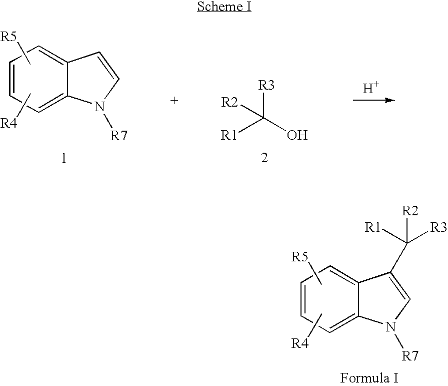

In Scheme I, the electrophilic aromatic substitution occurs by methods known in the art. For example, the appropriately substituted or unsubstituted indole, and the appropriately substituted or unsubstituted carbinol are first dissolved in a suitable solvent such as dichloromethane or acetic acid or methanol then treated with a suitable protic or Lewis acid such as trifluoroacetic acid, boron trifluoride etherate, hydrogen chloride or aluminum chloride. The reaction proceeds in anywhere from ten minutes to several days depending on the stability of the starting materials. The product of Formula I can then be isolated by normal phase chromatographic methods or recrystallization techniques commonly employed in the art.

Schemes II-IV provide procedures for the synthesis of carbinol reagents for use in the synthesis of compounds of Formula I.

Carbinols wherein R1 represents an aryl or substituted aryl group and R2 and R3 represent, for example, alkyl groups or aryl or substituted aryl groups may be synthesized according the procedures commonly known in the art and as described in Scheme II

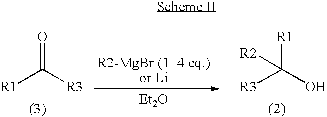

In Scheme II, secondary or tertiary carbinols are prepared by anion chemistry commonly used in the art. For example, one to four equivalents of an anion, such as a Grignard reagent or alkyl or aryl lithium species, is added to an electrophile of structure (3), such as an aldehyde, ketone, carboxylic acid or ester dissolved in a suitable solvent, such as diethyl ether or tetrahydrofuran, at temperatures ranging from −78° C. to room temperature. The reaction proceeds for about 1-24 hours. The product of structure (2) may be isolated by methods known in the art, such as a standard aqueous workup, and may or may not require purification via chromatography.

Carbinols wherein R1 represents a substituted aryl group and R2 and R3 represent, for example, alkyl groups may be synthesized according the procedures commonly known in the art and as described in Scheme II(a).

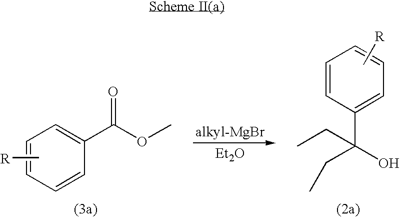

In Scheme II(a), a compound of general structure (3a) is first dissolved in ether and cooled to about 0° C. under an atmosphere or nitrogen. Structure (3a) is then treated with an alkylating agent, such as an alkyl-magnesium bromide, dropwise over about 10 minutes. The cooling bath is then removed and the reaction allowed to warn to ambient temperature The product of structure (2a) may be isolated by methods known in the art, such as a standard aqueous workup, and may then be purified via standard chromatography methods.

Carbinols wherein R1 represents a substituted aryl group may be synthesized according the procedures described in Scheme III.

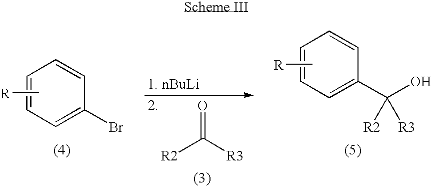

In Scheme III, the carbinol is prepared by conditions commonly employed in the art. For example, a substituted or unsubstituted aryl bromide of structure (4) (Wherein R represents an aryl substituent as described herein and above) is first dissolved in a suitable solvent such as diethyl ether or tetrahydrofuran, and cooled to about −78° C. Metal-halogen exchange occurs upon addition of alkyl lithium agent, such as n-butyl lithium, followed by quenching of the anion by addition of the appropriate electrophile of structure (3). The reaction proceeds for about 1-24 hours. The product may be isolated by methods known in the art, such as a standard aqueous workup, and may or may not require purification via chromatography.

Carbinols wherein, for example, R1 represents a substituted or unsubstituted alkyne and R2 and R3 represent straight or branched alkyl or cycloalkyl groups may be synthesized according the procedures described in Scheme IV

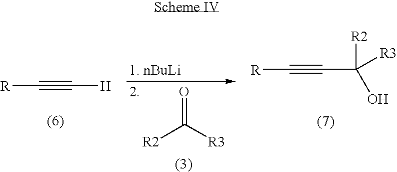

In Scheme IV, the carbinol is prepared by conditions commonly employed in the art. For example, a substituted alkyne of structure (6) (where in R represents a substituent) is first dissolved in a suitable solvent such as diethyl ether or tetrahydrofuran, and cooled to about −78° C. Deprotonation occurs upon addition of alkyl lithium agent, such as n-butyl lithium, followed by quenching of the anion by addition of the appropriate electrophile (3). The reaction proceeds for about 1-24 hours. The product of structure (7) may be isolated by methods known in the art, such as a standard aqueous workup, and may or may not require purification via chromatography.

Carbinols wherein, for example, R1 represents a substituted aryl group and R3 represents hydrogen may be synthesized according the procedures described in Scheme V.

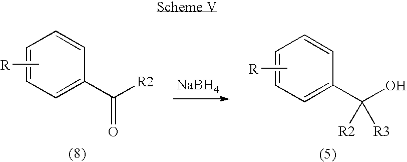

In Scheme V, the carbinol is prepared by reduction conditions commonly employed in the art. For example, a ketone of structure (8) is first dissolved in a suitable solvent, such as tetrahydrofuran, and a reducing agent, such as sodium borohydride or lithium aluminum hydride, is then added at 0° C. to room temperature. The reaction proceeds for about 1-24 hours. The product of structure (5) is isolated by methods known in the art, such as a standard aqueous workup, and may be purified via chromatography.

Carbinols wherein, for example, R1 represents a substituted or unsubstituted fused heterocycle and R2 and R3 represent straight or branched alkyl, or a cycloalkyl, may be synthesized according the procedures described in Scheme VI.

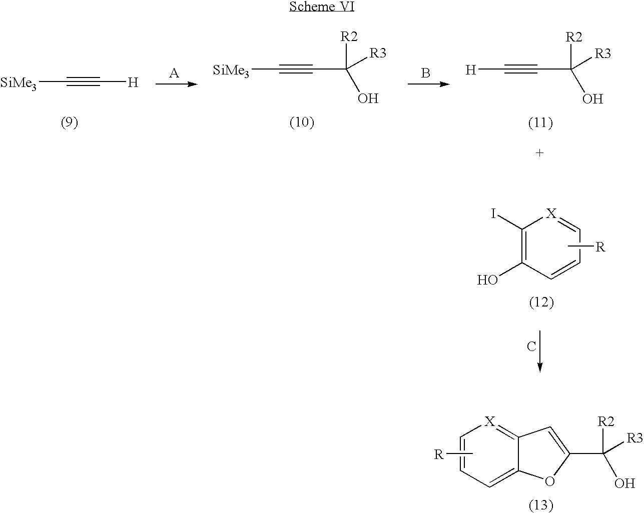

In Scheme VI, step A, the carbinol is prepared according to Scheme IV. Subsequent deprotection in step B typically entails dissolution in a suitable solvent such as an alcohol, water, diethyl ether, or tetrahydrofuran, followed by addition of base, for example, cesium or potassium carbonate, or cesium or potassium fluoride at 0° C. to room temperature. The reaction proceeds for about 1-24 hours. The product of structure (11) is isolated by methods known in the art, such as a standard aqueous workup, and may be purified via chromatography. The coupling and cyclization to afford compounds of structure (13) (where R represents a fused heterocycle substituent) proceed according to the published route found in Nordvall et al., J. Med. Chem. (1996), 39, 3269-3277.

The indoles for use in the synthesis of compounds of Formula I can be obtained from commercial sources or may be prepared according to procedures as described in Schemes VII-IX, below.

Indoles wherein R4 represents, for example, an amino, NHSO₂R⁸, N-acyl, or alkylamine group may be synthesized according to the procedures described in Scheme VII.

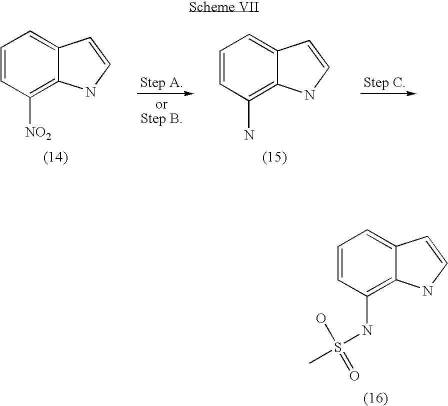

In Scheme VII, Step A or B, the nitro reduction occurs by methods commonly employed in the art. For example, in Step A the appropriate nitro indole of structure (14) is dissolved in a suitable solvent such as ethanol, and is reduced by hydrogenation conditions, such as Pd/C and a hydrogen source like hydrogen gas or ammonium formate. The reaction may occur at room temperature to refluxing conditions and the product of structure (15) may be isolated by standard techniques such as filtration or standard aqueous workup. Alternatively, in Step B, structure (14) is treated with a reducing agent, such as tin chloride dihydrate, at elevated temperatures. The reaction may proceed for about 1-24 hours. The product (structure (15)) may be isolated by methods known in the art, such as a standard aqueous workup, and be purified via chromatography.

In Scheme VII, Step C, the aniline intermediate of structure (15) is dissolved in dichloromethane and pyridine, then methanesulfonyl chloride is added. The reaction is stirred at room temperature for a minimum of six hours. The product of structure (16) may be isolated by methods known in the art, such as a standard aqueous workup, and may be purified via standard chromatography techniques.

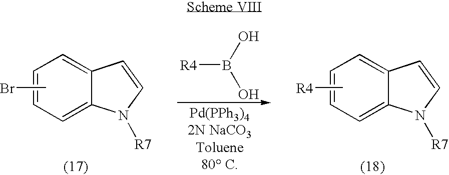

In Scheme VII, compounds of structure (11) are prepared according to standard Suzuki conditions as detailed in Chem. Rev. 1995, 95, 2457-2483.

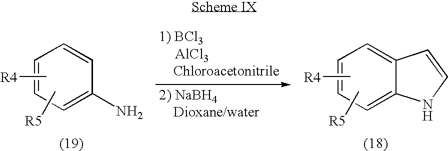

In Scheme IX, the appropriately substituted aniline of structure (19) is dissolved in a suitable solvent such as toluene or benzene, cooled to about 0° C. and pretreated with boron trichloride for about 5-30 min. Chloroacetornitrile is added followed by aluminum trichloride and the reaction is heated to the reflux temperature of the solvent for between 10 min. to 2 days. The reaction is cooled and worked up using standard methods known in the art. The residue is then dissolved in a dioxane/water mixture and sodium borohydride is added. This is heated to reflux for about 4-24 hrs. The product of structure (18) is isolated by methods known in the art, such as a standard aqueous workup, and may be purified via standard chromatography techniques.

Particular compounds of Formula I can be synthesized following the general procedures as described in Scheme X-XXI, below. Again, any subsequent modifications deemed necessary to produce the final product of Formula I, including but not limited to deprotection reactions, can be readily performed by one of ordinary skill in the art. The carbinols for use in the procedures of Schemes X-XXI are either purchased from commercial suppliers, or synthesized as described in Schemes II-VI, above. The indoles for use in the following procedures are also either purchased from commercial suppliers, or synthesized in the manner as described in Schemes VII-IX, above.

Compounds of Formula I wherein, for example, at least one of R1 and R2 represents an aryl group substituted with SO₂R⁹ or SOR⁹, and R4 represents NHSO₂R⁸ can be synthesized according to procedures as described in Scheme X.

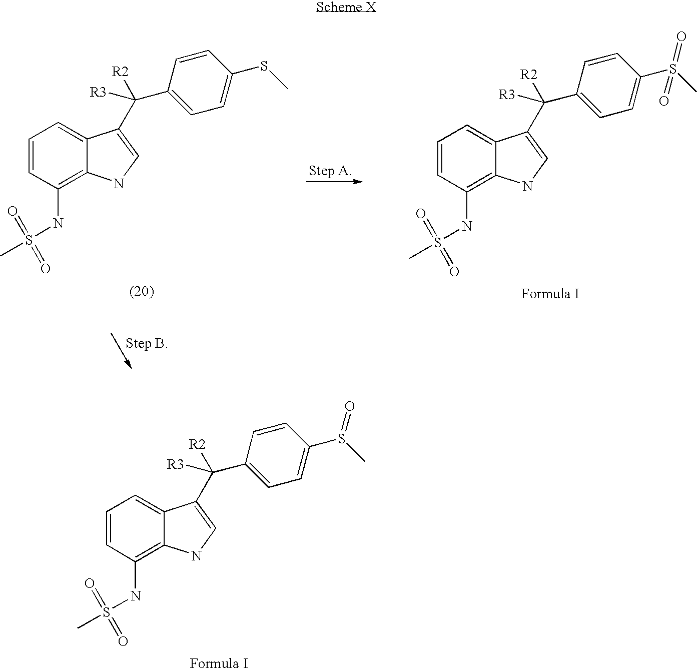

In Scheme X, sulfonyls and sulfinyls of Formula I are synthesized employing conditions as described in J. Am. Chem. Soc., 122, 4280-4285 (2000) using the sulfide of structure (20) (prepared for example according to procedures described in Scheme I employing the appropriately substituted indole (Scheme VII) and the appropriately substituted carbinol (Scheme II))

Compounds of Formula I wherein R7 represents a substituent other tan hydrogen (for example wherein R7 represents (C1-C4)alkyl-COOH or (C1-C4)alkyl-COOCH₃) can be synthesized according to general procedures as described in Scheme XI.

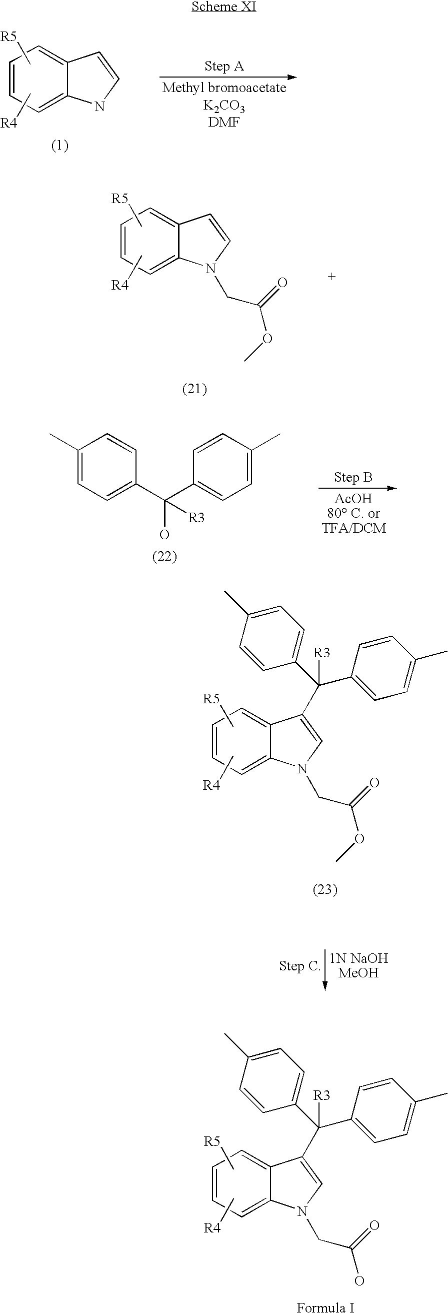

In Scheme XI, Step A, the appropriately substituted or unsubstituted indole is N-alkylated under conditions commonly employed in the art. For instance, an appropriately substituted indole is dissolved in a suitable solvent, such as tetrahydrofuran, diethyl ether, or dimethylformamide, and treated with a base, such as cesium or potassium carbonate, sodium hydride, and the like, and reacted with an electrophile such as methyl bromoacetate. The product can be obtained by methods commonly known in the art. In Step B, the indole is coupled according to conditions as described in Scheme I. In Step C, hydrolysis occurs under standard hydrolysis conditions. The ester is dissolved in a suitable solvent, such as methanol or ethanol, and treated with a base, such as sodium hydroxide. The reaction proceeds for about 1-24 hours at room temperature or elevated temperatures. The product can be obtained through acid/base workup or strong anion exchange technology commonly employed in the art to provide the compound of Formula I.

Scheme XII provides procedures for the synthesis of compounds of Formula I wherein R1 and R2 represent, for example, aryl or substituted aryl groups and R 7 represents alkyl-CONH₂:

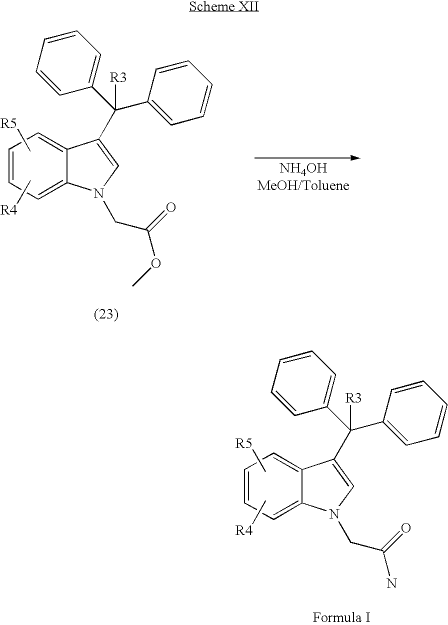

In Scheme XII, amidation of the ester of structure (23) (Scheme XI, Step A and B) occurs via conditions commonly employed in the art. For instance, the ester is dissolved in a suitable solvent, such as toluene, methanol, ethanol, or water, and an ammonia source is added, such as ammonium hydroxide or ammonia gas. The reaction proceeds at room or elevated temperatures for about 1-24 hours. The product can be isolated by standard methods; such as filtration or aqueous workup.

Scheme XIII provides procedures for the synthesis of compounds of Formula I wherein, for example, R1 and R2 represent substituted aryl groups and R7 represents hydrogen or a benzenesulfonyl group.

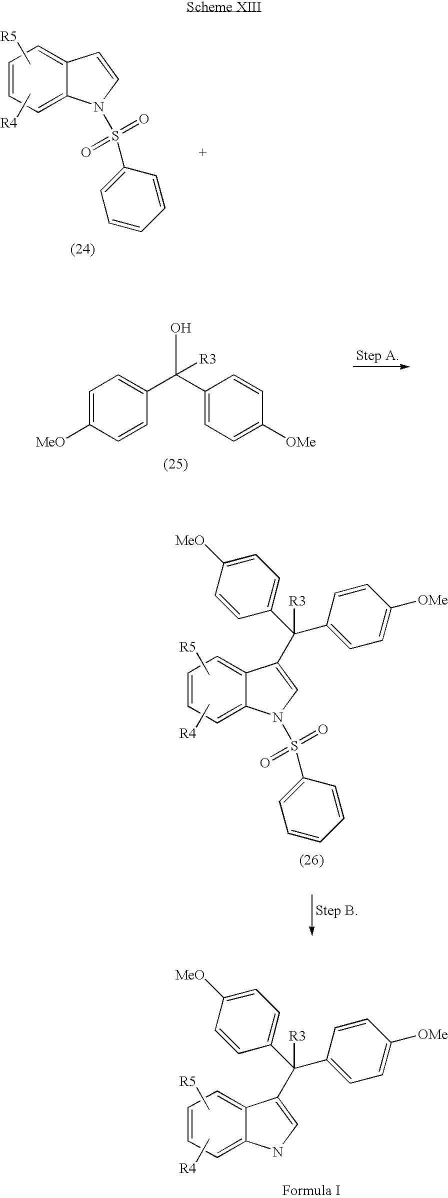

In Scheme XIII, Step A, the coupling conditions are as described in Scheme I. To the 1-benzenesulfonyl indole (24) and a dimethoxy benzhydrol (structure (25)) dissolved in dichloromethane, is added boron triflouride etherate. In Step B, the compound of structure (26) is deprotected using conditions commonly employed in the art. In general, the protected indole, such as 1-benzene sulfonyl indole, is dissolved in a suitable solvent, such as tetrahydrofuran, methanol, ethanol, or water, and reacted with a nucleophilic agent, such as tetrabutyl ammonium fluoride or sodium hydroxide. The product of Formula I can then be isolated by methods commonly employed in the art such as flash chromatography eluting with a suitable eluent such as toluene.

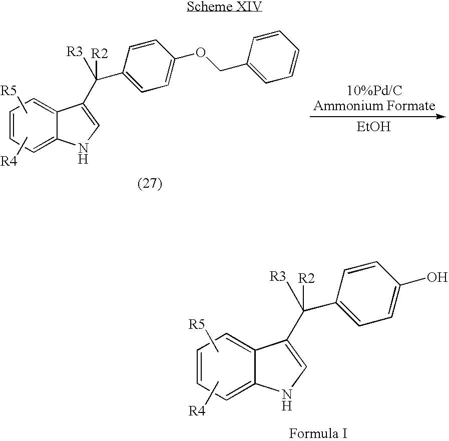

In Scheme XIV, phenols of Formula I are prepared using methods commonly employed in the art. For example, the benzyl ether derivative of structure (27) (prepared, for example, from the appropriately substituted indole (Scheme VII) and the appropriately substituted carbinol (Scheme II) according to Scheme I) is treated under hydrogenation condition commonly employed in the art and as generally described in Scheme VII. The product of Formula I can then be purified by standard methods such as flash chromatography, eluting with a suitable eluent.

Scheme XV provides yet additional procedures for the synthesis of compounds of Formula I wherein, for example, R1 and R2 represent aryl or substituted aryl groups.

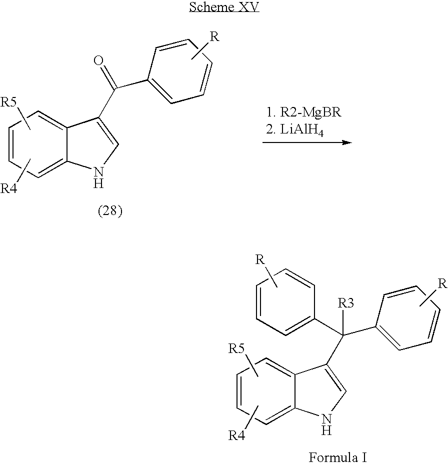

Briefly, a substituted or unsubstituted phenyl-(1H-indol-3-yl)-methanone is dissolved in a suitable solvent such as THF and stirred at ambient temperature under nitrogen. To this solution a phenyl magnesium bromide derivative is added dropwise. After addition the reaction is heated to reflux for about 2 hrs. The reaction is then cooled to ambient temperature and lithium aluminum hydride is added and the reaction mixture stirred for about 12 hrs. at about 50° C. The product of Formula I (wherein R represents aryl substituents as described herein and above) may be obtained by methods known in the art, such as aqueous workup and purified using standard methods such as normal phase chromatography.

Scheme XVI provides procedures for the synthesis of compounds of Formula I wherein, for example, R4 represents NH(C₁-C₄)alkylamine or N,N—(C₁-C₄)dialkylamine.

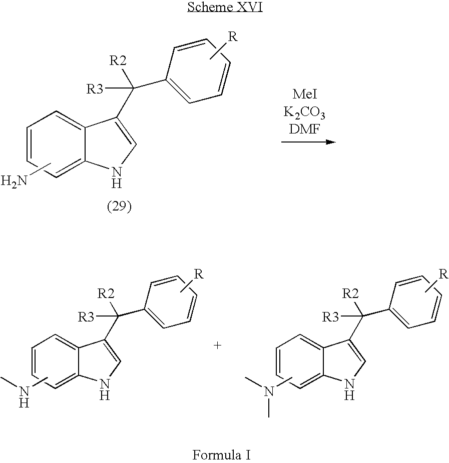

In Scheme XVI, the aniline nitrogen of structure (29), prepared for example as described in Scheme VII, is alkylated using procedures known in the art. For example, the aniline is first dissolved in a suitable solvent such as DMF then a suitable base, such as potassium carbonate, is added followed by the alkylating agent. The reaction is stirred at ambient temperature under a nitrogen atmosphere. The products of Formula I (wherein R represents aaryl substituents as described herein and above) may be obtained by methods known in the art, such as aqueous workup and normal phase chromatography.

Scheme XVII provides general procedures for the synthesis of compounds of Formula I wherein, for example, R2 represents nitro(C₁-C₆)alkyl.

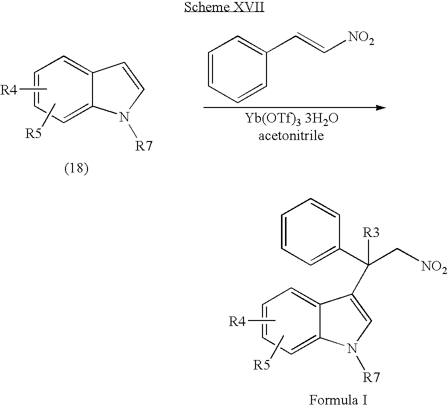

In Scheme XVII, the nitrostyrene is coupled to the appropriately substituted or unsubstituted indole of structure (18) by dissolving each in a suitable solvent, such as acetonitrile, and adding a suitable lewis acid, such as ytterbium triflate and heating between 0 and 100° C. for between 1 to 36 hrs. The product of Formula I may be obtained by methods known in the art, such as aqueous workup and normal phase chromatography.

Scheme XVII provides procedures for the synthesis of compounds of Formula I wherein, for example, R7 represents a carboxyl containing group.

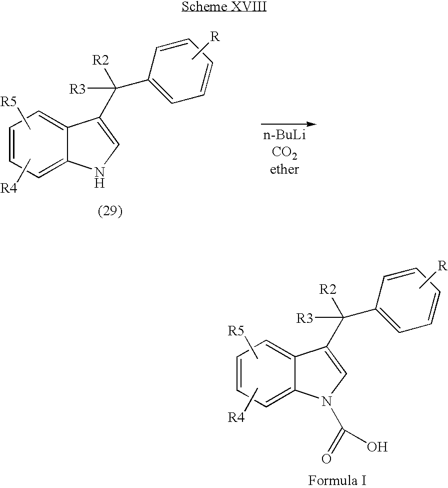

In Scheme XVIII, the appropriately substituted or unsubstituted indole of structure (29) is dissolved in an appropriate solvent, such as ether or THF, followed by the addition of an appropriate base, such as n-butyl lithium or sodium hydride, at between −78 and 0° C. After about 10 to 240 min. a suitable electrophile, such as carbon dioxide, is added and the reaction kept at between −78 and 0° C. for about 1-24 hrs. The product of Formula I (wherein R represents aryl substituents as described herein and above) may be obtained by methods known in the art, such as aqueous workup and normal phase chromatography.

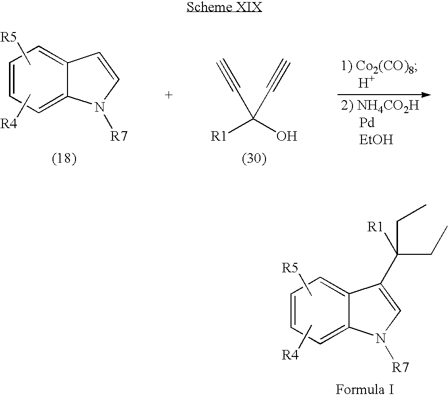

In Scheme XIX, the appropriately substituted carbinol is dissolved in a suitable solvent such as dichloromethane and stirred at ambient temperatures under an atmosphere of nitrogen. Dicobaltoctacarbonyl is added as the solid and the reaction continued until all gas evolution has ceased. The reaction is worked up using standard methods known in the art. The residue is then dissolved in ethanol and ammoniumformate along with a catalytic amount of paladium is added. This is heated to reflux for about 4-24 hrs. The product is isolated by methods known in the art, such as a standard aqueous workup, and may be purified via standard chromatography techniques.

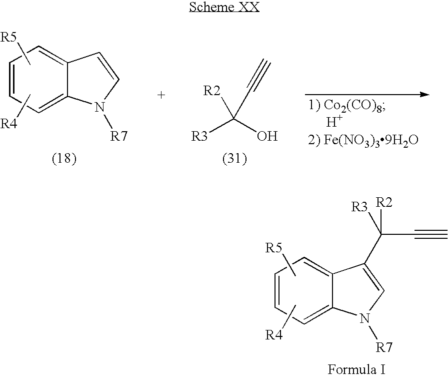

In Scheme XX, the appropriately substituted carbinol is dissolved in a suitable solvent such as dichloromethane and stirred at ambient temperatures under an atmosphere of nitrogen. Dicobaltoctacarbonyl is added as the solid and the reaction continued until all gas evolution has ceased. The reaction is worked up using standard methods known in the art. The residue is then dissolved in ethanol and iron(III)nitrate nonahydrate is added as the solid and the reaction stirred until all gas evolution has ceased. The product is isolated by methods known in the art, such as a standard aqueous workup, and may be purified via standard chromatography techniques.

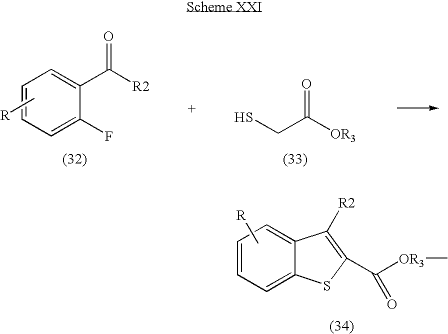

In Scheme XXI, a thioglycolate is dissolved in a suitable solvent, such as dimethylformamide, dimethylsulfoxide, or tetrahydrofuran, and treated with a base, such as triethylamine or sodium hydride. To this is added the appropriately substituted fluorophenylketone at room temperature and the reaction continues or is heated to 50-75° C. from 0-12 hours. The product of structure (34) is isolated by methods known in the art, such as a standard aqueous workup, and may be purified via standard chromatography techniques. This product may then be used in the synthesis of carbinol reagents using methods described herein and above.

Scheme XXII provides an alternative synthesis of compounds of Formula I wherein R1 represents a substituted aryl and R2 or R3 represents a cycloalkyl group.

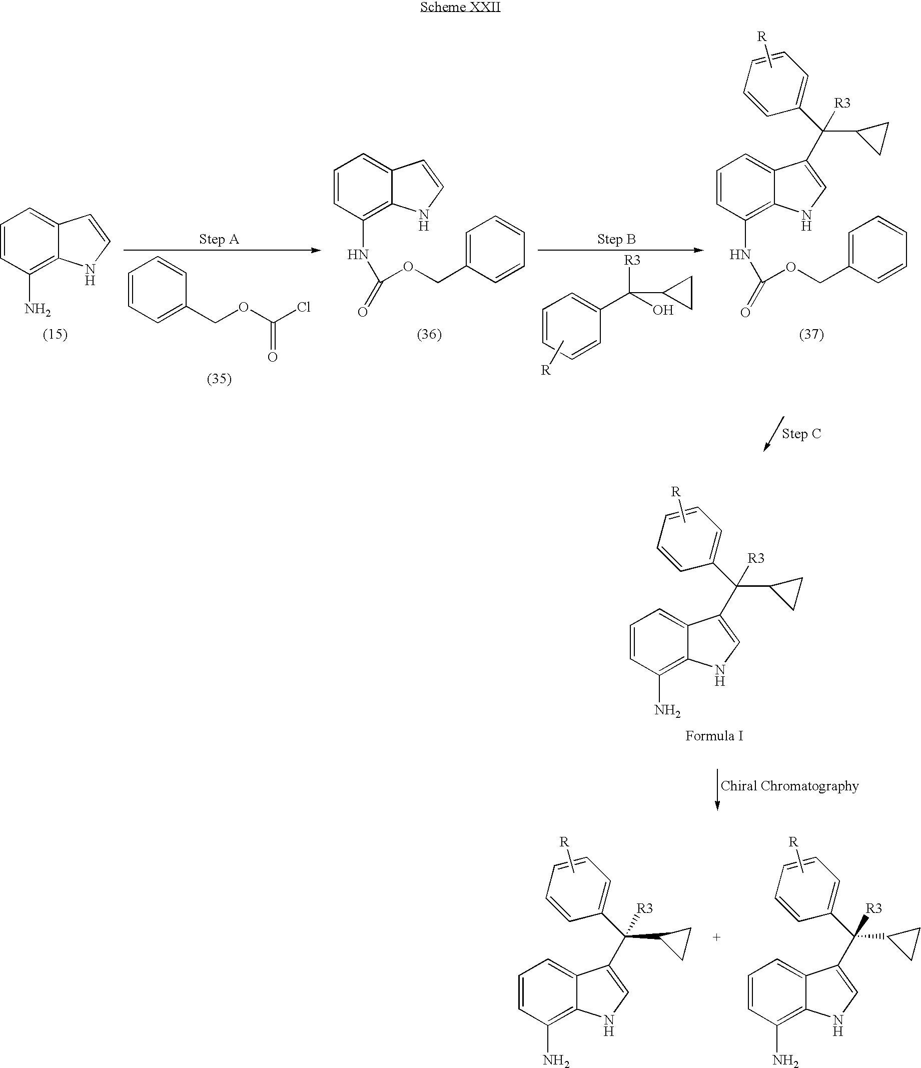

In Scheme XXII, Step A, the indole aniline of structure (15) is first dissolved in a suitable solvent, such as water and methanol, then cooled to 0° C. in a saltwater/ice bath. Sodium Carbonate is then added and the resulting slurry is stirred for about 5 minutes. A suitable nitrogen protecting group, such as benzy chloroformate (35) is then added and the mixture is stirred for about 30 minutes at 0° C. The reaction is then concentrated followed by extraction with a suitable solvent such as dichloromethane. The organics may then be dried (MgSO₄), filtered, and concentrated to provide the carbamate of structure (36).

In Step B, the carbamate of structure (36) and a suitable carbinol are dissolved in a suitable solvent such as dichloromethane. TFA is then added and the resulting solution is stirred for about 30 minutes ast room temperature. The reaction s is quenched with a suitable agent, such as saturated aqueous NaHCO₃. The aqueous layer may then be extracted with dichloromethane and the combined organics dried (MgSO₄), filtered, and concentrated to provide the compound of structure (37) (where R represents an aryl substituent).

In Scheme XXII, Step C, the coupled carbamate of structure (37) is deprotected by first dissolving in a suitable solvent such as ethanol, then reducing under standard conditions, such as addition of Pd/C (10 Wt. %) followed by hydrogenation at 40 psi and 40° C. overnight. The reaction is then cooled to about room temperature, the catalyst removed by filtration, and the filtrates concentrated to provide the compound of Formula I as a racemic mixture. The racemic mixture may then be separated by chiral chromatography techniques such as column chromatography eluting with a suitable eluent such as 20%/IPA/Hepatane (0.1% DMEA) (0.6 ml/min.)

The separated aniline isomers of Formula I may then be converted to the corresponding methansulfonamides according to procedures as described in Scheme VII, Step C, supra.

Determination of Biological Activity:

To demonstrate that compounds of the present invention have affinity for steroid hormone nuclear receptors, and thus have the capacity to modulate steroid hormone nuclear receptors, soluble MR and GR binding assays are performed. All ligands, radioligands, solvents, and reagents employed in the binding assays are readily available from commercial sources, or can be readily synthesized by the ordinarily skilled artisan.

Mineralocorticoid Receptor Binding Assay (Method 1):

The full length human MR gene is cloned from a human kidney or human brain cDNA library. Briefly, using synthetic oligonucleotide primers (Eli Lilly and Company, Indianapolis) directed to nucleotides 20-54 and 3700-3666 of the human MR, polymerase chain reaction (PCR) is performed under standard conditions using a human cDNA library. The PCR reaction is performed in a final volume of 50 μl containing about 1 μl of a 50× stock solution of polymerase; about 1 μl of a 50× stock solution of dNTP; about 5 μl of an appropriate PCR buffer; about 1 μl of each primer; about 5 μl of a H. kidney or H. brain cDNA library; and about 36 μl of water. The reaction is allowed to denature for about 30 seconds at 95 degrees Celsius, anneal for about 30 seconds at 55 degrees Celsius, and extend for about 5 minutes at 72 degrees Celsius, the sequence being repeated for a total of about 35 cycles. The desired PCR product (3.68 Kb) is confirmed by gel electrophoresis and subsequently cut from the gel and stored at about −20 degrees Celsius until extraction. To extract the cDNA product from the agarose gel, the QIAEX II Gel Extraction protocol (QIAGEN, Inc.) is employed according to the manufacturer's instructions. Following extraction, the MR cDNA is cloned into an appropriate cloning vector (Zero Blunt TOPO PCR Cloning Kit (Invitrogen, Inc.) and a pAcHLT-baculovirus transfer vector (B.D./Pharminogen), then expressed in SF9 insect cells, essentially according to manufacturer's instructions. Sf9 cells are grown at a scale where gram quantity cell pellets are obtained for subsequent use in the MR binding assay. Harvested cell pellets are lysed by repeated freeze-thaw cycles (about 4) in a suitable lysis buffer then centrifuged at about 1×10³ G (with the supernatant being saved for future assays).

MR binding assays are performed in a final total volume of about 250 μl containing about 20-25 μg of protein and 0.5 nM of [³H]-aldosterone plus varying concentrations of test compound or vehicle. The assay binding buffer consists of 30 mM sodium molybdate, 30 mM of TRIS-HCl, 5 mM sodium phosphate, 5 mM sodium pyrophosphate, and about 10% glycerol, pH=7.5.

Briefly, assays are prepared at RT in 96-well Falcon 3072 plates, each well containing 210 μl of binding buffer, 10 μl of [³H]-aldosterone, 10 μl of test compound/vehicle, and 20 μl of the resuspended receptor protein extract. Incubations are carried out at 4 degrees Celsius with shaking for about 16 hours. 200 μl aliquots of each incubation are filtered onto Millipore HA 0.45 micron 96-well filter plates, pre-moistened with cold 30 mM TRIS-HCl. The filter plates are suctioned dry with vacuum and immediately washed 3× with cold 30 mM TRIS-HCl. The plates are then punched out and the amount of receptor-ligand complex is determined by liquid scintillation counting using 4 ml of Ready Protein Plus™ liquid scintillation cocktail.

IC₅₀ values (defined as the concentration of test compound required to decrease [³H]-aldosterone binding by 50%) are then determined. Ki values for each respective test compound can then be calculated by application of the Cheng-Prusoff equation as described in Cheng et al., Relationship Between The Inhibition Constant (Ki) and The Concentration of Inhibitor Which Causes 50% Inhibition (IC₅₀) of an Enzymatic Reaction, Biochem. Pharmacol., 22: 3099-31088; (1973).

Glucocorticoid Receptor Binding Assay (Method 1):

To demonstrate the GR modulating potency of compounds of the present invention the following source of glucocorticoid receptor is employed. A549 human lung epithelial cells (ATCC) are grown at a scale where gram quantity cell pellets are obtained. Harvested cell pellets are washed twice in cold phosphate buffered saline, centrifuged, and resuspended in cold assay binding buffer. The assay binding buffer consists of 10% glycerol, 50 mM Tris-HCl (pH7.2), 75 mM sodium chloride, 1.5 mM magnesium chloride, 1.5 mM EDTA, and 10 mM sodium molybdate. Cell suspensions were lysed via sonication, centrifuged, and the “extract” supernatant is snap frozen and stored at −80 C until needed.

GR binding assays are performed in a final volume of 140 ul containing 50-200 ug of A549 cell extract and 1.86 nM [³H]-dexamethasone (Amersham) plus varying concentrations of test compound or vehicle. Briefly, assays are prepared at RT in 96-well Fisher 3356 plates, each well containing 100 ul of A549 cell extract, 20 ul of [³H]-dexamethasone, and 20 ul of test compound/vehicle. Incubations are carried out at 4 degrees Celsius for 16 hours. After incubation, 70 ul of 3× dextran-coated charcoal solution is added to each reaction, mixed, and incubated for 8 minutes at RT. 3×-dextran-coated charcoal solution consists of 250 ml assay binding buffer, 3.75 g Norit A charcoal (Sigma), and 1.25 g dextran T-70 (Amersham). Charcoal/unbound radioligand complexes are removed by centrifugation of the plate and 140 ul of supernatant from each well is transferred to another 96 well Optiplate (Packard Instruments). 200 ul of Microscint-20 scinillant (Packard Instruments) is added to each well and amount of receptor bound radioligand is determined using Packard Instruments TopCount instrument.

IC₅₀ values, defined as the concentration of test compound required to decrease [³H]-dexamethasone binding by 50%, are then determined. Ki values for each respective test compound can then be calculated by application of the Cheng-Prusoff equation as described in Cheng et al., Relationship Between The Inhibition Constant (Ki) and The Concentration of Inhibitor Which Causes 50% Inhibition (IC₅₀) of an Enzymatic Reaction, Biochem. Pharmacol., 22: 3099-31088; (1973).

Alternative Binding Assay Protocol for MR, GR, AR, and PR (Method 2):

Cell lysates from 293 cells overexpressing human GR (glucocorticoid receptor), AR (androgen receptor), MR (mineralocorticoid receptor) or PR (progesterone-receptor) are used for competition binding assays to determine Ki values for test compounds. Briefly, competition binding assays are run in a buffer containing 20 mM Hepes, pH 7.6, 0.2 mM EDTA, 75 mM NaCl, 1.5 mM MgCl2, 20% glycerol, 20 mM sodium molybdate, 0.2 mM DTT, 20 ug/ml aprotinin and 20 ug/ml leupeptin, using either 0.3 nM ³H-dexamethasone for GR binding, 0.36 nM ³H-methyltrienolone for AR binding, 0.25 nM ³H-aldosterone for MR binding, or 0.29 nM ³H-methyltrienolone for PR binding and either 20 ug 293-GR lysate, 22 ug 293-AR lysate, 20 ug 293-MR lysate or 40 ug 293-PR lysate per well. Competing compounds are added at various concentraions in half-log increments. Non-specific binding is determined in the presence of 500 nM dexamethasone for GR binding, 500 nM aldosterone for MR binding, or 500 nM methyltrienolone for AR and PR binding. The binding reaction (140 μl) is incubated for overnight at 4° C., then 70 μl of cold charcoal-dextran buffer (containing per 50 ml of assay buffer, 0.75 g of charcoal and 0.25 g of dextran) is added to each reaction. Plates are mixed 8 minutes on an orbital shaker at 4° C. Plates are then centrifuged at 3,000 rpm at 4° C. for 10 minutes. An aliquot of 120 μl of the mix is transferred to another 96-well plate and 175 μl of Wallac Optiphase “Hisafe 3” scintillation fluid is added to each well. Plates are sealed and shaken vigorously on an orbital shaker. After an incubation of 2 hrs, plates are read in a Wallac Microbeta counter. The data is used to calculate an IC₅₀ and % Inhibition at 10 μM. The K_{d} for ³H-dexamethasone for GR binding, ³H-methyltrienolone for AR binding, ³H-aldosterone for MR binding, or ³H-methyltrienolone for PR binding, is determined by saturation binding. The IC₅₀ values for compounds are converted to Kᵢ using Cheng-Prusoff equation and the K_{d} determined by saturation binding assay.

Binding assay protocols for PR, AR, and ER, similar to those described above for MR and GR, can be readily designed by the ordinarily skilled artisan. U.S. Pat. No. 6,166,013 provides examples of such protocols. Representative compounds of the present invention have a Ki in the MR or GR binding assay of ≦50 μM. Table I (see infra.) provides MR and GR binding data for a representative sample of the exemplified compounds of the present invention.

To demonstrate the ability of compounds of the present invention to modulate the activity of a steroid hormone receptor (i.e. either agonize, antagonize, partially agonize, or partially antagonize), bioassays are performed which detect modulation of target gene expression in cells transiently transfected with a nuclear receptor protein and a hormone response element-reporter gene construct. The solvents, reagents, and ligands employed in the functional assay are readily available from commercial sources, or can be synthesized by one of ordinary skill in the art.

Functional Assay of Mineralocorticoid Receptor Modulation (Method 1):

For the MR transient transfection assay, COS-7 cells are transfected with full length human MR and a 2×GRE-luciferase gene construct. Following transfection, the ability of test compounds to modulate expression of the luciferase reporter gene product is monitored. Briefly, on day one, COS cells are harvested from cell culture plates using standard procedures such as treatment with Trypsin-EDTA (GIBCO BRL). Culture medium is then added to the cells and the cell-medium mixture is plated in 96-well plates coated with poly-(d)-lysine (approximately 3×10⁴ cells/well). Cells are grown for about 4 hours then transfected with Fugene-6 reagent with plasmids containing human MR, previously cloned into pc.DNA 3.1 expression vector, and 2×GRE-reporter gene construct (GRE-luciferase), previously cloned into pTAL-luc vector. Transfection is carried out in DMEM with 5% fetal calf serum, charcoal treated. 24 hours later cells are exposed to various concentrations of aldosterone in the presence and absence of test compound and incubated for an additional 24 hours. The reaction is terminated by the addition of lysis buffer followed by luciferin (luciferase substrate). Luciferase expression, as an indicator of ligand induced MR transactivation, is monitored by chemiluminescence measured using a microtiter plate luminometer (MLX). The kinetic inhibition constant (K_{b} or Kₚ) can then be determined by analysis of dose-response curves for aldosterone, in the presence and absence of test compound, using standard techniques.

Alternative Functional Assay for MR, GR, PR, and AR Activity (Method 2):

Human embryonic kidney hEK293 cells are co-transfected using Fugene. Briefly, the reporter plasmid containing two copies of GRE (glucocorticoid response element ^{5′}TGTACAGGATGTTCT³) and TK promoter upstream of the luciferase reporter cDNA, is transfected with a plasmid constitutively expressing either human glucocorticoid receptor (GR), human mineralocorticoid receptor (MR), or human progesterone receptor (PR), using viral CMV promoter. The reporter plasmid containing two copies of probasin ARE (androgen response element ^{5′}GGTTCTTGGAGTACT^{3′}) and TK promoter upstream of the luciferase reporter cDNA, is transfected with a plasmid constitutively expressing human androgen receptor (AR) using viral CMV promoter. Cells are transfected in T150 cm² flasks in DMEM media with 5% charcoal-stripped Fetal Bovine Serum (FBS). After a overnight incubation, transfected cells are trypsinized, plated in 96 well dishes in DMEM media containing 5% charcoal-stripped FBS, incubated for 4 h and then exposed various concentrations of test compounds in half log increments. In the antagonist assays low concentrations of agonist for each respective receptor are added to the media (0.25 nM dexamethosone for GR, 0.3 nM of methyltrienolone for AR, 0.05 nM of progesterone for PR and 0.05 nM aldosterone). After 24 h of incubations with compounds, cells are lysed and luciferase activity is determined. Data is fit to a 4 parameter-fit logistics to determine EC50 values. The % efficacy is determined versus maximum stimulation obtained with 100 nM methyltrienolone for AR assay, with 30 nM progesterone for PR assay, with 30 nM aldosterone for MR assay and with 100 nM dexametasone for GR assay.

TABLE I
Mineralocorticoid and Glucocorticoid
Receptor Binding Assay Values

| Example No. | MR Ki (nM) Method 1 | GR Ki (nM) Method 1 |
|---|---|---|
| 54 | +++ | +++ |
| 55 | +++ | +++ |
| 57 | +++ | +++ |
| 59 | +++ | +++ |
| 1 | +++ | +++ |
| 2 | +++ | +++ |
| 58 | +++ | +++ |
| 56 | +++ | +++ |
| 62 | +++ | +++ |
| 61 | +++ | +++ |
| 60 | +++ | +++ |
| 3 | +++ | +++ |
| 5 | +++ | +++ |
| 4 | +++ | +++ |
| 63 | +++ | +++ |
| 64 | +++ | +++ |
| 6 | +++ | +++ |
| 8 | +++ | +++ |
| 7 | +++ | +++ |
| 45 | +++ | +++ |
| 65 | +++ | +++ |
| 66 | +++ | +++ |
| 67 | +++ | +++ |
| 69 | +++ | +++ |
| 70 | +++ | +++ |
| 71 | +++ | +++ |
| 68 | +++ | +++ |
| 9 | +++ | +++ |
| 10 | +++ | +++ |
| 72 | +++ | +++ |
| 12 | +++ | +++ |
| 73 | +++ | +++ |
| 74 | +++ | — |
| 13 | +++ | ++ |
| 43 | +++ | ++ |
| 14 | +++ | +++ |
| 11 | +++ | + |
| 15 | +++ | +++ |
| 18 | +++ | +++ |
| 19 | +++ | +++ |
| 75 | +++ | +++ |
| 16 | +++ | ++ |
| 76 | +++ | +++ |
| 44 | +++ | +++ |
| 20 | +++ | +++ |
| 21 | +++ | +++ |
| 22 | +++ | +++ |
| 23 | +++ | +++ |
| 46 | +++ | +++ |
| 78 | +++ | +++ |
| 24 | +++ | +++ |
| 25 | +++ | +++ |
| 79 | +++ | ++ |
| 49 | +++ | +++ |
| 26 | +++ | +++ |
| 27 | +++ | ++ |
| 50 | +++ | +++ |
| 28 | +++ | ++ |
| 47 | +++ | +++ |
| 29 | +++ | ++ |
| 30 | +++ | +++ |
| 31 | +++ | +++ |
| 32 | +++ | + |
| 33 | +++ | +++ |
| 34 | +++ | — |
| 35 | +++ | + |
| 36 | +++ | + |
| 37 | +++ | +++ |
| 17 | +++ | +++ |
| 38 | +++ | ++ |
| 39 | +++ | ++ |
| 40 | +++ | +++ |
| 41 | ++ | + |
| 51 | ++ | + |
| 52 | + | + |
| 42 | + | + |

| | MR Ki (nM) | GR Ki (nM) | MR Ki (nM) | GR Ki (nM) |
|---|---|---|---|---|
| Example No. | Method 1 | Method 1 | Method 2 | Method 2 |
| 164 | +++ | — | — | +++ |
| 165 | +++ | — | — | +++ |
| 166 | +++ | — | — | +++ |
| 167 | +++ | — | — | +++ |
| 168 | +++ | — | — | +++ |
| 169 | +++ | — | — | +++ |
| 170 | +++ | — | — | +++ |
| 171 | +++ | — | — | +++ |
| 172 | +++ | — | — | +++ |
| 173 | +++ | — | — | +++ |
| 174 | +++ | — | — | +++ |
| 175 | +++ | — | — | +++ |
| 176 | +++ | — | — | +++ |
| 177 | +++ | — | — | +++ |
| 178 | +++ | — | — | +++ |
| 179 | +++ | — | — | +++ |
| 180 | +++ | — | — | +++ |
| 181 | +++ | — | — | +++ |
| 182 | +++ | — | — | + |
| 183 | +++ | — | — | — |
| 184 | +++ | — | — | — |
| 185 | +++ | — | — | +++ |
| 186 | +++ | — | — | +++ |
| 187 | +++ | — | — | +++ |
| 188 | +++ | — | — | — |
| 189 | +++ | — | — | +++ |
| 190 | +++ | — | — | +++ |
| 191 | +++ | — | — | ++ |
| 192 | +++ | — | — | +++ |
| 193 | +++ | — | — | +++ |
| 194 | +++ | — | — | — |
| 195 | +++ | — | — | ++ |
| 196 | +++ | — | — | — |
| 197 | +++ | — | — | +++ |
| 198 | +++ | — | — | — |
| 199 | ++ | — | — | +++ |
| 200 | +++ | — | — | — |
| 123 | +++ | — | — | — |
| 124 | +++ | — | — | — |
| 125 | +++ | — | — | — |
| 126 | +++ | — | — | — |
| 127 | +++ | — | — | — |
| 128 | +++ | — | — | — |
| 129 | +++ | — | — | — |
| 130 | +++ | — | — | — |
| 131 | +++ | — | — | — |
| 133 | +++ | — | — | — |
| 134 | +++ | — | — | +++ |
| 135 | +++ | — | — | +++ |
| 136 | +++ | — | — | ++ |
| 137 | +++ | — | — | +++ |
| 138 | +++ | — | — | +++ |
| 139 | +++ | — | — | +++ |
| 140 | +++ | — | — | +++ |
| 141 | +++ | — | — | +++ |
| 142 | +++ | — | — | — |
| 143 | +++ | — | — | — |
| 144 | +++ | — | — | — |
| 145 | +++ | — | — | ++ |
| 146 | +++ | — | — | +++ |
| 147 | +++ | — | — | +++ |
| 148 | +++ | — | — | +++ |
| 149 | +++ | — | — | — |
| 150 | +++ | — | — | — |
| 151 | — | — | — | — |
| 152 | +++ | — | — | — |
| 153 | +++ | — | — | +++ |
| 154 | — | — | — | +++ |
| 155 | +++ | — | — | +++ |
| 156 | +++ | — | — | ++ |
| 157 | +++ | — | — | — |
| 158 | +++ | — | — | +++ |
| 159 | +++ | — | — | — |
| 161 | +++ | — | — | +++ |
| 162 | +++ | — | — | — |
| 163 | +++ | — | — | — |

Legend:
“+” represents a value of ≦ 10,000 nM
“++” represents a value of ≦ 1,000 nM
“+++” represents a value of ≦ 500 nM
“—” indicates the value was not determined

The following Preparations and Examples further illustrate the invention and represent typical synthesis of the compounds of Formula I, including any novel compounds, as described generally in the Schemes above. The reagents and starting materials are readily available from commercial suppliers or may be readily synthesized by one of ordinary skill in the art following the general procedures as described herein. Where the reagent or starting material is not explicitly stated, a reference to a representative Scheme describing procedures for the synthesis of said reagent or starting material is provided. It should be understood that the Preparations and Examples are set forth by way of illustration and not limitation, and that various modifications may be made by one of ordinary skill in the art. As used herein, the following terms have the meanings indicated: “i.v.” refers to intravenously; “p.o.” refers to orally; “i.p.” refers to intraperitoneally; “eq” or “equiv.” Refers to equivalents; “g” refers to grams; “mg” refers to milligrams; “L” refers to liters; “mL” refers to milliliters; “μL” refers to microliters; “mol” refers to moles; “mmol” refers to millimoles; “psi” refers to pounds per square inch; “mm Hg” refers to millimeters of mercury; “min” refers to minutes; “h” or “hr” refers to hours; “° C.” refers to degrees Celsius; “TLC” refers to thin layer chromatography; “HPLC” refers to high performance liquid chromatography; “R_{f}” refers to retention factor; “Rₜ” refers to retention time; “δ” refers to part per million down-field from tetramethylsilane; “THF” refers to tetrahydrofuran; “DMF” refers to N,N-dimethylformamide; “DMSO” refers to dimethyl sulfoxide; “aq” refers to aqueous; “EtOAc” refers to ethyl acetate; “iPrOAc” refers to isopropyl acetate; “MeOH” refers to methanol; “MTBE” refers to tert-butyl methyl ether, “PPh₃” refers to triphenylphosphine; “DEAD” refers to diethyl azodicarboxylate; “RT” refers to room temperature; “Pd—C” refers to palladium over carbon; “SAX” refers to strong anion exchange; “SCX” refers to strong cation exchange; NaBH(Oac)₃ refers to sodium triacetoxyborohydride; “Bn” refers to benzyl; “BnNH₂” refers to benzyl amine; m-CPBA refers to meta-chloroperoxybenzoic acid; H₂ refers to hydrogen; “Kᵢ” refers to the dissociation constant of an enzyme-antagonist complex and serves as an index of ligand binding; and “ID₅₀” and “ID₁₀₀” refer to doses of an administered therapeutic agent which produce, respectively, a 50% and 100% reduction in a physiological response.

Instrumental Analysis:

Unless otherwise indicated, ¹H NMR spectra are recorded on a Bruker 300 MHz spectrometer at ambient temperature. Data are reported as follows: chemical shift in ppm from internal standard tetramethylsilane on the δ scale, multiplicity (b=broad, s=singlet, d=doublet, t=triplet, m=multiplet), and integration. Positive and negative electrospray mass spectral data are obtained on a Micromass Platform LCZ equipped with an autosampler. Analytical thin layer chromatography (tic) is performed on EM Reagent 0.25-mm silica gel 60-F plates. Visualization is accomplished with UV light unless otherwise stated. HPLC analysis is performed on an Altima (C18) 5 m 4.6×150 mm column using a Hitachi L-6200 intelligent pump, a Hitachi L-4000 UV detector, a Hitachi AS-2000 autosampler, and a Hitachi D-2500 chromato-integrator. Acetonitrile and 0.5% ammonium phosphate in water, is used as the mobile phase. Melting points are determined on a Gallenkemp melting point apparatus. Combustion analysis are obtained on an Exeter CE-440.

### Preparation 1

### 3-(4-Fluoro-phenyl)-pentan-3-ol

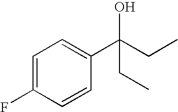

Utilizing the procedures of Scheme II: 4′-fluoropropiophenone (8 ml, 58 mmol) is dissolved in ether (200 ml) then cooled to 0° C. under nitrogen atmosphere. To this solution is added ethyl magnesium bromide (38.4 ml, 3M soln in hexanes, 115 mmol) dropwise over 20 min. The cold bath is then removed and the reaction allowed to warm to ambient temperature. After 12 hrs the reaction is quenched with water and extracted with ethyl acetate. The organics are dried over MgSO₄, filtered and evaporated. This gives 10 g of the product as a clear colorless oil (95%).

### Preparation 2

### 3-(4-Trifluoromethyl-phenyl)-pentan-3-ol

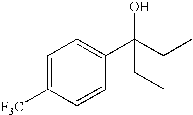

Utilizing the procedures of Scheme II: Methyl 4-(trifluoromethyl)benzoate (1 g, 4.9 mmol) is dissolved in ether (200 ml) then cooled to 0° C. under nitrogen atmosphere. To this solution is added ethyl magnesium bromide (3.59 ml, 3M soln in hexanes, 10.8 mmol) dropwise over 10 min. The cold bath is then removed and the reaction allowed to warm to ambient temperature. After 12 hrs the reaction is quenched with water and extracted with ethyl acetate. The organics are dried over MgSO₄; filtered and evaporated. This gives 1.12 g of the product as a clear colorless oil (98%).

### Preparation 3

### 3-(2-Fluoro-4-methyl-phenyl)-pentan-3-ol

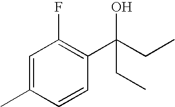

Utilizing the procedures of Scheme III. 4-Bromo-3-fluorotoluene (1 g, 5.3 mmol) is dissolved in ether (20 ml) then cooled to −78° C. under nitrogen atmosphere. To this solution is added n-BuLi (6.61 ml, 1.6M soln in hexanes, 10.6 mmol) dropwise over 10 min. This is stirred for 2 hrs then 3-pentanone (0.56 ml, 5.3 mmol) is added. The cold bath is removed and the reaction allowed to warm to ambient temperature. After 12 hrs the reaction is quenched with water and extracted with ethyl acetate. The organics are dried over MgSO₄, filtered and evaporated. The residue is purified via flash chromatography with 10% ethyl acetate in hexanes to give 801.2 mg of the product as a clear yellow oil (77%).

### Preparation 4

### 7-Butyl-1H-indole

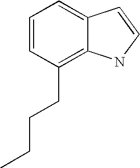

Utilizing the procedures of Scheme X: 2-butyl aniline (1 ml, 6.2 mmol) is dissolved in toluene (20 ml) and cooled to 0° C. To this is added boron trichloride (6.87 ml, 1M soln. In DCM, 6.8 mmol) and this is stirred for 10 min. Chloroacetonitrile is then added (1.58 ml, 24.8 mmol) followed by the aluminum trichloride (833 mg, 6.2 mmol), then the reaction is refluxed. After 12 hrs. the reaction is cooled and extracted with dichloromethane. The organic is dried over MgSO₄, filtered and the solvent evaporated. The residue is dissolved in a 10:1 mixture of dioxane and water and sodium borohydride (3.5 g) is added. This is then refluxed for 12 hrs. After this time the reaction is cooled and extracted with dichloromethane. The organic is dried over MgSO₄, filtered and the solvent evaporated to give 1.05 g of product as an off white solid (97%).

### Preparation 5

### 7-(4-Fluoro-phenyl)-1H-indole

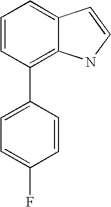

Utilizing the procedures of Scheme VIII: 7-bromoindole (250 mg, 1.3 mmol) is dissolved in toluene (5 ml). To this is added 4-fluorophenyl boronic acid (196.3 mg, 1.4 mmol) followed by Pd(PPh₃)₄ (147 mg, 0.13 mmol). 2N sodium carbonate solution is then added (1.28 ml) and the reaction heated to 80° C. After 24 hrs. the reaction is cooled and extracted with ethyl acetate. The organic is dried over MgSO₄, filtered and the solvent evaporated. The residue is purified by flash chromatography in 10% ethyl acetate in hexanes to give 128.9 mg of product as an off white solid (85%).

### Preparation 6

### (4-Fluoro-phenyl)-phenyl-methanol)

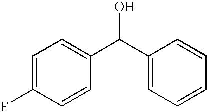

Utilizing the procedures of Scheme V: 4-fluorobenzophenone (5 g, 25 mmol) is dissolved in dichloromethane (50 ml) and methanol (2 ml). This is stirred at ambient temperature under nitrogen atmosphere. To this solution is added sodium borohydride (1.89 g, 50 mmol). After 2 hrs the reaction is quenched with saturated ammonium chloride and extracted with dichloromethane. The organics are dried over MgSO₄, filtered and evaporated. This gives 4.67 g of the product as a white solid (92%).

### Preparation 7

### N-(1H-Indol-7-yl)-methanesulfonamide

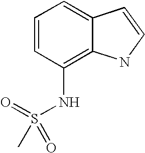

Utilizing 7-nitro indole and the procedures as described in Scheme VII: Utilizing the aniline intermediate from preparation 8 the title product was prepared by stirring this aniline with pyridine (1 eq) and methansulfonyl chloride (1 eq) in dichloromethane for 12 hrs. After this time the reaction is washed with 1N HCl and water before being dried over magnesium sulfate and evaporated. This residue is then recrystalized from isopropanol to provide the title product as a purple solid (94%). MS (ES⁺) 210 (M), MS (ES⁻) 209 (M−1). LC/MS shows 95% purity.

### Preparation 8

### 1H-Indol-7-ylamine

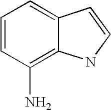

By following the procedures as described in Preparation 7 (Scheme VII, Step A) 7-nitro indole is dissolved in ethanol and to this mixture added ammonium formate (10 eq) and a catalytic amount of 10% palladium on carbon. This mixture is then heated to reflux for 1 hr before it is cooled, filtered through celite and evaporated to provide the product as a purple solid (99%).

### Preparation 9

### 3-Bromo-7-nitro-1H-indole)

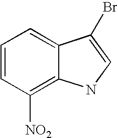

To 0.300 g 7-nitro indole dissolved in 10 mL dichloromethane and cooled to 0° C., is added 0.09 mL bromine. A precipitate slowly forms and after five minutes, is filtered and dried to give 0.302 (68%) title compound.

### Preparation 10

### Indol-1-yl-acetic acid methyl ester

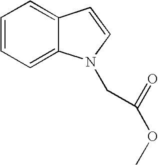

Utilizing the procedures of Scheme XI, Step A: To 2.0 g indole dissolved in 60 mL dimethylformamide is added 10.6 g potassium carbonate. The reaction is heated to 80° C. overnight, cooled to room temperature and concentrated in vacuo. The crude material is redissolved in ethyl acetate, gravity filtered, washed with water, brine, dried over sodium sulfate, filtered, and concentrated in vacuo. Flash chromatography eluting with 75% toluene:hexanes to 2% ethyl acetate:toluene provides 2.085 g (43.1%) product.

### Preparation 11

### 2-Cyclopropyl-4-trimethylsilanyl-but-3-yn-2-ol

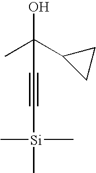

Utilizing the procedure of Scheme VI: To a solution of n-BuLi (63 ml, 101 mmol, 1.00 eq, 1.6 M in hexanes) in ether (50 ml) at −78° C. under nitrogen is added TMS acetylene (15.0 ml, 106 mmol, 1.05 eq) drop wise over 10 minutes and stirred for 1 hour. Cyclopropyl methyl ketone (10.0 ml, 101 mmol) is added drop wise and the reaction mixture is stirred at room temperature for 48 hours. The mixture is then diluted with ether, washed with water twice, 1N hydrochloric acid (2×), brine, dried over anhydrous sodium sulfate, and concentrated to furnish 2-cyclopropyl-4-trimethylsilanyl-but-3-yn-2-ol as clear colorless oil (18.48 g, 100%). NMR (400 MHz, CDCl₃): □ 0.14 (s, 9H, TMS), 0.41-0.62 (m, 4H), 1.11 (m, 1H), 1.55 (s, 3H), 2.00 (s, 1H, OH).

### Preparation 12

### 2-Cyclopropyl-but-3-yn-2-ol

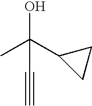

Utilizing the procedure of Scheme VI, step B: A mixture of 2-cyclopropyl-4-trimethylsilanyl-but-3-yn-2-ol (6.10 g, 33.5 mmol) and potassium carbonate (4.62 g, 33.5 mmol) in methanol (20 ml)/water (2 ml) is stirred at room temperature overnight. After diluting with ether, the solids are filtered and the organic phase is washed with water (2×), dried over anhydrous sodium sulfate, and concentrated to afford 2-cyclopropyl-but-3-yn-2-ol (3.47 g, 94%) as clear colorless oil. NMR (400 MHz, CDCl₃): □ 0.42-0.64 (m, 4H), 1.15 (m, 1H), 1.58 (s, 3H), 2.02 (s, 1H, OH), 2.36 (s, 1H).

### Preparation 13

### 4-Fluoro-2-iodo-phenol

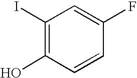

To a solution of 4-fluorophenol (1.90 g, 16.9 mmol) in concentrated ammonium hydroxide (20 ml) at room temperature was added a solution of iodine (4.30 g, 16.9 mmol) and potassium iodide (14.0 g, 84.5 mmol) in water (20 ml) and the resulting mixture was stirred for 2.5 hours. The solution is acidified to pH 2-3 with 1N hydrochloric acid, diluted with ether, washed with 1N hydrochloric acid (2×), dried over anhydrous sodium sulfate, and concentrated. The residue is purified on a 40 g silica column (0 to 100% ethyl acetate/hexanes over 25 minutes) to give 3.42 g of product. NMR analysis indicated an 8:2 mixture of monoiodo and diiodo product.

GC-MS m/z 238 (M⁺)

### Preparation 14

### 1-Cyclopropyl-1-(5-fluoro-benzofuran-2-yl)-ethanol

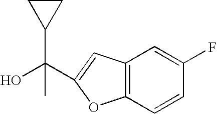

Utilizing the procedure of Scheme VI: A mixture of 4-fluoro-2-iodo-phenol (355 mg, 1.49 mmol), 2-cyclopropyl-but-3-yn-2-ol (246 mg, 2.24 mmol, 1.50 eq), copper(I)oxide (213 mg, 1.49 mmol, 1.00 eq) in anhydrous pyridine (5 ml) is refluxed at 110° C. overnight. After allowing to cool to room temperature, the mixture is diluted with ether, washed with water (2×), dried over anhydrous sodium sulfate, and concentrated to give a black residue (618 mg), which is purified on a 12 g silica column (0 to 100% ethyl acetate/hexanes over 25 minutes) to give the title compound as a yellow oil (151 mg, 46%).

LC-MS m/z 203.0 (M⁺−H₂O)

### Preparation 15

### 4-Chloro-benzo(b)thiophene-2-carboxylic acid methyl ester

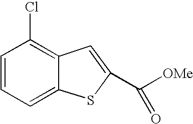

Utilizing the procedure of Scheme XXI: To 10 ml of dimethylsulfoxide is added 0.5 g sodium hydride, followed by methyl thioglycolate (0.72 ml, 8 mmol). Upon completion of gas evolution, the reaction is stirred for an additional 15 minutes whereupon the aldehyde (8 mmol in 2 ml of DMSO) is added rapidly. The reaction is quenched by pouring into ice/water and filtering off 0.53 g (29.27% yield) of the title compound.

¹H NMR, 400 MHz (CDCl3): δ8.1 (s,1H); 7.7 ppm (d,1H); 7.39 ppm (m, 4 h); 3.95 ppm (s,3H).

### Preparation 16

### 4-Fluoro-benzo[b]thiophene-2-carboxylic acid methoxy-methyl-amide

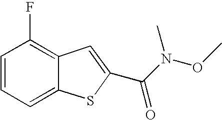

To 38.5 ml of THF is dissolved 2.7 g (12.8 mmol) of 4-Fluoro-benzo(b)thiophen-2-carboxylic acid and 2.57 g of 2-chloro-4,6 dimethoxy-1,3,5 triazine, followed by 4.23 ml (3 eq) of N-methyl-morpholine. This is allowed to stir for one hour before adding 1.35 gram (1 eq) of N,O Dimethylhydroxylamine hydrochloride and stirred overnight. The reaction is worked up between water and ethyl acetate, dried over sodium sulfate and evaporated to yield a crude solid. Flash chromatography using 4/1 hexanes/ethyl acetate yields 0.66 g of the title compound.

¹H NMR, 400 MHz (CDCl₃): δ8.29(1H,s); 7.6(1H,d); 7.39(m,1H); 7.05(t,1H); 3.80(s,3H); 3.40(s,3H).

### Preparation 17

### 1-(4-Fluoro-benzo(b)thiophen-2-yl)-ethanone

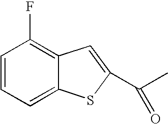

To 25 ml of THF is dissolved 1.98 gram (8.27 mmol) of 4-Fluorobenzo(b)thiophen-2-carboxylic acid methoxy-methyl-amide and cooled in an ice water bath. To this is added 3.03 ml of a 3M methylmagnesium bromide solution in ether. After 45 min, another 1.5 ml of the 3M methylmagnesium bromide solution is added. The reaction is quenched with ethyl acetate followed by the addition of 1N HCl. The organic layer is washed with brine followed by drying over sodium sulfate and evaporating to yield 1.2 g (6.17 mmol) of the title compound.

¹H NMR, 400 MHz (CDCl3): δ8.01(1H,s); 7.62(d,1H); 7.4(m,1H); 7.05(t,1H); 2.70(s,3H).

### EXAMPLE 1

### N-[3-(1-Methyl-1-p-tolyl-butyl)-1H-indol-7-yl]methane sulfonamide

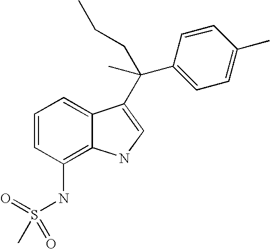

Utilizing the procedures of Scheme I: To 0.100 g N-(1H-Indol-7-yl)-methanesulfonamide, [prepared according to procedures as described in Preparation 7 (Scheme VII)] and 0.085 g of the appropriate carbinol [prepared according to procedures as described in Preparation 1 (Scheme II)] dissolved in 10 mL dichloromethane, 0.055 mL trifluoroacetic acid is added. After 10 minutes, the reaction is concentrated in vacuo. Flash chromatography eluting with a step gradient from 5-10% ethyl acetate:toluene provides 0.09 g (51%) of the title compound.

Analysis calculated for C₂₁H₂₆N₂O₂S: C, 68.0762; H, 7.0731; N, 7.5606. Found: C, 67.58; H, 6.54; N, 7.35.

MS m/z: 369.2 (M⁻−1).

Examples 2-17 below are made following procedures essentially as described in Example 1 above. That is, employing the procedures of Scheme I, and utilizing the appropriate indole and the appropriate carbinol, each of which may be obtained from commercial sources or prepared according to procedures as described in the Preparations herein, the title compounds of Examples 2-17 are prepared.

### EXAMPLE 2

### N-[3-(1-Benzofuran-2-yl-1-ethyl-propyl)-1H-indol-7-yl]-methanesulfonamide

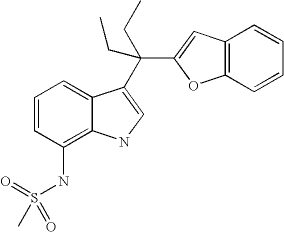

Flash chromatography eluting with a step gradient from 5-10% ethyl acetate:toluene provides 0.119 g (63%) of the product.

MS m/z: 395.1 (M⁻−1).

### EXAMPLE 3

### N-{3-[1-(2,3-Dihydro-benzofuran-5-yl)-1-ethyl-propyl]-1H-indol-7-yl}-methanesulfonamide

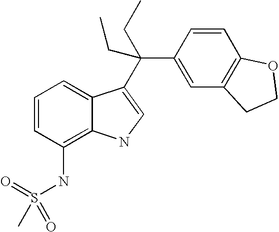

Flash chromatography eluting with 10% ethyl acetate:toluene provides 0.098 g (52%) of the product.

MS m/z: 397.2 (M⁻−1).

### EXAMPLE 4

### N-{3-[1-Ethyl-1-(7-methoxy-benzofuran-2-yl)-propyl]-1H-indol-7-yl}-methanesulfonamide

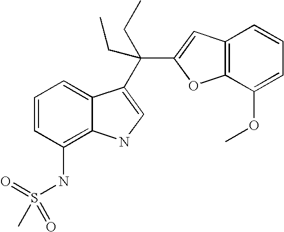

Flash chromatography eluting with a step gradient from 5-10% ethyl acetate:toluene provides 0.316 g (67.5%) of the product.

MS m/z: 425.1 (M⁻−1).

### EXAMPLE 5

### N-{3-[1-Cyclopropyl-1-(4-fluoro-phenyl)-ethyl]-1H-indol-7-yl}-methanesulfonamide

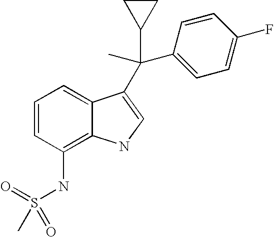

Flash chromatography eluting with 5% ethyl acetate:toluene followed by crystallization with carbon tetrachloride provides 0.05 g (28%) of the product.

MS m/z: 371.1 (M⁻−1).

### EXAMPLE 6

### N-[3-(1-Benzo[b]thiophen-2-yl-1-ethyl-propyl)-1H-indol-7-yl]-methanesulfonamide

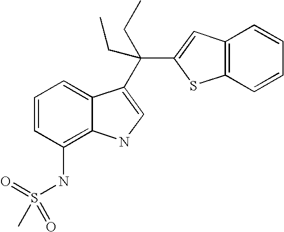

Flash chromatography eluting with 5% ethyl acetate:toluene followed by crystallization with carbon tetrachloride provides 0.068 g (23%) of the product.

MS m/z: 411.1 (M⁻−1).

### EXAMPLE 7

### N-{3-[1-(4-Fluoro-phenyl)-1-methyl-butyl]-1H-indol-7-yl}-methanesulfonamide

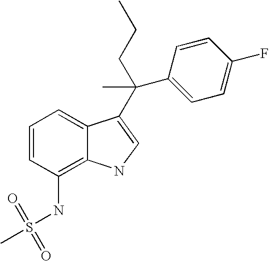

Flash chromatography eluting with 5% ethyl acetate:toluene provides 0.056 g (23%) of the product.

MS m/z: 373.2 (M⁻−1).

### EXAMPLE 8

### N-{3-[1-Ethyl-1-(4-methylsulfanyl-phenyl)-propyl]-1H-indol-7-yl}-methanesulfonamide

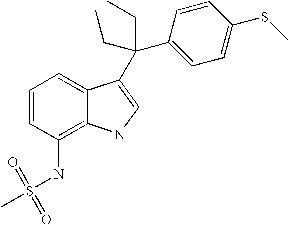

Flash chromatography eluting with a step gradient from 5-10% ethyl acetate:toluene provides 0.611 g (63.8%) of the product.

MS m/z: 403 (M⁺+1); 401 (M⁻−1).

### EXAMPLE 9

### N-[3-(1-Benzofuran-2-yl-1-methyl-ethyl)-1H-indol-7-yl]-methanesulfonamide

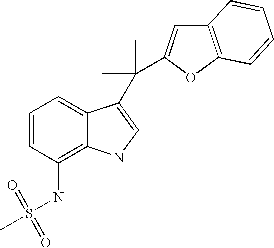

Flash chromatography eluting with 10% ethyl acetate:toluene provides 0.049 g (61%) of the product.

MS m/z: 367.1 (M⁻−1).

### EXAMPLE 10

### N-[3-(1-Methyl-1-thiophen-3-yl-butyl)-1H-indol-7-yl]-methanesulfonamide

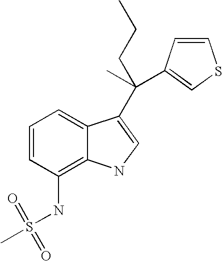

Flash chromatography eluting with 5% ethyl acetate:toluene provides 0.074 g (43%) of the product.

MS m/z: 361.1 (M⁻−1).

### EXAMPLE 11

### N-{3-[1-(5-Chloro-benzofuran-2-yl)-1-ethyl-propyl]-1H-indol-7-yl}-methanesulfonamide

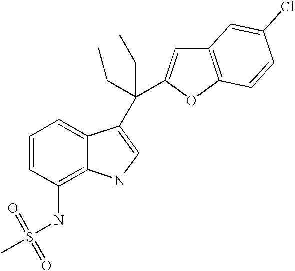

Flash chromatography eluting with a step gradient from 5-10% ethyl acetate:toluene followed by crystallization with carbon tetrachloride provides 0.37 g (59%) of the product.

MS m/z: 429 (M⁻−1).

### EXAMPLE 12

### N-[3-(1-Methyl-1-p-tolyl-ethyl)-1H-indol-7-yl]-methanesulfonamide

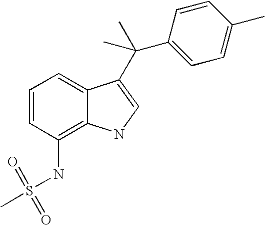

Flash chromatography eluting with 5% ethyl acetate:toluene provides 0.064 g (39.3%) of the product.

MS m/z: 341.2 (M⁻−1).

### EXAMPLE 13

### N-[3-(1-Benzo[b]thiophen-2-yl-1-methyl-ethyl)-1H-indol-7-yl]-methanesulfonamide

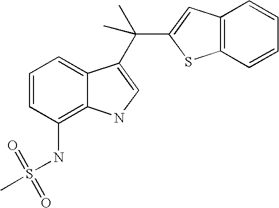

Flash chromatography eluting with 10% ethyl acetate:toluene provides 0.108 g (25%) of the product.

MS m/z: 383.1 (M⁻−1).

### EXAMPLE 14

### N-[3-(1-Benzo[b]thiophen-3-yl-1-methyl-ethyl)-1H-indol-7-yl]-methanesulfonamide

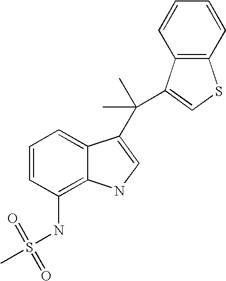

Flash chromatography eluting with 10% ethyl acetate:toluene provides 0.062 g (68%) of the product.

MS m/z: 383.1 (M⁻−1).

### EXAMPLE 15

### N-[3-(1-Ethyl-1-thiophen-2-yl-propyl)-1H-indol-7-yl]-methanesulfonamide

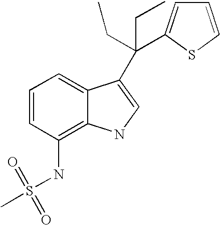

Flash chromatography eluting with 5% ethyl acetate:toluene followed by crystallization with carbon tetrachloride provides 0.11 g (64%) of the product.

MS m/z: 361.1 (M⁻−1).

### EXAMPLE 16

### N-[3-(1-Phenylcyclohexyl)-1H-indol-7-yl]-methanesulfonamide

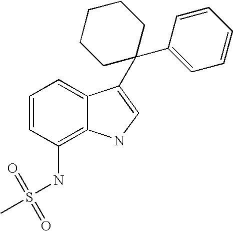

Flash chromatography eluting with 5% ethyl acetate:toluene followed by crystallization with carbon tetrachloride provides 0.08 g (46%) of the product.

MS m/z: 370.2 (M⁺+1); 367.1 (M⁻−1).

### EXAMPLE 17

### N-{3-[1-(4-Benzyloxy-phenyl)-1-ethyl-propyl]-1H-indol-7-yl}-methanesulfonamide

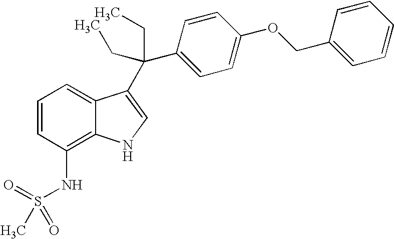

Flash chromatography eluting with a step gradient from 5-10% ethyl acetate:toluene provides 0.794 g (67.9%) of the product.

MS m/z: 461.2 (M⁻−1).

### EXAMPLE 18

### N-{3-[1-Ethyl-1-(4-hydroxy-phenyl)-propyl]-1H-indol-7-yl}-methanesulfonamide

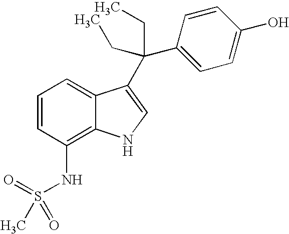

Utilizing procedures as described in Scheme XIV: To 0.64 g of the benzyl ether from Example 17, dissolved in 20 mL ethanol, a catalytic amount of 10% palladium on carbon and excess of ammonium formate is added. The reaction is heated to 45° C. until gas evolution occurrs and then the heat is removed. Celite is added and the reaction filtered and concentrated in vacuo. The residue is redissolved in ethyl acetate and water. The organic layer is then separated and washed with brine, dried over sodium sulfate, filtered, and concentrated in vacuo. The residue post workup is slurried in carbon tetrachloride and filtered to give 0.418 g (81.2%) product.

MS m/z: 373.1 (M⁺+1); 371.1 (M⁻−1).

### EXAMPLE 19

### 7-Ethyl-3-[1-(4-methoxy-phenyl)-1-methyl-butyl]-1H-indole

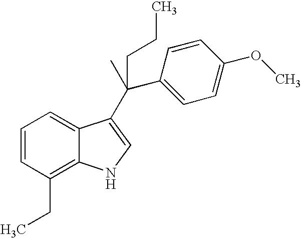

Utilizing 7-ethyl indole and the appropriate carbinol, prepared essentially as described in Preparation 1 (Scheme II), the title compound is prepared according to procedures as described in Example 1 (Scheme I). Filter chromatography eluting with toluene, followed by recrystallization with methyl alcohol provides 0.37 g (76.8%).

Analysis calculated for C₂₂H₂₇NO: C, 82.1999; H, 8.4659; N, 4.3571. Found: C, 81.48; H, 8.71; N, 4.50.

MS m/z: 320.2 (M⁻−1).

Examples 20-23 below are made following procedures essentially as described in Example 1 above. That is, employing the procedures of Scheme I, and utilizing the appropriate indole and the appropriate carbinol, each of which may be obtained from commercial sources or prepared according to procedures as described in the Preparations herein, the title compounds of Examples 20-23 are prepared.

### EXAMPLE 20

### 3-[1-(4-Methoxy-phenyl)-1-methyl-butyl]-7-methyl-1H-indole

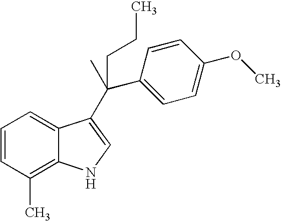

Flash chromatography eluting with 1:1 toluene:hexanes provides 0.168 g (71.8%).

MS m/z: 306.2 (M⁻−1).

### EXAMPLE 21

### N-[3-(1-Phenyl-cyclopentyl)-1H-indole-7-yl]-methanesulfonamide

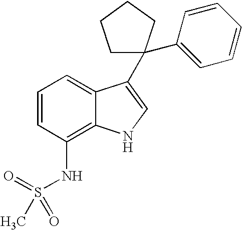

Flash chromatography eluting with 10% Ethyl Acetate:Toluene provides 0.06 g (71.4%) of the product.

MS m/z: 353.1 (M⁻−1).

### EXAMPLE 22

### N-{3-[1-(2,3-Dihydro-benzofuran-5-yl)-1-methyl-ethyl]-1H-indol-7-yl}-methanesulfonamide

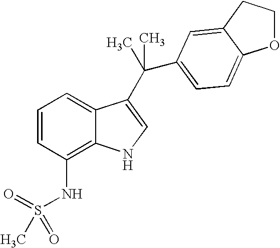

Flash chromatography eluting with 10% ethyl acetate:toluene provides 0.134 g (76%) of the product.

MS m/z: 369.1 (M⁻−1).

### EXAMPLE 23

### 3-[1-(4-Methoxy-phenyl)-1-methyl-pentyl]-7-methyl-1H-indole

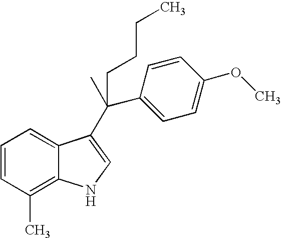

Filter chromatography eluting with toluene provides 0.204 g (83%) of the product.

MS m/z: 320.2 (M⁻−1).

Examples 24-25 below are also made following procedures essentially as described in Example 1 above. That is, employing the procedures of Scheme I, utilizing the commercially available indole and the appropriate carbinol, obtained from commercial sources or prepared according to procedures as the Preparations herein, the title compounds of Examples 24-25 are prepared.

### EXAMPLE 24

### 3-[1-Cyclopropyl-1-(4-methoxy-phenyl)-ethyl]-7-methyl-1H-indole

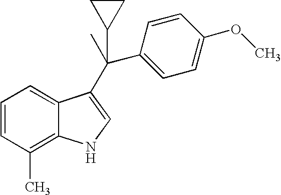

Flash chromatography eluting with 50% hexane:toluene provides 0.196 g (84.1%) of the product.

MS m/z: 304.1 (M⁻−1).

### EXAMPLE 25

### 7-Ethyl-3-[1-(4-methoxy-phenyl-1-phenyl-ethyl]-1H-indole

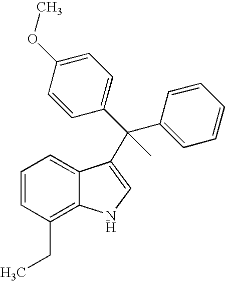

Flash chromatography eluting with Toluene provides 0.16 g (52.8%) of the product.

Analysis calculated for C₂₄H₂₃NO₂: C, 84.4704; H, 7.0887; N, 3.9402. Found: C, 83.7; H, 7.06; N, 3.67.

MS m/z: 354.4 (M⁻−1).

Example 26 below is made following procedures essentially as described in Example 1 above. That is, employing the procedures of Scheme I, utilizing the commercially available indole and the appropriate carbinol, obtained from commercial sources or prepared according to procedures as described in the Preparations herein, the title compounds of Example 26 is prepared.

### EXAMPLE 26

### 3-[1-(4-Fluoro-phenyl)-1-methyl-butyl]-7-methyl-1H-indole

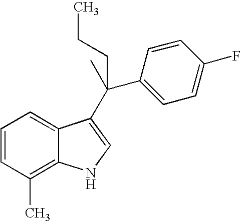

Flash chromatography eluting with 50% hexanes:toluene provides 0.254 g (78.4%) of the product.

MS m/z: 294.2 (M⁻−1).

Example 27 is made following procedures essentially as described in Example 18 above. That is, employing the procedures of Scheme XIV, utilizing the commercially available indole and the appropriate carbinol, obtained from commercial sources or prepared according to procedures as described in the Preparations herein, the benzyl ether intermediate for Example 27 is first prepared according to the procedures of Example 1 (Scheme I). The title compoud is then prepared according to the procedures described in Example 18 (Scheme XIV).

### EXAMPLE 27

### 4-[1-(7-Ethyl-1H-indole-3-yl)-1-(4-fluoro-phenyl)-ethyl]-phenol

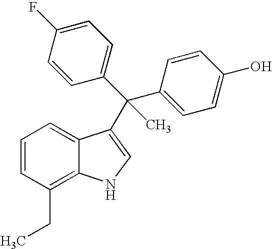

The title compound is prepared according to Scheme XIV, Example 18 to give 0.074 g (33.8%) product.

MS m/z: 358.3 (M⁻−1).

### EXAMPLE 28

### 3-[1-(4-Methoxy-phenyl)-1-phenyl-ethyl]-7-methyl-1H-indole

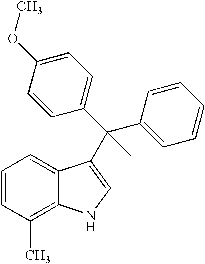

Utilizing 7-methyl indole and the appropriate carbinol, prepared according to procedures as described in Preparation 1 (Scheme II), the title compound is prepared according to Example I, Scheme I. Flash chromatography eluting with Toluene provides 0.230 g (68%) of the product.

MS m/z: 340.3 (M⁻−1).

### EXAMPLE 29

### N-{3-[1-Ethyl-1-(5-methoxy-benzofuran-2-yl)-propyl]-1H-indol-7-yl}-methanesulfonamide

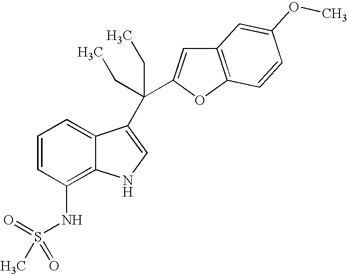

Utilizing the appropriate indole, prepared according to procedures as described in Preparation 7 (Scheme VII), and the appropriate carbinol, prepared according to procedures as described in Preparation 1 (Scheme II), the title compound is prepared according to Example I, Scheme I. Flash chromatography eluting with a step gradient from 5-10% ethyl acetate:toluene followed by crystallization with carbon tetrachloride provides 0.331 g (71%) of the product.

MS m/z: 425 (M⁻−1).

### EXAMPLE 30

### N-[3-(1-Ethyl-1-furan-2-yl-propyl)-1H-indol-7-yl]-methanesulfonamide

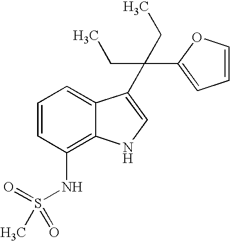

Utilizing the appropriate indole prepared according to procedures as described in Preparation 7 (Scheme VII) and the appropriate carbinol, prepared according to procedures as described in Preparation 1 (Scheme II), the title compound is prepared according to Example I, Scheme I. Flash chromatography eluting with a step gradient from 5-10% ethyl acetate:toluene provides 0.079 g (48%) of the product.

MS m/z: 345.1 (M⁻−1).

### EXAMPLE 31

### 3-[1-(4-Methoxy-phenyl)-1-methyl-propyl]-7-methyl-1H-indole

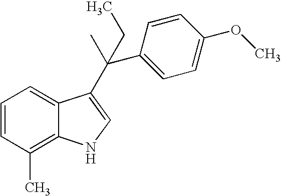

Utilizing 7-methyl indole and appropriate carbinol, prepared according to procedures as described in Preparation 1 (Scheme II), the title compound is prepared according to Example I, Scheme I. Flash chromatography eluting with toluene provides 0.190 g (64.8%) of the product.

MS m/z: 294 (M⁺+1); 292.4 (M⁻−1).

### EXAMPLE 32

### 3-[1-(4-Fluoro-phenyl)-cyclohexyl]-7-methyl-1H-indole

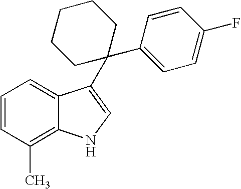

Utilizing 7-methyl indole and appropriate carbinol, prepared according to procedures as described in Preparation 1 (Scheme II), the title compound, is prepared according to Example I, Scheme I. Flash chromatography eluting with 30% hexanes:toluene provides 0.052 g (21.4%) of the product.

MS m/z: 308 (M⁺+1); 306 (M⁻−1).

### EXAMPLE 33

### N-[3-(4-Phenyl-tetrahydro-pyran-4-yl)-1H-indol-7-yl]-methanesulfonamide

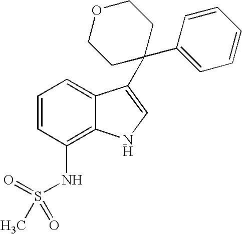

Utilizing the appropriate indole, prepared according to procedures as described in Preparation 7 (Scheme VII), and the appropriate carbinol, prepared according to procedures as described in Preparation 1 (Scheme II), the title compound is prepared according to Example I, Scheme I. Flash chromatography eluting with 0.5% Methyl Alcohol:Chloroform provides 0.012 g (6.8%) of the product.

MS m/z: 369.2 (M⁻−1).

### EXAMPLE 34

### N-[3-(1-Biphenyl-2-yl-1-methyl-ethyl)-1H-indol-7-yl]-methanesulfonamide

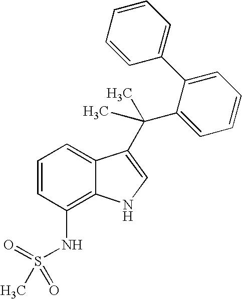

Utilizing the appropriate indole prepared according to procedures as described in Preparation 7 (Scheme VII) and the appropriate carbinol, prepared according to procedures as described in Preparation 1 (Scheme II), the title compound is prepared according to Example 1 (Scheme I).

MS m/z: 403.2 (M⁻−1).

### EXAMPLE 35

### 3-[1-Methyl-1-(4-trifluoromethoxy-phenyl-butyl]-1H-indol-7-ylamine

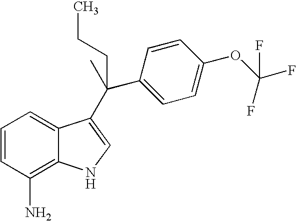

Utilizing 7-nitro indole and the appropriate carbinol, prepared according to procedures as described in Preparation 1 (Scheme II), the nitro intermediate is prepared according to Example 1 (Scheme I). The title compound is prepared utilizing conditions as described in Example 18 (Scheme XIV (Scheme VII, Step A)) to give 0.52 g (87%) product.

MS m/z: 363.3 (M⁺+1); 361.2 (M⁻−1).

### EXAMPLE 36

### N-{3-[1-(4′-Fluoro-biphenyl-3-yl)-1-methyl-ethyl]-1H-indol-7-yl}-methanesulfonamide

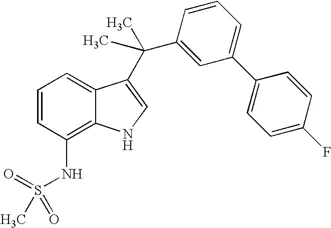

Utilizing the indole prepared according to procedures as described in Preparation 7 (Scheme VII) and the approprioate carbinol from Scheme II, the title compound is prepared according to Example 1 (Scheme I).

MS m/z: 421.2 (M⁻−1).

### EXAMPLE 37

### 7-Methyl-3-(1-methyl-1-phenyl-butyl)-1H-indole

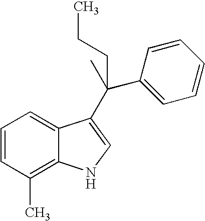

Utilizing 7-methyl indole and the appropriate carbinol, prepared according to procedures as described in Preparation 1 (Scheme II), the title compound is prepared according to Example 1 (Scheme I). Flash chromatography eluting with 50% hexanes:toluene provides 0.311 g (92%) of the product.

MS m/z: 276.2 (M⁻−1).

### EXAMPLE 38

### 3-[1-(3-Methoxy-phenyl)-1-methyl-butyl]-1H-indol-7-ylamine

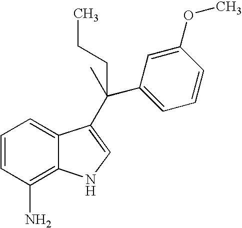

Utilizing 7-nitro indole and the appropriate carbinol, prepared according to procedures as described in Preparation 1 (Scheme II), the nitro intermediate is prepared according to Example 1 (Scheme 1). The title compound is prepared utilizing conditions described in Example 18 (Scheme XIV (Scheme VII, Step A)) to give 0.31 g (97.5%) product.

MS m/z: 309.3 (M⁺+1); 307.2 (M⁻−1).

### EXAMPLE 39

### N-[3-(1-Biphenyl-4-yl-1-methyl-ethyl)-1H-indol-7-yl]-methanesulfonamide

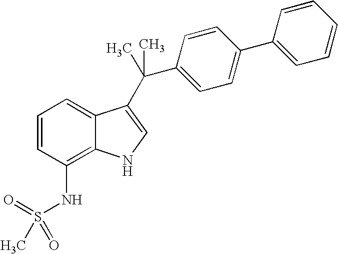

Utilizing the appropriate indole, prepared according to procedures as described in Preparation 7 (Scheme VII) and the appropriate carbinol, prepared according to procedures as described in Preparation 1 (Scheme II), the title compound is prepared according to Example 1 (Scheme 1). Flash chromatography eluting with 5% ethyl acetate:toluene followed by crystallization with carbon tetrachloride provides 0.16 g (83%) of the product.

MS m/z: 403.2 (M⁻−1).

### EXAMPLE 40

### 3-[1-Cyclopropyl-1-(4-fluoro-phenyl)-ethyl]-7-methyl-1H-indole

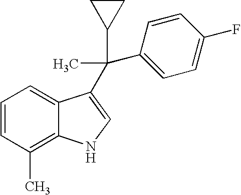

Utilizing 7-methyl indole and the commercially available carbinol, the title compound is prepared according to procedures as described in Example 1 (Scheme I).

MS m/z: 292.2 (M⁻−1).

### EXAMPLE 41

### 3-(1-Methyl-1-p-tolyl-ethyl)-1H-indol-7-ylamine

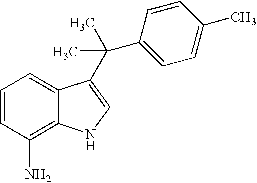

Utilizing 7-nitro indole and the commercially available carbinol, the nitro intermediate is prepared according to Example 1 (Scheme I). The title compound is prepared utilizing conditions as described in Example 18 (Scheme XIV (Scheme VII, Step A)). Filter chromatography eluting with ethyl acetate followed by flash chromatography eluting with 5-25% ethyl acetate:toluene provides 0.028 g (2.4%) product.

MS m/z: 265.1 (M⁺+1); 263.1 (M⁻−1).

### EXAMPLE 42

### [3-(1,1-Diphenyl-ethyl)-indol-1-yl]-acetic acid

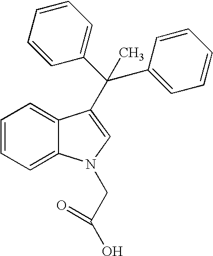

Utilizing indoleacetic acid and the appropriate carbinol, prepared according to procedures as described in Preparation 1 (Scheme II), the title compound is prepared according to procedures as described in Example 1 (Scheme I). Crude product purified via SAX, washing with ethyl acetate followed by 10% acetic acid:ethyl acetate to give 0.073 g (35.9%) product.

MS m/z: 373.3 (M⁺+18); 354.1 (M⁻−1).

### EXAMPLE 43

### N-{3-[1-Methyl-1-(4-trifluoromethoxy-phenyl-butyl]-1H-indol-7-yl}-methanesulfonamide

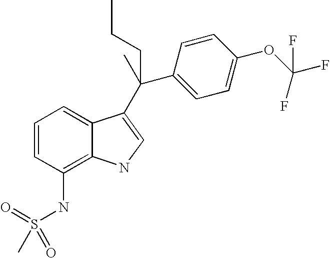

Using 7-nitro indole and the appropriate carbinol, prepared according to procedures as described in Preparation 1 (Scheme II), the nitro intermediate is prepared according to Example 1 (Scheme I). Using the nitro intermediate, the corresponding aniline intermediate is prepared according to conditions described in Example 18 (Scheme XIV (Scheme VII, Step A)) Utilizing procedures as described in Scheme VII, Step C, to 0.49 g of the aniline intermediate dissolved in 20 mL dichloromethane and 0.22 mL pyridine, is added 0.11 mL methanesulfonyl chloride. The reaction is stirred at room temperature for a minimum of six hours. Upon completion, the reaction is concentrated in vacuo. The residue is redissolved in ethyl acetate and washed with water followed by brine, dried over sodium sulfate, filtered and concentrated in vacuo to give 0.375 g of the title compound.

Analysis calculated for C₂₁H₂₃F₃N₂O₃S: C, 57.2624; H, 5.2631; N, 6.3596. Found: C, 57.07; H, 4.94; N, 6.17.

### EXAMPLE 44

### N-{3-[1-(2-Methoxy-phenyl)-1-methyl-butyl]-1H-indol-7-yl}-methanesulfonamide

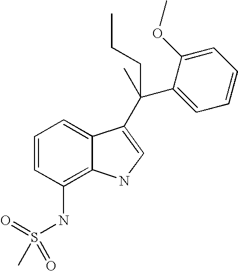

Utilizing 7-nitro indole and the appropriate carbinol, prepared according to procedures as described in Preparation 1 (Scheme I), the nitro intermediate is prepared according to procedures as described in Example 1 (Scheme I). Using the nitro intermediate, the corresponding aniline intermediate is prepared according to conditions described in Example 18 (Scheme XIV (Scheme VII, Step A)). The title compound is then prepared according to procedures as described in Example 43 (Scheme VII, Step C) to give 0.538 g (38%).

Analysis calculated for C₂₁H₂₆N₂O₃S: C, 65.2582; H, 6.7804; N, 7.2477. Found: C, 64.68; H, 6.60; N, 7.18.

### EXAMPLE 45

### N-{3-[1-(4-Methoxy-phenyl)-1-methyl-butyl]-1H-indol-7-yl}-methanesulfonamide

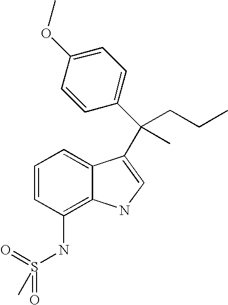

Utilizing 7-nitro indole and the appropriate carbinol, prepared according to procedures as described in Preparation 1 (Scheme II), the nitro intermediate is prepared according to procedures as described in Example 1 (Scheme D. Using the nitro intermediate, the corresponding aniline intermediate is prepared according to procedures as described in Scheme VII, Step B. The title compound is then prepared according to procedures as described in Example 43 (Scheme VII, Step C). Flash chromatography eluting with 5% Ethyl Acetate/Toluene provides 0.14 g (71%) product.

Analysis calculated for C₂₁H₂₆N₂O₃S: C, 65.2582; H, 6.7804; N, 7.2477. Found: C, 65.53; H, 6.73; N, 7.16.

### EXAMPLE 46

### N-{3-[1-(3-Methoxy-phenyl)-1-methyl-butyl]-1H-indol-7-yl}-methanesulfonamide

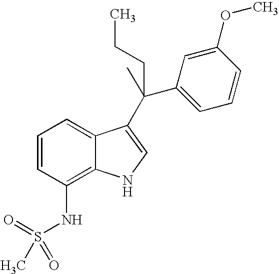

Utilizing 7-nitro indole and the appropriate carbinol, prepared according to procedures as described in Preparation 1 (Scheme II), the nitro intermediate is prepared according to procedures as described in Example 1 (Scheme I). Using the nitro intermediate, the corresponding aniline intermediate is prepared according to conditions described in Example 18 (Scheme XIV (Scheme VII, Step A)). The title compound is then prepared according to procedures as described in Example 43 (Scheme. VII, Step C). Flash chromatography eluting with a step gradient from 5-10% Ethyl Acetate:Toluene provides 0.091 g (27%) of the product.

MS m/z: 385.2 (M⁻−1).

### EXAMPLE 47

### N-[3-(1-Methyl-1-quinolin-6-yl-ethyl)-1H-indol-7-yl]-methanesulfonamide

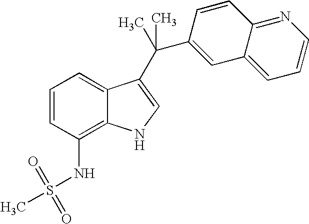

Using the nitro intermediate prepared in Example 53, the aniline intermediate is prepared according to conditions described in Example 18 (Scheme XIV (Scheme VII, Step A)). The title compound is prepared according to procedures as described in Example 43 (Scheme VII, Step C). The material post workup is slurried in 50% carbon tetrachloride:diethyl ether to give 0.051 g (57%) product.

MS m/z: 380.1 (M⁺+1); 378.1 (M⁻−1).

### EXAMPLE 48

### N-{3-[1-Ethyl-1-(4-methanesulfonyl-phenyl)-propyl]-1H-indol-7-yl}-methanesulfonamide

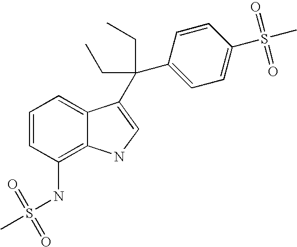

Utilizing the procedures as described in Scheme X, Step A, 0.050 g of the sulfide intermediate (prepared according to the procedures of Example 1 (Scheme I) utilizing the appropriate indole, prepared according to procedures as described in Preparation 7 (Scheme VII) and the appropriate carbinol, prepared according to procedures as described in Preparation 1 (Scheme II)), dissolved in 5 mL dichloromethane, is added 0.53 g silica gel followed by 0.03 mL tert-butyl hydroperoxide. The reaction is stirred at room-temperature overnight. Another 2 mL dichloromethane and 0.03 mL tert-butyl hydroperoxide is added and the reaction stirred for about four more hours then concentrated in vacuo. Flash chromatography eluting with a step gradient from 10-50% Ethyl Acetate:Toluene followed by slurrying in carbon tetrachloride provides 0.027 g (50%) of the product.

MS m/z: 433 (M⁻−1).

### EXAMPLE 49

### N-{3-[1-Ethyl-1-(4-methanesulfinyl-phenyl)-propyl]-1H-indol-7-yl}-methanesulfonamide

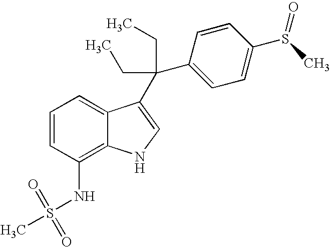

Utilizing the procedures as described in Scheme X, Step B, to 0.200 g of the sulfide intermediate (prepared according to the procedures of Example 1 (Scheme I) utilizing the appropriate indole, prepared according to procedures as described in Preparation 7 (Scheme VII) and the appropriate carbinol, prepared according to procedures as described in Preparation 1 (Scheme II)) dissolved in 5 mL dichloromethane, is added 2 g silica gel and 0.07 mL tert-butyl hydroperoxide. The reaction stirred at room temperature for four hours. Ethyl acetate was then added and the silica gel filtered with multiple ethyl acetate washes. It was then concentrated in vacuo to give 0.11 g (53%) of the title compound.

### EXAMPLE 50

### 3-[1-(4-Methoxy-phenyl)-1-methyl-butyl]-1H-indol-7-ylamine

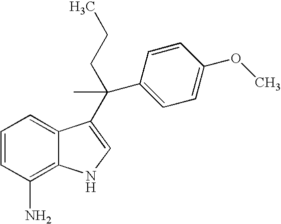

Utilizing 7-nitro indole and the appropriate carbinol, prepared according to procedures as described in Preparation 1 (Scheme II), the nitro intermediate is prepared according to procedures as described in Example 1 (Scheme I). Using the nitro intermediate, the corresponding aniline intermediate is prepared according to procedures as described in Scheme VII, Step B. Flash chromatography eluting with 15% Ethyl Acetate:Toluene provides 0.207 g (72.60%) of the title compound.

Analysis calculated for C₂₀H₂₄N₂O: C, 77.8865; H, 7.8434; N, 9.0827. Found: C, 77.61; H, 7.83; N, 8.91.

MS m/z: 307.4 (M⁻−1).

### EXAMPLE 51

### (3-Trityl-indol-1-yl)-acetic acid

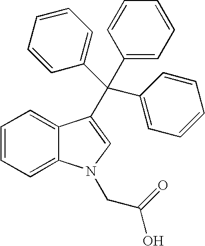

Utilizing the indole from Preparation 11 and commercially available triphenyl methanol, the intermediate ester is prepared according to procedures as described in Scheme XI, Step B. The title compound is prepared according to procedures as described in Scheme XI, Step C to give 0.053 g (96.4%) product.

MS m/z: 417.2 (M⁻−1).

### EXAMPLE 52

### 3-(3-Trityl-indol-1-yl)-propionic acid

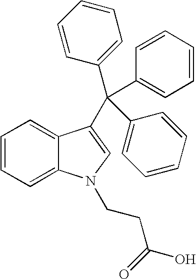

The title compound is prepared utilizing procedures as described in Scheme XI, Steps A-C to give 0.138 g (71.1%) product.

MS m/z: 430.1 (M⁻−1).

### EXAMPLE 53

### 6-[1-Methyl-1-(7-nitro-1H-indol-3-yl)-ethyl]-quinoline

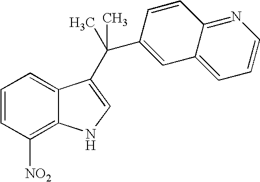

Following procedures as described in Scheme I, to 0.200 g of nitro indole and 0.230 g carbinol (prepared according to Scheme II) dissolved in 5 mL glacial acetic acid is added 0.13 mL concentrated sulfuric acid. After two hours, another 0.13 mL sulfuric acid is added and the reaction stirred for 72 hours. Upon completion, water is added and the reaction basified with 5N sodium hydroxide solution, extracted with ethyl acetate, and the organics washed with brine, dried over sodium sulfate, filtered and concentrated in vacuo. Flash chromatography eluting with 10% ethyl acetate:toluene provides 0.078 g (19%) of the product.

MS m/z: 419 (M⁺+1); 417 (M⁻−1).

MS m/z: 482.2 (M⁺−1).

### EXAMPLE 54

### N-[3-(1-Ethyl-1-p-tolyl-propyl)-1H-indol-7-yl]-methanesulfonamide

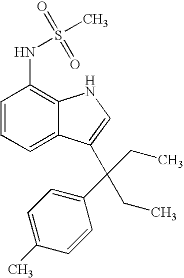

Utilizing the procedures as described in Example 1 (Scheme I): N-(1H-Indol-7-yl)-methanesulfonamide (100 mg, 0.476 mmol)(Preparation 7) is dissolved in dichloromethane (5 ml) then stirred at ambient temperature. 3-p-Tolyl-pentan-3-ol (84.8 mg, 0.476 mmol), prepared according to procedures as described in Preparation 1, is added followed by trifluoroacetic acid (0.037 ml, 0.476 mmol). The reaction is monitored by tlc (1:1 hexanes:ethyl acetate) until starting material is consumed. The reaction is concentrated and the residue purified via flash chromatography in 25% ethyl acetate in hexanes to give 91.8 mg of the product as a white solid (52%). MS (ES⁻) 369 (M−1).

Examples 55-57 below are made following procedures essentially as described in Example 54 above. That is, employing the procedures of Scheme I, and utilizing the appropriate indole and the appropriate carbinol, each of which may be obtained from commercial sources or prepared according to procedures as described in the Preparations herein, the title compounds of Examples 55-57 are prepared.

### EXAMPLE 55

### N-{3-[1-(4-Methoxy-phenyl)-1-methyl-ethyl]-1H-indol-7-yl}-methanesulfonamide

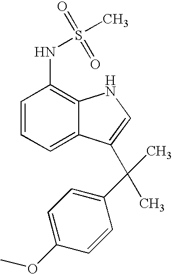

Title product is prepared as a white solid (45%). MS (ES⁺) 387 (M+1), MS (ES⁻) 385 (M−1).

### EXAMPLE 56

### N-{3-[1-(4-Chloro-phenyl)-1-ethyl-propyl]-1H-indol-7-yl}-methanesulfonamide

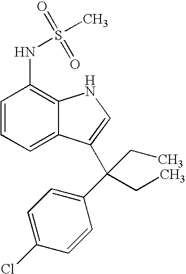

Title product is prepared as a white solid (45%). MS (ES⁺) 391 & 393 (M+1), MS (ES⁻) 389 &391 (M−1). EA: theory (% C=61.4482, % H=5.9302, % N=7.1657), experimental (% C=61.11, % H=6.06, % N=7.04).

### EXAMPLE 57

### N-{3-[1-Ethyl-1-(2-fluoro-4-methyl-phenyl)-propyl]-1H-indol-7-yl}-methanesulfonamide

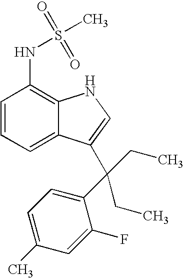

Title product is prepared as a off white solid (80%). MS (ES⁺) 389 (M+1), MS (ES⁻) 387 (M−1). EA: theory (% C=64.9238, % H=6.4862, % N=7.2105), experimental (% C=64.46, % H=6.36, % N=6.73).

Examples 58-68 below are made following procedures essentially as described in Example 54 above. That is, employing the procedures of Scheme I, and utilizing the appropriate indole and the appropriate carbinol, each of which may be obtained from commercial sources or prepared according to procedures as described in the Preparations herein, the title compounds of Examples 58-68 are prepared.

### EXAMPLE 58

### N-{3-[1-(3,4-Dimethyl-phenyl)-1-ethyl-propyl]-1H-indol-7-yl}-methanesulfonamide

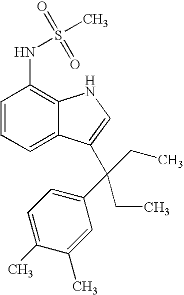

Title product is prepared as a white solid (83%). MS (ES⁻) 383 (M−1). LC/MS shows 95% purity.

### EXAMPLE 59

### N-{3-[1-Ethyl-1-(4-fluoro-phenyl)-propyl]-1H-indol-7-yl}-methanesulfonamide

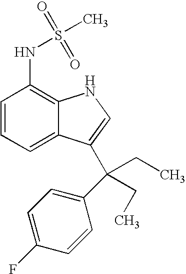

Title product is prepared as a white solid (25%). MS (ES⁺) 375 (M+1), MS (ES⁻) 373 (M−1). HPLC shows 97.8% purity (65% acetonitrile). MP=137-138° C.

### EXAMPLE 60

### N-{3-[1-(2,4-Dimethyl-phenyl)-1-ethyl-propyl]-1H-indol-7-yl}-methanesulfonamide

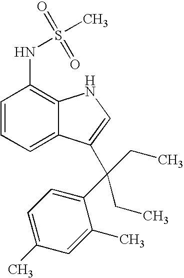

Title product is recystalized from 1:1 ether:pentane to give the product as a white solid (1%). MS (ES⁺) 385 (M+1), MS (ES⁻) 383 (M−1).

### EXAMPLE 61

### N-[3-(1-Ethyl-1-phenyl-propyl)-1H-indol-7-yl]-methanesulfonamide

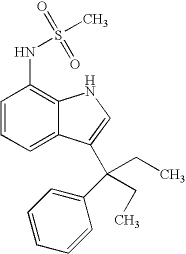

Title product is prepared as a white solid (72%). MS (ES⁺) 357 (M+1), MS (ES⁻) 355 (M−1).

### EXAMPLE 62

### N-{3-[1-(2,4-Difluoro-phenyl)-1-ethyl-propyl]-1H-indol-7-yl}-methanesulfonamide

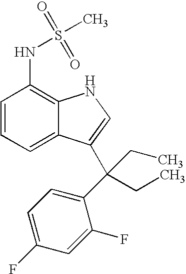

Title product is prepared as a white solid (75%). MS (ES⁺) 393 (M+1), MS (ES⁻) 391 (M−1). MP=144-147° C.

### EXAMPLE 63

### N-{3-[1-Ethyl-1-(4-trifluoromethyl-phenyl)-propyl]-1H-indol-7-yl}-methanesulfonamide

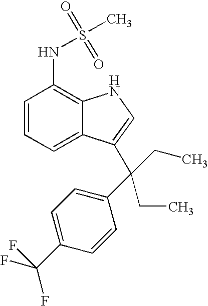

Title product is prepared as a white solid (12%). MS (ES⁻) 423 (M−1). LC/MS shows 100% purity.

### EXAMPLE 64

### N-{3-[1-(3,4-Difluoro-phenyl)-1-ethyl-propyl]-1H-indol-7-yl}-methanesulfonamide

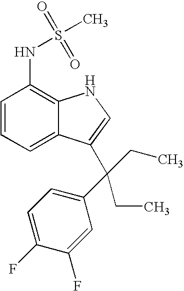

Title product is prepared as a white solid (54%). MS (ES⁺) 393 (M+1), MS (ES⁻) 391 (M−1). EA: theory (% C=61.2078, % H=5.6502, % N=7.1377), experimental (% C=61.05, % H=5.64, % N=6.98).

### EXAMPLE 65

### N-[3-(1-Methyl-1-p-tolyl-propyl)-1H-indol-7-yl]-methanesulfonamide

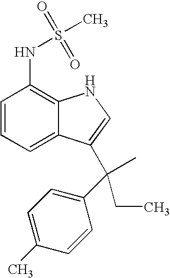

Title product is prepared as a white solid (78%). MS (ES⁻) 355 (M−1). LC/MS shows 93% purity.

### EXAMPLE 66

### N-{3-[1-Ethyl-1-(4-ethyl-phenyl)-propyl]-1H-indol-7-yl}-methanesulfonamide

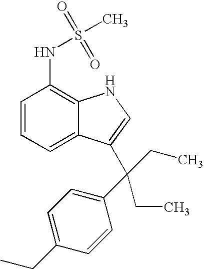

Title product is prepared as a white solid (54%). MS (ES⁺) 385 (M+1), MS (ES⁻) 383 (M−1).

### EXAMPLE 67

### N-[3-(1-Ethyl-1-o-tolyl-propyl)-1H-indol-7-yl]-methanesulfonamide

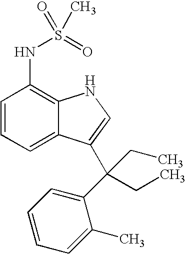

Title product is prepared as a white solid (16%). MS (ES⁺) 371 (M+1), MS (ES⁻) 369 (M−1).

### EXAMPLE 68

### N-{3-[1-Ethyl-1-(2-fluoro-phenyl)-propyl]-1H-indol-7-yl}-methanesulfonamide

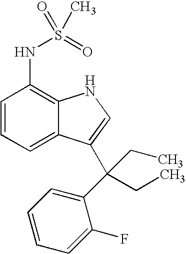

Title product is prepared as a white solid (26%). MS (ES⁺) 371 (M+1), MS (ES⁻) 369 (M−1). EA: theory (% C=64.1483, % H=6.1908, % N=7.4806), experimental (% C=64.00, % H=6.41, % N=7.43). MP=144-146° C.

Examples 69-72 below are made following procedures essentially as described in Example 54 above. That is; employing the procedures of Scheme I, and utilizing the appropriate indole and the appropriate carbinol, each of which may be obtained from commercial sources or prepared according to procedures as described in the Preparations herein, the title compounds of Examples 69-72 are prepared.

### EXAMPLE 69

### N-{3-[1-(4-Methoxy-phenyl)-1-propyl-butyl]-1H-indol-7-yl}-methanesulfonamide

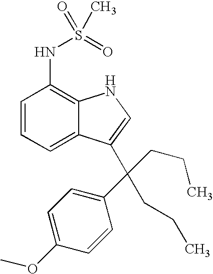

Title product is prepared as a white solid (71%). MS (ES⁺) 415 (M+1), MS (ES⁻) 413 (M−1).

### EXAMPLE 70

### N-[3-(1-Methyl-1-phenyl-butyl)-1H-indol-7-yl]-methanesulfonamide

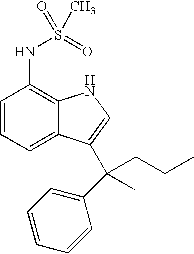

Title product is prepared as a white solid (51%). MS (ES⁺) 357 (M+1), MS (ES⁻) 355 (M−1).

### EXAMPLE 71

### N-[3-(1-Ethyl-1-m-tolyl-propyl)-1H-indol-7-yl]-methanesulfonamide

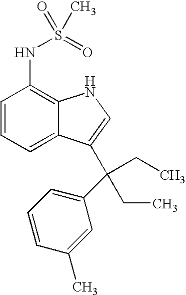

Title product is prepared as a white solid (77%). MS (ES⁺) 371 (M+1), MS (ES⁻) 369 (M−1).

### EXAMPLE 72

### N-{3-[1′-Ethyl-1-(3-fluoro-phenyl)-propyl]-1H-indol-7-yl}-methanesulfonamide

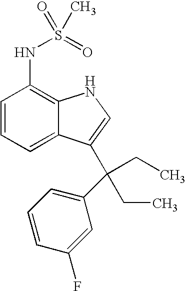

Title product is prepared as a white solid (49%). MS (ES⁺) 375 (M+1), MS (ES⁻) 373 (M−1).

Examples 73-78 below are made following procedures essentially as described in Example 54 above. That is, employing the procedures of Scheme 1, and utilizing the appropriate indole and the appropriate carbinol, each of which may be obtained from commercial sources or prepared according to procedures as described in the Preparations herein, the title compounds of Examples 73-78 are prepared.

### EXAMPLE 73

### N-{3-[1-(4-Fluoro-phenyl)-1-methyl-propyl]-1H-indol-7-yl}-methanesulfonamide

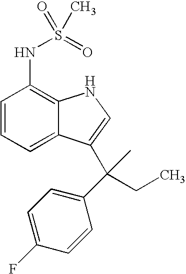

Title product is prepared as a white solid (70%). MS (ES⁺) 361 (M+1), MS (ES⁻) 359 (M−1).

### EXAMPLE 74

### N-[3-(1-Methoxymethyl-1-phenyl-propyl)-1H-indol-7-yl]-methanesulfonamide

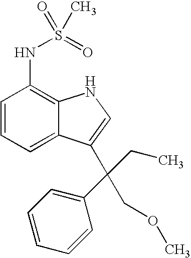

After preparative tlc purification (10% ethyl acetate in hexanes) title product is prepared as a light tian solid (3.4%). MS (ES⁺) 373 (M+1), MS (ES⁻) 371 (M−1).

### EXAMPLE 75

### 3-[1-Ethyl-1-(4-fluoro-phenyl)-propyl]-7-nitro-1H-indole

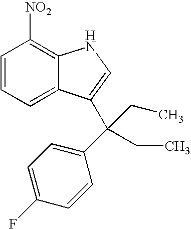

Title product is prepared as an orange crystalline solid (28%). ¹HNMR (CDCl₃) □: 9.82 (b, 1H), 8.08 (d, 1H), 7.40 (d, 1H), 7.25 (m, 2H), 7.16 (d, 1H), 6.91 (m, 3H), 2.16 (m, 4H), 0.66 (t, 6H).

### EXAMPLE 76

### N-{3-[1-(4-Fluoro-phenyl)-1-methyl-ethyl]-1H-indol-7-yl}-methanesulfonamide

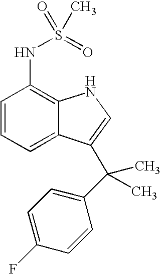

Title product is prepared as a white solid (29%). MS (ES⁺) 347 (M+1), MS (ES⁻) 345 (M−1).

### EXAMPLE 77

### N-{3-[1-(4-Methoxy-phenyl)-1-methyl-ethyl]-1H-indol-7-yl}-methanesulfonamide

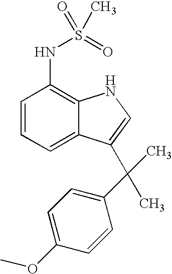

Title product is prepared as a white solid (58%). MS (ES⁺) 359 (M+1), MS (ES⁻) 357 (M−1).

### EXAMPLE 78

### N-[3-(1-Methyl-1-phenyl-ethyl)-1H-indol-7-yl]-methanesulfonamide

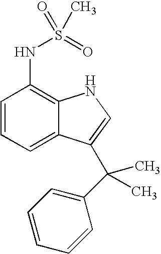

Title product is prepared as a white solid (34%). MS (ES⁺) 329 (+1), MS (ES⁻) 327 (M−1).

### EXAMPLE 79

### 3-[1-Ethyl-1-(4-fluoro-phenyl)-propyl]-1H-indol-7-ylamine

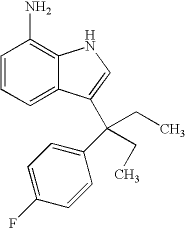

Utilizing 7-aminoindole and 3-(4-Fluoro-phenyl)-pentan-3-ol in procedures as described in Scheme VII provides the product as a purple solid (85%). MS (ES⁻) 295 (M−1).

### EXAMPLE 80

### N-{3-[1-Ethyl-1-(4-fluoro-phenyl)-propyl]-1-methyl-1H-indol-7-yl}-methanesulfonamide

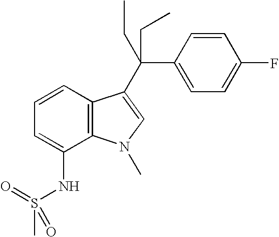

A. Combine 1-Methyl-7-nitro-1H-indole (0.831 g, 4.72 mmol) with 3-(4-Fluoro-phenyl)-pentan-3-ol (0.860 g, 4.72 mmol) in dichloromethane (50 mL) under a nitrogen atmosphere. Add trifluoroacetic acid (0.36 mL, 4.72 mmol) and stir at room temperature for 18 hours. Add a solution of saturated sodium bicarbonate (150 mL) and ethyl acetate (150 mL). Separate the layers, dry the organic layer with sodium sulfate, and concentrate. Purify the resulting compound on silica gel eluting with a gradient from 50% to 90% toluene in hexanes to give 0.221 g (14%) of 3-[1-Ethyl-1-(4-fluoro-phenyl)-propyl]-1-methyl-7-nitro-1H-indole:

¹H NMR(CDCl₃):7.67 (dd, 1H), 7.21-7.17 (m, 2H), 7.09 (s, 1H), 7.03 (dd, 1H), 6.95-6.91 (m, 2H), 6.80 (ap t, 3H), 3.84 (s, 13H), 2.18-2.05 (m, 41), 0.64 (t, 6H).

B. Dissolve 3-[1-Ethyl-1-(4-fluoro-phenyl)-propyl]-1-methyl-7-nitro-1H-indole (0.221 g, 0.649 mmol) in ethyl acetate (5 mL), and add a slurry of 10% Pd/C (0.220 g) in ethyl acetate (5 mL). Evacuate the reaction vessel, and place under an atmosphere of hydrogen. Stir at room temperature for 1.5 hours. Filter the reaction through a Celite pad and concentrate the filtrate. Purify the resulting compound on silica gel eluting with a gradient from 30% to 50% ethyl acetate in hexanes to give 0.191 g (100%) of 3-[1-Ethyl-1-(4-fluoro-phenyl)-propyl]-1-methyl-1H-indol-7-ylamine:

¹H NMR(CDCl₃):7.24-7.21 (m, 2H), 6.93-6.88 (m, 2H), 6.87 (s, 1H), 6.62-6.58 (m, 2H), 6.44-6.42 (m, 1H), 2.19-2.11 (m, 2H), 2.06-1.97 (m, 2H); 0.62 (t, 4H).

C. Dissolve 3-[1-Ethyl-1-(4-fluoro-phenyl)-propyl]-1-methyl-1H-indol-7-ylamine (0.191 g, 0.615 mmol) in dichloromethane (1 mL) under a nitrogen atmosphere. Add methanesulfonyl chloride (0.057 mL, 0.738 mmol) and pyridine (0.060 mL, 6.738 mmol). Stir for one hour at room temperature. Add a solution of saturated sodium bicarbonate (50 mL) and ethyl acetate (50 mL). Separate the layers, dry the organic layer with sodium sulfate, and concentrate. Purify the resulting compound on silica gel eluting with a gradient from 30% to 40% ethyl acetate in hexanes to give 0.174 g (73%) of the title compound: mass spectrum (ES+) m/z=389 (M+1).

### EXAMPLE 81

### N-[3-(1-Ethyl-1-phenyl-propyl)-1H-indol-7-yl]-acetamide

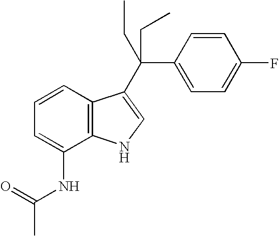

Combine N-(1H-Indol-7-yl)-acetamide (0.191 g, 1.10 mmol) with 3-(4-Fluoro-phenyl)-pentan-3-ol (0.200 g, 1.10 mmol) in dichloromethane (10 mL) under a nitrogen atmosphere. Add trifluoroacetic acid (0.13 mL, 1.64 mmol) and stir at room temperature for 18 hours. Add a solution of saturated sodium bicarbonate (50 mL) and ethyl acetate (50 mL). Separate the layers, dry the organic layer with sodium sulfate, and concentrate. Purify the resulting compound on silica gel eluting with a gradient from 50% to 70% ethyl acetate in hexanes to give 0.199 g (53%) of the title compound: mass spectrum (ES+) m/z=339 (M+1).

### EXAMPLE 82

### N-{3-[1-Ethyl-1-(4-fluoro-phenyl)-propyl]-1H-indol-7-yl}-N-methyl-methanesulfonamide

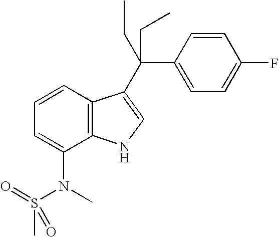

A. Dissolve N-(1H-Indol-7-yl)-methanesulfonamide (0.235 g, 1.12 mmol) in N,N-dimethylformamide (2 mL). Add potassium carbonate (0.170 g, 1.23 mmol) and stir at room temperature for 5 minutes. Add iodomethane (0.077 mL, 1.23 mmol) and stir at room temperature overnight. Partition the reaction between diethyl ether (60 mL) and water (60 ml) and separate the layers. Dry the organic layer with sodium sulfate and concentrate. Purify the resulting compound on silica gel eluting with a gradient from 40% to 50% ethyl acetate in toluene to give 0.169 g (67%) of N-(1H-Indol-7-yl)-N-methyl-methanesulfonamide:

¹H NMR(CDCl₃):8.89 (br s, 1H), 7.61 (dd, 1H), 7.28-7.25 (m, 1H), 7.14-7.06 (m, 2H), 6.58-6.56 (m, 1H), 3.40 (s, 3H), 2.93 (s, 3H).

B. Combine N-(1H-Indol-7-yl)-N-methyl-methanesulfonamide (0.165 g, 0.737 mmol) with 3-(4-Fluoro-phenyl)-pentan-3-ol (0.134 g, 0.737 mmol) in dichloromethane (5 mL) under a nitrogen atmosphere. Add trifluoroacetic acid (0.085 mL, 1.10 mmol) and stir at room temperature for 16 hours. Add a solution of saturated sodium bicarbonate (50 mL) and ethyl acetate (50 mL). Separate the layers, dry the organic layer with sodium sulfate, and concentrate. Purify the resulting compound on silica gel eluting with 10% ethyl acetate in toluene to give 0.179 g (62%) of the title compound: mass spectrum (ES+) m/z=389 (M+1).

### EXAMPLE 83

### N-{3-[1-Ethyl-1-(4-fluoro-phenyl)-propyl]-1H-indol-7-yl}-benzenesulfonamide

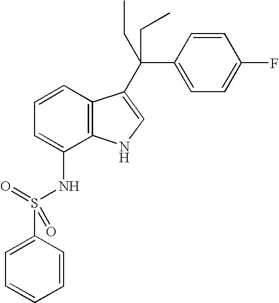

Dissolve 3-[1-Ethyl-1-(4-fluoro-phenyl)-propyl]-1H-indol-7-ylamine (0.216 g, 72.9 mmol) in dichloromethane (3 mL) under a nitrogen atmosphere. Add benzenesulfonyl chloride (0.102 mL, 80.2 mmol) and pyridine (0.065 mL, 80.2 mmol). Stir for one hour at room temperature. Add a solution of saturated sodium bicarbonate (50 mL) and ethyl acetate (50 mL). Separate the layers, dry the organic layer with sodium sulfate, and concentrate. Purify the resulting compound on silica gel eluting with 20% ethyl acetate in hexanes to give 0.216 g (68%) of the title compound: mass spectrum (ES+) m/z=437 (M+1).

### EXAMPLE 84

### Ethanesulfonic acid {3-[1-ethyl-1-(4-fluoro-phenyl)-propyl]-1H-indol-7-yl}-amide

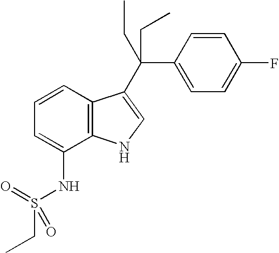

Dissolve 3-[1-Ethyl-1-(4-fluoro-phenyl)-propyl]-1H-indol-7-ylamine (0.206 g, 69.5 mmol) in dichloromethane (2 mL) under a nitrogen atmosphere. Add ethanesulfonyl chloride (0.079 mL, 83.4 mmol) and pyridine (0.067 mL, 83.4 mmol). Stir for one hour at room temperature. Add a solution of saturated sodium bicarbonate (50 mL) and ethyl acetate (50 mL). Separate the layers, dry the organic layer with sodium sulfate, and concentrate. Purify the resulting compound on silica gel eluting with 20% ethyl acetate in hexanes to give 0.154 g (57%) of the title compound: mass spectrum (ES+) m/z=389 (M+1).

### EXAMPLE 85

### Propane-2-sulfonic acid {3-[1-ethyl-1-(4-fluoro-phenyl)-propyl]-1H-indol-7-yl}-amide

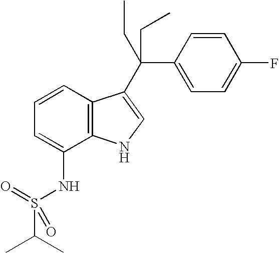

Dissolve 3-[1-Ethyl-1-(4-fluoro-phenyl)-propyl]-1H-indol-7-ylamine (0.246 g, 0.83 mmol) in dichloromethane (2 mL) under a nitrogen atmosphere. Add isopropylsulfonyl chloride (0.11 mL, 0.10 mmol) and pyridine (0.081 mL, 0.100 mmol). Stir for one hour at room temperature. Add a solution of saturated sodium bicarbonate (50 mL) and ethyl acetate (50 mL). Separate the layers, dry the organic layer with sodium sulfate, and concentrate. Purify the resulting compound on silica gel eluting with 20% ethyl acetate in hexanes to give 0.132 g (40%) of the title compound: mass spectrum (ES+) m/z=403 (M+1).

### EXAMPLE 86

### 3-[1-Ethyl-1-(4-fluoro-phenyl)-propyl]-1H-indole-7-carbaldehyde

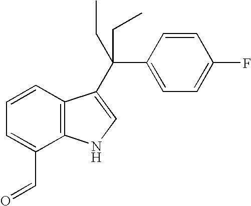

Combine 1H-Indole-7-carbaldehyde (0.534 g, 3.68 mmol) with 3-(4-Fluoro-phenyl)-pentan-3-ol (0.671 g, 3.68 mmol) in dichloromethane (3 mL) under a nitrogen atmosphere. Add trifluoroacetic acid (0.425 mL, 5.52 mmol) and stir at room temperature for 36 hours. Add a solution of saturated sodium bicarbonate (100 mL) and ethyl acetate (100 mL). Separate the layers, dry the organic layer with sodium sulfate, and concentrate. Purify the resulting compound on silica gel eluting with 70% dichloromethane in hexanes to give 0.845 g (74%) of the title compound: mass spectrum (ES+) m/z=310 (M+1).

### EXAMPLE 87

### {3-[1-Ethyl-1-(4-fluoro-phenyl)-propyl]-1H-indol-7-yl}-methanol

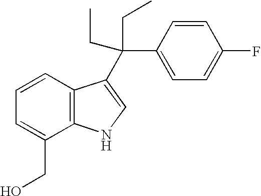

Dissolve 3-[1-Ethyl-1-(4-fluoro-phenyl)-propyl]-1H-indole-7-carbaldehyde (0.825 g 2.67 mmol) in a mixture of methanol (5 mL) and tetrahydrofuran (2 mL) under a nitrogen atmosphere. Add sodium borohydride (0.101 g, 2.67 mmol) and stir at room temperature for 45 minutes. Add water (100 mL) and ethyl acetate (100 mL). Separate the layers. Dry the organic layer with sodium sulfate and concentrate. Purify the resulting compound on silica gel eluting with 35% ethyl acetate in hexanes to give 0.643 g (77%) of the title compound: mass spectrum (ES+) m/z=312 (M+1).

### EXAMPLE 88

### 3-[1-Ethyl-1-(4-fluoro-phenyl)-propyl]-7-methanesulfonyl-1H-indole

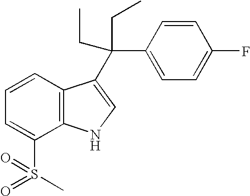

Dissolve 3-[1-Ethyl-1-(4-fluoro-phenyl)-propyl]-7-methylsulfanyl-1H-indole (0.670 g, 2.05 mmol) in dichloromethane (15 mL) under a nitrogen atmosphere. Add m-CPBA (1.01 g, 4.50 mmol) and stir the reaction at room temperature for 1.5 hours. Add a solution of saturated sodium bicarbonate (100 mL) and ethyl acetate (100 mL). Separate the layers. Dry the organic layer with sodium sulfate and concentrate. Purify the resulting compound on silica gel eluting with 20% ethyl acetate in hexanes to give 0.443 g (60%) of the title compound: mass spectrum (ES−) m/z=358 (M−1).

### EXAMPLE 89

### N-{3-[1-Ethyl-1-(4-fluoro-phenyl)-propyl]-2-methyl-1H-indol-7-yl}-methanesulfonamide

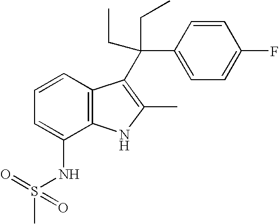

A. Combine 7-Bromo-2-methyl-1H-indole (0.905 g, 4.31 mmol) with 3-(4-Fluoro-phenyl)-pentan-3-ol (0.785 g, 4.31 mmol) in dichloromethane (20 mL) under a nitrogen atmosphere. Add trifluoroacetic acid (0.498 mL, 6.47 mmol) and stir at room temperature for 18 hours. Add a solution of saturated sodium bicarbonate (100 mL) and ethyl acetate (100 mL). Separate the layers, dry the organic layer with sodium sulfate, and concentrate. Purify the resulting compound on silica gel eluting with 50% dichloromethane in hexanes to give 0.457 g (28%) of 7-Bromo-3-[1-ethyl-1-(4-fluoro-phenyl)-propyl]-2-methyl-1H- indole.

B. Dissolve 7-Bromo-3-[1-ethyl-1-(4-fluoro-phenyl)-propyl]-2-methyl-1H-indole (1.34 g, 3.58 mmol) in tetrahydrofuran and cool the reaction to −78° C. Add 1.6 M nBuLi in hexanes (6.71 mL, 10.7 mmol). Warm to 0° C. for 30 minutes, and then cool to −78° C. Add diphenylphosphoryl azide (1.54 mL, 7.16 mmol) and stir at −78° C. for one hour. Warm to −40° C. and add Red-Al (5.4 mL, 17.9 mmol). Stir the reaction for one hour at 0° C. Add water at 0° C. and filter the resulting solid. Wash the solid with water and ethyl acetate and combine the filtrates. Separate the layers, dry the organic layer with sodium sulfate, and concentrate. Purify the resulting compound on silica gel eluting with 40% ethyl acetate in hexanes to give 0.393 g (60%) of 3-[1-Ethyl-1-(4-fluoro-phenyl)-propyl]-2-methyl-1H-indol-7-ylamine: mass spectrum (ES⁺) m/z=310 (M+1).

C. Dissolve 3-[1-Ethyl-1-(4-fluoro-phenyl)-propyl]-2-methyl-1H-indol-7-ylamine (0.120 g, 0.387 mmol) in dichloromethane (2 mL) under a nitrogen atmosphere. Add methanesulfonyl chloride (0.033 mL, 0.425 mmol) and pyridine (0.034 mL, 0.425 mmol). Stir the reaction at room temperature for two hours. Add a solution of saturated sodium bicarbonate (50 mL) and ethyl acetate (50 mL). Separate the layers, dry the organic layer with sodium sulfate, and concentrate. Purify the resulting compound on silica gel eluting with 25% ethyl acetate in hexanes to give 0.086 g (57%) of the title compound: mass spectrum (ES+) m/z=389 (M+1).

### EXAMPLE 90

### 3-[1-Ethyl-1-(4-fluoro-phenyl)-propyl]-1H-indole

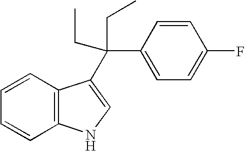

Combine indole (0.150 g, 1.28 mmol) with 3-(4-Fluoro-phenyl)-pentan-3-ol (0.233 g, 1.28 mmol) in dichloromethane (2 mL) under a nitrogen atmosphere. Add trifluoroacetic acid (0.15 mL, 1.92 mmol) and stir at room temperature for 18 hours. Add a solution of saturated sodium bicarbonate (50 mL) and ethyl acetate (50 mL). Separate the layers, dry the organic layer with sodium sulfate, and concentrate. Purify the resulting compound on silica gel eluting with toluene to give 0.227 g (63%) of the title compound: mass spectrum (ES−) m/z=280 (M−1).

### EXAMPLE 91

### 3-[1-Ethyl-1-(4-fluoro-phenyl)-propyl]-5-fluoro-1H-indole

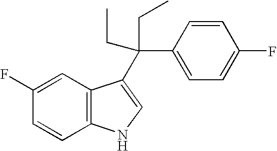

Combine 5-fluoroindole (0.255 g, 1.89 mmol) with 3-(4-Fluoro-phenyl)-pentan-3-ol (0.379 g, 2.08 mmol) in dichloromethane (8 mL) under a nitrogen atmosphere. Add trifluoroacetic acid (0.22 mL, 2.84 mmol) and stir at room temperature for 18 hours. Add a solution of saturated sodium bicarbonate (50 mL) and ethyl acetate (50 mL). Separate the layers, dry the organic layer with sodium sulfate, and concentrate. Purify the resulting compound on silica gel eluting with a gradient from 5% to 10% ethyl acetate in hexanes to give 0.337 g (60%) of the title compound: mass spectrum (ES−) m/z=298 (M−1).

### EXAMPLE 92

### 3-[1-Ethyl-1-(4-fluoro-phenyl)-propyl]-5-methoxy-1H-indole

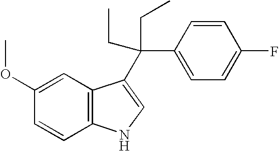

Combine 5-methoxyindole (0.255 g, 1.73 mmol) with 3-(4-Fluoro-phenyl)-pentan-3-ol (0.347 g, 1.91 mmol) in dichloromethane (10 mL) under a nitrogen atmosphere. Add trifluoroacetic acid (0.20 mL, 2.60 mmol) and stir at room temperature for 18 hours. Add a solution of saturated sodium bicarbonate (50 mL) and ethyl acetate (50 mL). Separate the layers, dry the organic layer with sodium sulfate, and concentrate. Purify the resulting compound on silica gel eluting 5% ethyl acetate in hexanes to give 0.350 g (65%) of the title compound: mass spectrum (ES+) m/z=312 (M+1).

Examples 93-98 below are made following procedures essentially as described in Example 1 above. That is, employing the procedures of Scheme I, and utilizing the appropriate indole and the appropriate carbinol, each of which may be obtained from commercial sources or prepared according to procedures as described in the Preparations herein, the title compounds of Examples 93-98 are prepared.

### EXAMPLE 93

### N-{3-[1-Cyclopropyl-1-(5-fluoro-benzofuran-2-yl)-ethyl]-1H-indol-7-yl}-methanesulfonamide

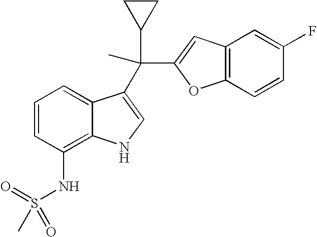

Flash chromatography eluting with a gradient (0 to 100 ethyl acetate/hexanes over 25 minutes) provides the title compound as a white solid (210 mg, 82%).

LC-MS m/z 413.1 (M⁺+1)

### EXAMPLE 94

### N-{3-[1-(5-Chloro-7-fluoro-benzofuran-2-yl)-1-cyclopropyl-ethyl]-1H-indol-7-yl}-methanesulfonamide

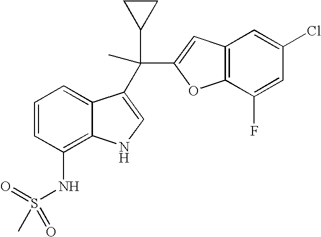

Flash chromatography eluting with a gradient (0 to 100 ethyl acetate/hexanes over 25 minutes) provides the title compound as a white solid (1.15 g, 74%). ¹H NMR (CDCl₃, 400 MHz): d 0.37 (m, 2H), 0.57 (m, 2H), 1.65 (m, 1H), 1.72 (s, 3H), 3.03 (s, 3H), 6.52 (d, 1H), 6.69 (s, 1H), 6.89 (m, 2H), 6.95 (dd, 1H), 7.20 (dd, 1H), 7.24 (d, 1H), 7.31 (d, 1H), 9.14 (broad s, 1H).

### EXAMPLE 95

### N-[3-(1-Cyclopropyl-1-furo[3,2-b]pyridin-2-yl-ethyl)-1H-indol-7-yl]-methanesulfonamide

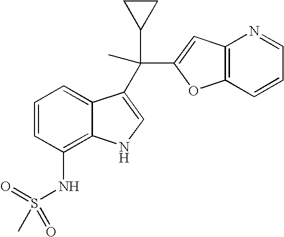

A solution of 1-cyclopropyl-1-furo[3,2-b]pyridin-2-yl-ethanol (380 mg, 1.87 mmol, 1.10 eq), N-(1H-indol-7-yl)-methanesulfonamide (357 mg, 1.70 mmol), and TFA (0.39 ml) in dichloromethane (6 ml) is stirred at 50° C. overnight. The solution is diluted with ether, washed with water, dried over anhydrous sodium sulfate, and concentrated. The brown residue (918 mg) is purified on a 40 g silica column (0 to 100 ethyl acetate/hexanes over 25 minutes) to give the title compound as a pale yellow solid (559 g, 83%).

LC-MS m/z 396.0 (M⁺+1).

### EXAMPLE 96

### N-[3-(1-Cyclopropyl-1-methyl-3-trimethylsilanyl-prop-2-ynyl)-1H-indol-7-yl]-methanesulfonamide

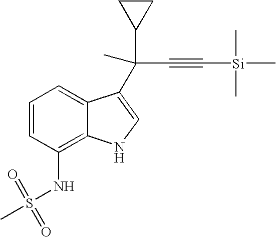

Flash chromatography on 40 g of silica eluting with a gradient (0 to 100 ethyl acetate/hexanes over 30 minutes) provides the title compound as a yellow solid (0.84 g, 60%).

LC-MS m/z 375.2 (M⁺+1).

### EXAMPLE 97

### N-{3-[1-(2,2-Difluoro-benzo[1,3]dioxol-5-yl)-1-ethyl-propyl]-1H-indol-7-yl}-methanesulfonamide

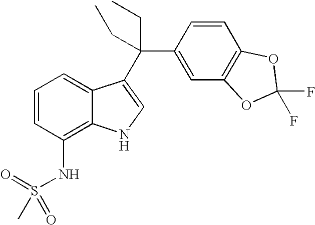

Flash chromatography eluting with a gradient (0 to 100 ethyl acetate/hexanes over 25 minutes) provides the title compound as a white solid (477 mg, 42%).

LC-MS m/z 437.1 (M⁺+1).

### EXAMPLE 98

### N-[3-(1-Benzo[1,3]dioxol-5-yl-1-ethyl-propyl)-1H-indol-7-yl]-methanesulfonamide

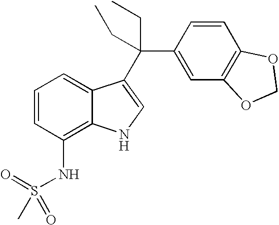

Flash chromatography eluting with a gradient (0 to 100 ethyl acetate/hexanes over 25 minutes) provides the title compound as a white solid (1.01 g, 100%).

LC-MS m/z 401.1 (M⁺+1).

### EXAMPLE 99

### N-{3-[1-Cyclopropyl-1-(7-fluoro-benzofuran-2-yl)-ethyl]-1H-indol-7-yl}-methanesulfonamide

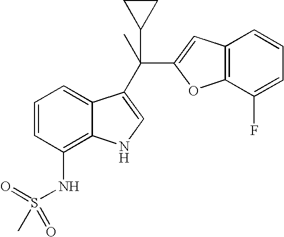

A mixture of N-{3-[1-(5-Chloro-7-fluoro-benzofuran-2-yl)-1-cyclopropyl-ethyl]-1H indol-7-yl}-methanesulfonamide (0.93 g, 2.08 mmol), 5% Pd/C (164 mg), triethyl amine (0.6 ml) in THF (4 ml)/ethanol (95 ml) is hydrogenated at 60 psi overnight. The mixture is then filtered through celite and concentrated to provide the title compound (0.79 g, 92%).

LC-MS m/z 413.1 (M⁺+1)

### EXAMPLE 100

### N-[3-(1-Cyclopropyl-1-methyl-prop-2-ynyl)-1H-indol-7-yl]-methanesulfonamide

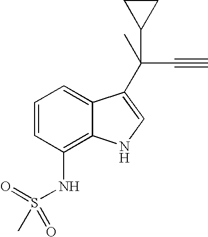

A solution of N-[3-(1-Cyclopropyl-1-methyl-3-trimethylsilanyl-prop-2-ynyl)-1H-indol-7-yl]-methanesulfonamide (0.84 g, 2.24 mmol) and potassium carbonate (0.8 g) in methanol (10 ml)/water (0.5 ml) is stirred at 45° C. for 48 hours. The solution is diluted with water/ether, the organic phase is washed with water (2×), dried over anhydrous sodium sulfate, and concentrated. The beige residue (0.54 g) is purified on a 40 g silica column (0 to 100 ethyl acetate/hexanes over 25 minutes) to provide the title compound as a white solid (0.47 g, 69%).

LC-MS m/z 303.0 (M⁺+1).

Examples 101-114 below are made following procedures essentially as described in Example 1 above. That is, employing the procedures of Scheme I, and utilizing the appropriate indole and the appropriate carbinol, each of which may be obtained from commercial sources or prepared according to procedures as described in the Preparations herein, the title compounds of Examples 101-114 are prepared.

### EXAMPLE 101

### N-(3-(1-4-Chloro-benzo(b)thiophen-2-yl)-1-ethyl-propyl)-1H-indol-7-yl)-methanesulfonamide

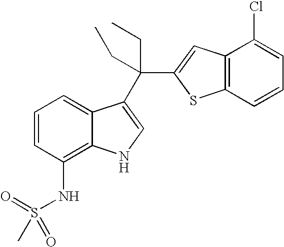

Flash chromatography eluting with 1:1 hexanes:ethyl acetate provides 0.229 g (35%) of the title compound.

MS m/z: 445.2, 447.2 (ES−)

### EXAMPLE 102

### N-(3-(1-(6-Trifluoromethyl-benzo(b)thiophen-2-yl)-1-ethyl-propyl)-1H-indol-7-yl)-methanesulfonamide

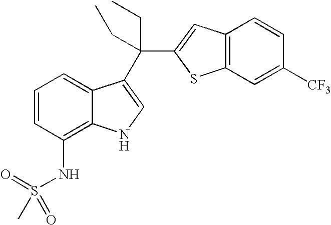

Flash chromatography eluting with 1:1 hexanes:ethyl Acetate provides 0.279 g (62%) of the title compound.

MS m/z: 479.2 (ES−)

### EXAMPLE 103

### N-(3-(1-(5-Fluoro-benzo(b)thiophen-2-yl)-1-ethyl-propyl)-1H-indol-7-yl)-methanesulfonamide

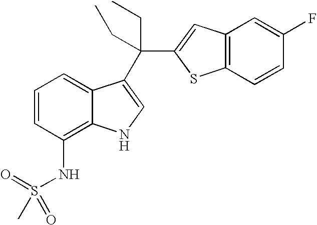

Flash chromatography eluting with 1:1 hexaneseEthyl Acetate, followed by recrystalization in ether hexane provides 0.020 g (9.4%) of the title compound.

MS m/z: 429.3 (ES−)

### EXAMPLE 104

### N-(3-(1-(4-Trifluoromethyl-benzo(b)thiophen-2-yl)-ethyl-propyl-1H-indol-7-yl)-methanesulfonamide

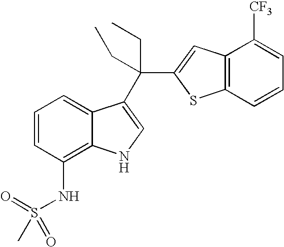

Flash chromatography eluting with 1:1 hexanes:ethyl acetate provides 0.3 g (40%) of the title compound.

MS m/z: 479.2 (ES−)

### EXAMPLE 105

### N-3-(1-(4-Fluoro-benzo(b)thiophen-2-yl)-1-ethyl-propyl)-1H-indol-7-yl)methanesulfonamide

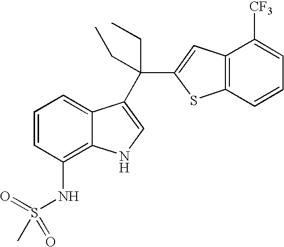

Flash Chromatography eluting with 55/45 hex/etoac followed by recrystalization in ether/hexane/trace of ethyl acetate provides 0.129 g (5%) of the title compound.

MS m/z: 429.2 (ES−)

### EXAMPLE 106

### N-(3-(1-(6-Chloro-benzo(b)thiophen-2-yl)-1-ethyl-propyl)-1H-indol-7-yl)methanesulfonamide

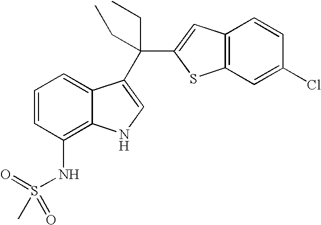

Flash chromatography eluting with 1/1 hexanes/ethyl acetate followed by recrystalization in ether/hexane provides 0.060 g (8%) of the title compound.

MS m/z: 445.2, 447.2 (chloro pattern) (ES−)

### EXAMPLE 107

### N-(3-1-(7-Fluoro-benzo(b)thiophen-2-yl)-1-ethyl-propyl)-1H-indol-7-yl)methanesulfonamide

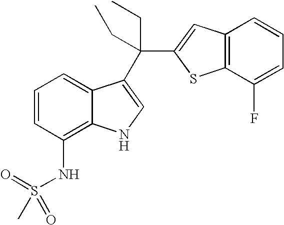

Recrystalization in ethyl acetate/hexane provides 0.110 g (15.76% yield) of the title compound.

MS m/z: 429.2 (ES−)

### EXAMPLE 108

### N-(3-(1-(7-Trifluoromethyl-benzo(b)thiophen-2-yl)-1-ethyl-propyl)-1H-indol-7-yl-methanesulfonamide

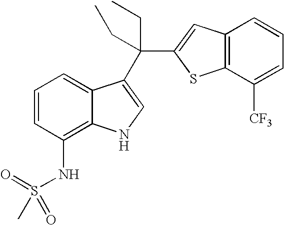

Flash chromatography with 2/1 hexanes/ethyl acetate increasing polarity to 1/1 hexanes/ethyl acetate followed by recrystalization in heaxane/ether provides 0.101 g (20%) of the title compound.

MS m/z: 479.2 (ES−)

### EXAMPLE 109

### N-(3-(1-(4-Chloro-benzo(b)thiophen-2-yl)-1-methyl-ethyl)-1H-indol-7-yl)-methanesulfonamide

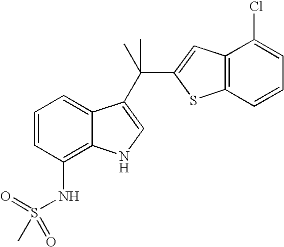

Flash chromatography using 2/1 heaxanes/ethyl acetate followed by recrystalization in ether/hexane provides 20 mg (10.8% yield)

MS m/z: 417.1, 419.1 (chloro pattern) (ES−)

### EXAMPLE 110

### N-(3-(1-(5-Trifluoromethyl-benzo(b)thiophen-2-yl)-1-ethyl-propyl)-1H-indol-7-yl)-methanesulfonamide

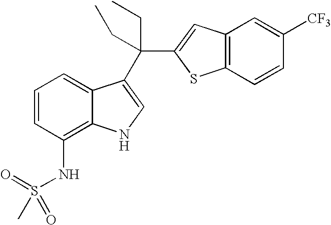

Flash chromatography using 1/1 hexanes/ethyl acetate followed by recrystalization in ether/hexane provides 0.110 g (18% yield) of the title compound.

MS m/z: 479.2 (ES−)

### EXAMPLE 111

### N-(3-(1-(3-Methyl-4-Fluoro-benzo(b)thiophen-2-yl)-1-ethyl-propyl)-1H-indol-7-yl)-methanesulfonamide

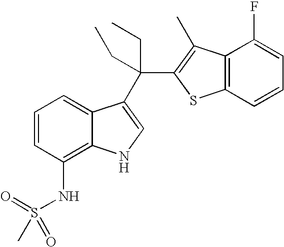

Flash chromatography eluting with 2/1 hexanes/ethyl acetate and increasing polarity to 1/1 hexanes/ethyl acetae followed by recrystalization in ether/hexane/ethyl acetate provides 0.100 g (22.8%) of the title compound.

MS m/z: 443.1 (ES−)

### EXAMPLE 112

### N-(3-(1-(3 methyl-7-fluoro-benzo(b)thiophen-2-yl)-1-ethyl-propyl)-1H-indol-7-yl)methanesulfonamide

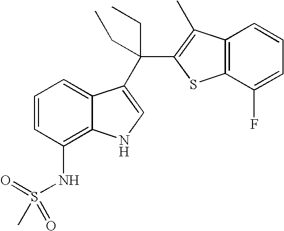

Flash chromatography eluting with 2/1 hexanes/ethyl acetate followed by recrystalization in ether and hexane and ethyl acetate provides 0.660 g (155%) of the title compound.

MS m/z: 443.2 (ES−)

### EXAMPLE 113

### N-(3-(1-cyclopropyl-1-(4-fluoro-benzo(b)thiophen-2-yl)-ethyl)-1H-indol-7-yl)-methanesulfonamide

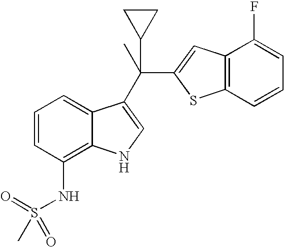

Flash chromatography eluting with 3/1 hexanes/ethyl acetate and increasing polarity gradually to 1/1 hexanes/ethyl acetate provides 0.240 g (20%) of the Title compound.

MS m/z: 427.1 (ES−)

### EXAMPLE 114

### N-(3-(1-cyclopropyl-1-(7-fluoro-benzo(b)thiophen-2-yl)-ethyl)-1H-indol-7-yl)-methanesulfonamide

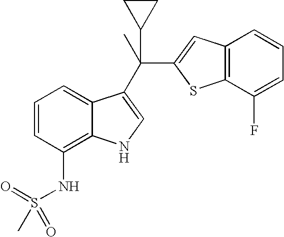

Flash chromatography eluting with 2/1 hexanes/ethyl acetate followed by recrystalization in ether/hexane/trace ethyl acetate provides 0.303 g (26%) of the Title compound.

MS m/z: 427.1 (ES−)

### EXAMPLE 115

### N-[3-(1-Ethyl-1-pyridin-3-yl-propyl)-1H-indol-7-yl]-methanesulfonamide

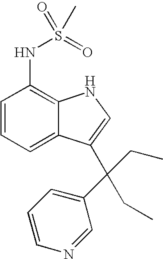

Following procedures as described in Scheme XIX: 3-Pyridin-3-yl-penta-1,4-diyn-3-ol (206 mg, 1.3 mmol), prepared according to procedures as described in Preparation 1, is dissolved in dichloromethane (5 ml) then stirred at ambient temperature under nitrogen atmosphere. Dicobaltoctacarbonyl (447 mg, 1.3 mmol) is added and the reaction stirred until gas evolution ceased (30 min.). To this mixture is then added N-(1H-indol-7-yl)-methanesulfonamide (250 mg, 1.2 mmol) followed by trifluoroacetic acid (0.275 ml, 3.6 mmol). The reaction is monitored by tlc (1:1 hexanes:ethyl acetate) until starting material is consumed. The reaction is concentrated and the residue is dissolved in ethanol. To this solution is added ammonium formate (742 mg, 11.8 mmol) and 10% Pd on carbon (100 mg). The reaction is heated to reflux for 24 hrs. After this time it is filtered through celite and evaporated. The residue is purified via flash chromatography in 5% methanol in dichlormethane to give 125 mg of the product as a white solid (29%). MS (ES⁺) 358 (M+1), MS (ES⁻) 356 (M−1).

### EXAMPLE 116

### N-[3-(1,1-Diethyl-prop-2-ynyl)-1H-indol-7-yl]-methanesulfonamide

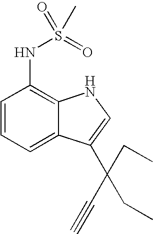

Utilizing the procedures as described in Scheme XX: 3-Ethyl-pent-1-yn-3-ol (1 g, 8.9 mmol), prepared according to procedures as described in Preparation 3 (using 3-pentanone and acetylene), is dissolved in dichloromethane (20 ml) then stirred at ambient temperature under nitrogen atmosphere. Dicobaltoctacarbonyl (3.05 g, 8.9 mmol) is added and the reaction stirred until gas evolution ceased (30 min.). To this mixture is then added N-(1H-Indol-7-yl)-methanesulfonamide (1.87 mg, 8.9 mmol) and cooled to 0° C. Then boron trifluoride diethyl eherate is added (2.26 ml, 17.8 mmol) and the reaction is monitored by tlc (1:1 hexanes:ethyl acetate) until starting material is consumed. The reaction is concentrated and the residue is dissolved in ethanol (20 ml). To this solution is added iron(III)nitrate nonahydrate (18 g, 44.5 mmol) and the reaction stirred until gas evolution ceased. After this time it is filtered through celite, washed with water, dried over magnesium sulfate and evaporated. The residue is purified via flash chromatography in 20% ethyl acetate in hexanes to give 471 mg of the product as a white solid (17%). MS (ES⁺) 305 (M+1), MS (ES⁻) 303 (M−1).

### EXAMPLE 117

### N-{3-[1-Ethyl-1-(1H-indol-3-yl)-propyl]-1H-indol-7-yl}-methanesulfonamide

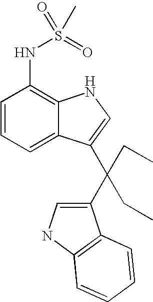

Following procedures as described in Scheme I and using 3-[1-(Toluene-4-sulfonyl)-1H-indol-3-yl]-pentan-3-ol (340 mg, 0.95 mmol) [prepared according to procedures as described in Preparation 1 (using 1-(Toluene-4-sulfonyl)-1H-indole-3-carboxylic acid ethyl ester and ethyl grinard)] and N-(1H-Indol-7-yl)-methanesulfonamide (200 mg, 0.95 mmol). The reaction is monitored by tlc (1:1 hexanes:ethyl acetate) until starting material is consumed. The reaction is concentrated and the residue is dissolved in methanol (15 ml) and water (5 ml). To this solution is added potassium carbonate (251 mg, 4.75 mmol) and the reaction stirred at reflux for 24 hrs. After this time it is partitioned in water/ethyl acetate and the organic is washed with brine, dried over magnesium sulfate and evaporated. The residue is purified via flash chromatography in 20% ethyl acetate in hexanes to give 88 mg of the product as a white solid (54%). MS (ES⁺) 396 (M+1), MS (ES⁻) 394 (M−1).

### EXAMPLE 118

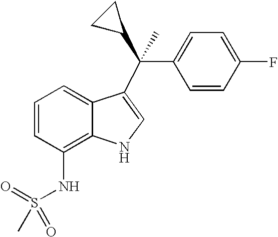

### (S)-(+)-N-{3-[1-cyclopropyl-1-(4-fluro-phenyl)-ethyl]-1H indol-7-yl}methansulfonamide

A. Preparation of

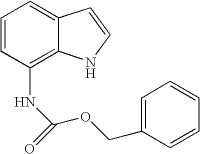

Utilizing the procedures of Scheme XXII, Step A: Indole aniline (800 mg, 6.05 mmol) is dissolved in water (7.5 mL) and methanol (7.5 mL). The resulting solution is cooled to 0° C. in a saltwater/ice bath. Sodium carbonate (1.28 g, 12.1 mmol) is added and the resulting slurry stirred for 5 minutes. Benzyl chloroformate (1.04 mL, 7.26 mmol) is added and the reaction stirred at 0° C. for 30 minutes. The reaction mixture is then concentrated on the buchi to remove the methanol. The aqueous layer is extracted with CH₂Cl₂ (2×15 mL). The combined organics are dried (MgSO₄), filtered and concentrated to provide the intermediate as a purple solid (II) (1.53 g, 5.75 mmol, 95%):

¹H NMR (DMSO-d₆) δ 10.8 (broad s, 1H), 9.4 (broad s, 1H), 6.9-7.5 (m, 8H), 6.9 (t, 1H, J=7.8 Hz), 6.4 (q, 1H, J=1.8 Hz), 5.2 (s, 2H); mass spectrum (m+1): 267.2 found.
B. Preparation of:

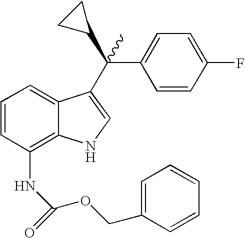

Utilizing the procedures of Scheme XXII, Step B: The carbamate product of Step A above (1.47 g, 5.52 mmol) and the appropriate tertiary alcohol (1.1 g, 6.07 mmol) are dissolved in CH₂Cl₂ (75 mL). Trifluoroacetic acid (510 μL, 6.67 mmol) is added and the resulting solution is stirred at rt for 30 minutes. The reaction is then quenched with saturated aqueous NaHCO₃ (75 mL). The aqueous layer is extracted with CH₂Cl₂ (25 mL). The combined organics are dried (MgSO₄), filtered and concentrated to provide the intermediate as a purple foam (2.53 g, 5.9 mmol, 107% recovery): ¹H NMR (DMSO-d₆) δ 10.6 (broad s, 1H), 9.3 (broad s, 1H), 7.3-7.4 (m, 9H), 7.0 (t, 2H, J=8.5 Hz), 6.6 (t, 1H, J=7.5 Hz), 6.4 (d, 1H, J=8.0 Hz), 5.2 (s, 2H), 1.5 (m, 1H), 1.48 (s, 3H), 0.47 (m, 1H), 0.39 (m, 1H), 0.17 (m, 1H), 0.06 (m, 1H); mass spectrum (m+1): 429.2 found.
C. Preparation of:

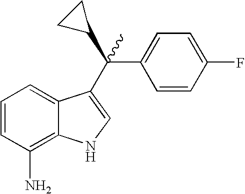

Utilizing the procedures of Scheme XXII, Step C: The coupled carbamate intermediate of Step B above (640 mg, 1.49 mmol) is dissolved in ethanol (50 mL). 10 wt. % Pd/C (64 mg, 10 wt. %) is added and the reaction is hydrogenated at 40 psi and 40° C. overnight. The reaction is then cooled to rt and the catalyst is filtered off and washed with ethanol. The filtrate is concentrated to provide the aniline as a purple oil (400 mg, 1.36 mmol, 91%): ¹H NMR (DMSO-d₆) δ 10.4 (broad s, 1H), 7.3 (m, 3H), 7.0 (t, 2H, J=9 Hz), 6.4 (t, 1H, J=8.1 Hz), 6.2 (AB, 1H, J=6.6 Hz, 0.9 Hz), 5.9 (d, 1H, J=8.1 Hz), 5.0 (broad s, 2H), 1.5 (m, 1H), 1.46 (s, 3H), 0.39 (m, 2H), 0.15 (m, 1H), 0.08 (m, 1H); mass spectrum (m+1) 295.3 found.
D. Preparation of:

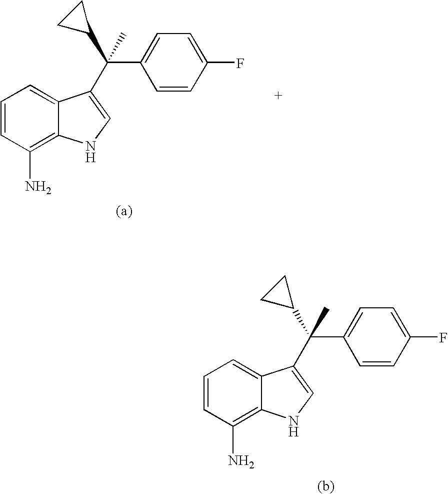

Using chiral chromatography methods, the racemic mixture of Step C above is resolved into the corresponding enantiomers. Conditions for the chiral chromatography:

Column: 4.6×150 mm Chiralcel OD

Eluent: 20% IPA/Heptane 0.01% dmea

Flow: 0.6 mL/min

Uv: 286 nm

Ms: 374 mz

E. Preparation of Final Title Compound (Example 118):

Enantiomer (a) from Step D above (1.817 g, 6.17 mmol) is dissolved in CH₂Cl₂ (20 mL). Pyridine (600 μL, 7.41 mmol) followed by Methanesulfonyl Chloride (525 μL, 6.79 mmol) is added and the reaction is stirred at rt overnight. The reaction is then quenched with 1 M HCl (20 mL). The organic layer is concentrated to an oil and then redissolved in ethyl acetate (30 mL) and washed with 1 M HCl (20 mL), water (20 mL), and saturated aqueous NaCl (20 mL). The organic layer is dried (MgSO₄), filtered and concentrated to a brown foam (2.48 g, 6.66 mmol, 108% recovery). The foam is adsorbed onto silica (3 g) and loaded onto 8 g silica. It is then eluted with 50% ethyl acetate/hexanes. Fractions containing product are collected and concentrated to an orange oil. The oil is slurried in ethyl acetate/hexanes to precipitate out solid. The slurry is filtered and washed with hexanes and orange crystals are collected. The solid is given two methanol/activated charcoal treatments and the Title compound collected as white crystals.

(1.3 g, 3.49 mmol, 57%): ¹H NMR (CDCl₃) δ 9.0 (broad s, 1H), 7.3 (m, 3H), 6.9 (m, 2H), 6.8 (m, 3H), 6.4 (broads, 1H), 3.0 (s, 3H), 1.6 (s, 3H), 1.5 (m, 1H), 0.5 (m, 2H), 0.3 (m, 1H), 0.1 (m, 1H); mass spectrum (m+1) 373.2 found.

Examples 119-133 below are made following procedures essentially as described in Example 1 above. That is, employing the procedures of Scheme I-IIA, and utilizing the appropriate indole and the appropriate carbinol, each of which may be obtained from commercial sources or prepared according to procedures as described in the Preparations herein, the title compounds of Examples 119-133 are prepared.

### EXAMPLE 119

### N-{3-[1-(3-Chloro-4-methoxy-phenyl)-1-ethyl-propyl]-1H-indol-7-yl}-methanesulfonamide

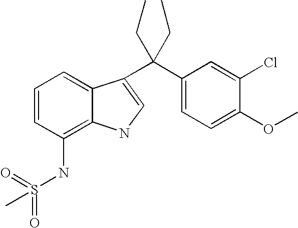

Flash chromatography eluting with a gradient (0 to 100 ethyl acetate/hexanes over 25 minutes) provides the title compound as a white solid (2.50 g, 100%).

LC-MS m/z 421.0 (M⁺+1).

### EXAMPLE 120

### N-{3-[1-Ethyl-1-(3-fluoro-4-methoxy-phenyl)-propyl]-1H-indol-7-yl}-methanesulfonamide

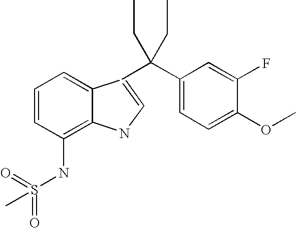

Flash chromatography eluting with a gradient (0 to 100 ethyl acetate/hexanes over 25 minutes) provides the title compound as a white solid (2.22 g, 93%).

LC-MS m/z 405.0 (M⁺+1).

### EXAMPLE 121

### N-{3-[1-Ethyl-1-(4-fluoro-3-methoxy-phenyl)-propyl]-1H-indol-7-yl}-methanesulfonamide

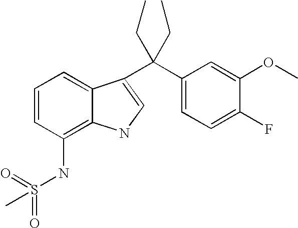

Flash chromatography eluting with a gradient (30 to 100 ethyl acetate/hexanes over 30 minutes) provides the title compound as a white solid (2.36 g, 99%).

LC-MS m/z 405.0 (M⁺+1).

### EXAMPLE 122

### N-{3-[1-(4-Chloro-3-methoxy-phenyl)-1-ethyl-propyl]-1H-indol-7-yl}-methanesulfonamide

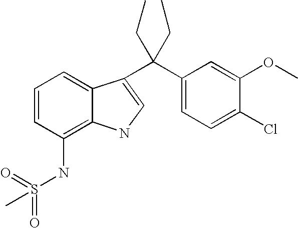

Flash chromatography eluting with a gradient (30 to 100 ethyl acetate/hexanes over 30 minutes) provides the title compound as a white solid (2.25 g, 98%).

LC-MS m/z 422.0 (M⁺+1).

### EXAMPLE 123

### N-{3-[1-Cyclopropyl-1-(3-fluoro-4-methoxy-phenyl)-ethyl]-1H-indol-7-yl)}-methanesulfonamide

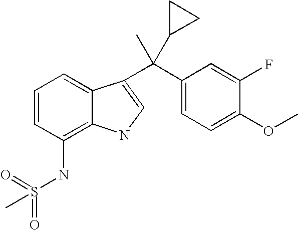

Flash chromatography eluting with a gradient (20 to 100 ethyl acetate/hexanes over 30 minutes) provides the title compound as a white solid (5.45 g, 93%).

LC-MS m/z 403.0 (M⁺+1).

### EXAMPLE 124

### N-{3-[1-(4-Chloro-3-methoxy-phenyl)-1-cyclopropyl-ethyl]-1H-indol-7-yl}-methanesulfonamide

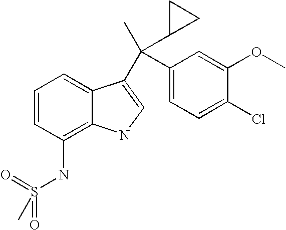

Flash chromatography eluting with a gradient (0 to 100 ethyl acetate/hexanes over 25 minutes) provides the title compound as a white solid (4.41 g, 82%).

¹H NMR (400 MHz, CDCl3): δ 0.21 (m, 1H), 0.33 (m, 1H), 0.49 (m, 1H), 0.58 (m, 1H), 1.59 (m, 1H), 1.61 (s, 1H), 3.05 (s, 3H), 3.78 (s, 3H), 6.69 (s, 1H), 6.82-6.95 (m, 4H), 6.98 (s, 1H), 7.23 (d, 1H), 7.37 (s, 1H), 9.07 (s, 1H).

### EXAMPLE 125

### N-{3-[1-Cyclopropyl-1-(4-fluoro-3-methoxy-phenyl)-ethyl]-1H-indol-7-yl}-methanesulfonamide

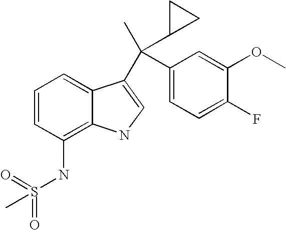

Flash chromatography eluting with a gradient (0 to 100 ethyl acetate/hexanes over 30 minutes) provides the title compound as a white solid (0.43 g, 64%).

¹H NMR (400 MHz, CDCl3): δ 0.21 (m, 1H), 0.32 (m, 1H), 0.50 (m, 1H), 0.55 (m, 1H), 1.59 (m, 1H), 1.61 (s, 1H), 3.05 (s, 3H), 3.79 (s, 3H), 6.51 (s, 1H), 6.82-7.02 (m, 6H), 7.38 (s, 1H), 9.06 (s, 1H).

### EXAMPLE 126

### Ethanesulfonic acid {3-[1-cyclopropyl-1-(2,4-difluoro-phenyl)-ethyl]-1H-indol-7-yl}-amide

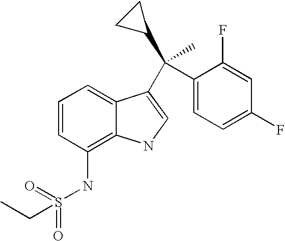

Utilizing the procedures of Scheme V, flash chromatography eluting with a gradient (0 to 100 ethyl acetate/hexanes over 25 minutes) provides the title compound as a white solid (0.64 g, 86%).

LC-MS m/z 405.0 (M⁺+1).

### EXAMPLE 127

### N-{3-[1-Cyclobutyl-1-(4-fluoro-phenyl)-ethyl]-1H-indol-7-yl}-methanesulfonamide

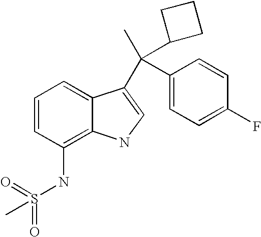

Flash chromatography eluting with a gradient (0 to 100 ethyl acetate/hexanes over 25 minutes) provides the title compound as a white solid (5.23 g, 96%).

LC-MS m/z 387.0 (M⁺+1).

### EXAMPLE 128

### N-{3-[1-(2,3-Dihydro-benzo[1,4]dioxin-6-yl)-1-ethyl-propyl]-1H-indol-7-yl}-methanesulfonamide

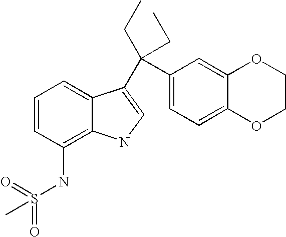

Flash chromatography eluting with a gradient (0 to 100 ethyl acetate/hexanes over 25 minutes) provides the title compound as a white solid (435 mg, 87%).

LC-MS m/z 415.0 (M⁺+1).

### EXAMPLE 129

### N-{3-[1-Cyclopropyl-1-(2,3-dihydro-benzo[1,4]dioxin-6-yl)-ethyl]-1H-indol-7-yl}-methanesulfonamide

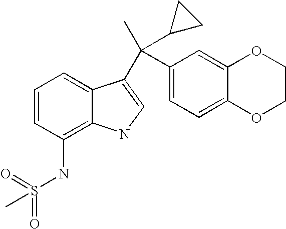

Flash chromatography eluting with a gradient (0 to 100 ethyl acetate/hexanes over 25 minutes) provides the title compound as a white solid (1.02 g, 80%).

LC-MS m/z 413.0 (M⁺+1).

### EXAMPLE 130

### N-{3-[1-Cyclopropyl-1-(3,4-dihydro-2H-benzo[b][1,4]dioxepin-7-yl)-ethyl]-1H-indol-7-yl}-methanesulfonamide

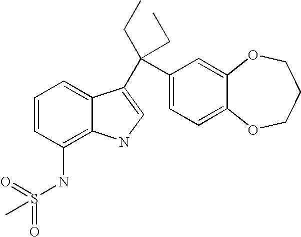

Flash chromatography eluting with a gradient (0 to 70 ethyl acetate/hexanes over 20 minutes, then hold at 70% ethyl acetate/hexanes for 10 minutes) provides the title compound as a white solid (2.00 g, 100%).

¹H NMR (400 MHz, CDCl3): δ 0.65 (t, 6H), 2.02-2.22 (m, 6H), 3.03 (s, 3H), 4.19 (m, 4H), 6.48 (s, 1H), 6.77-6.93 (m, 5H), 6.95 (s, 1H), 7.22 (s, 1H), 9.01 (s, 1H).

### EXAMPLE 131

### N-[3-(1-Benzo[1,3]dioxol-5-yl-1-cyclopropyl-ethyl)-1H-indol-7-yl]-methanesulfonamide

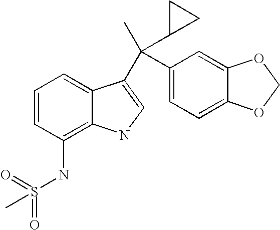

Flash chromatography eluting with a gradient (0 to 100 ethyl acetate/hexanes over 25 LC-MS m/z 399.0 (M⁺+1).

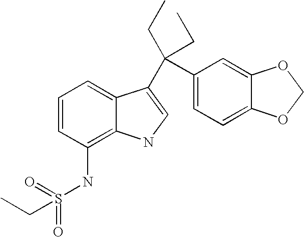

Flash chromatography eluting with a gradient (0 to 100 ethyl acetate/hexanes over 25 minutes) provides the title compound as a white solid (1.90 g, 99%).

LC-MS m/z 415.0 (M⁺+1).

### EXAMPLE 133

### N-{3-[1-(2,3-Dihydro-benzo[1,4]dioxin-6-yl)-1-ethyl-propyl]-1H-indol-7-yl}-ethanesulfonamide

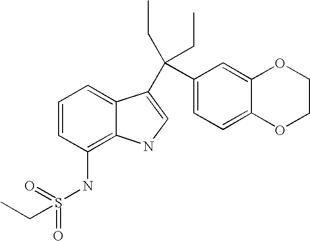

Flash chromatography eluting with a gradient (0 to 100 ethyl acetate/hexanes over 25 minutes) provides the title compound as a white solid (2.12 g, 100%).

LC-MS m/z 429.0 (M⁺+1).

Examples 134-163 below are made following procedures essentially as described in Examples 1-133 above. That is, employing the general procedures of Schemes I-XXII, and utilizing the appropriate indole and the appropriate carbinol, each of which may be obtained from commercial sources or prepared according to procedures as described in the Preparations herein, the title compounds of Examples 134-163 are prepared.

As used herein, the term “APCI MS” refers to atmospheric pressurized chemical ionization. “ESI” refers to electrospray ionization. “° C. dec.” refers to the temperature in Celsius degrees at which the compound decomposed.

Instrumental Analysis for Examples 134-163:

The TLC data was recorded on silica gel. ¹H NMR data was recorded at 300 MHz using tetramethyl silane as the internal standard. Melting points are uncorrected. HPLC methods are outlined below.

Method A: Waters Symmetry C18, 60 Å column (4.6×250 mm). The elution system consists of an isocratic elution of 95:5 (0.1% TFA in H₂O)/(0.1% TFA in CH₃CN) for 5 min, followed by a gradient of 95:5 to 0:100 (0.1% TFA in H₂O)/(0.1% TFA in CH₃CN) over 15 min, followed by (0.1% TFA in CH₃CN) isocratic elution for 5 min. The flow rate is 1 mL/min. UV detection is performed at 254 n.

Method B: Waters Symmetry C18, 60 Å column (4.6×250 mm). The elution system consists of a gradient of 90:10 to 0:100 (0.1% TFA in H₂O)/(0.1% TFA in CH₃CN) over 15 min, followed by (0.1% TFA in CH₃CN) isocratic elution for 10 min. The flow rate is 1 mL/min. UV detection is performed at 254 nm.

Method C: Waters Symmetry C18, 60 Å column (4.6×250 mm). The elution system consists of an isocratic elution of 95:5 (0.1% TFA in H₂O)/(0.1% TFA in CH₃CN) for 5 min, followed by a gradient of 95:5 to 0:100 (0.1% TFA in H₂O)/(0.1% TFA in CH₃CN) over 15 min, followed by (0.1% TFA in CH₃CN) isocratic elution for 5 min. The flow rate is 1 mL/min. UV detection is performed at 220 nm.

Method D: Waters Symmetry C18, 60 Å column (4.6×250 mm). The elution system consists of an isocratic elution of 95:5H₂O/CH₃CN for 5 min, followed by a gradient of 95:5 to 0:100H₂O/CH₃CN over 15 min, followed by CH₃CN isocratic elution for 5 min. The flow rate is 1 mL/min. WV detection is performed at 254 nm.

Method E: Waters Symmetry C18, 60 Å column (4.6×250 mm). The elution system consists of a gradient of 90:10 to 0:100H₂O/CH₃CN over 15 min, followed by isocratic CH₃CN elution for 10 min. The flow rate is 1 mL/min. UV detection is performed at 254 nm.

Method F: Waters Symmetry C18, 60 Å column (4.6×250 mm). The elution system consists of an isocratic elution of 97:3 (0.1% TFA in H₂O)/(0.1% TFA in CH₃CN) for 5 min, followed by a gradient of 97:3 to 0:100 (0.1% TFA in H₂O)/(0.1% TFA in CH₃CN) over 15 min, followed by (0.1% TFA in CH₃CN) isocratic elution for 5 min. The flow rate is 1 mL/min. UV detection is performed at 254 nm.

### EXAMPLE 134

### N-{3-[1-(1H-Benzoimidazol-5-yl)-1-ethyl-propyl]-1H-indol-7-yl}-methanesulfonamide

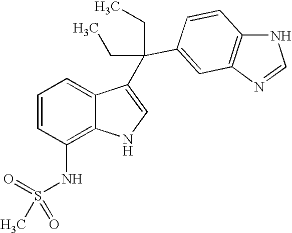 A. Preparation of:

### 3-(1H-Benzoimidazol-5-yl)-pentan-3-ol

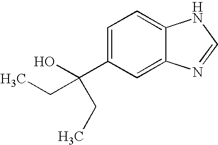

Ethylmagnesium bromide (3 M in Et₂O, 4.73 mL, 14.2 mmol) is added dropwise to a 0° C. suspension of methyl 1H-benzimidazole-5-carboxylate (500 mg, 2.84 mmol) in THF (14 mL). The ice bath is removed after stirring for 2 h and the reaction is left to stir overnight. The reaction is quenched with H₂O (30 mL) and saturated aqueous NH₄Cl (30 mL), and the reaction mixture is diluted with EtOAc (200 mL). The organic layer is washed with brine (30 mL) then dried (MgSO₄), filtered and concentrated to afford the sub-title compound (567 mg, 98%) as a light brown oil which is used without further purification.

R_{f} 0.09 (95:5:0.5 CH₂Cl₂/MeOH/NH₄OH).

¹H NMR (300 MHz, CD₃OD) δ 0.75 (t, J=7.2 Hz, 6H), 1.79-1.95 (sym m, 4H), 7.28 (dd, J=1.7, 8.5 Hz, 1H), 7.54 (d, J=8.5 Hz, 1H), 7.69 (d, J=1.1 Hz, 1H), 8.11 (s, 1H).

B. Preparation of:

### N-{3-[1-(H-Benzoimidazol-5-yl)-1-ethyl-propyl]-1H-indol-7-yl}-methanesulfonamide

To a suspension of 3-(1H-benzoimidazol-5-yl)-pentan-3-ol (566 mg, 2.78 mmol) in CH₂Cl₂ (28 mL) is added N-(1H-indol-7-yl)-methanesulfonamide (784 mg, 2.78 mmol) followed by TFA (950 mg, 8.34 mmol). After stirring the reaction for 16 h at room temperature, the reaction appears incomplete by TLC, and TFA (950 mg, 8.34 mmol) is added. After a further 24 h, TFA (315 mg, 2.76 mmol) is added and the reaction is stirred for 6 d. The reaction mixture is diluted with EtOAc (200 mL) and washed with saturated NaHCO₃ (2×50 mL) and brine (50 mL). The organic layer is dried (MgSO₄), filtered and concentrated. The reaction residue is subjected to flash chromatography (silica gel, 95:5:0.5 CH₂Cl₂/MeOH/NH₄OH) to afford the title compound (581 mg, 53%) as an off-white solid.

R_{f} 0.39 (90:10:1 CH₂Cl₂/MeOH/NH₄H).

mp 150-165° C.

¹H NMR (300 MHz, CD₃OD) δ 0.65 (t, J=7.3 Hz, 6H), 2.14-2.36 (sym m, 4H), 2.94 (s, 3H), 6.58-6.66 (m, 2H), 6.91 (d, J=6.8 Hz, 1H), 7.16 (d, J=8.6 Hz, 1H), 7.33 (s, 1H), 7.40 (d, J=8.6 Hz, 1H), 7.61 (s, 1H), 8.08 (s, 1H).

ESI MS m/z 397 [C₂₁H₂₄N₄O₂S+H]⁺.

HPLC (Method A) 97.4% (area percent), t_{R}=15.7 min.

### EXAMPLE 135

### N-[3-(1-Benzo[b]thiophen-5-yl-1-ethyl-propyl)-1H-indol-7-yl]-methanesulfonamide

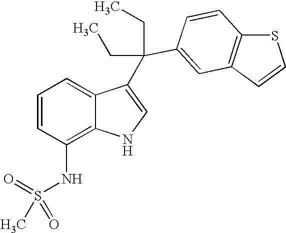 A. Preparation of:

### 3-Benzo[b]thiophen-5-yl-pentan-3-ol

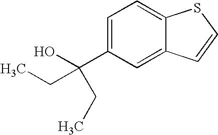

To a pre-dried flask equipped with a condenser is added magnesium (568 mg, 23.4 mmol) and Et₂O (5 mL). To this is added ˜ 1/10^{th} of a solution of iodomethane (1.66 g, 11.7 mmol) and 5-bromo-benzo[b]thiophene (500 mg, 2.34 mmol) in Et₂O (8 mL). After adding 2-3 crystals of iodine, the reaction mixture is heated to reflux using a hot water bath. After a few minutes the iodine coloration fades and another portion (˜0.5 mL) of the iodomethane/5-bromo-benzo[b]thiophene solution is added. The water bath is removed and further additions (˜0.5 mL) are added such that reflux is sustained. After complete addition, reflux is maintained for 30 min using a hot water bath. The Grignard solution is then cooled to 0° C. and 3-pentanone (1.20 g, 14.0 mmol) is added dropwise. After 30 min, the ice is removed and the reaction mixture is stirred for 2 h. After cooling to 0° C., the reaction is quenched with H₂O (10 mL) and saturated aqueous NH₄Cl (15 mL) and is diluted with Et₂O (100 mL). The organic layer is washed with brine (35 mL), dried (MgSO₄), filtered and concentrated. The reaction residue is subjected to flash chromatography (silica gel, 90:10 petroleum ether/Et₂O) to afford impure sub-title compound (˜500 mg). Most of the impurity is removed under high vacuum (˜2 d) to yield slightly impure sub-title compound (323 mg, ˜62%).

R_{f} 0.43 (4:1 Hex/EtOAc).

¹H NMR (300 MHz, CDCl₃) δ 0.77 (t, J=7.4 Hz, 6H), 1.70 (s, 1H), 1.80-1.98 (sym m, 4H), 7.32 (d, J=5.4 Hz, 1H), 7.34 (dd, J=1.6, 8.5 Hz, 1H), 7.42 (d, J=5.4 Hz, 1H), 7.82 (d, J=8.5 Hz, 1H), 7.87 (d, J=1.6 Hz, 1H).

B. Preparation of:

### N-[3-(1-Benzo[b]thiophen-5-yl-1-ethyl-propyl)-1H-indol-7-yl]-methanesulfonamide

To a solution of 3-benzo[b]thiophen-5-yl-pentan-3-ol (323 mg, 1.47 mmol) in CH₂Cl₂ (6 mL) is added N-(1H-indol-7-yl)-methanesulfonamide (257 mg, 1.22 mmol) followed by TFA (417 mg, 3.66 mmol). The reaction mixture turns green-black in color shortly after adding the TFA. After stirring for 16 h at room temperature, the reaction is removed. The solvent is evaporated under reduced pressure and the resulting residue is subjected to flash chromatography (silica gel, 3:1 Hex/EtOAc) to afford the title compound (432 mg, 86%) as a white solid.

R_{f} 0.67 (1:1 EtOAc/Hex).

mp 85-95° C.

¹H NMR (300 MHz, CDCl₃) δ 0.66 (t, J=7.3 Hz, 6H), 2.14-2.31 (sym m, 4H), 3.01 (s, 3H), 6.37 (s, 1H), 6.65-6.80 (m, 3H), 7.19 (dd, J=1.7, 8.5 Hz, 1H), 7.28-7.30 (m, 2H), 7.38 (d, J=5.4 Hz, 1H), 7.67 (d, J=8.5 Hz, 1H), 7.85 (d, J=1.6 Hz, 1H), 9.01 (br s, 1H).

ESI MS (Negative Mode) m/z 411 [C₂₂H₂₄N₂O₂S₂—H]⁻.

HPLC (Method B) 96.2% (area percent), t_{R}=18.8 min.

### EXAMPLE 136

### N-{3-[1-Ethyl-1-(2-methyl-benzooxazol-6-yl)-propyl]-1H-indol-7-yl}-methanesulfonamide

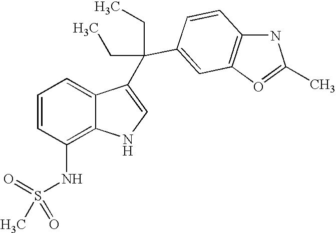 A. Preparation of:

### 2-Amino-5-(1-ethyl-1-hydroxy-propyl)-phenol

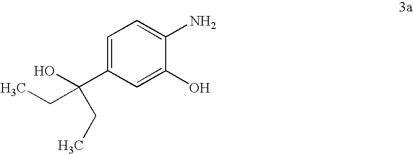

To a 0° C. solution of 4-amino-3-hydroxy-benzoic acid methyl ester (2.00 g, 12.0 mmol) in THF (100 mL) is added ethylmagnesium bromide (3 M in Et₂O, 27.9 mL, 83.7 mmol) fast dropwise over ˜5 min. After 2 h, the ice bath is removed and the reaction stirred at room temperature for 3 d. The reaction mixture is cooled to 0° C. and quenched with H₂O (40 mL) and saturated aqueous NH₄Cl (40 mL). The reaction mixture is extracted with EtOAc (2×150 mL) and the combined organic layer is dried (MgSO₄), filtered and concentrated. The red oily suspension is subjected to flash chromatography (silica gel, 96:4:0.5 CH₂Cl₂/MeOH/NH₄OH) to afford the sub-title compound (1.36 g, 58%) as a pink solid.

R_{f} 0.37 (95:5:0.5 CH₂Cl₂/MeOH/NH₄OH).

mp 100-102° C.

¹H NMR (300 MHz, CD₃OD) δ 0.74 (t, J=7.4 Hz, 6H), 1.65-1.80 (sym m, 4H), 6.64-6.71 (m, 2H), 6.77 (d, J=1.7 Hz, 1H).

APCI MS (Negative Mode) m/z 194 [C₁₁H₁₇NO₂−H]⁻.

B. Preparation of:

### N-[4-(1-Ethyl-1-hydroxy-propyl)-2-hydroxy-phenyl]-acetamide

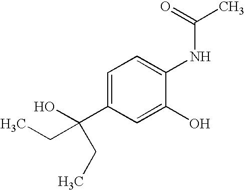

To a 0° C. suspension of 2-amino-5-(1-ethyl-1-hydroxy-propyl)-phenol (500 mg, 2.56 mmol) in EtOAc (6 mL) is added acetic anhydride (588 mg, 5.76 mmol). The ice bath is removed after 2 h and the reaction mixture stirred at room temperature for 30 min and H₂O (30 mL) is added. The reaction mixture is diluted with EtOAc (100 mL) and the organic layer is dried (MgSO₄), filtered and concentrated. The reaction residue is subjected to flash chromatography (silica gel, 4:1 Hex/EtOAc) to afford the sub-title compound (536 mg, 88%).

R_{f} 0.11 (1:1 EtOAc/Hex).

¹H NMR (300 MHz, CD₃OD) δ 0.74 (t, J=7.4 Hz, 6H), 1.70-1.81 (sym m, 4H), 2.16 (s, 3H), 6.81 (dd, J=1.9, 8.4 Hz, 1H), 6.93 (d, J=1.9 Hz, 1H), 7.47 (d, J=8.4 Hz, 1H).

APCI MS (Negative Mode) m/z 236 [C₁₃H₁₉NO₃−H]⁻.

C. Preparation of:

### N-{4-[1-Ethyl-1-(7-methanesulfonylamino-1H-indol-3-yl)-propyl]-2-hydroxy-phenyl}-acetamide

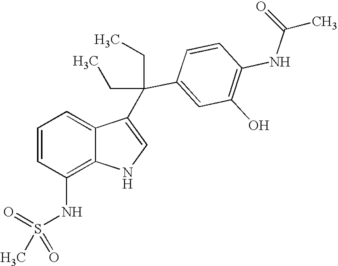

To a solution of N-[4-(1-ethyl-1-hydroxy-propyl)-2-hydroxy-phenyl]-acetamide (536 mg, 2.26 mmol) in CH₂Cl₂ (22 mL) is added N-(1H-indol-7-yl)-methanesulfonamide (640 mg, 3.04 mmol) followed by TFA (773 mg, 6.78 mmol). The reaction mixture changes color from red to green-black over several minutes. After stirring for 15 min, TLC indicates that the reaction is complete. The reaction mixture is quenched with saturated aqueous NaHCO₃ (200 mL) and diluted with EtOAc (1 L). The organic layer is washed with brine (100 mL), dried (MgSO₄), filtered and concentrated. The residue is subjected to flash chromatography (silica gel, 60:40 to 100:0 EtOAc/Hex) to afford the sub-title compound (853 mg, 88%) as an off-white solid.

R_{f} 0.37 (95:5:0.5 CH₂Cl₂/MeOH/NH₄OH).

mp 248-250° C.

¹H NMR (300 MHz, DMSO-d₆) δ 0.56 (t, J=7.1 Hz, 6H), 1.95-2.15 (m, 7H), 2.99 (s, 3H), 6.63-6.74 (m, 4H), 6.92 (dd, J=1.4, 6.7 Hz, 1H), 7.31 (d, J=2.2 Hz, 1H), 7.52 (d, J=8.3 Hz, 1H), 9.22-9.23 (m, 2H), 9.43 (s, 1H), 10.59 (s, 1H).

APCI MS m/z 430 [C₂₂H₂₇N₃O₄S+H]⁺.

D. Preparation of:

### N-{3-[1-Ethyl-1-(2-methyl-benzooxazol-6-yl)-propyl]-1H-indol-7-yl}-methanesulfonamide

A solution of N-{4-[1-ethyl-1-(7-methanesulfonylamino-1H-indol-3-yl)-propyl]-2-hydroxy-phenyl}-acetamide (609 mg, 1.42 mmol) in HOAc (20 mL) is heated to reflux for 24 h. After cooling to room temperature, the solvent is removed under reduced pressure and the reaction residue is subjected to flash chromatography (silica gel, 6:4 to 1:1 Hex/EtOAc) to afford the title compound (483 mg, 83%) as a pink solid.

R_{f} 0.52 (4:1 EtOAc/Hex).

mp 98-105° C.

¹H NMR (300 MHz, CDCl₃) δ 0.64 (t, J=7.3 Hz, 6H), 2.15-2.25 (sym m, 4H), 2.59 (s, 3H), 3.02 (s, 3H), 6.60 (s, 1H), 6.69-6.75 (m, 2H), 6.80 (dd, J=1.5, 6.8 Hz, 1H), 7.21-7.29 (m, 2H), 7.44-7.47 (m, 2H), 9.05 (br s, 1H).

APCI MS m/z 412 [C₂₂H₂₅N₃O₃S+H]⁺.

HPLC (Method B) 98.3% (area percent), t_{R}=16.7 min.

### EXAMPLE 137

### N-[3-(1-Benzooxazol-6-yl-1-ethyl-propyl)-1H-indol-7-yl]-methanesulfonamide

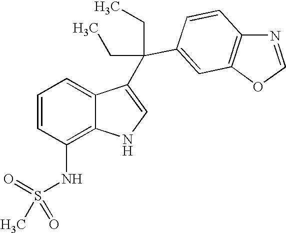 A. Preparation of:

### N-{3-[1-(4-Amino-3-hydroxy-phenyl)-1-ethyl-propyl]-1H-indol-7-yl}-methanesulfonamide

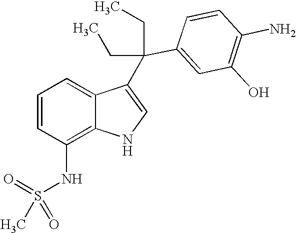

To a solution of 2-amino-5-(1-ethyl-1-hydroxy-propyl)-phenol (200 mg, 1.02 mmol) in CH₂Cl₂ (10 mL) is added N-(1H-indol-7-yl)-methanesulfonamide (215 mg, 1.02 mmol) followed by TFA (465 mg, 4.00 mmol). The reaction mixture turnes green-black in color shortly after adding the TFA. After stirring for 2 h at room temperature, N-(1H-indol-7-yl)-methanesulfonamide (25 mg, 0.19 mmol) is added and the reaction is stirred for 4 d. The solvent is removed under reduced pressure and the resulting reaction residue is diluted with EtOAc (500 mL) and CHCl₃ (50 mL). The organic layer is washed with saturated aqueous NaHCO₃ (2×50 mL) and brine (50 mL) then is dried (MgSO₄), filtered and concentrated. The resulting red oil is subjected to flash chromatography (silica gel, 97:3:0.5 to 96:4:0.5 CH₂Cl₂/MeOH/NH₄OH) to afford the sub-title compound ('341 mg, 86%) as a light purple solid.

R_{f} 0.29 (95:5:0.5 CH₂Cl₂/MeOH/NH₄OH).

mp 120-130° C.

¹H NMR (300 MHz, CD₃OD) δ 0.66 (t, J=7.2 Hz, 6H), 2.00-2.22 (sym m, 4H), 2.94 (s, 3H), 6.58-6.70 (m, 4H), 6.80 (d, J=7.2 Hz, 1H), 6.92 (d, J=7.2 Hz, 1H), 7.24 (s, 1H).

APCI MS (Negative Mode) m/z 386 [C₂₀H₂₅N₃O₃S−H]⁻.

B. Preparation of:

### N-[3-(1-Benzooxazol-6-yl-1-ethyl-propyl)-1H-indol-7-yl]-methanesulfonamide

A solution of N-{3-[1-(4-amino-3-hydroxy-phenyl)-1-ethyl-propyl]-1H-indol-7-yl}-methanesulfonamide (325 mg, 0.839 mmol) in triethylorthoformate (5 mL) is heated to 140° C. for 3 h. After cooling to room temperature, the solvent is removed under reduced pressure and the reaction residue is subjected to flash chromatography (silica gel, 6:4 Hex/EtOAc) to afford the title compound (235 mg, 71%) as a yellow solid.

R_{f} 0.62 (4:1 EtOAc/Hex).

mp 211-213° C.

¹H NMR (300 MHz, CD₃OD) δ 0.66 (t, J=7.3 Hz, 6H), 2.15-2.38 (sym m, 4H), 2.96 (s, 3H), 6.63-6.68 (m, 2H), 6.94 (dd, J=2.3, 5.8 Hz, 1H), 7.33 (d, J=8.4 Hz, 1H), 7.36 (s, 1H), 7.54 (d, J=8.4 Hz, 1H), 7.64 (s, 1H), 8.38 (s, 1H).

ESI MS m/z 398 [C₂₁H₂₃N₃O₃S+H]⁺.

HPLC (Method E) 97.6% (area percent), t_{R}=16.4 min.

### EXAMPLE 138

### N-{3-[1-Ethyl-1-(1H-indazol-5-yl)-propyl]-1H-indol-7-yl}-methanesulfonamide

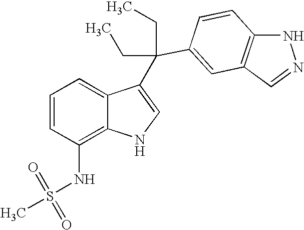 A. Preparation of:

### 3-(1H-Indazol-5-yl)-pentan-3-ol

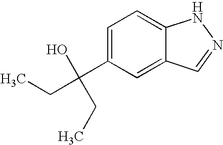

To a 0° C. solution of 1H-indazole-5-carboxylic acid ethyl ester (200 mg, 1.05 mmol) in THF (5 mL) is added ethylmagnesium bromide (3 M in Et₂₀, 1.75 mL, 5.25 mmol) dropwise. The reaction is left to slowly warm to room temperature overnight (˜16 h) and is quenched with saturated aqueous NH₄Cl (10 mL) and H₂O (10 mL). The reaction mixture is diluted with EtOAc (150 mL) and the organic layer is washed with brine (30 mL) then is dried (MgSO₄), filtered and concentrated to afford the sub-title compound (158 mg, 74%) which is used without any further purification.

R_{f} 0.28 (95:5:0.5 CH₂Cl₂/MeOH/NH₄OH).

mp 132-135° C.

¹H NMR (300 MHz, CD₃OD) δ 0.75 (t, J=7.4 Hz, 6H), 1.81-1.95 (sym m, 4H), 7.42 (dd, J=1.5, 8.8 Hz, 1H), 7.48 (d, J=8.8 Hz, 1H), 7.80 (d, J=1.5 Hz, 1H), 8.00 (s, 1H).

ESI MS m/z 205 [C₁₂H₁₆N₂O+H]⁺.

B. Preparation of:

### N-{3-[1-Ethyl-1-(1H-indazol-5-yl)-propyl]-1H-indol-7-yl}-methanesulfonamide

To a solution of 3-(1H-indazol-5-yl)-pentan-3-ol (150 mg, 0.734 mmol) in CH₂Cl₂ (5 mL) is added N-(1H-indol-7-yl)-methanesulfonamide (154 mg, 0.734 mmol) followed by TFA (251 mg, 2.20 mmol). The reaction mixture turns green-black in color shortly after adding the TFA. After stirring overnight at room temperature, the reaction is removed and the solvent evaporated under reduced pressure. The reaction residue is subjected to flash chromatography (silica gel, 97:3:0.5 CH₂Cl₂/MeOH/NH₄OH) to afford the title compound (210 mg, 72%) as an off-white solid.

R_{f} 0.43 (90:10:1 CH₂Cl₂/MeOH/NH₄OH).

mp 123-128° C.

¹H NMR (300 MHz, CD₃OD) δ 0.65 (t, J=7.3 Hz, 6H), 2.16-2.34 (sym m, 4H), 2.95 (s, 3H), 6.58-6.68 (m, 2H), 6.91 (dd, J=1.1, 7.2 Hz, 1H), 7.19 (dd, J=1.5, 8.9 Hz, 1H), 7.28 (d, J=8.9 Hz, 1H), 7.33 (s, 1H), 7.82 (s, 1H), 7.98 (d, J=0.7 Hz, 1H).

ESI MS m/z 397 [C₂₁H₂₄N₄O₂S+H]⁺.

HPLC (Method A) 96.9% (area percent), t_{R}=18.6 min.

### EXAMPLE 139

### N-[3-(1-Benzo[b]thiophen-6-yl-1-ethyl-propyl)-1H-indol-7-yl]-methanesulfonamide

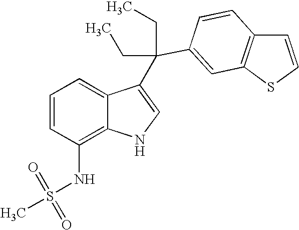 A. Preparation of:

### (3-Bromo-phenylsulfanyl)-acetic acid

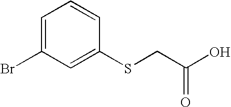

To a solution of NaOH (5.28 g, 0.132 mol) in H₂O (40 mL) is added 3-bromothiophenol (2.50 g, 13.2 mmol). A solution of 2-chloroacetic acid (1.49 g, 15.8 mmol) in H₂O (5 mL) is added dropwise to the vigorously stirred biphasic reaction mixture. After stirring for 30 min at room temperature, the reaction mixture is refluxed for 1.5 h then cooled to room temperature. The reaction is acidified to ˜pH 1 using 2 M HCl and extracted with Et₂O (3×200 mL). The combined organic layer is dried (MgSO₄), filtered and concentrated to afford the sub-title compound (2.53 g, 78%) as a white solid which is used without further purification.

mp 79-82° C.

¹H NMR (300 MHz, CDCl₃) δ 3.68 (s, 2H), 7.17 (t, J=7.9 Hz, 1H), 7.28-7.43 (m, 2H), 7.55 (t, J=1.8 Hz, 1H), −8.80-11.00 (br s, 1H).

APCI MS (Negative Mode) m/z 245 [C₈H₇BrO₂S−H]⁻.

B. Preparation of:

### 6-Bromo-benzo[b]thiophen-3-one and 4-bromo-benzo[b]thiophen-3-one

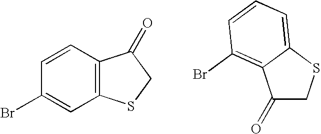

A solution of (3-bromo-phenylsulfanyl)-acetic acid (2.45 g, 9.91 mmol) in thionyl chloride (7.5 mL) is heated to reflux for 2 h. The reaction mixture is cooled to room temperature and the solvent removed under reduced pressure. The residual solvent is removed under high vacuum for 30 min. The resulting orange oil is dissolved in 1,2-dichlorobenzene (10 mL) and AlCl₃ (1.68 g, 12.6 mmol) is added in 4 portions over ˜5 min. Gas evolution occurs during the addition. The resulting green reaction mixture is heated to 45° C. for 1 h then cooled to room temperature. The reaction is poured into ice-H₂O and basified to ˜pH 12 using 2 M NaOH whereupon all of the solid is dissolved.

The reaction mixture is extracted with Et₂O (2×100 mL) and the aqueous layer is reacidified to ˜pH 1 and extracted with EtOAc (200 mL). The EtOAc layer is dried (MgSO₄), filtered and concentrated to afford an inseparable mixture (˜2.8:1) of the sub-title compounds (1.58 g, 70%) as a pink solid.

R_{f} (mixture) 0.14 (1:1 EtOAc/Hex).

¹H NMR (major regioisomer, subtracted from mixture) (300 MHz, CD₃OD) δ 3.89 (s, 2H), 7.41 (dd, J=1.5, 8.2 Hz, 1H), 7.60 (d, J=8.2 Hz, 1H), 7.75 (d, J=1.5 Hz, 1H).

¹H NMR (minor regioisomer, subtracted from mixture) (300 MHz, CD₃OD) δ 3.93 (s, 2H), 7.40-7.52 (m, 3H).

APCI MS (Negative Mode) (mixture) m/z 229 [C₈H₅BrOS−H]⁻.

C. Preparation of:

### 6-Bromo-2,3-dihydro-benzo[b]thiophen-3-ol (i) and 4-bromo-2,3-dihydro-benzo[b]thiophen-3-ol (ii)

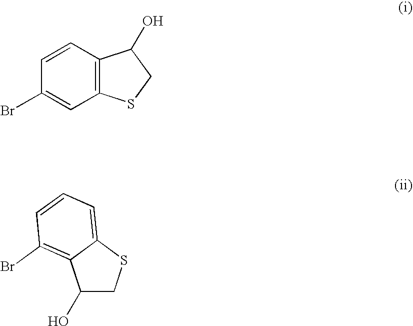

To a 0° C. suspension of 6-bromo-benzo[b]thiophen-3-one and 4-bromo-benzo[b]thiophen-3-one (˜2.8:1 mixture) (1.02 g, 4.45 mmol) in MeOH (40 mL) is added sodium borohydride (210 mg, 5.56 mmol). After 30 min, the reaction is warmed to room temperature and stirred for 45 min. The reaction mixture is quenched with H₂O (10 mL) and saturated aqueous NH₄Cl (15 mL) and the pH is adjusted to ˜3 using 3 M HCl then extracted with Et₂O (2×100 mL). The organic layer is dried (MgSO₄), filtered and concentrated. The reaction residue is subjected to flash chromatography (silica gel, 1:1:1 to 2:1:1 Et₂O/pentane/petroleum ether) to afford the sub-title compounds (640 mg, 62% and 255 mg, 25%) as pink solids.

Major Regioisomer (i):

R_{f} 0.34 (1:1 Hex/EtOAc).

¹H NMR (300 MHz, CDCl₃) δ 2.05 (d, J=8.5 Hz, 1H), 3.30 (dd, J=3.8, 12.0 Hz, 1H), 3.61 (dd, J=6.2, 12.0 Hz, 1H), 5.31 (m, 1H), 7.22 (s, 2H), 7.38 (s, 1H).

Minor Regioisomer (ii):

R_{f} 0.50 (1:1 Hex/EtOAc).

¹H NMR (300 MHz, CDCl₃) δ 2.32 (d, J=6.0 Hz, 1H), 3.34 (dd, J=1.3, 12.6 Hz, 1H), 3.66 (dd, J=6.0, 12.6 Hz, 1H), 5.51 (dt, J=1.0, 6.0 Hz, 1H), 7.10 (m, 1H), 7.19 (d, J=7.0 Hz, 1H), 7.23 (dd, J=1.1, 7.7 Hz, 1H).

D. Preparation of:

### 6-Bromo-benzo[b]thiophene

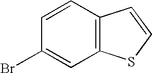

To a room temperature solution of 6-bromo-2,3-dihydro-benzo[b]thiophen-3-ol (785 mg, 3.39 mmol) in HOAc (7 mL) is added boron trifluoride diethyl etherate (1.44 g, 10.2 mmol) and the reaction mixture is placed into a 120° C. oil bath. After 5 min, the reaction is cooled to room temperature and basified to ˜pH 11 using 2 M NaOH. The aqueous suspension is extracted with Et₂O (2×200 mL) and the combined organic layer is dried (MgSO₄), filtered and concentrated to afford the sub-title compound (689 mg, 95%) as an off-white solid.

R_{f} 0.70 (4:1 Hex/EtOAc).

mp 48-50° C.

¹H NMR (300 MHz, CDCl₃) δ 7.29 (d, J=5.4 Hz, 1H), 7.41 (d, J=5.4 Hz, 1H), 7.46 (dd, J=1.8, 8.5 Hz, 1H), 7.66 (d, J=8.5 Hz, 1H), 8.01 (m, 1H).

E. Preparation of:

### 3-Benzo[b]thiophen-6-yl-pentan-3-ol

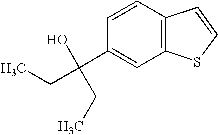

To a pre-dried flask equipped with a condenser is added magnesium (363 mg, 14.9 mmol) and Et₂O (5 mL). To this is added ˜ 1/10^{th} of a solution of iodomethane (1.32 g, 9.35 mmol) and 6-bromo-benzo[b]thiophene (400 mg, 1.87 mmol) in Et₂O (5 mL). After adding 2-3 crystals of iodine, the reaction mixture is heated to reflux using a hot water bath. After a few minutes the iodine coloration fades and another portion (˜0.5 mL) of the iodomethane/6-bromo-benzo[b]thiophene solution is added. The water bath is removed and further additions (˜0.5 mL) are added such that reflux is sustained. After complete addition, reflux is maintained for 30 min using a hot water bath. The Grignard solution is then cooled to 0° C. and 3-pentanone (966 mg, 11.2 mmol) is added dropwise. After 30 min, the ice is removed and the reaction mixture stirred for 1.5 h. Another portion of 3-pentanone (122 mg, 1.42 mmol) is added and the reaction is stirred for 1 h. After cooling to 0° C., the reaction is quenched with H₂O (10 mL) and saturated aqueous NH₄Cl (15 mL) and is diluted with Et₂O (100 mL). The organic layer is dried (MgSO₄), filtered and concentrated. The reaction residue is subjected to flash chromatography (silica gel, 90:10 petroleum ether/Et₂O) to afford impure sub-title compound. Most of the impurity is removed under high vacuum (˜24 h) to afford slightly impure sub-title compound (243 mg, ˜59%).

¹H NMR (300 MHz, CDCl₃) δ 0.77 (t, J=7.4 Hz, 6H), 1.70 (s, 1H), 1.80-2.00 (sym m, 4H), 7.29-7.34 (m, 2H), 7.40 (d, J=5.4 Hz, 1H), 7.76 (d, J=8.4 Hz, 1H), 7.95 (s, 1H).

F. Preparation of:

### N-[3-(1-Benzo[b]thiophen-6-yl-1-ethyl-propyl)-1H-indol-7-yl]-methanesulfonamide

To a solution of 3-benzo[b]thiophen-6-yl-pentan-3-ol (243 mg, 1.10 mmol) in CH₂Cl₂ (6 mL) is added N-(1H-indol-7-yl)-methanesulfonamide (289 mg, 1.38 mmol) followed by

TFA (376 mg, 3.30 mmol). The reaction mixture turns green-black in color shortly after adding the TFA. After stirring for 24 h at room temperature, N-(1H-indol-7-yl)-methanesulfonamide (92 mg, 0.43 mmol) and TFA (123 mg, 1.08 mmol) are added to the reaction mixture. After ˜6 h, the reaction is removed and the solvent is evaporated under reduced pressure. The residue is diluted with EtOAc (100 mL) and washed with saturated aqueous NaHCO₃ (2×25 mL) and brine (25 mL) then is dried (MgSO₄), filtered and concentrated. The light purple oil is subjected to flash chromatography (silica gel, 55:45 Hex/EtOAc) to afford the title compound (223 mg, 49%) as a white solid.

R_{f} 0.66 (1:1 EtOAc/Hex).

mp 97-107° C.

¹H NMR (300 MHz, CD₃OD) δ 0.65 (t, J=7.3 Hz, 6H), 2.14-2.37 (sym m, 4H), 2.96 (s, 3H), 6.60-6.69 (m, 2H), 6.92 (dd, J=1.6, 6.8 Hz, 1H), 7.20-7.25 (m, 2H), 7.34 (s, 1H), 7.44 (d, J=5.4 Hz, 1H), 7.62 (d, J=8.4 Hz, 1H), 7.87 (s, 1H).

ESI MS (Negative Mode) m/z 411 [C₂₂H₂₄N₂O₂S₂−H]⁻.

HPLC (Method B) >99% (area percent), t_{R}=18.6 min.

### EXAMPLE 140

### N-{3-[1-Ethyl-1-(2-methyl-benzothiazol-5-yl)-propyl]-1H-indol-7-yl}-methanesulfonamide

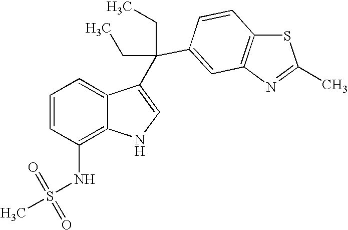 A. Preparation of:

### 3-(2-Methyl-benzothiazol-5-yl)-pentan-3-ol

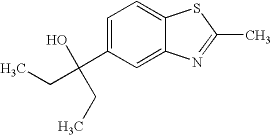

To a pre-dried flask equipped with a condenser is added magnesium (425 mg, 17.5 mmol) and Et₂O (5 mL). To this is added ˜ 1/10^{th} of a solution of iodomethane (1.55 g, 10.9 mmol) and 5-bromo-2-methyl-benzothiazole (500 mg, 2.19 mmol) in Et₂O (10 mL). After adding 2-3 crystals of iodine, the reaction mixture is heated to reflux using a hot water bath. After a few minutes the iodine coloration fades and another portion (˜0.5 mL) of the iodomethane/5-bromo-2-methyl-benzothiazole solution is added. The water bath is removed and further additions (˜0.5 mL) are added such that reflux is sustained. After complete addition, reflux is maintained for 30 min using a hot water bath. The Grignard solution is then cooled to 0° C. and 3-pentanone (1.13 g, 13.1 mmol) is added dropwise. After 15 min, the ice is removed and the reaction mixture is stirred for 2.5 h. After cooling to 0° C., the reaction is quenched with H₂O (15 mL) and saturated aqueous NH₄Cl (25 mL) and is diluted with Et₂O (150 mL). The organic layer is dried (MgSO₄), filtered and concentrated. The reaction residue is subjected to flash chromatography (silica gel, 75:25 Hex/EtOAc) to afford the sub-title compound (125 mg, 24%).

mp 120-122° C.

¹H NMR (300 MHz, CDCl₃)δ 0.77 (t, J=7.4 Hz, 6H), 1.80 (s, 1H), 1.80-2.05 (sym m, 4H), 2.84 (s, 3H), 7.41 (dd, J=1.7, 8.4 Hz, 1H), 7.77 (d, J=8.4 Hz, 1H), 7.96 (d, J=1.7 Hz, 1H).

ESI MS m/z 236 [C₁₃H₁₇NOS+H]⁺.

B. Preparation of:

### N-{3-[1-Ethyl-1-(2-methyl-benzothiazol-5-yl)-propyl]-1H-indol-7-yl}-methanesulfonamide

To a solution of 3-(2-methyl-benzothiazol-5-yl)-pentan-3-ol (354 mg, 1.49 mmol) in CH₂Cl₂ (7.5 mL) is added N-(1H-indol-7-yl)-methanesulfonamide (282 mg, 1.34 mmol) followed by TFA (509 mg, 4.47 mmol). After stirring the reaction for 48 h at room temperature, the reaction appears incomplete by TLC, and TFA (509 mg, 4.47 mmol) is added. After a further 24 h, N-(1H-indol-7-yl)-methanesulfonamide (156 mg, 0.742 mmol) and TFA (169 mg, 1.48 mmol) are added and the reaction is stirred for 3 d. N-(1H-indol-7-yl)-methanesulfonamide (63 mg, 0.30 mmol) and TFA (169 mg, 1.48 mmol) are added and the reaction is stirred at room temperature for another 3 d. The solvent is evaporated under reduced pressure and the residual solvent and TFA are removed under high vacuum (˜12 h). The reaction residue is subjected to flash chromatography (silica gel, 60:40 Hex/EtOAc) to afford impure title compound (˜300 mg, 53%). The impure title compound is subjected to preparative HPLC (Waters Symmetry C18 column, 7 □m, 77×230 mm, 55:45 CH₃CN/H₂O, 0.1% TFA, 250 mL/min, δ=254 nm) to afford the title compound (245 mg, 43%) as a white solid.

R_{f} 0.22 (1:1 EtOAc/Hex).

mp 112-117° C.

¹H NMR (300 MHz, DMSO-d₆) δ 0.57 (t, J=7.2 Hz, 6H), 2.06-2.28 (sym m, 4H), 2.73 (s, 3H), 2.97 (s, 3H), 6.51-6.62 (m, 2H), 6.89 (dd, J=0.7, 7.2 Hz, 1H), 7.19 (dd, J=1.6, 8.4 Hz, 1H), 7.38 (d, J=2.4 Hz, 1H), 7.74-7.80 (m, 2H), 9.22 (s, 1H), 10.66 (s, 1H).

ESI MS (Negative Mode) m/z 426 [C₂₂H₂₅N₃O₂S₂−H]⁻.

HPLC (Method A) >99% (area percent), t_{R}=20.5 min.

### EXAMPLE 141

### N-{3-[1-(2-Amino-benzothiazol-5-yl)-1-ethyl-propyl]-1H-indol-7-yl}-methanesulfonamide

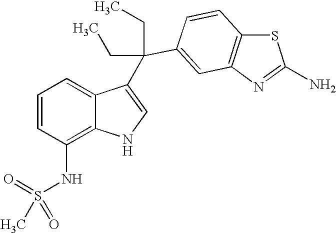 A. Preparation of:

### 3-Nitro-4-thiocyanato-benzoic acid

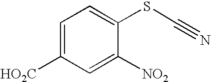

An ice-cold solution of sodium nitrite (1.25 g, 18.1 mmol) in H₂O (6 mL) is added dropwise to a 5° C. suspension of 4-amino-3-nitrobenzoic acid (2.00 g, 11.0 mmol) in H₂O (50 mL) and concentrated H₂SO₄ (25 mL). The temperature does not rise above 10° C. during the addition. The reaction mixture is filtered through a sintered glass funnel containing diatomaceous earth. The ice-cooled filtrate is added with stirring to a solution of potassium thiocyanate (2.50 g, 25.7 mmol) and iron(III)chloride (2.00 g, 12.3 mmol) in H₂O (20 mL) resulting in nitrogen evolution. After stirring at room temperature for 3 h, the reaction mixture is filtered through a sintered-glass funnel, washing with ice-cold H₂O (10 mL). The precipitate is dissolved in EtOAc (150 mL) and the organic layer is dried (MgSO₄), filtered and concentrated to afford the sub-title compound (1.75 g, 71%) as a yellow-orange solid which is used without further purification.

R_{f} 0.53 (90:10:1 CH₂Cl₂/MeOH/HOAc).

mp 210-214° C. dec.

¹H NMR (300 MHz, CD₃OD) δ 8.17 (d, J=8.5 Hz, 1H), 8.45 (dd, J=1.7, 8.5 Hz, 1H), 8.94 (d, J=1.7 Hz, 1H).

ESI MS (Negative Mode) m/z 223 [C₈H₄N₂O₄S−H]⁻.

B. Preparation of:

### 3-Nitro-4-thiocyanato-benzoic acid methyl ester

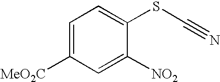

To a solution of 3-nitro-4-thiocyanato-benzoic acid (6.91 g, 30.8 mmol) in 1:1 MeOH/Et₂O (300 mL) is added (trimethylsilyl)diazomethane (2 M in Hex, 19.25 mL, 38.5 mmol). After stirring at room temperature for 2 h, additional (trimethylsilyl)diazomethane (2 M in Hex, 19.25 mL, 38.5 mmol) is added. After stirring for 30 min, the reaction is incomplete, monitoring by TLC. (Trimethylsilyl)diazomethane (2 M in Hex, 19.25 mL, 38.5 mmol) is added and the reaction mixture is stirred for 30 min. The reaction is quenched by the addition of HOAc (˜5 mL) and stirred at room temperature for 2 h. The solvent is evaporated under reduced pressure to afford the sub-title compound (7.39 g, ˜100%) as a yellow-brown solid.

mp 93-96° C.

¹H NMR (300 MHz, DMSO-d₆) δ 3.94 (s, 3H), 8.15 (d, J=8.5 Hz, 1H), 8.45 (dd, J=1.7, 8.5 Hz, 1H), 8.75 (d, J=1.7 Hz, 1H).

IR (neat) 1731 (s), 2254 (vs).

FAB MS m/z 238 [C₉H₆N₂O₄S]⁺.

C. Preparation of:

### 2-Amino-benzothiazole-5-carboxylic acid methyl ester

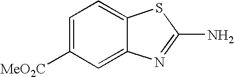

To a solution of 3-nitro-4-thiocyanato-benzoic acid methyl ester (7.39 g, 31.0 mmol) in HOAc (110 mL) is added palladium (10 wt % on activated carbon, 4.00 g) under a nitrogen atmosphere. The reaction mixture is hydrogenated (˜55 psi) for 3 d then filtered through a sintered-glass funnel containing diatomaceous earth, washing with MeOH (3×40 mL). The solvent is evaporated under reduced pressure to afford crude sub-title compound (6.3 g, >100%). The solid is dissolved in EtOAc (700 mL) and washed with saturated aqueous NaHCO₃ (250 mL). The organic layer is dried (MgSO₄), filtered and concentrated to afford the sub-title compound (5.12 g, 79%) as a yellow solid.

R_{f} 0.58 (90:10:1 CH₂Cl₂/MeOH/NH₄OH).

mp 204-206° C.

¹H NMR (300 MHz, DMSO-d₆) δ 3.85 (s, 3H), 7.60 (dd, J=1.6, 8.2 Hz, 1H), 7.71 (s, 2H), 7.80 (d, J=8.2 Hz, 1H), 7.84 (d, J=1.6 Hz, 1H).

ESI MS (Negative Mode) m/z 207 [C₉H₈N₂O₂S−H]⁻.

D. Preparation of:

### 3-(2-Amino-benzothiazol-5-yl)-pentan-3-ol

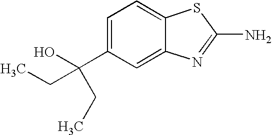

To a room temperature solution of 2-amino-benzothiazole-5-carboxylic acid methyl ester (500 mg, 2.40 mmol) in dimethoxyethane (80 mL) is slowly added ethylmagnesium bromide (3 M in Et₂O, 4.80 mL, 14.4 mmol) over 5 min. The reaction mixture becomes a suspension during the addition and stirring with a magnetic stir bar is stopped. The reaction is heated to 100° C. for 4 h then cooled to room temperature. The reaction is ˜50% complete by TLC. Ethylmagnesium bromide (3 M in Et₂O, 2.00 mL, 6.00 mmol) is added and the reaction heated to 100° C. for ˜12 h. The reaction is cooled to room temperature and quenched with saturated aqueous NH₄Cl (100 mL). The reaction mixture is diluted with EtOAc (200 ml) and H₂O (50 mL) and the organic layer is dried (MgSO₄), filtered and concentrated to afford crude sub-title compound (550 mg, ˜97%) which is ˜75% pure by ¹H NMR. The reaction residue is combined with another crude reaction residue and subjected to flash chromatography (silica gel, 97:3:0.3 CH₂Cl₂/MeOH/NH₄OH) to afford the sub-title compound (516 mg, 42% combined yield) as a yellow oil.

R_{f} 0.40 (90:10:1 CH₂Cl₂/MeOH/NH₄OH).

¹H NMR (300 MHz, CD₃OD) δ 0.76 (t, J=7.4 Hz, 6H), 1.77-1.90 (sym m, 4H), 7.11 (dd, J=1.8, 8.3 Hz, 1H), 7.46 (d, J=1.8 Hz, 1H), 7.50 (d, J=8.3 Hz, 1H).

IR (neat) 1533 (s), 1627 (m), 3000-3500 (m).

APCI MS m/z 237 [C₁₂H₁₆N₂OS+H]⁺.

E. Preparation of:

### N-{3-[1-(2-Amino-benzothiazol-5-yl)-1-ethyl-propyl]-1H-indol-7-yl}-methanesulfonamide

To a solution of 3-(2-amino-benzothiazol-5-yl)-pentan-3-ol (250 mg, 1.06 mmol) in CH₂Cl₂ (7.5 mL) is added N-(1H-indol-7-yl)-methanesulfonamide (223 mg, 1.06 mmol) followed by TFA (483 mg, 4.24 mmol). After stirring the reaction for 16 h at room temperature, the reaction is ˜15% complete by ¹H NMR. Trifluoroacetic acid (368 mg, 3.23 mmol) and N-(1H-indol-7-yl)-methanesulfonamide (89 mg, 0.42 mmol) are added and the reaction is stirred for 24 h. The solvent is evaporated under reduced pressure and the reaction residue is subjected to flash chromatography (silica gel, 95:5:0.5 CH₂Cl₂/MeOH/NH₄OH) to afford the title compound (242 mg, 53%) as a white solid.

R_{f} 0.44 (90:10:1 CH₂Cl₂/MeOH/NH₄OH).

mp 260-263° C. dec.

¹H NMR (300 MHz, DMSO-d₆) δ 0.56 (t, J=7.2 Hz, 6H), 1.99-2.20 (sym m, 4H), 2.96 (s, 3H), 6.59-6.65 (m, 2H), 6.85-6.91 (m, 2H), 7.21 (d, J=1.3 Hz, 1H), 7.30 (s, 2H), 7.33 (s, 1H), 7.42 (d, J=8.3 Hz, 1H), 9.21 (s, 1H), 10.59 (br s, 1H).

ESI MS (Negative Mode) m/z 427 [C₂₁H₂₄N₄O₂S₂−H]⁻.

HPLC (Method A) 96.3% (area percent), t_{R}=16.1 min.

### EXAMPLE 142

### N-[3-(1-Benzothiazol-5-yl-1-ethyl-propyl)-1H-indol-7-yl]-methanesulfonamide

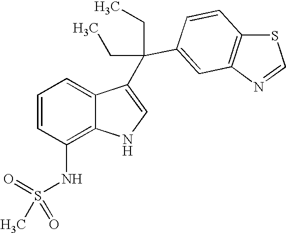 A. Preparation of:

### 3-Benzothiazol-5-yl-pentan-3-ol

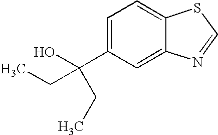

To a solution of 3-(2-amino-benzothiazol-5-yl)-pentan-3-ol (495 mg, 2.09 mmol) in DMF (14 mL) is added isoamyl nitrite (612 mg, 5.23 mmol) dropwise. The reaction mixture is first heated to 60° C. for 15 min followed by heating at 80° C. for 15 min. The cooled reaction mixture is quenched with saturated aqueous NaHCO₃ (50 mL) and diluted with EtOAc (400 mL) and H₂O (50 L). The aqueous layer is extracted with EtOAc (100 mL) and the combined organic layer is washed with saturated aqueous NaHCO₃ (2×35 mL) and brine (35 mL) then is dried (MgSO₄), filtered and concentrated. The orange-red residue is subjected to flash chromatography (silica gel, 7:3 Hex/EtOAc) to afford the sub-title compound (317 mg, 62%) as a yellow-orange solid.

R_{f} 0.44 (1:1 EtOAc/Hex).

mp 73-74° C.

¹H NMR (300 MHz, CDCl₃) δ 0.78 (t, J=7.4 Hz, 6H), 1.78 (s, 1H), 1.83-2.00 (sym m, 4H), 7.50 (dd, J=1.7, 8.4 Hz, 1H), 7.91 (d, J=8.4 Hz, 1H), 8.16 (d, J=1.7 Hz, 1H), 9.00 (s, 1H).

ESI MS m/z 222 [C₁₂H₁₅NOS+H]⁺.

B. Preparation of:

### N-[3-(1-Benzothiazol-5-yl-1-ethyl-propyl)-1H-indol-7-yl]-methanesulfonamide

To a suspension of 3-benzothiazol-5-yl-pentan-3-ol (307 mg, 1.39 mmol) in CH₂Cl₂ (10 mL) is added N-(1H-indol-7-yl)-methanesulfonamide (350 mg, 1.66 mmol) followed by TFA (792 mg, 6.95 mmol). After stirring the reaction for 24 h at room temperature, the reaction is ˜15% complete by ¹H NMR, and TFA (792 mg, 6.95 mmol) is added. After a further 24 h, the reaction is ˜33% complete by ¹H NMR. The reaction is heated to reflux overnight and the solvent is evaporated under reduced pressure. The reaction residue is diluted with EtOAc (200 mL) and is washed with saturated aqueous NaHCO₃ (2×25 mL) then dried (MgSO₄), filtered and concentrated. The brown residue is subjected to flash chromatography (silica gel, 98:2:0.25 CH₂Cl₂/MeOH/NH₄OH) to afford impure title compound (˜460 mg). The impure compound is subjected to flash chromatography (silica gel, 70:30 Hex/EtOAc) to afford the slightly impure title compound (250 mg, 55%) as an off-white solid.

R_{f} 0.53 (95:5:0.5 CH₂Cl₂/MeOH/NH₄OH).

mp 105-115° C. dec.

¹H NMR (300 MHz, DMSO-d₆) δ 0.58 (t, J=7.2 Hz, 6H), 2.12-2.29 (sym m, 4H), 2.97 (s, 3H), 6.52-6.62 (m, 2H), 6.89 (dd, J=1.1, 7.2 Hz, 1H), 7.28 (dd, J=1.5, 8.5 Hz, 1H), 7.40 (d, J=2.5 Hz, 1H), 7.93 (d, J=8.5 Hz, 1H), 7.99 (d, J=1.4 Hz, 1H), 9.23 (br s, 1H), 9.31 (s, 1H), 10.68 (br s, 1H).

ESI MS (Negative Mode) m/z 412 [C₂₁H₂₃N₃O₂S₂−H]⁻.

HPLC (Method A) 98.6% (area percent), t_{R}=20.1 min.

### EXAMPLE 143

### N-{3-[1-(3-Amino-benzo[d]isoxazol-6-yl)-1-ethyl-propyl]-1H-indol-7-yl}-methanesulfonamide

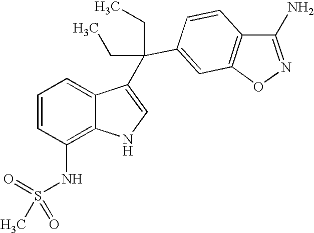 A. Preparation of:

### 4-Cyano-3-fluoro-benzoic acid methyl ester

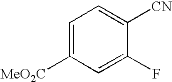

A mixture of 4-bromo-2-fluoro-benzonitrile (4.00 g, 20.0 mmol), Et₃N (3.94 g, 38.9 mmol), palladium(II)acetate (314 mg, 1.40 mmol), triphenylphosphine (214 mg, 0.816 mmol) in 4:1 CH₃CN/MeOH (50 mL) is purged with a stream of nitrogen for 15 min in a sealed tube equipped with gas inlet/outlet valves. The reaction is flushed with carbon monoxide (3×60 psi), releasing the pressure between each addition. The reaction is left under an atmosphere of carbon monoxide (60 psi) and heated to ˜50° C. overnight. The pressure is released and the reaction mixture is filtered through a sintered-glass funnel containing diatomaceous earth, washing with MeOH (20 mL). The reaction residue is subjected to flash chromatography (silica gel, 9:1 Hex/EtOAc) to afford the sub-title compound (2.13 g, 59%) as a white solid.

R_{f} 0.33 (4:1 Hex/EtOAc).

mp 61-62° C.

¹H NMR (300 MHz, CDCl₃) δ 3.97 (s, 3H), 7.72 (dd, J=6.2, 8.0 Hz, 1H), 7.86 (dd, J=1.3, 9.2 Hz, 1H), 7.93 (dd, J=1.3, 8.0 Hz, 1H).

B. Preparation of:

### 4-Cyano-3-isopropylideneaminooxy-benzoic acid methyl ester

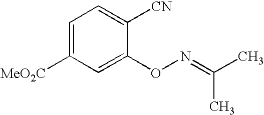

To a solution of acetone oxime in THF (20 mL) is added potassium t-butoxide (516 mg, 4.60 mmol) and the resulting light yellow suspension is stirred for 30 min. To the reaction mixture is added 4-cyano-3-fluoro-benzoic acid methyl ester (750 mg, 4.19 mmol). After stirring for 1.5 h, the reaction is quenched by the addition of saturated aqueous NH₄Cl (20 mL) and H₂O (30 mL). The reaction mixture is diluted with Et₂O (150 mL) and the organic layer washed with brine (25 mL) then dried (MgSO₄), filtered and concentrated. The solvent is evaporated under reduced pressure to afford the sub-title compound (695 mg, 71%) as a white solid which is used without further purification.

R_{f} 0.68 (1:1 Hex/EtOAc).

mp 104-106° C.

¹H NMR (300 MHz, CDCl₃) δ 2.09 (s, 3H), 2.17 (s, 3H), 3.95 (s, 3H), 7.60 (d, J=8.0 Hz, 1H), 7.68 (dd, J=1.4, 8.0 Hz, 1H), 8.17 (d, J=1.4 Hz, 1H).

APCI MS m/z 233 [C₁₂H₁₂N₂O₃+H]⁺.

C. Preparation of:

### 3-Amino-benzo[d]isoxazole-6-carboxylic acid methyl ester hydrochloride

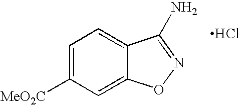

A solution of 4-cyano-3-isopropylideneaminooxy-benzoic acid methyl ester (530 mg, 2.28 mmol) in saturated HCl in MeOH (20 mL) is stirred for 2 d. Saturated HCl in MeOH (10 mL) is added and the reaction stirred for a further 24 h. The solvent is evaporated under reduced pressure and the reaction residue is diluted with EtOAc (150 mL) and washed with saturated aqueous NaHCO₃ (50 mL). The aqueous layer is extracted with EtOAc (50 mL) and the combined organic layer is dried (MgSO₄), filtered and concentrated. The reaction residue is subjected to flash chromatography (silica gel, 95:5:0.5 CH₂Cl₂/MeOH/NH₄OH) to afford the sub-title compound (463 mg, 88%) as a light yellow solid.

R_{f} 0.35 (95:5:0.5 CH₂Cl₂/MeOH/NH₄OH).

mp 180-183° C.

¹H NMR (300 MHz, DMSO-d₆) δ 3.90 (s, 3H), 6.58 (br s, 2H), 7.84 (dd, J=1.4, 8.1 Hz, 1H), 7.95 (d, J=8.1 Hz, 1H), 7.97 (s, 1H).

APCI MS (Negative Mode) m/z 191 [C₉H₈N₂O₃−H]⁻.

D. Preparation of:

### 3-(3-Amino-benzo[d]isoxazol-6-yl)-pentan-3-ol

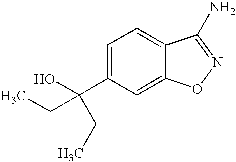

To a 0° C. solution of 3-amino-benzo[d]isoxazole-6-carboxylic acid methyl ester hydrochloride (129 mg, 0.564 mmol) in THF (7 mL) is added ethylmagnesium bromide (3 M in Et₂O, 1.10 mL, 3.35 mmol) dropwise. The reaction mixture is left to warm to room temperature overnight then quenched with saturated aqueous NH₄Cl (20 mL) and H₂O (20 mL) and is diluted with EtOAc (75 mL). The aqueous layer is extracted with EtOAc (75 mL) and the combined organic layer is washed with brine (20 mL) then dried (MgSO₄), filtered and concentrated. The reaction residue is subjected to flash chromatography (silica gel, 97:3:0.3 CH₂Cl₂/MeOH/NH₄OH) to afford the sub-title compound (51 mg, 41%) as a yellow oil.

R_{f} 0.56 (90:10:1 CH₂Cl₂/MeOH/NH₄OH).

¹H NMR (300 MHz, CD₃OD) δ 0.74 (t, J=7.3 Hz, 6H), 1.78-2.00 (sym m, 4H), 7.25 (dd, J=1.3, 8.3. Hz, 1H), 7.46 (d, J=1.3 Hz, 1H), 7.66 (d, J=8.3 Hz, 1H).

ESI MS m/z 221 [C₁₂H₁₆N₂O₂+H]⁺.

E. Preparation of:

### N-{3-[1-(3-Amino-benzo[d]isoxazol-6-yl)-1-ethyl-propyl]-1H-indol-7-yl}-methanesulfonamide

To a solution of 3-(3-amino-benzo[d]isoxazol-6-yl)-pentan-3-ol (152 g, 0.690 mmol) in CH₂Cl₂ (5 mL) is added N-(1H-indol-7-yl)-methanesulfonamide (188 mg, 0.897 mmol) followed by TFA (236 mg, 2.07 mmol). After stirring the reaction for 24 h at room temperature, there is no reaction by ¹H NMR, and TFA (393 mg, 3.45 mmol) is added. After a further 5 d, there is no reaction by ¹H NMR. After heating the reaction mixture to reflux for 24 h, the reaction is 25% complete by ¹H NMR. Additional N-(1H-indol-7-yl)-methanesulfonamide (58 mg, 0.276 mmol) and TFA (236 mg, 2.07 mmol) are added and the reaction heated to reflux for 4 d. The reaction is quenched by the addition of saturated aqueous NaHCO₃ (30 mL) and is diluted with EtOAc (100 mL). The organic layer is washed with brine (20 mL) then dried (MgSO₄), filtered and concentrated. The reaction residue is subjected to flash chromatography (silica gel, 97.5:2.5:0.25 CH₂Cl₂/MeOH/NH₄OH) to afford impure title compound (˜245 mg). The impure compound is resubjected to flash chromatography (silica gel, 90:8:1.8:0.2 CH₂Cl₂/CHCl₃/MeOH/NH₄OH) to afford impure title compound (˜89 mg). The impure title compound is subjected to preparative HPLC (Waters Symmetry C18 column, 7 μm, 19×300 mm, 60:40H₂O/CH₃CN, 0.1% TFA, 17 mL/min, 5=254 nm) to afford the title compound (28 mg, 10%) as a white solid.

R_{f} 0.31 (95:5:0.5 CH₂Cl₂/MeOH/NH₄OH).

mp 95-105° C.

¹H NMR (300 MHz, DMSO-d₆) δ 0.56 (br s, 6H), 2.07-2.20 (sym m, 4H), 2.97 (s, 3H), 6.24 (br s, 2H), 6.50-6.70 (m, 2H), 6.91 (d, J=7.1 Hz, 1H), 7.04 (d, J=7.8 Hz, 1H), 7.34-7.38 (m, 2H), 7.57 (d, J=8.2 Hz, 1H), 9.23 (br s, 1H), 10.66 (br s, 1H).

ESI MS m/z 413 [C₂₁H₂₄N₄O₃S+H]⁺.

HPLC (Method A) >99% (area percent), t_{R}=18.4 min.

### EXAMPLE 144

### N-{3-[1-(2-Amino-benzothiazol-6-yl)-1-ethyl-propyl]-1H-indol-7-yl}-methanesulfonamide

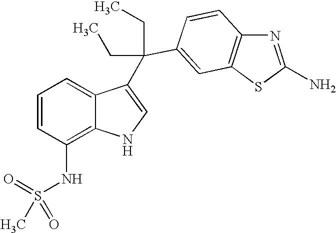 A. Preparation of:

### 3-(2-Amino-benzothiazol-6-yl)-pentan-3-ol

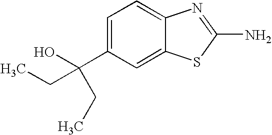

2-Amino-benzothiazole-6-carboxylic acid ethyl ester (2.50 g, 11.2 mmol) is dissolved in dioxane (225 mL), then ethylmagnesium bromide (18.7 mL of 3.0 M in Et₂O, 56.2 mmol) is added via syringe, and the reaction is refluxed overnight. An additional amount of ethylmagnesium bromide (18.7 mL of 3.0 M in Et₂O, 56.2 mmol) is added, and the reaction is held at reflux overnight. Upon cooling to room temperature, saturated aqueous NH₄Cl (150 mL) is added. The layers are separated, and the organic layer is extracted with EtOAc (150 mL). The combined organic layers are dried (MgSO₄), filtered and concentrated under reduced pressure. The residue is subjected to flash chromatography (silica gel, 95:5:0.5 CH₂Cl₂/MeOH/NH₄OH) to afford the sub-title compound (1.16 g, 44%) as an off-white solid.

¹H NMR (300 MHz, CD₃OD) δ0.75 (t, J=7.4 Hz, 6H), 1.78-1.87 (m, 4H), 7.25 (dd, J=1.8, 8.4 Hz, 1H), 7.34 (d, J=8.4 Hz, 1H), 7.64 (d, J=1.7 Hz, 1H).

APCI MS m/z 237 [C₁₂H₁₆N₂OS+H]⁺.

B: Preparation of:

### N-{3-[1-(2-Amino-benzothiazol-6-yl)-1-ethyl-propyl]-1H-indol-7-yl}-methanesulfonamide

3-(2-Amino-benzothiazol-6-yl)-pentan-3-ol (899 mg, 3.80 mmol) is combined with N-(1H-indol-7-yl)-methanesulfonamide (1.05 g, 5.01 mmol) in CH₂Cl₂ (38 mL). Trifluoroacetic acid (1.17 mL, 15.2 mmol) is added, and the reaction is stirred overnight at room temperature, then is concentrated under reduced pressure, redissolved in CH₂Cl₂ (100 mL), and washed with saturated aqueous NaHCO₃ (3×50 mL). The combined organic phases are dried (MgSO₄), filtered and concentrated under reduced pressure. The residue is subjected to flash chromatography (silica gel, 95:5 CH₂Cl₂/MeOH) to afford the title compound (820 mg, 50%) as a white solid.

R_{f} 0.49 (9:1 CH₂Cl₂/MeOH).

mp 155-160° C.

¹H NMR (300 MHz, DMSO-d₆) δ 0.56 (t, J=7.1 Hz, 6H), 2.05-2.18 (m, 4H), 2.98 (s, 3H), 6.61-6.64 (m, 2H), 6.90 (m, 1H), 7.06 (m, 1H), 7.16 (d, J=8.4 Hz, 1H), 7.30 (s, 2H), 7.33 (d, J=2.1 Hz, 1H), 7.53 (s, 1H), 9.22 (s, 1H), 10.60 (s, 1H).

APCI MS mz/z 429 [C₂₁H₂₄N₄O₂S₂+H]⁺.

HPLC (Method A) 97.2% (AUC), t_{R}=16.2 min.

### EXAMPLE 145

### N-{3-[1-Ethyl-1-(2-methyl-benzooxazol-5-yl)-propyl]-1H-indol-7-yl}-methanesulfonamide

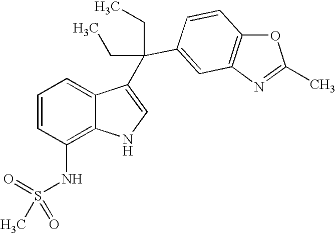 A. Preparation of:

### 3-Amino-4-hydroxy-benzoic acid methyl ester

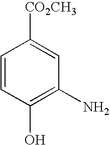

4-Hydroxy-3-nitro-benzoic acid methyl ester (500 mg, 2.54 mmol) is dissolved in MeOH (10 mL), then 10% palladium on carbon (50 mg of 50% wet) is added, and the reaction is placed under 1 atm of H₂ overnight. The mixture is filtered through Celite® to remove the catalyst and the filtrate is concentrated under reduced pressure to afford the sub-title compound (435 mg, >100%) which is used without further purification.

¹H NMR (300 MHz, DMSO-d₆) δ 3.74 (s, 3H), 4.78 (br s, 2H), 6.70 (d, J=8.2 Hz, 1H), 7.09 (dd, J=2.1, 8.2 Hz, 1H), 7.24 (d, J=2.1 Hz, 1H), ˜10 (br s, 1H).

APCI MS m/z 168 [C₈H₉NO₃+H]⁺.

B. Preparation of:

### N-[5-(1-Ethyl-1-hydroxy-propyl)-2-hydroxy-phenyl]-acetamide

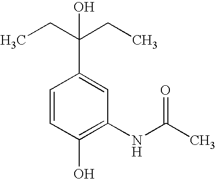

Ethylmagnesium bromide (55.8 mL of 3.0 M in Et₂O, 168 mmol) is added to THF (60 mL), chilled to 0° C., and a solution of 3-amino-4-hydroxy-benzoic acid methyl ester (4.00 g, 23.9 mmol) in THF (60 mL) is added dropwise. The reaction is warmed to room temperature and stirred overnight, after which saturated aqueous NH₄Cl (50 mL) is added, followed by H₂O (250 mL) and EtOAc (250 mL). The resulting emulsion is filtered through diatomaceous earth, the layers are separated, and the aqueous phase is extracted with EtOAc (2×150 mL). The combined organic phases are dried (MgSO₄), filtered and concentrated under reduced pressure. Flash chromatography (silica gel, 96:4:0.5 CH₂Cl₂/MeOH/NH₄OH) followed by trituration with CH₂Cl₂, then flash chromatography (silica gel, 3:2 EtOAc/Hex) affords 2-amino-4-(1-ethyl-1-hydroxy-propyl)-phenol (852 mg). A portion of this (200 mg, 1.02 mmol) is suspended in EtOAc (1.1 mL) then, acetic anhydride (0.22 mL, 2.30 mmol) is added. The reaction is stirred at room temperature overnight, then is diluted with EtOAc, and washed with H₂O (3×25 mL). The organic phase is dried (MgSO₄), filtered and concentrated under reduced pressure to afford the sub-title compound as an off-white solid (219 mg, 16%), which is used without further purification.

¹H NMR (300 MHz, DMSO-d₆) δ 0.63 (t, J=7.3 Hz, 6H), 1.60-1.67 (m, 4H), 2.08 (s, 3H), 4.33 (s, 1H), 6.76 (d, J=8.4 Hz, 1H), 6.94 (dd, J=2.0, 8.4 Hz, 1H), 7.51 (d, J=1.7 Hz, 1H), 9.47 (s, 1H), 9.49 (s, 1H).

ESI MS (negative mode) m/z 236 [C₁₃H₁₉NO₃−H]⁻.

C. Preparation of:

### N-{5-[1-Ethyl-1-(7-methanesulfonylamino-1H-indol-3-yl)-propyl]-2-hydroxy-phenyl}-acetamide

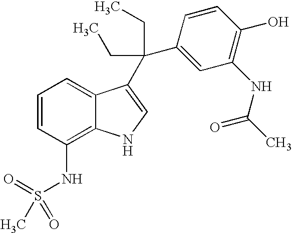

N-[5-(1-Ethyl-1-hydroxy-propyl)-2-hydroxy-phenyl]-acetamide (200 mg, 0.84 mmol) and N-(1H-indol-7-yl)-methanesulfonamide (235 mg, 1.12 mmol) are combined in CH₂Cl₂ (8.4 mL), and TFA (259 δL, 3.36 mmol) is added. The solution is allowed to stir at room temperature for 3 d, then CH₂Cl₂ (25 mL) and saturated aqueous NaHCO₃ (25 mL) are added. A precipitate forms which is filtered off and dried in vacuo to afford the sub-title compound (304 mg, 84%).

¹H NMR (300 MHz, DMSO-d₆) δ 0.55 (t, J=6.9 Hz, 6H), 1.91-2.14 (m, 4H), 2.03 (s, 3H), 2.80 (s, 3H), 3.10-3.70 (br s, 1H), 6.37 (d, J=7.8 Hz, 1H), 6.52 (t, J=7.7 Hz, 1H), 6.69 (d, J=8.6 Hz, 1H), 6.78 (d, J=7.3 Hz, 1H), 6.86 (m, 1H), 7.13 (s, 1H), 7.41 (s, 1H), 8.95-9.80 (br s, 1H), 9.48 (s, 1H), 10.34 (s, 1H).

CI MS (negative mode) m/z 428 [C₂₂H₂₇N₃O₄S−H]⁻.

D. Preparation of:

### N-{3-[1-Ethyl-1-(2-methyl-benzooxazol-5-yl)-propyl]-1H-indol-7-yl}-methanesulfonamide

N-{5-[1-Ethyl-1-(7-methanesulfonylamino-1H-indol-3-yl)-propyl]-2-hydroxy-phenyl}-acetamide is dissolved in HOAc (8 mL), refluxed for 20 h, and concentrated under reduced pressure. The residue is subjected to flash chromatography (silica gel, 98:2 CH₂Cl₂/MeOH) to afford the title compound (224 mg, 80%) as a white solid.

R_{f} 0.46 (95:5 CH₂Cl₂/MeOH).

mp 152-160° C.

¹H NMR (300 MHz, DMSO-d₆) δ 0.57 (t, J=7.2 Hz, 6H), 2.07-2.24 (m, 4H), 2.56 (s, 3H), 2.98 (s, 3H), 6.50 (d, J=7.8 Hz, 1H), 6.61 (t, J=7.8 Hz, 1H), 6.91 (dd, J=0.7, 7.5 Hz, 1H), 7.16 (dd, J=1.7, 8.6 Hz, 1H), 7.38 (d, J=2.5 Hz, 1H), 7.43 (d, J=8.6 Hz, 1H), 7.53 (d, J=1.5 Hz, 1H), 9.24 (s, 1H), 10.65 (s, 1H).

ESI MS m/z 412 [C₂₂H₂₅N₃O₃S+H]⁺.

HPLC (Method A) 96.3% (AUC), t_{R}=20.2 min.

### EXAMPLE 146

### N-[3-(1-Benzooxazol-5-yl-1-ethyl-propyl)-1H-indol-7-yl]-methanesulfonamide

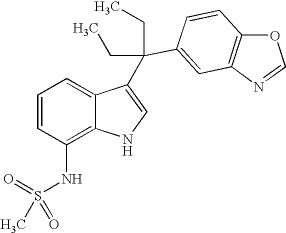 A. Preparation of:

### N-{3-[1-(3-Amino-4-hydroxy-phenyl)-1-ethyl-propyl]-1H-indol-7-yl}-methanesulfonamide

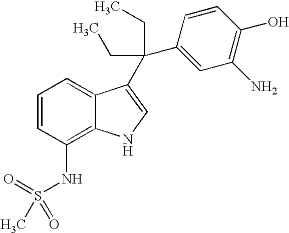

2-Amino-4-(1-ethyl-1-hydroxy-propyl)-phenol (440 mg, 2.25 mmol) and N-(1H-indol-7-yl)-methanesulfonamide (631 mg, 3.00 mmol) are combined in CH₂Cl₂ (20 mL), then

TFA (0.69 mL, 9.00 mmol) is added. After stirring overnight at room temperature, a precipitate is filtered off, dissolved in ˜10% MeOH in CH₂Cl₂, and washed with saturated aqueous NaHCO₃ (2×30 mL). The organic layer is dried (MgSO₄), filtered and concentrated under reduced pressure, and the residue is subjected to flash chromatography (silica gel, 95:5. CH₂Cl₂/MeOH) to afford the sub-title compound (510 mg, 58%).

¹H NMR (300 MHz, DMSO-d₆) δ 0.54 (t, J=7.2 Hz, 6H), 1.90-2.10 (m, 4H), 2.98 (s, 3H), 4.24 (br s, 2H), 6.37 (dd, J=2.1, 8.1 Hz, 1H), 6.45 (d, J=2.0 Hz, 1H), 6.52 (d, J=8.1 Hz, 1H), 6.62-6.72 (m, 2H), 6.91 (dd, J=1.1, 7.1 Hz, 1H), 7.24 (d, J=2.4 Hz, 1H), 8.61 (br s, 1H), 9.19 (br s, 1H), 10.48 (br s, 1H).

ESI MS m/z 388 [C₂₀H₂₅N₃O₃S+H]⁺.

B. Preparation of:

### N-[3-(1-Benzooxazol-5-yl-1-ethyl-propyl)-1H-indol-7-yl]-methanesulfonamide

N-{3-[1-(3-Amino-4-hydroxy-phenyl)-1-ethyl-propyl]-1H-indol-7-yl}-methanesulfonamide (478 mg, 1.23 mmol) is refluxed for 3 h in triethyl orthoformate (5.00 mL, 30.0 mmol). The mixture is cooled and then concentrated under reduced pressure. The residue is subjected multiple times to flash chromatography (silica gel, 98.5:1.5 CH₂Cl₂/MeOH) to afford the title compound (279 mg, 57%).

R_{f} 0.44 (95:5 CH₂Cl₂/MeOH).

mp 132-135° C.

¹H NMR (300 MHz, DMSO-d₆) δ 0.57 (t, J=7.1 Hz, 6H), 2.12-2.24 (m, 4H), 2.98 (s, 3H), 6.52 (d, J=7.9 Hz, 1H), 6.61 (t, J=7.9 Hz, 1H), 6.91 (d, J=7.3 Hz, 1H), 7.25 (dd, J=1.5, 8.6 Hz, 1H), 7.40 (d, J=2.4 Hz, 1H), 7.56 (d, J=8.6 Hz, 1H), 7.69 (d, J=1.2 Hz, 1H), 8.65 (s, 1H), 9.23 (s, 1H), 10.67 (s, 1H).

ESI MS (negative mode) m/z 396 [C₂₁H₂₃N₃O₃S−H]⁻.

HPLC (Method D) 98.2% (AUC), t_{R}=19.9 min.

### EXAMPLE 147

### N-{6-[1-Ethyl-1-(7-methanesulfonylamino-1H-indol-3-yl)-propyl]-benzothiazol-2-yl}-acetamide

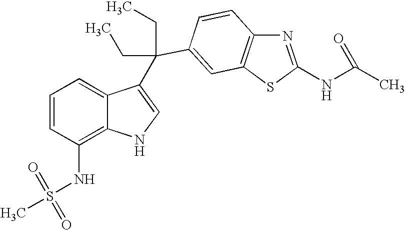 A. Preparation of:

### N-[6-(1-Ethyl-1-hydroxy-propyl)-benzothiazol-2-yl]-acetamide

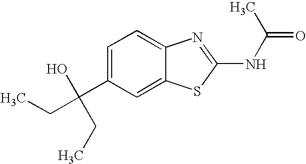

3-(2-Amino-benzothiazol-6-yl)-pentan-3-ol (400 mg, 1.69 mmol; above) is suspended in EtOAc (1.9 mL) then acetic anhydride (0.36 mL, 3.81 mmol) is added. After sting overnight at room temperature, EtOAc (10 mL) is added, and the reaction is washed with saturated aqueous NaHCO₃ (2×10 mL). The organic layer is dried (MgSO₄), filtered and concentrated under reduced pressure to afford the sub-title compound (461 mg, 98%) which is used without further purification.

¹H NMR (300 MHz, DMSO-d₆) δ 0.64 (t, J=7.2 Hz, 6H), 1.72-1.99 (m, 4H), 2.19 (s, 3H), 4.62 (s, 1H), 7.39 (dd, J=1.6, 8.5 Hz, 1H), 7.64 (d, J=8.5 Hz, 1H), 7.93 (d J=1.4 Hz, 1H), 12.27 (s, 1H).

ESI MS m/z 279 [C₁₄H₁₈N₂O₂S+H]⁺.

B. Preparation of:

### N-{6-[1-Ethyl-1-(7-methanesulfonylamino-1H-indol-3-yl)-propyl]-benzothiazol-2-yl}-acetamide

N-[6-(1-Ethyl-1-hydroxy-propyl)-benzothiazol-2-yl]-acetamide (400 mg, 1.44 mmol) and N-(1H-indol-7-yl)-methanesulfonamide (393 mg, 1.87 mmol) are combined in CH₂Cl₂ (15 mL), then TFA (0.55 mL, 7.20 mmol) is added. After stirring overnight at room temperature, the reaction is concentrated under reduced pressure and redissolved in CH₂Cl₂ (30 mL) and washed with saturated aqueous NaHCO₃ (30 mL). A precipitate then forms which after addition of Hex (30 mL) is filtered off. The solids are tritrated with 4:1 CH₂Cl₂/Hex, then subjected to flash chromatography (silica gel, 98:2 CH₂Cl₂/MeOH) to afford the title compound as a light pink solid (218 mg, 32%).

R_{f} 0.46 (9:1 CH₂Cl₂/MeOH).

mp 286-288° C.

¹H NMR (300 MHz, DMSO-d₆) δ 0.57 (t, J=7.1 Hz, 6H), 2.08-2.26 (m, 4H), 2.17 (s, 3H), 2.98 (s, 3H), 6.55-6.64 (m, 2H), 6.91 (m, 1H), 7.21 (dd, J=1.4, 8.5 Hz, 1H), 7.38 (d, J=2.2 Hz, 1H), 7.53 (d, J=8.5. Hz, 1H), 7.92 (m, 1H), 9.23 (s, 1H), 10.64 (s, 1H), 12.22 (s, 1H).

ESI MS m/z 471 [C₂₃H₂₆N₄O₃S₂+H]⁺.

HPLC (Method A) 96.6% (AUC), t_{R}=18.9 min.

### EXAMPLE 148

### N-{3-[1-(2-Chloro-benzothiazol-6-yl)-1-ethyl-propyl]-1H-indol-7-yl}-methanesulfonamide

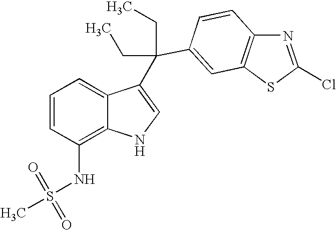 A. Preparation of:

### 3-(2-Chloro-benzothiazol-6-yl)-pentan-3-ol

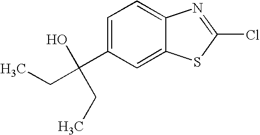

Copper(II)chloride (102 mg, 0.76 mmol) is dissolved in CH₃CN (3.2 mL) then tert-butyl nitrite (125 □L, 0.95 mmol) is added. The reaction is heated to 60° C., then 3-(2-amino-benzothiazol-6-yl)-pentan-3-ol (150 mg, 0.63 mmol; from Example 11, step (i) above) is added. After stirring 20 min, the reaction is diluted with Et₂O (20 mL) and poured into 2 M HCl (20 mL). The layers are separated, and the aqueous layer is extracted with Et₂O (2×10 mL). The combined organic extracts are dried (MgSO₄), filtered and concentrated under reduced pressure. The residue is subjected to flash chromatography (silica gel, 9:1 Hex/EtOAc) to afford the sub-title compound (105 mg, 65%).

¹H NMR (300 MHz, DMSO-d₆) δ 0.63 (t, J=7.3 Hz, 6H), 1.71-1.79 (m, 4H), 4.75 (br s, 1H), 7.53 (dd, J=1.8, 8.6 Hz, 1H), 7.88 (d, J=8.6 Hz, 1H), 8.09 (d, J=1.6 Hz, 1H).

ESI MS m/z 256, 258 [C₁₂H₁₄ClNOS+H]⁺.

B. Preparation of:

### N-{3-[1-(2-Chloro-benzothiazol-6-yl)-1-ethyl-propyl]-1H-indol-7-yl}-methanesulfonamide

3-(2-Chloro-benzothiazol-6-yl)-pentan-3-ol (224 mg, 0.88 mmol) and N-(1H-indol-7-yl)-methanesulfonamide (240 mg, 1.14 mmol) are combined in CH₂Cl₂ (6 mL), then TFA (0.33 mL, 4.40 mmol) is added. After stirring overnight at room temperature, the reaction is concentrated under reduced pressure and redissolved in CH₂Cl₂ (50 mL) and washed with saturated aqueous NaHCO₃ (3×25 mL). The organic layer is dried (MgSO₄), filtered and concentrated under reduced pressure. The residue is subjected to preparative HPLC (Waters Symmetry C18 column, 7 μm, 77×230 mm, 7:3 CH₃CN/H₂O, 0.1% TFA, 250 mL/min, δ=254 nm) to afford the title compound (78 mg, 20%).

R_{f} 0.57 (95:5 CH₂Cl₂/MeOH).

mp 180-190° C.

¹H NMR (300 MHz, DMSO-d₆) δ 0.57 (t, J=7.2 Hz, 6H), 2.08-2.32 (m, 4H), 2.98 (s, 3H), 6.53 (d, J=7.9 Hz, 1H), 6.63 (t, J=7.9 Hz, 1H), 6.92 (d, J=7.0 Hz, 1H), 7.34 (dd, J=1.7, 8.6 Hz, 1H), 7.41 (d, J=2.4 Hz, 1H), 7.77 (d, J=8.6 Hz, 1H), 8.09 (d, J=1.5 Hz, 1H), 9.24 (s, 1H), 10.68 (s, 1H).

APCI MS m/z 448 [C₂₁H₂₂ClN₃O₂S₂+H]⁺.

HPLC (Method A) >99%, (AUC); t_{R}=18.7 min.

### EXAMPLE 149

### N-[3-(1-Benzothiazol-6-yl-1-ethyl-propyl)-1H-indol-7-yl]-methanesulfonamide

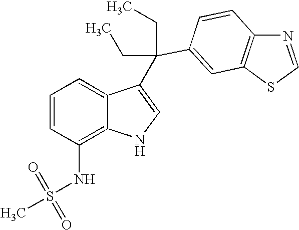 A. Preparation of:

### 3-Benzothiazol-6-yl-pentan-3-ol

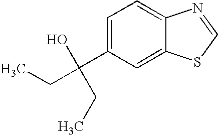

3-(2-Amino-benzothiazol-6-yl)-pentan-3-ol (150 mg, 0.63 mmol;) is dissolved in DMF (2.1 mL) then isoamyl nitrite (212 μL, 1.58 mmol) is added and the reaction is heated to 60° C. for 2 h. Upon cooling, EtOAc (20 mL) is added, and the reaction is washed with saturated aqueous NaHCO₃ (2×10 mL). The layers are separated, and the organic layer is dried (MgSO₄), filtered and concentrated under reduced pressure. The residue is subjected to flash chromatography (7:3 Hex/EtOAc) to afford the sub-title compound (75 mg, 54%) as a pale yellow solid.

¹H NMR (300 MHz, DMSO-d₆) δ 0.65 (t, J=7.3 Hz, 6H), 1.76-1.86 (m, 4H), 4.71 (s, 1H), 7.53 (dd, J=1.7, 8.6 Hz, 1H), 8.01 (d, J=8.5 Hz, 1H), 8.15 (d, J=1.6 Hz, 1H), 9.32 (s, 1H).

ESI MS m/z 222 [C₁₂H₁₅NOS+H]⁺.

B. Preparation of:

### N-[3-(1-Benzothiazol-6-yl-1-ethyl-propyl)-1H-indol-7-yl]-methanesulfonamide

3-Benzothiazol-6-yl-pentan-3-ol (169 mg, 0.76 mmol) and N-(1H-indol-7-yl)-methanesulfonamide (213 mg, 1.01 mmol) are combined in CH₂Cl₂ (5 mL), then TFA (0.47 mL, 6.10 mmol) is added. After stirring 2 d at room temperature, more N-(1H-indol-7-yl)-methanesulfonamide (100 mg, 0.48 mmol) and TFA (0.2 mL, 2.60 mmol) are added. After stirring an additional 3 d at room temperature, the reaction is diluted with CH₂Cl₂ (20 mL) and washed with saturated aqueous NaHCO₃ (2×30 mL). The organic layer is dried (MgSO₄), filtered and concentrated under reduced pressure. The residue is subjected to preparative HPLC (Waters Symmetry C18 column, 7 □m, 77×230 mm, 55:45 CH₃CN/H₂O, 0.1% TFA, 250 mL/min, δ=254 nm) to afford the title compound (83 mg, 26%).

R_{f} 0.38 (95:5 CH₂Cl₂/MeOH).

mp 117-120° C.

¹H NMR (300 MHz, DMSO-d₆) δ 0.56 (t, J=7.2 Hz, 6H), 2.12-2.24 (m, 4H), 2.97 (s, 3H), 6.52 (d, J=7.4 Hz, 1H), 6.59 (d, J=7.4 Hz, 1H), 6.89 (dd, J=0.9, 7.3 Hz, 1H), 7.30 (dd, J=1.8, 8.6 Hz, 1H), 7.39 (d, J=2.5 Hz, 1H), 7.87 (d, J=8.6 Hz, 1H), 8.12 (d, J=1.6 Hz, 1H), 9.23-9.27 (m, 2H), 10.67 (s, 1H).

APCI MS m/z 414 [C₂₁H₂₃N₃O₂S₂+H]⁺.

HPLC (Method A) >99% (AUC), t_{R}=16.6 min.

### EXAMPLE 150

### N-{5-[1-Ethyl-1-(7-methanesulfonylamino-1H-indol-3-yl)-propyl]-benzothiazol-2-yl}-acetamide

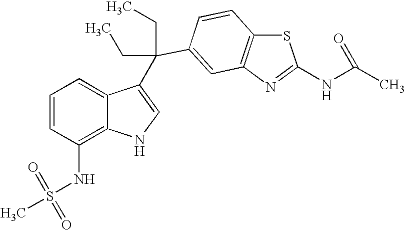 A. Preparation of:

### N-[5-(1-Ethyl-1-hydroxy-propyl)-benzothiazol-2-yl]-acetamide

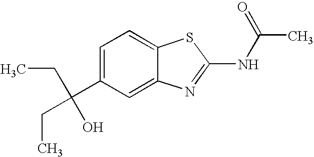

3-(2-Amino-benzothiazol-5-yl)-pentan-3-ol (351 mg, 1.49 mmol) is dissolved in EtOAc (1.7 mL) then acetic anhydride (317 μL, 3.35 mmol) is added. After stirring overnight at room temperature, a small amount of EtOAc is added, and the solution is washed with saturated aqueous NaHCO₃ (2×20 mL). The organic layer is dried (MgSO₄), filtered and concentrated under reduced pressure. The residue is subjected to flash chromatography (silica gel, 95:5 CH₂Cl₂/MeOH) to afford the sub-title compound (395 mg, 95%).

¹H NMR (300 MHz, DMSO-d₆) δ 0.63 (t, J=7.3 Hz, 6H), 1.65-1.82 (m, 4H), 2.17 (s, 3H), 4.57 (s, 1H), 7.25 (dd, J=1.5, 8.3 Hz, 1H), 7.73 (d, J=1.4 Hz, 1H), 7.81 (d, J=8.3 Hz, 1H), 12.25 (s, 1H).

ESI MS m/z 279 [C₁₄H₁₈N₂O₂S+H]⁺.

B. Preparation of:

### N-{5-[1-Ethyl-1-(7-methanesulfonylamino-1H-indol-3-yl)-propyl]-benzothiazol-2-yl}-acetamide

N-[5-(1-Ethyl-1-hydroxy-propyl)-benzothiazol-2-yl]-acetamide (386 mg, 1.39 mmol) and N-(1H-indol-7-yl)-methanesulfonamide (378 mg, 1.80 mmol) are combined in CH₂Cl₂ (14 mL), then TFA (535 μL, 6.95 mmol) is added. After stirring overnight at room temperature, more TFA (˜0.5 μL) is added and the reaction is stirred for 5 d at room temperature. Methylene chloride (20 μL) and saturated aqueous NaHCO₃ (20 mL) are added and the layers are separated. The organic layer is dried (MgSO₄), filtered and concentrated under reduced pressure. The residue is subjected to preparative HPLC (Waters Symmetry C18 column, 7 μm, 77×230 mm, 55:45 CH₃CN/H₂O; 0.1% TFA, 250 mL/min, δ=254 nm) to afford the title compound (126 mg, 19%).

R_{f} 0.25 (95:5 CH₂Cl₂/MeOH).

mp 266-268° C.

¹H NMR (300 MHz, DMSO-d₆) δ 0.57 (t, J=7.1 Hz, 6H), 2.06-2.25 (m, 4H), 2.17 (s, 3H), 2.96 (s, 3H), 6.54-6.63 (m, 2H), 6.89 (dd, J=1.3, 7.0 Hz, 1H), 7.10 (dd, J=1.5, 8.4 Hz, 1H), 7.37 (d, J=2.4 Hz, 1H), 7.63 (d, J=1.3 Hz, 1H), 7.71 (d, J=8.4 Hz, 1H), 9.21 (br s, 1H), 10.63 (s, 1H), 12.17 (s, 1H).

ESI MS (negative mode) m/z 469 [C₂₃H₂₆N₄O₃S₂−H]⁻.

HPLC (Method A) >99% (AUC), t_{R}=19.1 min.

### EXAMPLE 151

### N-{3-[1-Ethyl-1-(2-trifluoromethyl-3H-benzoimidazol-5-yl)-propyl]-1H-indol-7-yl}-methanesulfonamide

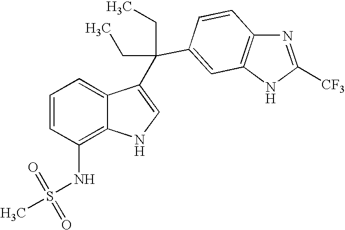 A. Preparation of:

### 2-Trifluoromethyl-3H-benzoimidazole-5-carboxylic acid methyl ester

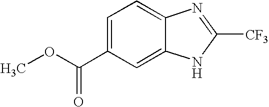

3,4-Diamino-benzoic acid methyl ester (1.50 g, 9.02 mmol) is dissolved in TFA (25 mL) and refluxed for approximately 1.5 h. Upon cooling, the reaction is quenched with approximately 400 mL saturated aqueous NaHCO₃ and extracted with CH₂Cl₂ (2×200 mL). The organic layer is dried (MgSO₄), filtered and concentrated under reduced pressure to afford the sub-title compound (2.14 g, 97%) as an off-white solid which is used without further purification.

¹H NMR (300 MHz, DMSO-d₆) δ 3.90 (s, 3H), 7.82 (m, 1H), 7.99 (m, 1H), 8.33 (m, 1H), 14.33 (br s, 1H).

¹⁹F NMR (282 MHz, DMSO-d₆) δ −63.56.

ESI MS (negative mode) m/z 243 [C₁₀H₇F₃N₂O₂−H]⁻.

B. Preparation of:

### 3-(2-Trifluoromethyl-3H-benzoimidazol-5-yl)-pentan-3-ol

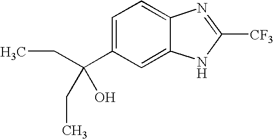

2-Trifluoromethyl-3H-benzoimidazole-5-carboxylic acid methyl ester (2.13 g, 8.72 mmol) is dissolved in dioxane (80 mL) and chilled in an ice bath then, ethylmagnesium bromide (8.72 mL of 3.0 M in Et₂O, 26.2 mmol) is slowly added via syringe. The ice bath is removed after several minutes, and reaction is allowed to warm to room temperature. After stirring overnight at room temperature, the reaction is heated to 50° C. for 5.75 h. Upon cooling to 0° C., the reaction is quenched with 2 M HCl (100 mL), extracted with EtOAc (3×100 mL), dried (MgSO₄), filtered and concentrated under reduced pressure. The residue is subjected to flash chromatography (silica gel, 98:2 CH₂Cl₂/MeOH) to afford the sub-title compound (697 mg, 29%).

¹H NMR (300 MHz, DMSO-d₆) δ 0.63 (t, J=7.3 Hz, 6H), 1.71-1.88 (m, 4H), 4.62 (s, 1H), 7.13-7.91 (br m, 3H), 13.72 (br s, 1H).

¹⁹F NMR (282 MHz, DMSO-d₆) δ −63.07.

ESI MS m/z 273 [C₁₃H₁₅F₃N₂O+H]⁺.

C. Preparation of:

### N-{3-[1-Ethyl-1-(2-trifluoromethyl-3H-benzoimidazol-5-yl)-propyl]-1H-indol-7-yl}-methanesulfonamide

3-(2-Trifluoromethyl-3H-benzoimidazol-5-yl)-pentan-3-ol (500 mg, 1.83 mmol) and N-(1H-indol-7-yl)-methanesulfonamide (256 mg, 1.22 mmol) are combined in CH₂Cl₂ (15 mL), then TFA (282 μL, 3.66 mmol) is added. After stirring overnight at room temperature, more N-(1H-indol-7-yl)-methanesulfonamide (128 mg, 0.61 mmol) is added and the reaction is stirred for several more hours, after which saturated aqueous NaHCO₃ (20 mL) is added. The layers are separated and the aqueous layer extracted with CH₂Cl₂ (2×20 mL). The combined organic layers are dried (MgSO₄), filtered and concentrated under reduced pressure. The residue is subjected twice to flash chromatography (silica gel, 99:1 CH₂C₂/MeOH) to afford the title compound (413 mg, 49%).

R_{f} 0.28 (95:5 CH₂Cl₂/MeOH).

mp 225-235° C.

¹H NMR (300 MHz, DMSO-d₆) δ 0.56 (t, J=7.1 Hz, 6H), 2.10-2.23 (m, 4H), 2.97 (s, 3H), 6.50-6.61 (m, 2H), 6.87-6.90 (m, 1H), 7.18 (dd, J=1.4, 8.7 Hz, 1H), 7.38 (d, J=2.4 Hz, 1H), 7.50 (br m, 2H), 9.22 (s, 1H), 10.64 (s, 1H), 13.66 (s, 1H).

ESI MS m/z 465 [C₂₂H₂₃F₃N₄O₂S+H]⁺.

HPLC (Method A) 98.5% (AUC), t_{R}=19.3 min.

### EXAMPLE 152

### N-{3-[1-(3-Amino-benzo[d]isoxazol-5-yl)-1-ethyl-propyl]-1H-indol-7-yl}-methanesulfonamide

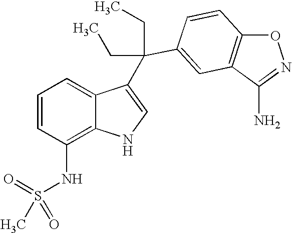 A. Preparation of:

### 3-Cyano-4-fluoro-benzoic acid methyl ester

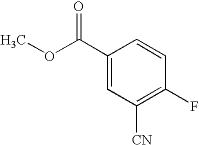

To CH₃CN (100 mL) and MeOH (25 mL) in a sealed tube apparatus is added 5-bromo-2-fluorobenzonitrile (5.00 g, 25.0 mmol), TEA (6.97 mL, 50.0 mmol), palladium(II)acetate (393 mg, 1.75 mmol), and triphenylphosphine (269 mg, 1.03 mmol). The reaction is sealed, purged with carbon monoxide twice, and placed under 60 psi of carbon monoxide at 60° C. for 4 d. Upon cooling, the reaction is filtered through diatomaceous earth, washing with MeOH. The filtrate is concentrated under reduced pressure, and the residue suspended in 4:1 EtOAc/Hex (100 mL) then filtered. The filtrate is concentrated under reduced pressure then subjected to flash chromatography (silica gel, CHCl₃) to afford the sub-title compound (1.91 g, 43%).

¹H NMR (300 MHz, DMSO-d₆) δ 3.87 (s, 3H), 7.66 (t, J=9.6 Hz, 1H), 8.30 (ddd, J=2.2, 5.3, 8.6 Hz, 1H), 8.43 (dd, J=2.2, 6.3 Hz, 1H).

FAB MS m/z 154 [C₉H₆FNO₂+H−HCN]⁺, 136 [C₉H₆FNO₂+H−CO₂]⁺.

B. Preparation of:

### 3-Cyano-4-isopropylideneaminooxy-benzoic acid methyl ester

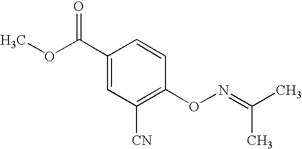

Acetone oxime (449 mg, 6.14 mmol) is dissolved in THF (30 mL) then potassium tert-butoxide (689 mg, 6.14 mmol) is added. After stirring at room temperature for 30 min, 3-cyano-4-fluoro-benzoic acid methyl ester (1.00 g, 5.58 mmol) is added. After stirring 2 h at room temperature, saturated aqueous NH₄Cl (20 mL), H₂O (30 mL), and EtOAc (100 mL) are added. The layers are separated, and the organic layer is dried (MgSO₄), filtered and concentrated under reduced pressure to afford the sub-title compound (1.23 g, 95%) which requires no further purification.

¹H NMR (300 MHz, DMSO-d₆) δ 2.06 (s, 3H), 2.13 (s, 3H), 3.85 (s, 3H), 7.64 (d, J=8.9 Hz, 1H), 8.21 (dd, J=1.8, 8.9 Hz, 1H), 8.27 (d, J=1.8 Hz, 1H).

ESI MS m/z 233 [C₁₂H₁₂N₂O₃+H]⁺.

C. Preparation of:

### 3-Amino-benzo[d]isoxazole-5-carboxylic acid methyl ester

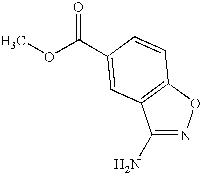

3-Cyano-4-isopropylideneaminooxy-benzoic acid methyl ester (1.20 g, 5.17 mmol) is dissolved in a saturated solution of HCl in MeOH (35 mL). After stirring at room temperature for 2 d, the reaction is concentrated under reduced pressure, then added saturated aqueous NaHCO₃ (100 mL) and EtOAc (100 mL) are added. The layers are separated, and the organic layer dried (MgSO₄), filtered and concentrated under reduced pressure. The crude residue is triturated with CH₂Cl₂ (30 mL), and the filtrate concentrated under reduced pressure and triturated with 7:3 CH₂Cl₂ (6 mL). The solids are combined to afford the sub-title compound (832 mg, 84%).

¹H NMR (300 MHz, DMSO-d₆) δ 3.88 (s, 3H), 6.64 (br s, 2H), 7.56 (dd, J=0.6, 8.8 Hz, 1H), 8.10 (dd, J=1.7, 8.8 Hz, 1H), 8.62 (dd, J=0.6, 1.7 Hz, 1H).

ESI MS m/z 193 [C₉H₈N₂O₃+H]⁺.

D. Preparation of:

### 3-(3-Amino-benzo[d]isoxazol-5-yl)-pentan-3-ol

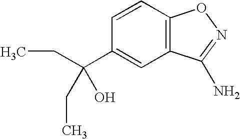

3-Amino-benzo[d]isoxazole-5-carboxylic acid methyl ester (700 mg, 3.64 mmol) is dissolved in THF (35 mL) and chilled in an ice bath. A solution of ethylmagnesium bromide (6.07 mL of 3.0 M in Et₂O, 18.2 mmol) is added. After stirring several minutes, the ice bath is removed and the reaction is allowed to stir overnight at room temperature, after which 2 M HCl (30 mL) is added, followed by extraction with EtOAc (3×30 mL). The combined organic layers are dried (MgSO₄), filtered and concentrated under reduced pressure. The residue is subjected to flash chromatography (silica gel, 98.5:1.5 CH₂Cl₂/MeOH). The product is found to be unstable to silica therefore an impure mixture of the sub-title compound (211 mg, ˜20%) is immediately carried to the next step.

E. Preparation of:

### N-{3-[1-(3-Amino-benzo[d]isoxazol-5-yl)-1-ethyl-propyl]-1H-indol-7-yl}-methanesulfonamide

An impure mixture containing 3-(3-amino-benzo[d]isoxazol-5-yl)-pentan-3-ol (200 mg, ˜1.0 mmol) is combined with N-(1H-indol-7-yl)-methanesulfonamide (420 mg, 2.00 mmol) and dissolved in CH₂Cl₂ (10 mL), followed by addition of TFA (0.39 mL, 5.00 mmol). After stirring at room temperature for 3 d, saturated aqueous NaHCO₃ (30 mL) and CH₂Cl₂ are added. The layers are separated, and the aqueous layer is extracted once with CH₂Cl₂ (30 mL). The combined organic layers are dried (MgSO₄), filtered and concentrated under reduced pressure. The residue is subjected to flash chromatography (silica gel, 99:1 to 98:2 CH₂Cl₂/MeOH) to afford the title compound (61 mg, 15%).

R_{f} 0.28 (95:5 CH₂Cl₂/MeOH).

mp 155-161° C.

¹H NMR (300 MHz, CD₃OD) δ 0.65 (t, J=7.3 Hz, 6H), 2.16-2.34 (m, 4H), 2.96 (s, 3H), 6.64-6.67 (m, 2H), 6.92-6.95 (m, 1H), 7.16 (d, J=8.8 Hz, 1H), 7.34-7.39 (m, 2H), 7.84 (d, J=1.3 Hz, 1H).

ESI MS m/z 413 [C₂₁H₂₄N₄O₃S+H]⁺.

HPLC (Method A) 96.1% (AUC), t_{R}=18.4 min.

### EXAMPLE 153

### N-{3-[1-(3H-Benzotriazol-5-yl)-1-ethyl-propyl]-1H-indol-7-yl}-methanesulfonamide

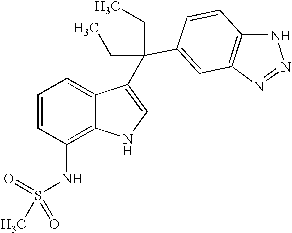 A. Preparation of:

### 3-(1H-Benzotriazol-5-yl)-pentan-3-ol

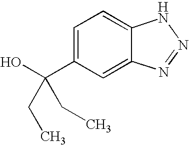

To a pre-dried round-bottomed flask containing 4-methyl-benzotriazole ester (1.20 g, 6.78 mmol) under a nitrogen atmosphere is added anhydrous THF (70 mL). Ethyl magnesium bromide (3 M in Et₂O, 11.3 mL, 33.9 mmol) is then slowly added to the solution at room temperature. The reaction mixture is then heated to reflux and allowed to stir for 1 h. Upon completion, the reaction is cooled to room temperature then quenched with saturated aqueous NH₄Cl (15 mL). The reaction contents are then diluted with H₂O (100 mL) and extracted with Et₂O (3×75 mL). The combined organic layers are dried (MgSO₄), filtered and concentrated to give the sub-title compound (1.30 g, 93%) as a semicrystalline brown residue, which is used without further purification.

R_{f} 0.28 (1:1 Hex/EtOAc).

¹H NMR (300 MHz, acetone-d₆) δ 0.70 (t, J=7.0 Hz, 6H), 1.85-2.10 (m, 4H), 3.91 (br s, 1H), 7.51 (d, J=7.2 Hz, 1H), 7.83 (d, J=7.4 Hz, 1H), 8.05 (br s, 1H).

APCI MS m/z 206 [C₁₁H₁₅N₃O+H]⁺.

B. Preparation of:

### N-{3-[1-(3H-Benzotriazol-5-yl)-1-ethyl-propyl]-1H-indol-7-yl}-methanesulfonamide

To a room temperature solution of N-(1H-indol-7-yl)-methanesulfonamide (325 mg, 1.54 mmol) in CH₂Cl₂ (15 mL) is added 3-(1H-benzotriazol-5-yl)-pentan-3-61 (100 mg, 0.49 mmol) and TFA (0.26 mL, 3.57 mmol). The reaction is then heated to reflux and allowed to stir for 6 h. A second portion of 3-(1H-benzotriazol-5-yl)-pentan-3-ol (100 mg, 0.49 mmol) and TFA (0.26 mL, 3.57 mmol) is then added. Upon addition, the reaction is allowed to slowly cool to room temperature and stirred overnight. The reaction is quenched with saturated aqueous NaHCO₃ (˜50 mL), then extracted with EtOAc (3×25 mL). The combined organic layers are dried (MgSO₄), filtered and concentrated to dryness. The resultant residue is subjected to flash column chromatography (silica gel, 95:5:0.5 CH₂Cl₂/MeOH/NH₄OH) to give the title compound (235 mg, 60%) as a yellow powder.

R_{f} 0.55 (90:10:0.5 CH₂Cl₂/MeOH/NH₄OH).

mp 165-172° C.

¹H NMR (300 MHz, CD₃OD) δ 0.66 (t, J=7.3 Hz, 6H), 2.14-2.37 (m, 4H), 3.31 (s, 3H), 6.63 (br s, 2H), 6.93 (br s, 1H), 7.29 (d, J=9.5 Hz, 1H), 7.38 (s, 1H), 7.61 (d, J=8.9 Hz, 1H), 7.89 (s, 1H).

ESI MS m/z 398 [C₂₀H₂₃N₅O₂S+H]⁺.

HPLC (Method F) 95.6% (area percent), t_{R}=17.8 min.

### EXAMPLE 154

### N-{3-[1-Ethyl-1-(2-methyl-3H-benzoimidazol-5-yl)-propyl]-1H-indol-7-yl}-methanesulfonamide

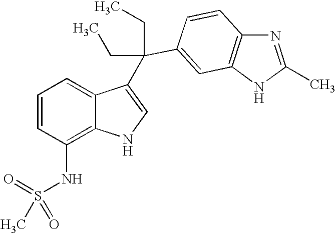 A. Preparation of:

### 2-Methyl-3H-benzoimidazole-5-carboxylic acid methyl ester

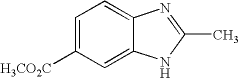

A suspension of 2-methyl-3H-benzoimidazole-5-carboxylic acid (2.00 g, 11.36 mmol) in MeOH (30 mL) is treated with H₂SO₄ (1 mL), then heated to reflux and allowed to stir for 3 d. Upon completion, the reaction is cooled to room temperature, quenched with saturated aqueous NaHCO₃ (˜75 mL), then extracted with EtOAc (3×75 mL). The combined organic layers are dried (MgSO₄), filtered and concentrated to give the sub-title compound (1.80 g, 84%) as a white solid, which is used without further purification.

R_{f} 0.47 (95:5:0.5 CH₂Cl₂/MeOH/NH₄OH).

mp 163-165° C.

¹H NMR (300 MHz, acetone-d₆) δ 2.55 (s, 3H), 3.88 (s, 3H), 7.52 (d, J=7.5 Hz, 1H), 7.84 (d, J=7.4 Hz, 1H), 8.19 (s, 1H).

APCI MS (negative mode) m/z 189 [C₁₀H₁₀N₂O₂−H]⁻.

B. Preparation of:

### 3-(2-Methyl-3H-benzoimidazol-5-yl)-pentan-3-ol

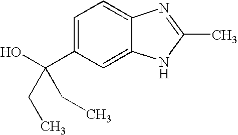

To a pre-dried round-bottomed flask containing 2-methyl-3H-benzoimidazole-5-carboxylic acid methyl ester (1.59 g, 8.41 mmol) under a nitrogen atmosphere is added anhydrous THF (50 mL). Ethyl magnesium bromide (3 M in Et₂O, 16.8 mL, 50.5 mmol) is then slowly added to the solution at room temperature. The reaction mixture is heated to reflux and allowed to stir for 90 min. The reaction is cooled to room temperature then quenched with saturated aqueous NaHCO₃ (15 mL). The reaction contents are diluted with H₂O (100 mL), then extracted with Et₂O (3×75 mL). The combined organic layers are washed with brine (75 mL), dried (MgSO₄), filtered and concentrated to give the sub-title compound (1.45 g, 79%) as a white solid, which is used without further purification.

R_{f} 0.35 (95:5:0.5 CH₂Cl₂/MeOH/NH₄OH).

mp 155-160° C.

¹H NMR (300 MHz, CDCl₃) δ 0.76 (t, J=7.4 Hz, 6H), 1.69 (br s, 1H), 1.81-1.99 (m, 4H), 2.62 (s, 3H), 7.21 (d, J=8.6 Hz, 1H), 7.49-7.60 (m, 2H), 9.10 (br s, 1H).

APCI MS (negative mode) m/z 217 [C₁₃H₁₈N₂O−H]⁻.

C. Preparation of:

### N-{3-[1-Ethyl-1-(2-methyl-3H-benzoimidazol-5-yl)-propyl]-1H-indol-7-yl}-methanesulfonamide

To a room temperature solution of N-(1H-indol-7-yl)-methanesulfonamide (872 mg, 4.15 mmol) in CH₂Cl₂ (20 mL) is added 3-(2-methyl-3H-benzoimidazol-5-yl)-pentan-3-ol (200 mg, 4.15 mmol) in 1 mL of CH₂Cl₂ and TFA (0.61 mL, 8.29 mmol). The reaction is then allowed to stir at room temperature overnight. Two additional portions of 3-(2-methyl-3H-benzoimidazol-5-yl)-pentan-3-ol (200 mg, 4.15 mmol) in CH₂Cl₂ (1 mL) are added in 24 h periods. After 72 h the reaction is quenched with saturated aqueous NaHCO₃ (˜75 mL) then extracted with EtOAc (3×75 mL). The combined organic layers are dried (MgSO₄), filtered and concentrated to dryness. The resultant crude product is subjected to flash column chromatography (silica gel, 95:5:0.5 CH₂Cl₂/MeOH/NH₄OH) to give the title compound (860 mg, 76%) as a pale white solid.

R_{f} 0.48 (90:10:0.5 CH₂Cl₂/MeOH/NH₄OH).

mp 188-192° C.

¹H NMR (300 MHz, CD₃OD) δ 0.64 (t, J=7.2 Hz, 6H), 2.14-2.28 (m, 4H), 2.52 (s, 3H), 2.94 (s, 3H), 6.60-6.63 (m, 2H), 6.91 (d, J=6.9 Hz, 1H), 7.08 (d, J=8.5 Hz, 1H), 7.25-7.31 (m, 2H), 7.46 (s, 1H).

APCI MS m/z 411 [C₂₂H₂₆N₄O₂S+H]⁺.

HPLC (Method A) 96.7% (area percent), t_{R}=15.8 min.

### EXAMPLE 155

### N-{3-[1-Ethyl-1-(2-methyl-benzofuran-5-yl)-propyl]-1H-indol-7-yl}-methanesulfonamide

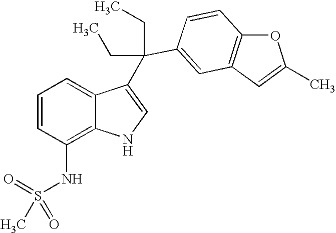 A. Preparation of:

### 3-(2-Methyl-benzofuran-5-yl)-pentan-3-ol

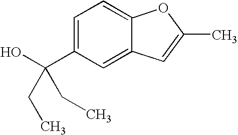

To a pre-dried round-bottomed flask containing 2-methyl-benzofuran-5-carboxylic acid methyl ester (2.26 g, 11.89 mmol) [Heterocycles 1994, 39, 371] under a nitrogen atmosphere is added anhydrous THF (100 mL). Ethyl magnesium bromide (3 M in Et₂O, 20.0 mL, 50.5 mmol) is then slowly added and the reaction is allowed to stir at room temperature for 2 h. Upon completion, the reaction is quenched with saturated aqueous NH₄Cl (100 mL) and the resultant mixture is extracted with Et₂O (3×100 mL). The combined organic layers are washed with brine (100 mL), dried (MgSO₄), filtered and concentrated to give the sub-title compound (2.54 g, 98%) as a yellow oil, which is used without further purification.

R_{f} 0.58 (1:1 Hex/EtOAc).

¹H NMR (300 MHz, acetone-d₆) δ 0.65 (t, J=8.5 Hz, 6H), 1.75-1.98 (m, 4H), 2.39 (s, 3H), 6.43 (s, 1H), 7.22-7.35 (m, 2H), 7.59 (s, 1H).

B. Preparation of:

### N-{3-[1-Ethyl-1-(2-methyl-benzofuran-5-yl)-propyl]-1H-indol-7-yl}-methanesulfonamide

To a room temperature solution of N-(1H-indol-7-yl)-methanesulfonamide (868 mg, 4.13 mmol) in CH₂Cl₂ (20 mL) is added TFA (0.61 ml, 8.25 mmol) and 3-(2-methyl-benzofuran-5-yl)-pentan-3-ol (300 mg, 1.38 mmol). The reaction is allowed to stir at room temperature for 5 h then an additional amount of 3-(2-methyl-benzofuran-5-yl)-pentan-3-ol (300 mg, 1.38 mmol) is added. The reaction is stirred for another 3 h at room temperature. Upon completion, the reaction is then quenched with saturated aqueous NaHCO₃ (100 mL) and extracted with EtOAc (3×100 mL). The combined organic layers are washed with brine (100 mL), dried (MgSO₄), filtered and concentrated to dryness. The crude product is subjected to flash column chromatography (silica gel, 7:3 Hex/EtOAc) to give the title compound (850 mg, 77%) as a white solid.

R_{f} 0.56 (1:1 Hex/EtOAc).

mp 100-105° C.

¹H NMR (300 MHz, CD₃OD) δ 0.63 (t, J=7.4 Hz, 6H), 2.14-2.28 (m, 4H), 2.39 (s, 3H), 3.30 (s, 3H), 6.32 (s, 1H), 6.61-6.64 (m, 2H), 6.92 (d, J=6.3 Hz, 1H), 7.06 (d, J=8.7 Hz, 1H), 7.16 (d, J=8.6 Hz, 1H), 7.31 (s, 1H), 7.42 (s, 1H).

ESI MS (negative mode) m/z 409 [C₂₃H₂₆N₂O₃S−H]⁻.

HPLC (Method C) 97.3% (area percent), t_{R}=18.9 min.

### EXAMPLE 156

### N-{3-[1-(1-Acetyl-1H-indol-5-yl)-1-ethyl-propyl]-1H-indol-7-yl}-methanesulfonamide

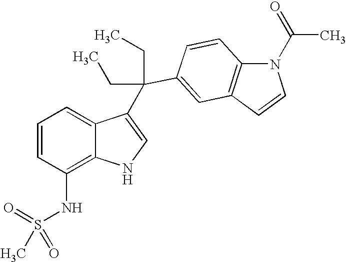 A. Preparation of:

### 3-(2-Methyl-1H-indol-5-yl)-pentan-3-ol

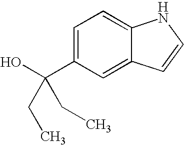

To a pre-dried round-bottomed flask containing 2-methyl-1H-indole-5-carboxylic acid methyl ester (2.00 g, 11.40 mmol) under a nitrogen atmosphere is added anhydrous THF (80 mL). Ethyl magnesium bromide (3 M solution in Et₂O, 23.0 mL, 68.5 mmol) is then slowly added to the solution and the reaction mixture is allowed to stir at room temperature for 3 h. Upon completion, the reaction is quenched with saturated aqueous NH₄Cl (100 mL) and extracted with EtOAc (3×100 mL). The combined organic layers are then washed with brine (100 mL), dried (MgSO₄), filtered and concentrated to give the sub-title compound (2.20 g, 95%) as a light yellow oil, which is used without further purification.

R_{f} 0.77 (1:1 Hex/EtOAc).

¹H NMR (300 MHz, acetone-d₆) δ 0.70 (t, J=8.5 Hz, 6H), 1.70-1.92 (m, 5H), 6.42 (s, 1H), 7.17 (d, J=7.5 Hz, 1H), 7.25 (s, 1H), 7.33 (d, J=7.5 Hz, 1H), 7.68 (s, 1H), 10.05 (br s, 1H).

B. Preparation of:

### Acetic acid 1-(1-acetyl-2-methyl-1H-indol-5-yl)-1-ethyl-propyl ester

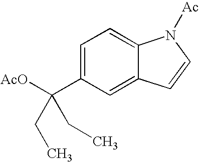

Crude 3-(2-methyl-1H-indol-5-yl)-pentan-3-ol (1.18 g, 5.81 mmol) is dissolved in CH₂Cl₂ (15 mL) then treated with acetic anhydride (1.26 mL, 13.40 mmol), DMAP (71 mg, 0.58 mmol) and TEA (0.89 mL, 6.39 mmol). The resultant reaction mixture is allowed to stir at room temperature for 7 d. Upon completion, the reaction is diluted with H₂O (100 mL) and extracted with EtOAc (3×50 mL). The combined organic layers are washed with brine (3×50 mL), dried (MgSO₄), filtered and concentrated to dryness. The crude residue is subjected to flash column chromatography (silica gel, 4:1 Hex/EtOAc) to give sub-title compound (600 mg, 36%) as a yellow oil.

R_{f} 0.34 (4:1 Hex/EtOAc).

¹H NMR (300 MHz, DMSO-d₆) δ 0.61 (t, J=8.5 Hz, 6H), 2.05-2.19 (m, 5H), 2.25-2.40 (m, 2H), 2.66 (s, 3H), 6.72 (s, 1H), 7.30 (d, J=7.5 Hz, 1H), 7.59 (s, 1H), 7.88 (s, 1H), 8.27 (d, J=7.7 Hz, 1H).

C. Preparation of:

### N-{3-[1-(1-Acetyl-1H-indol-5-yl)-1-ethyl-propyl]-1H-indol-7-yl}-methanesulfonamide

A room temperature solution of acetic acid 1-(1-acetyl-2-methyl-1H-indol-5-yl)-1-ethyl-propyl ester (530 mg, 1.85 mmol) in CH₂Cl₂ (25 mL) is treated with N-(1H-indol-7-yl)-methanesulfonamide (582 mg, 2.77 mmol) and TFA (0.41 mL, 5.54 mmol). The resultant reaction mixture is allowed to stir at room temperature for 48 h. Upon completion, the reaction is quenched with saturated aqueous NaHCO₃ (100 mL) then extracted with EtOAc (2×100 mL). The combined organic layers are then dried (MgSO₄), filtered and concentrated to dryness. The crude product is subjected to flash column chromatography (silica gel 1:1 Hex/EtOAc) to give the title compound (600 mg, 74%) as a white solid.

R_{f} 0.25 (1:1 Hex/EtOAc).

mp 125-130° C.

¹H NMR (300 MHz, CD₃OD) δ 0.64 (t, J=7.4 Hz, 6H), 2.14-2.30 (m, 4H), 2.61 (s, 3H), 2.95 (s, 3H), 6.59-6.63 (m, 3H), 6.91 (d, J=6.5 Hz, 1H), 7.20 (d, J=8.8 Hz, 1H), 7.33 (s, 1H), 7.54 (s, 1H), 7.59 (s, 1H), 8.14 (d, J=8.7 Hz, 1H).

ESI MS (negative mode) m/z 436 [C₂₄H₂₇N₃O₃S−H]⁻.

HPLC (Method C) 97.6% (area percent), t_{R}=17.3 min.

### EXAMPLE 157

### N-{3-[1-Ethyl-1-(1H-indol-5-yl)-propyl]-1H-indol-7-yl}-methanesulfonamide

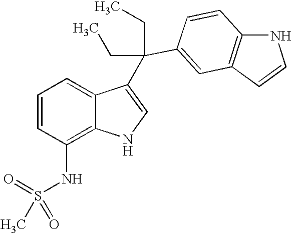

A room temperature solution of N-{3-[1-(1-acetyl-1H-indol-5-yl)-1-ethyl-propyl]-1H-indol-7-yl}-methanesulfonamide (470 mg, 1.08 mmol;) in a 1:1:1 mixture of MeOH/THF/H₂O (10 mL) is treated with LiOH (52 mg, 2.15 mmol). The resultant reaction mixture is then heated to reflux and allowed to stir overnight. Upon completion, the reaction is cooled to room temperature, diluted with H₂O (75 mL) and extracted with EtOAc (3×75 mL). The combined organic layers are then dried (MgSO₄), filtered and concentrated to dryness. The crude product is subjected to flash column chromatography (silica gel, 4:1 to 1:1 Hex/EtOAc) to give the title compound (309 mg, 72%) as a white solid.

R_{f} 0.44 (1:1 Hex/EtOAc).

mp 115-120° C.

¹H NMR (300 MHz, CD₃OD) δ 0.64 (t, J=7.4 Hz, 6H), 2.14-2.32 (m, 4H), 2.95 (s, 3H), 6.36 (s, 1H), 6.60 (d, J=6.6 Hz, 1H), 6.70 (d, J=8.1 Hz, 1H), 6.89-6.95 (m, 2H), 7.14-7.17 (m, 2H), 7.29 (s, 1H), 7.57 (s, 1H).

ESI MS (negative mode) m/z 394 [C₂₂H₂₅N₃O₂S−H]⁻.

HPLC (Method C) 97.6% (area percent), t_{R}=17.1 min.

### EXAMPLE 158

### N-{3-[1-(1-Acetyl-1H-indol-6-yl)-1-ethyl-propyl]-1H-indol-7-yl}-methanesulfonamide

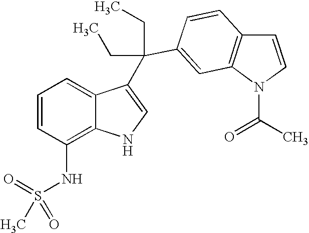 A. Preparation of:

### 3-(1H-Indol-6-yl)-pentan-3-ol

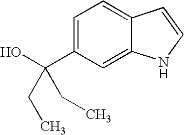

To a pre-dried round-bottomed flask containing 1H-indole-6-carboxylic acid methyl ester (1.00 g, 5.71 mmol) under a nitrogen atmosphere is added anhydrous THF (40 mL). Ethyl magnesium bromide (3 M solution in Et₂O, 11.4 mL, 34.25 mmol) is then slowly added to the solution and the resultant reaction mixture is allowed to stir at room temperature overnight. The reaction is then quenched with saturated aqueous NH₄Cl (150 mL) and extracted with EtOAc (2×150 mL). The combined organic layers are then dried (MgSO₄), filtered and concentrated to give sub-title compound (1.08 g, 93%) as a light yellow oil, which is used without further purification;

R_{f} 0.20 (4:1 Hex/EtOAc)

¹H NMR (300 MHz, CD₃OD) δ 0.72 (t, J=8.0 Hz, 6H), 1.76-1.98 (m, 4H), 6.39 (s, 1H), 7.05 (d, J=7.5 Hz, 1H), 7.18 (s, 1H), 7.47 (br s, 2H).

B. Preparation of:

### Acetic acid 1-(1-acetyl-1H-indol-6-yl)-1-ethyl-propyl ester

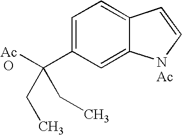

Crude 3-(1H-indol-6-yl)-pentan-3-ol (1.04 g, 5.12 mmol) is dissolved in CH₂Cl₂ (10 mL) and treated with acetic anhydride (0.60 mL, 6.15 mmol), DMAP (62 mg, 0.51 mmol) and TEA (0.79 mL, 5.63 mmol). The resultant reaction mixture is then allowed to stir at room temperature for 72 h. Upon completion, the reaction is diluted with H₂O (100 mL) and extracted with EtOAc (3×100 mL). The combined organic layers are then washed with brine (2×100 mL), dried (MgSO₄), filtered and concentrated to dryness. The crude residue is subjected to flash column chromatography (silica gel, 9:1 to 4:1 Hex/EtOAc) to give the sub-title compound (200 mg, 14%) as a yellow oil.

R_{f} 0.54 (4:1 Hex/EtOAc).

¹H NMR (300 MHz, CD₃OD) δ 0.71 (t, J=8.3 Hz, 6H), 2.11 (s, 3H), 2.12-2.27 (m, 2H), 2.32-2.49 (m, 2H), 2.57 (s, 3H), 6.62 (s, 1H), 7.25 (d, J=7.4 Hz, 1H), 7.51 (d, J=7.6 Hz, 1H), 7.58 (s, 1H), 8.48 (s, 1H).

C. Preparation of:

### N-{3-[1-(1-Acetyl-1H-indol-6-yl)-1-ethyl-propyl]-1H-indol-7-yl}methanesulfonamide

A room temperature solution of acetic acid 1-(1-acetyl-1H-indol-6-yl)-1-ethyl-propyl ester (200 mg, 0.70 mmol) in CH₂Cl₂ (10 mL) is treated with N-(1H-indol-7-yl)-methanesulfonamide (218 mg, 1.04 mmol) and TFA (0.16 mL, 2.09 mmol). The resultant reaction is then allowed to stir at room temperature overnight. Upon completion, the reaction is quenched with saturated aqueous NaHCO₃ (100 mL) then extracted with EtOAc (3×50 mL). The combined organic layers are then washed with brine (50 mL), dried (MgSO₄), filtered and concentrated to dryness. The crude product is subjected to flash-column chromatography (silica gel, 7:3 Hex/EtOAc) to give the title compound (244 mg, 80%) as a colorless oil. The title compound is then dissolved in CH₂Cl₂ and concentrated to dryness to yield a white solid.

R_{f} 0.42 (1:1 Hex/EtOAc).

mp 216-218° C.

¹H NMR (300 MHz, DMSO-d₆) δ 0.57 (t, J=7.2 Hz, 6H), 2.09-2.24 (m, 4H), 2.59 (s, 3H), 2.98 (s, 3H), 6.52-6.65 (m, 3H), 6.89 (d, J=7.2 Hz, 1H), 7.10 (d, J=8.3 Hz, 1H), 7.38 (s, 2H), 7.75 (s, 1H), 8.43 (s, 1H), 9.24 (br s, 1H), 10.63 (br s, 1H).

APCI MS (negative mode) m/z 436 [C₂₄H₂₇N₃O₃S−H]⁻.

HPLC (Method C) 98.8% (area percent), t_{R}=17.3 min.

### EXAMPLE 159

### N-{3-[1-Ethyl-1-(1H-indol-6-yl)-propyl]-1H-indol-7-yl}-methanesulfonamide

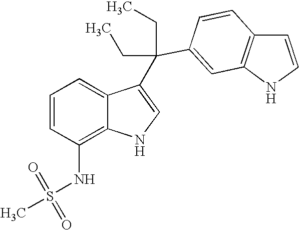

A room temperature solution of N-{3-[1-(1-acetyl-1H-indol-6-yl)-1-ethyl-propyl]-1H-indol-7-yl}-methanesulfonamide (544 mg, 1.27 mmol) in a 1:1:1 mixture of MeOH/THF/H₂O (15 mL) is treated with LiOH (61 mg, 2.54 mmol). The resultant reaction mixture is then heated to reflux and allowed to stir for 6 h. Upon completion, the reaction is cooled to room temperature, diluted with H₂O (100 mL) and extracted with EtOAc (2×75 mL). The combined organic layers are then dried (MgSO₄), filtered and concentrated to dryness. The crude product is subjected to flash column chromatography (silica gel, 7:3 Hex/EtOAc) to give the title compound (309 mg, 62%) as a colorless oil. The compound is then dissolved in CH₂Cl₂ and concentrated to give a white solid.

R_{f} 0.39 (1:1 Hex/EtOAc).

mp 110-115° C.

¹H NMR (300 MHz, CD₃OD) δ 0.64 (t, J=7.4 Hz, 6H), 2.14-2.28 (m, 4H), 2.93 (s, 3H), 6.32 (br s, 1H), 6.60 (d; J=7.6 Hz, 1H), 6.71 (d, J=7.2 Hz, 1H), 6.90 (d, J=7.9 Hz, 2H), 7.11 (br s, 1H), 7.29-7.39 (m, 3H).

APCI MS (negative mode) m/z 394 [C₂₂H₂₅N₃O₂S−H]⁻.

HPLC (Method C) >99% (area percent), t_{R}=17.0 min.

### EXAMPLE 160

### N-{3-[1-Ethyl-1-(2-methyl-benzofuran-4-yl)-propyl]-1H-indol-7-yl}-methanesulfonamide

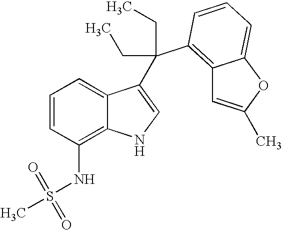 A. Preparation of:

### 3-Benzofuran-4-yl-pentan-3-ol

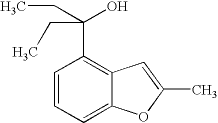

To a pre-dried round-bottomed flask containing 2-methyl-benzofuran-4-carboxylic acid methyl ester (525 mg, 2.76 mmol) [Heterocycles 1994, 39, 374] under a nitrogen atmosphere is added anhydrous THF (20 mL). Ethyl magnesium bromide (3 M in Et₂O, 5.5 mL, 16.58 mmol) is slowly added to the solution and the reaction mixture is then stirred at room temperature for 4 h. Upon completion, the reaction is quenched with saturated aqueous NH₄Cl (100 mL) and extracted with EtOAc (2×100 mL). The combined organic layers are dried (MgSO₄), filtered and concentrated. The resultant residue is subjected to column chromatography (silica gel, 9:1 Hex/EtOAc) to give the sub-title compound (444 mg, 66%) as a light yellow oil.

R_{f} 0.59 (4:1 Hex/EtOAc).

¹H NMR (300 MHz, CDCl₃) δ 0.73 (t, J=7.0 Hz, 6H), 1.82-2.10 (m, 5H), 2.41 (s, 3H), 6.64 (s, 1H), 7.15 (br s, 2H), 7.29 (br s, 1H).

B. Preparation of:

### N-{3-[1-Ethyl-1-(2-methyl-benzofuran-4-yl)-propyl]-1H-indol-7-yl}-methanesulfonamide

A room temperature solution of 3-benzofuran-4-yl-pentan-3-ol (415 mg, 1.90 mmol) in CH₂Cl₂ (15 mL) is treated with N-(1H-indol-7-yl)-methanesulfonamide (600 mg, 2.86 mmol) and TFA (0.43 mL, 5.71 mmol). The reaction mixture is then stirred at room temperature overnight. Upon completion, the reaction is quenched with saturated aqueous NH₄Cl (100 mL) and extracted with EtOAc (2×100 mL). The combined organic layers are washed with brine (100 mL), dried (MgSO₄), filtered and concentrated. The resultant product is subjected to flash column chromatography (silica gel, 98:2 CH₂Cl₂/MeOH) to give impure title compound (600 mg, 77%) as a white solid. The impure title compound is subjected to preparative HPLC (Waters Symmetry C18 column, 7 μm, 77×230 mm, 80:20 CH₃CN/H₂O, 0.1% TFA, 250 mL/min, δ=254 nm) to afford the title compound (193 mg, 25%) as a white solid.

R_{f} 0.59 (1:1 Hex/EtOAc).

mp 85-90° C.

¹H NMR (300 MHz, CD₃OD) δ 0.62 (t, J=7.4 Hz, 6H), 2.12 (s, 3H), 2.25-2.35 (m, 4H), 2.93 (s, 3H), 5.85 (s, 1H), 6.53-6.61 (m, 2H), 6.88 (d, J=7.0 Hz, 1H), 7.20 (br s, 2H), 7.35 (s, 1H), 7.38-7.41 (m, 1H).

APCI MS (negative mode) m/z 409 [C₂₃H₂₆N₂O₃S−H]⁻.

HPLC (Method C) >99% (area percent), t_{R}=18.9 min.

### EXAMPLE 161

### N-{3-[1-(2-Chloro-benzothiazol-5-yl)-1-ethyl-propyl]-1H-indol-7-yl}-methanesulfonamide

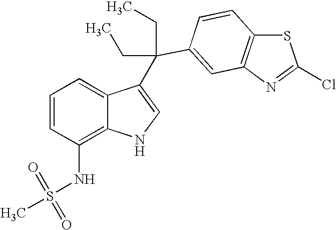 A. Preparation of:

### 3-(2-Chloro-benzothiazol-5-yl)-pentan-3-ol

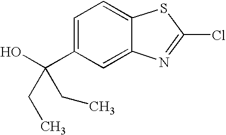

3-(2-Amino-benzothiazol-5-yl)-pentan-3-ol (434 mg, 1.84 mmol;) is added to a heated (60° C.) suspension of copper(II)chloride (297 mg, 2.21 mmol), t-butylnitrite (0.33 mL, 2.75 mmol) and CH₃CN (10 mL) in portions over a 5 min period. The reaction mixture is cooled to room temperature after stirring at 60° C. for 1 h. The reaction contents are then poured into 2 M HCl (75 mL) and extracted with Et₂O (2×75 mL). The combined organic layers are then washed with brine (75 mL), dried (MgSO₄), filtered and concentrated to give the sub-title compound (423 mg, 90%) as an orange solid, which is used without further purification.

R_{f} 0.57 (1:1 Hex/EtOAc).

¹H NMR (300 MHz, CD₃OD) δ 0.72 (t, J=7.5 Hz, 6H), 1.78-1.95 (m, 4H), 7.51 (d, J=8.1 Hz, 1H), 7.86 (d, J=8.2 Hz, 1H), 7.99 (s, 1H).

B. Preparation of:

### N-{3-[1-(2-Chloro-benzothiazol-5-yl)-1-ethyl-propyl]-1H-indol-7-yl}-methanesulfonamide

Crude 3-(2-chloro-benzothiazol-5-yl)-pentan-3-ol (400 mg, 1.57 mmol) is dissolved in CH₂Cl₂ (10 mL) then treated with N-(1H-indol-7-yl)-methanesulfonamide (495 mg, 2.36 mmol) and TFA acid (0.35 mL, 4.71 mmol). The resultant reaction mixture is allowed to stir at room temperature overnight. Upon completion, the reaction is quenched with saturated aqueous NaHCO₃ (75 mL) then extracted with EtOAc (3×75 mL). The combined organic layers are washed with brine (75 mL), dried (MgSO₄), filtered and concentrated to dryness. The resultant residue is subjected to flash column chromatography (7:3 Hex/EtOAc) to give impure title compound (584 mg, 84%) as a white solid. The impure title compound is subjected to preparative HPLC (Waters Symmetry C18 column, 7 μm, 77×230 mm, 70:30 CH₃CN/H₂O, 0.1% TFA, 250 mL/min, δ=254 nm) to afford the title compound (184 mg, 26%) as a white solid.

R_{f} 0.43 (1:1 Hex/EtOAc).

mp 217-220° C.

¹H NMR (300 MHz, DMSO-d₆) δ 0.58 (t, J=7.1 Hz, 6H), 2.11-2.28 (m, 4H), 2.99 (s, 3H), 6.53-6.65 (m, 2H), 6.92 (d, J=7.2 Hz, 1H), 7.32 (d, J=8.7 Hz, 1H), 7.41 (s, 1H), 7.87-7.90 (m, 2H), 9.23 (s, 1H), 10.71 (s, 1H).

APCI MS (negative mode) m/z 446 [C₂₁H₂₂ClN₃O₂S₂−H]⁻.

HPLC (Method C) >99% (area percent), t_{R}=19.0 min.

### EXAMPLE 162

### N-{3-[1-(1,2-Dimethyl-1H-benzoimidazol-5-yl)-1-ethyl-propyl]-1H-indol-7-yl}-methanesulfonamide

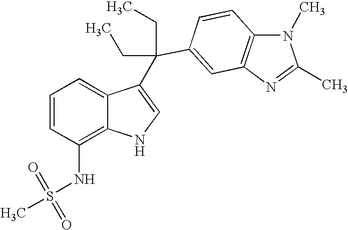 A. Preparation of:

### 1,2-Dimethyl-1H-benzoimidazole-5-carboxylic acid methyl ester

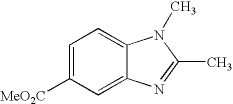

A room temperature solution of 1,2-dimethyl-1H-benzoimidazole-5-carboxylic acid (1.00 g, 5.26 mmol) in MeOH (20 mL) is treated with H₂SO₄ (0.6 mL). The reaction mixture is then heated to reflux and allowed to stir overnight. Upon completion, the reaction is cooled to room temperature then quenched with saturated aqueous NaHCO₃ (100 mL) and extracted with EtOAc (3×100 mL). The combined organic layers are dried (MgSO₄), filtered and concentrated to give the sub-title compound (810 mg, 75%) as a yellow oil.

R_{f} 0.69 (85:15 CH₂Cl₂/MeOH).

¹H NMR (300 MHz, CDCl₃) δ 2.61 (s, 3H), 3.72 (s, 3H), 3.92 (s, 3H), 7.27 (d, J=8.5 Hz, 1H), 7.98 (d, J=8.2 Hz, 1H), 8.35 (s, 1H).

APCI MS m/z 205 [C₁₁H₁₂N₂O₂+H]⁺.

B. Preparation of:

### 3-(1,2-Dimethyl-1H-benzoimidazol-5-yl)-pentan-3-ol

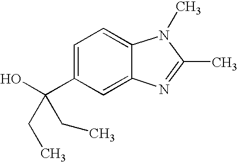

To a pre-dried round-bottomed flask containing 1,2-dimethyl-1H-benzoimidazole-5-carboxylic acid methyl ester (600 mg, 2.94 mmol) under a nitrogen atmosphere is added anhydrous THF (30 mL). Ethyl magnesium bromide (3 M in Et₂O, 5.88 mL, 17.64 mmol) is slowly added to the solution then the reaction is allowed to stir at room temperature overnight. Upon completion, the reaction is quenched with saturated aqueous NH₄Cl (100 mL) and then extracted with Et₂O (2×100 mL) and EtOAc (100 mL). The combined organic layers are washed with brine (100 mL), dried (MgSO₄), filtered and concentrated to give the sub-title compound (560 mg, 82%) as a yellow solid, which is used without futher purification.

R_{f} 0.38 (85:15 CH₂Cl₂/MeOH)

¹H NMR (300 MHz, CD₃OD) δ 0.73 (t, J=7.5 Hz, 6H), 1.74-1.93 (m, 4H), 2.58 (s, 3H), 3.76 (s, 3H), 7.22-7.38 (m, 2H), 7.61 (s, 1H).

C. Preparation of:

### N-{3-[1-(1,2-Dimethyl-1H-benzoimidazol-5-yl)-1-ethyl-propyl]-1H-indol-7-yl}-methanesulfonamide

Crude 3-(1,2-dimethyl-1H-benzoimidazol-5-yl)-pentan-3-ol (300 mg, 1.29 mmol) is dissolved in CH₂Cl₂ (10 mL) then treated with N-(1H-indol-7-yl)-methanesulfonamide (408 mg, 1.94 mmol) and TFA (0.58 mL, 7.74 mmol). The reaction is allowed to stir at room temperature for 4 d. Upon completion, the reaction is quenched with saturated aqueous NaHCO₃ (100 mL) then extracted with CH₂Cl₂ (3×100 mL). The combined organic layers are dried (MgSO₄), filtered and concentrated to dryness. The crude product is subjected to flash column chromatography (95:5 Hex/EtOAc) to give impure title compound (310 mg, 57%) as a pink solid. Impure title compound is subjected to a second flash column chromatography (7:3 acetone/Hex) to give analytically pure title compound (63 mg, 12%) as a white solid.

R_{f} 0.33 (95:5 CH₂Cl₂/MeOH).

mp 275-278° C.

¹H NMR (300 MHz, CD₃OD) δ 0.64 (t, J=7.3 Hz, 6H), 2.15-2.33 (m, 4H), 2.55 (s, 3H), 2.94 (s, 3H), 3.71 (s, 3H), 6.55-6.62 (m, 2H), 6.90 (d, J=6.8 Hz, 1H), 7.12-7.22 (m, 2H), 7.33 (s, 1H), 7.56 (s, 1H).

APCI MS (negative mode) m/z 423 [C₂₃H₂₈N₄O₂S−H]⁻.

HPLC (Method A) >99% (area percent), t_{R}=15.9 min.

### EXAMPLE 163A AND 163B

### N-{3-[1-Ethyl-1-(2-methyl-benzofuran-4-yl)-propyl]-1H-indol-7-yl}-methanesulfonamide (163A) & N-{3-[1-Ethyl-1-(2-methyl-benzofuran-6-yl)-propyl]-1H-indol-7-yl}-methanesulfonamide (163B)

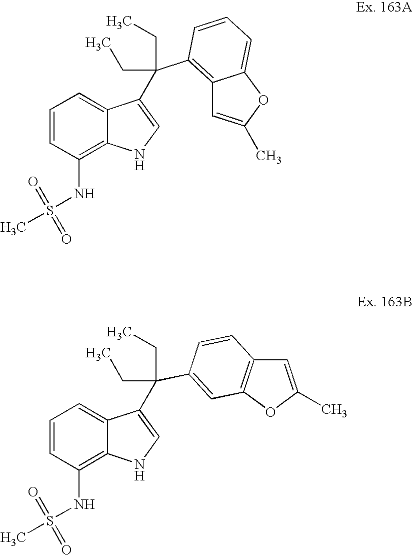 A. Preparation of:

### 3-Benzofuran-4-yl-pentan-3-ol (a) and 3-Benzofuran-6-yl-pentan-3-ol (b)

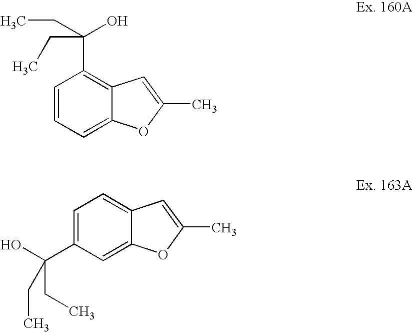

To a pre-dried round-bottomed flask containing a 4:1 mixture of 2-methyl-benzofuran-4-carboxylic acid methyl ester and 2-methyl-benzofuran-6-carboxylic acid methyl ester (2.00 g, 10.52 mmol) [Heterocycles 1994, 39, 371] under a nitrogen atmosphere is added anhydrous THF (50 mL). Ethyl magnesium bromide (3 M in Et₂O, 21 mL, 63.16 mmol) is slowly added to the solution and the reaction mixture is then stirred at room temperature overnight. Upon completion, the reaction is quenched with saturated aqueous NH₄Cl (100 mL) and extracted with Et₂O (2×100 mL). The combined organic layers are washed with brine (100 mL), dried (MgSO₄), filtered and concentrated to dryness. The resultant residue is subjected to column chromatography (silica gel, 9:1 Hex/EtOAc) to give the sub-title compound (4:1 mixture of 3-Benzofuran-4-yl-pentan-3-ol: 3-Benzofuran-4-yl-pentan-3-ol & 3-Benzofuran-6-yl-pentan-3-ol, 1.96 g, 85%) as a light yellow oil.

R_{f} (mixture) 0.59 (4:1 Hex/EtOAc).

¹H NMR (major regioisomer (Ex. 160 A) subtracted from mixture)-(300 MHz, CDCl₃) δ 0.73 (t, J=7.0 Hz, 6H), 1.82-2.10 (m, 5H), 2.41 (s, 3H), 6.64 (s, 1H), 7.15 (br s, 2H), 7.29 (br s, 1H).

¹H NMR (minor regioisomer (Ex. 163A), subtracted from mixture) (300 MHz, CDCl₃) δ 0.73 (t, J=7.0 Hz, 6H), 1.82-2.10 (m, 5H), 2.41 (s, 3H), 6.32 (s, 1H), 7.15 (br s, 1H), 7.40 (d, J=8.0 Hz, 1H), 7.49 (s, 1H).

B. Preparation of:

### N-{3-[1-Ethyl-1-(2-methyl-benzofuran-4-yl)-propyl]-1H-indol-7-yl}-methanesulfonamide (i) & N-{3-[1-Ethyl-1-(2-methyl-benzofuran-6-yl)-propyl]-1H-indol-7-yl}-methanesulfonamide (ii)

A room temperature solution of 3-benzofuran-4-yl-pentan-3-ol and 3-benzofuran-6-yl-pentan-3-ol (4:1 mixture of regioisomers, 500 mg, 2.29 mmol) in CH₂Cl₂ (15 mL) is treated with N-(1H-indol-7-yl)-methanesulfonamide (722 mg, 3.44 mmol) and TFA (0.51 mL, 6.87 mmol). The reaction mixture is then stirred at room temperature overnight. Upon completion, the reaction is quenched with saturated aqueous NaHCO₃ (75 mL) and extracted with EtOAc (3×75 mL). The combined organic layers are washed with brine (75 mL), dried (MgSO₄), filtered and concentrated to dryness. The resultant product is subjected to flash column chromatography (silica gel, 1:1 Hex/EtOAc) to give impure title compound (4:1 mixture of N-{3-[1-Ethyl-1-(2-methyl-benzofuran-6-yl)-propyl]-1H-indol-7-yl}-methanesulfonamide, 708 mg, 75%) as a white solid.

R_{f} (mixture) 0.59 (1:1 Hex/EtOAc).

¹H NMR (major regioisomer (i) subtracted from mixture) (300 MHz, CD₃OD) δ 0.62 (t, J=7.4 Hz, 6H), 2.12 (s, 3H), 2.25-2.35 (m, 4H), 2.93 (s, 3H), 5.85 (s, 1H), 6.53-6.61 (m, 2H), 6.88 (d, J=7.0 Hz, 1H), 7.20 (br s, 2H), 7.35 (s, 1H), 7.38-7.41 (m, 1H).

¹HNMR (minor regioisomer (ii) subtracted from mixture) (300 MHz, CD₃OD) δ 0.62 (t, J=7.4 Hz, 6H), 2.25-2.35 (m, 4H), 2.38 (s, 3H), 2.92 (s, 3H), 6.30 (s, 1H), 6.89-7.11 (m, 3H), 7.21-7.40 (m, 3H), 7.49 (d, J=6.0 Hz, 1H).

APCI MS (mixture) m/z 411 [C₂₃H₂₆N₂O₃S+H]⁺.

Examples 164-199, as provided in Table II below are made following procedures essentially as described in the Examples above. That is, employing the procedures as described in the Schemes herein, and utilizing the appropriate indole and the appropriate carbinol, each of which may be obtained from commercial sources or prepared according to procedures as described in the Preparations herein, the title compounds of Examples 164-199 are prepared. In the Table, “Ex. No.” refers to the example number of the title compound prepared; “Ref. Ex. No.” refers to the Example herein which provides procedures for the synthesis of the title compound prepared in the Table; “Structure” refers to the molecular structure corresponding to the title compound prepared; and “MS Data”/“HPLC” refers to the Mass Spectroscopy or HPLC data, respectively, for the title compound prepared.

TABLE II

| Ex. | Ref. | | MS Data/ |
|---|---|---|---|
| No. | Ex. No. | Structure | HPLC |
| 164 | 118 | 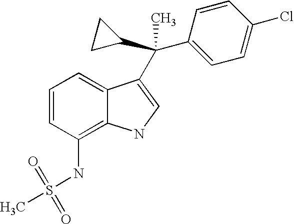 | 387 (M − 1) |
| 165 | 1 | 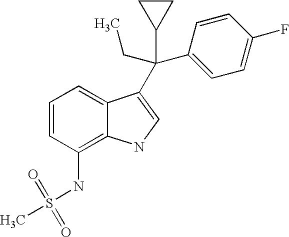 | 387 (M + 1) 385 (M − 1) |
| 166 | 1 | 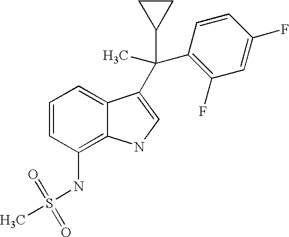 | 389 (M − 1) |
| 167 | 1 | 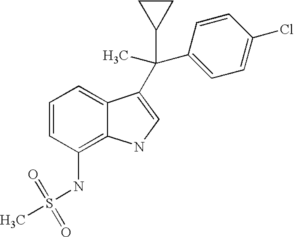 | 387 (M − 1) |
| 168 | 1 | 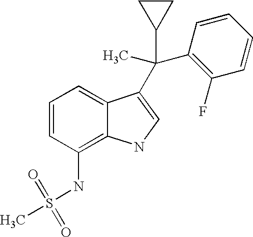 | 371 (M − 1) |
| 169 | 1 | 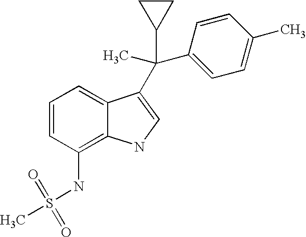 | 367 (M − 1) |
| 170 | 1 | 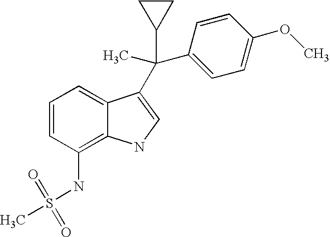 | 383 (M − 1) |
| 171 | 1 | 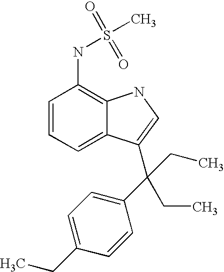 | 385 (M + 1) 383 (M − 1) |
| 172 | 118 | 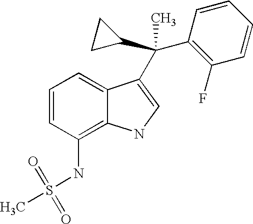 | 373 (M + 1) 371 (M − 1) |
| 173 | 118 | 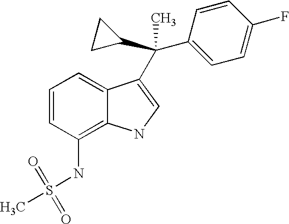 | 371 (M − 1) |
| 174 | 118 | 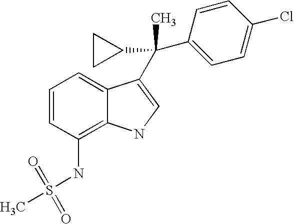 | 387 (M − 1) |
| 175 | 1 | 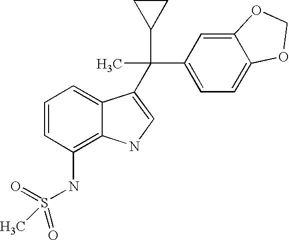 | 399 (M + 1) |
| 176 | 1 | 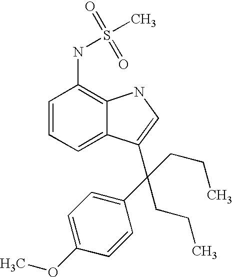 | 415 (M + 1) 413 (M − 1) |
| 177 | 1 | 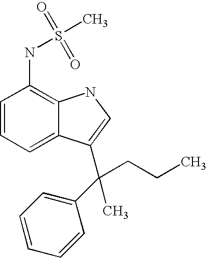 | 387 (M + 1) 385 (M − 1) |
| 178 | 1 | 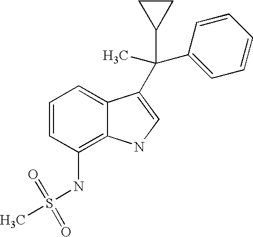 | 353 (M − 1) |
| 179 | 1 | 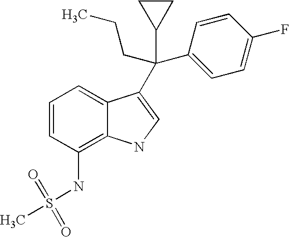 | 401 (M + 1), 399 (M − 1) |
| 180 | 118 | 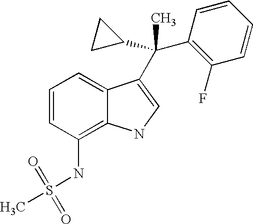 | 373 (M + 1) 371 (M − 1) |
| 181 | 3 | 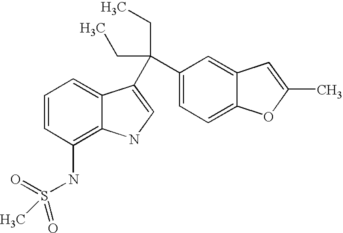 | 411 (M + 1) |
| 182 | 1 | 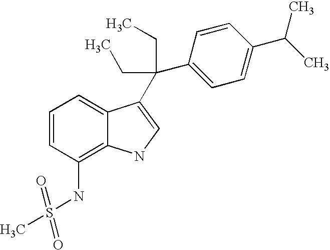 | 397 (M − 1) |
| 183 | 117 | 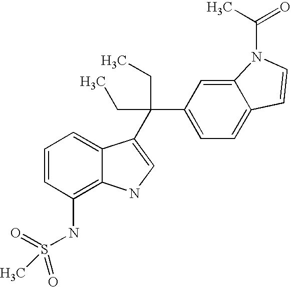 | 436 (M − 1) |
| 184 | 3 | 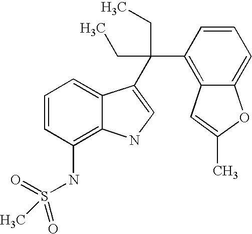 | 411 (M + 1) |
| 185 | 1 | 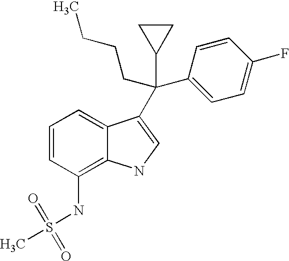 | 413 (M − 1) |
| 186 | 118 | 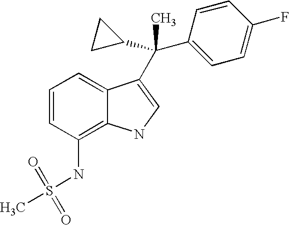 | 371 (M − 1) |
| 187 | 1 | 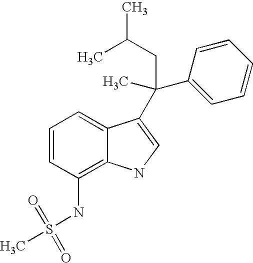 | 369 (M − 1) |
| 188 | 3 | 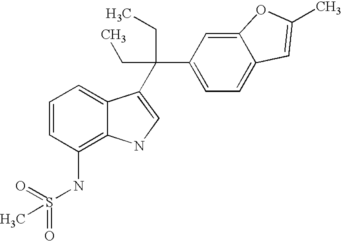 | 411 (M + 1), 409 (M − 1) |
| 189 | 32 | 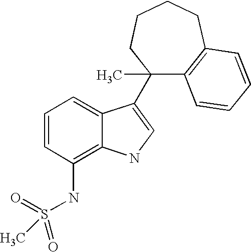 | 367 (M − 1) |
| 190 | 3 | 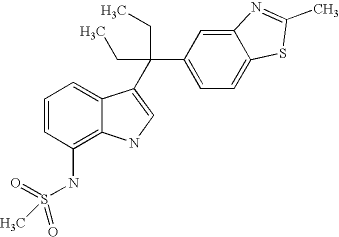 | 426 (M − 1) |
| 191 | 3 | 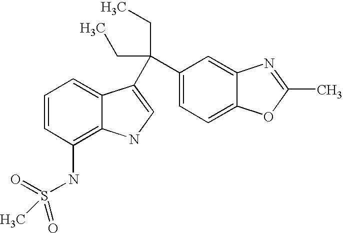 | 412 (M + 1) |
| 192 | 32 | 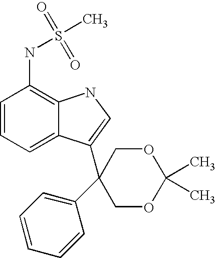 | 329 (M + 1) 327 (M − 1) |
| 193 | 3 | 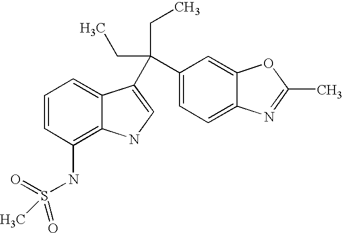 | 412 (M + 1) |
| 194 | 118 | 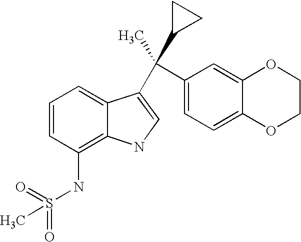 | 413 (M + 1) |
| 195 | 117 | 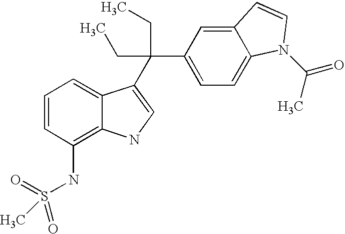 | 436 (M − 1) |
| 196 | 3 | 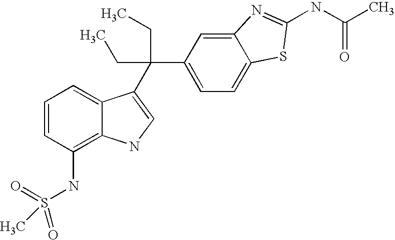 | 469 (M − 1) |
| 197 | 1 | 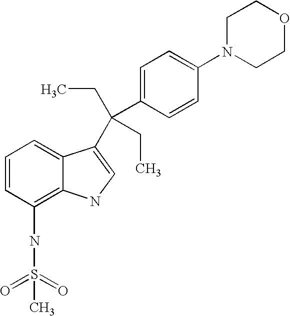 | 442 (M + 1) |
| 198 | 3 | 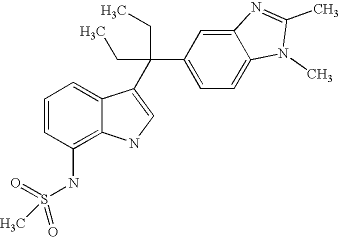 | 423 (M − 1) |
| 199 | 3 | 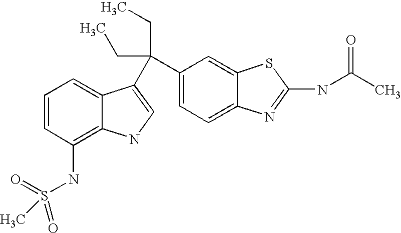 | 471 (M + 1) |
| 200 | 118 | 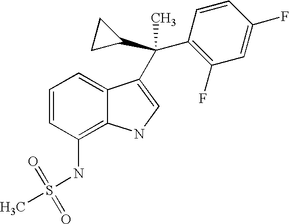 | 391 (M + 1) 389 (M − 1) |

1. A compound of the formula: 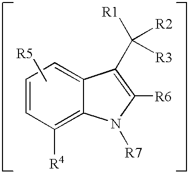
wherein,
R¹ represents (C₃-C₇)cycloalkyl, (C₂-C₆)alkynyl, aryl, heterocycle, fused heterocycle, or a substituted aryl, heterocycle, or fused heterocycle;
R² represents (C₁-C₆)alkyl, (C₃-C₇)cycloalkyl, aryl, substituted aryl, heterocycle, substituted heterocycle, (C₁-C₄)alkyl-(C₃-C₇)cycloalkyl, (C₁-C₄)alkyl-heterocycle, (C₁-C₄)alkyl-substituted heterocycle, (C₁-C₄)alkyl-aryl, (C₁-C₄)alkyl-substituted aryl, halo(C₁-C₆)alkyl, (C₁-C₄)alkyl-(C₁-C₆)alkoxy, (C₂-C₆)alkenyl, (C₂-C₆)alkynyl, cyano(C₁-C₆)alkyl, nitro(C₁-C₆)alkyl, amino(C₁-C₆)alkyl, NH(C₁-C₄)alkylamine, N,N—(C₁-C₄)dialkylamine(C₁-C₄)alkyl-NH(C₁-C₄)alkylamine, or (C₁-C₄)alkyl-N,N—(C₁-C₄)dialkylamine;
R³ represents (C₁-C₆)alkyl, halo(C₁-C₆)alkyl, (C₃-C₇)cycloalkyl, (C₁-C₄)alkyl-(C₃-C₇)cycloalkyl, (C₁-C₆)alkoxy, (C₁-C₄)alkyl-(C₁-C₆)alkoxy, aryl or R² and R³ together with the carbon atom to which they are attached form a (C₃-C₇)cycloalkyl or heterocycle group, with the proviso that where R¹ through R³ all represent aryl, then at least one of R⁴, R⁵ or R⁷ is other than hydrogen;
R⁴ represents halo, amino, nitro, (C₁-C₆)alkyl, hydroxy(C₁-C₆)alkyl, (C₁-C₆)alkoxy, NHSO₂R⁸, N(CH₃)SO₂R⁸, NHCOR¹², SO₂R⁹ or CHO;
R⁵ represents hydrogen, halo, hydroxyl, amino, nitro, cyano, difluoromethyl, triflouromethyl, difluoromethoxy, triflouromethoxy, (C₁-C₆)alkyl, or OR¹¹;
R⁶ represents hydrogen, halo, (C₁-C₆)alkyl, or (C₃-C₇)cycloalkyl;
R⁷ represents hydrogen, (C₁-C₆)alkyl, or (C₁-C₄)alkyl-COOH;
R⁸ and R⁹ each independently represent at each occurrence amino, (C₁-C₆)alkyl, (C₃-C₇)cycloalkyl, aryl, substituted aryl, (C₁-C₄)alkyl-aryl, (C₁-C₄)alkyl-substituted aryl, heterocycle, substituted heterocycle, (C₁-C₄)alkyl-heterocycle, (C₁-C₄)alkyl-substituted heterocycle, NH(C₁-C₄)alkylamine, or N,N—(C₁-C₄)dialkylamine;
R¹⁰ and R¹¹ each independently represent (C₃-C₇)cycloalkyl, aryl, substituted aryl, (C₁-C₄)alkyl-aryl, (C₁-C₄)alkyl-substituted aryl, heterocycle, substituted heterocycle, (C₁-C₄)alkyl-heterocycle, or (C₁-C₄)alkyl-substituted heterocycle; and
R¹² represents (C₁-C₆)alkyl,
provided that where R¹ through R³ all represent aryl, then at least one of R⁴, R⁵ or R⁷ is other than hydrogen;
or a pharmaceutically acceptable salt thereof.

2. (canceled)

3. (canceled)

4. The compound according to claim 1 wherein R⁷ represents hydrogen, (C1-C6)alkyl, CH2-COOH or CH2CH2-COOH.

5. The compound according to claim 1 wherein R⁶ represents hydrogen, halo, or (C₁-C₆)alkyl.

6. The compound according to claim 5 wherein R⁶ represents hydrogen, fluoro, or methyl.

7. The compound according to claim 1 wherein R⁵ represents hydrogen, halo, hydroxyl, amino, difluoromethyl, triflouromethyl, difluoromethoxy, triflouromethoxy, or (C₁-C₆)alkyl.

8. The compound according to claim 7 wherein R⁵ represents hydrogen, halo, or hydroxyl.

9. (canceled)

10. (canceled)

11. (canceled)

12. The compound according to claim 1 wherein R⁴ represents fluoro, amino, nitro, methyl, ethyl, hydroxymethyl, methoxy, ethoxy, NHCOR¹², NHSO₂R⁸, N(CH₃)SO₂R⁸, SO₂R⁹, or CHO.

13. The compound according to claim 12 wherein R¹² represents methyl.

14. The compound according to claim 12 wherein R⁸ represents individually at each occurrence methyl, ethyl, propyl, isopropyl, or phenyl.

15. The compound according to claim 12 wherein R⁹ represents methyl.

16. The compound according to claim 1 wherein R³ represents (C₁-C₆)alkyl, halo(C₁-C₆)alkyl, (C₃-C₇)cycloalkyl, or aryl.

17. The compound according to claim 16 wherein R³ represents (C₁-C₆)alkyl, halo(C₁-C₆)alkyl, or aryl.

18. The compound according to claim 17 wherein R³ represents methyl, ethyl, propyl, isopropyl, butyl, or phenyl.

19. The compound according to claim 1 wherein R² represents (C₁-C₆)alkyl, (C₃-C₇)cycloalkyl, aryl, substituted aryl, heterocycle, substituted heterocycle, (C₁-C₄)alkyl-(C₃-C₇)cycloalkyl, (C₁-C₄)alkyl-heterocycle, (C₁-C₄)alkyl-substituted heterocycle, (C₁-C₄)alkyl-aryl, (C₁-C₄)alkyl-substituted aryl, halo(C₁-C₆)alkyl, (C₁-C₄)alkyl-(C₁-C₆)alkoxy, nitro(C₁-C₆)alkyl, amino(C₁-C₆)alkyl, NH(C₁-C₄)alkylamine, N,N—(C₁-C₄)dialkylamine(C₁-C₄)alkyl-NH(C₁-C₄)alkylamine, or (C₁-C₄)alkyl-N,N—(C₁-C₄)dialkylamine.

20. The compound according to claim 19 wherein R² represents (C₁-C₆)alkyl, (C₃-C₇)cycloalkyl, aryl, substituted aryl, heterocycle, substituted heterocycle, halo(C₁-C₆)alkyl, (C₁-C₄)alkyl-(C₁-C₆)alkoxy, nitro(C₁-C₆)alkyl, amino(C₁-C₆)alkyl, NH(C₁-C₄)alkylamine, or N,N—(C₁-C₄)dialkylamine.

21. The compound according to claim 20 wherein R² represents (C₁-C₆)alkyl, (C₃-C₇)cycloalkyl, aryl, substituted aryl, heterocycle, substituted heterocycle, halo(C₁-C₆)alkyl, or (C₁-C₄)alkyl-(C₁-C₆)alkoxy.

22. The compound according to claim 21 wherein R² represents (C₁-C₆)alkyl, (C₃-C₇)cycloalkyl, aryl, substituted aryl, halo(C₁-C₆)alkyl, or (C₁-C₄)alkyl-(C₁-C₆)alkoxy.

23. The compound according to claim 22 wherein R² represents methyl, ethyl, propyl, isopropyl, butyl, cyclopropyl, phenyl, 4-methyl phenyl, 4-methoxy phenyl, 3-methoxy phenyl, 4-fluoro phenyl, 3-fluoro phenyl, 2-fluoro phenyl, 3,5-dimethyl phenyl, difluoromethyl, trifluoromethyl, or methoxy methyl.

24. The compound according to claim 1 wherein R¹ represents represents phenyl, (C₂-C₆)alkynyl, heterocycle, fused heterocycle, or a substituted phenyl, heterocycle, or fused heterocycle.

25. The compound according to clam 24 wherein R¹ represents phenyl, ethynyl, propynyl, thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzodioxine, benzodioxepine, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline.

26. The compound according to claim 25 wherein R¹ represents phenyl, ethynyl, or propynyl.

27. The compound according to claim 24 wherein R¹ represents thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzodioxine, benzodioxepine, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline.

28. The compound according to claim 27 wherein R¹ represents thiophenyl, furanyl, pyridinyl, benzofuranyl, 2,3 dihydro-benzofuranyl, furopyridinyl, benzothiophenyl, indolyl, benzodioxole, quinolinyl, benzoxazole, benzimidazole, benzothiophene, benzothiazole, indazole, benzoisoxazole, benzotriazole, benzodioxine, or benzodioxepine.

29. The compound according to claim 28 wherein R¹ represents thiophen-3-yl, thiophen-2-yl, furan-2-yl, furan-3-yl, pyridin-3-yl, pyridin-2-yl, benzofuran-2-yl, 2,3-dihydro-benzofuran-5-yl, benzo[b]thiophen-2-yl, benzo[b]thiophen-3-yl, quinolin-6-yl, furo[3,2-b]pyridin-2-yl, benzo[1,3]dioxol-5-yl, 1H-indol-3-yl, 1H-Benzoimidazol-5-yl, 1-Benzo[b]thiophen-5-yl, 1-Benzooxazol-6-yl, 1H-indazol-5-yl, 1-Benzo[b]thiophen-6-yl, 1-Benzothiazol-5-yl, 1-Benzooxazol-5-yl, 1-Benzothiazol-6-yl, 3H-Benzotriazol-5-yl, 1H-indol-5-yl, 1H-indol-6-yl, 2,3-Dihydro-benzo[1,4]dioxin-6-yl, or 3,4-dihydro-2H-benzo[b][1,4]dioxepin-7-yl.

30. The compound according to claim 24 wherein R¹ represents a substituted phenyl.

31. The compound according to claim 30 wherein R¹ represents phenyl substituted one or two times with a moiety selected from the group consisting of (C₁-C₆)alkyl, hydroxy, halo, (C₁-C₆)alkoxy, (C₁-C₄)alkylsulfonyl, (C₁-C₄)alkylsulfinyl, (C₁-C₄)alkylthio, aryl(C₁-C₆)alkoxy, trifluoromethyl, difluoromethyl, trifluoromethoxy, difluoromethoxy, phenyl, and halophenyl

32. The compound according to claim 31 wherein R¹ represents 2-methyl phenyl, 3-methyl-phenyl, 4-methyl phenyl, 4-ethyl phenyl, 2,4-dimethyl phenyl, 3,4-dimethyl phenyl, 3-hydroxy phenyl, 4-hydroxy phenyl, 3,5-dimethyl-4-hydroxy phenyl, 2-fluoro phenyl, 3-fluoro phenyl. 4-fluoro phenyl, 2,4-difluoro phenyl, 3,4-difluorophenyl, 4-methyl 2-fluoro phenyl, 4-chloro phenyl, 2-methoxy phenyl, 3-methoxy phenyl, 4-methoxy phenyl, 4-methanesulfonyl phenyl, 4-methanesulfinyl phenyl, 4-methanesulfanyl phenyl, 4-trifluoromethyl phenyl, 4-trifluoromethoxy phenyl, 2-biphenyl, 4-biphenyl, 3-(4-fluorophenyl)phenyl, 4-benzyloxy phenyl; 3-Chloro-4-methoxy-phenyl, 3-fluoro-4-methoxy-phenyl, 4-fluoro-3-methoxy-phenyl, or 4-Chloro-3-methoxy-phenyl.

33. The compound according to claim 24 wherein R¹ represents a substituted thiophenyl, furanyl, tetrahydrofuryl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyridinyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, benzoxazole, benzimidazole, benzofuran, dihydrobenzofuran, furopyridine, benzothiophene, benzothiazole, azaindole, indole, isoindole, azaisoindole, indazole, benzoisoxazole, benzoisothiazole, benzthiadiazole, benzoxadiazole, benztriazole, benzodioxole, benzodioxine, benzodioxepine, benzooxathiole, dihydroindole, dihydrobenzothiophene, azabenzofuran, azabenzothiophene, azabenzoxazole, azabenzthiazole, azabenzimidazole azaindazole, azabenzoisooxazole, azabenzoisothiazole, or quinoline.

34. The compound according to claim 33 wherein R¹ represents a substituted thiophenyl, furanyl, pyridinyl, benzofuranyl, 2,3 dihydro-benzofuranyl, furopyridinyl, benzothiophenyl, indolyl, benzodioxole, quinolinyl, benzoxazole, benzimidazole, benzothiophene, benzothiazole, indazole, benzoisoxazole, benzotriazole, benzodioxine, or benzodioxepine.

35. The compound according to claim 34 wherein R¹ represents thiophenyl, furanyl, pyridinyl, benzofuranyl, 2,3 dihydro-benzofuranyl, furopyridinyl, benzothiophenyl, indolyl, benzodioxole, quinolinyl, benzoxazole, benzimidazole, benzothiophene, benzothiazole, indazole, benzoisoxazole, benzotriazole, or benzodioxine substituted one or two times with a moiety selected from the group consisting of halo, (C₁-C₆)alkyl, (C₁-C₆)alkoxy, trifluoromethyl, acyl, and amino.

36. The compound according to claim 35 wherein R¹ represents 5-chloro-benzofuran-2-yl, 5-methoxy benzofuran-2-yl, 7-methoxy benzofuran-2-yl, 7-fluoro benzofuran-2-yl, 5-fluoro benzofuran-2-yl, 5-chloro-7-fluoro benzofuran-2-yl, 2,2-difluoro-benzo[1,3]dioxol-5-yl, 6-chloro benzo(b)thiophen-2-yl, 4-chloro benzo(b)thiophen-2-yl, 4-trifluoromethyl benzo(b)thiophen-2-yl, 5-trifluoromethyl benzo(b)thiophen-2-yl, 6-trifluoromethyl benzo(b)thiophen-2-yl, 7-trifluoromethyl benzo(b)thiophen-2-yl, 4-fluoro benzo(b)thiophen-2-yl, 5-fluoro benzo(b)thiophen-2-yl, 7-fluoro benzo(b)thiophen-2-yl, 3-methyl-4-fluoro benzo(b)thiophen-2-yl, 3-methyl-7-fluoro benzo(b)thiophen-2-yl, 2-methyl-benzooxazol-6-yl, 2-methyl-benzothiazol-5-yl, 2-Amino-benzothiazol-5-yl, 3-Amino-benzo[d]isoxazol-6-yl, 2-Amino-benzothiazol-6-yl, 2-methyl-benzooxazol-5-yl, 2-Chloro-benzothiazol-6-yl, 2-trifluoromethyl-3H-benzoimidazol-5-yl, 3-Amino-benzo[d]isoxazol-5-yl, 2-methyl-3H-benzoimidazol-5-yl, 2-methyl-benzofuran-5-yl, 1-Acetyl-1H-indol-5-yl, 1-Acetyl-1H-indol-6-yl, or, 2-methyl-benzofuran-4-yl, 2-Chloro-benzothiazol-5-yl, 1,2-Dimethyl-1H-benzoimidazol-5-yl, or 2-methyl-benzofuran-6-yl.

37. A pharmaceutical composition comprising the compound according to claim 1 in combination with a pharmaceutically acceptable carrier, diluent, or excipient.

38. A method of treating a disorder selected from the group consisting of Conn's Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Bartter's Syndrome, disorders associated with excess catecholamine levels, diastolic and systolic congestive heart failure (CHF), peripheral vascular disease, diabetic nephropathy, cirrhosis with edema and ascites, esophageal varicies, Addison's Disease, muscle weakness, increased melanin pigmentation of the skin, weight loss, hypotension, hypoglycemia, Cushing's Syndrome, obesity, hypertension, glucose intolerance, hyperglycemia, diabetes mellitus, osteoporosis, polyuria, polydipsia, inflammation, autoimmune disorders, tissue rejection associated with organ transplant, malignancies such as leukemias and lymphomas, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hyperglycemia, acute adrenal insufficiency, chronic primary adrenal insufficiency, secondary adrenal insufficiency, congenital adrenal hyperplasia, cerebral edema, thrombocytopenia, and Little's syndrome, systemic inflammation, inflammatory bowel disease, systemic lupus erythematosus, discoid lupus erythematosus, polyartitis nodosa, Wegener's granulomatosis, giant cell arthritis, rheumatoid arthritis, osteoarthritis, hay fever, allergic rhinitis, contact dermatitis, atopic dermatitis, exfoliative dermatitis, urticaria, angioneurotic edema, chronic obstructive pulmonary disease, asthma, tendonitis, bursitis, Crohn's disease, ulcerative colitis, autoimmune chronic active hepatitis, hepatitis, cirrhosis, inflammatory scalp alopecia, panniculitis, psoriasis, inflamed cysts, pyoderma gangrenosum, pemphigus vulgaris, bullous pemphigoid, dermatomyositis, eosinophilic fasciitis, relapsing polychondritis, inflammatory vasculitis, sarcoidosis, Sweet's disease, type 1 reactive leprosy, capillary hemangiomas, lichen planus, erythema nodosum, acne, hirsutism, toxic epidermal necrolysis, erythema, multiform, cutaneous T-cell lymphoma, psychoses, cognitive disorders, memory disturbances, mood disorders, depression, bipolar disorder, anxiety disorders, and personality disorders, comprising administering to a patient in need thereof a compound as claimed in claim 1, or a pharmaceutically acceptable salt thereof.

39. The method according to claim 38 wherein the disorder is selected from the group consisting of is diastolic or systolic congestive heart failure, inflammation, rheumatoid arthritis, an autoimmune disorder, asthma, or chronic obstructive pulmonary disease.

40. The method according to claim 39 wherein the disorder is diastolic or systolic congestive heart failure, inflammation, or rheumatoid arthritis.

41. (canceled)

42. (canceled)


SEQUENCE LISTING (2 sequences)

SEQ ID NO: 1 - DNA, 15 bp, Artificial
  Synthetic Construct
tgtacaggatgttct

SEQ ID NO: 2 - DNA, 15 bp, Artificial
  Synthetic Construct
ggttcttggagtact